SEQUENCE LISTING (4 sequences)

SEQ ID NO: 1 - DNA, 51 bp, Artificial Sequence
  Primer
tatatcatgagccatcatcatcatcatcacatggcggctgccgcacaatcc

SEQ ID NO: 2 - DNA, 54 bp, Artificial Sequence
  Primer
tatataccatgggccatcatcatcatcatcatgagagcgccggggccgacacgc

SEQ ID NO: 3 - DNA, 47 bp, Artificial Sequence
  Primer
cagccggatcctcgagcatatggctctaaatgttgacggtcttggcc

SEQ ID NO: 4 - DNA, 39 bp, Artificial Sequence
  Primer
gcgcgcgccatatgctacttggcatcaggcttcttgtct

(19) United States
(12) Patent Application Publication
(10) Pub. No.: US 2005/0153381 A1
(43) Pub. Date: Jul. 14, 2005

(54) Immunocapture of mitochondrial protein complexes

(76) Inventors: Michael F. Marusich, Eugene, OR (US); Roderick A. Capaldi, Eugene, OR (US); Devin Oglesbee, Rochester, MA (US)

Correspondence Address: KLARQUIST SPARKMAN, LLP, 121 SW SALMON STREET, SUITE 1600, PORTLAND, OR, 97204, US

(21) Appl. No.: 10/997,819

(22) Filed: Nov. 24, 2004

Related U.S. Application Data
(63) Continuation-in-part of application No. 10/917,254, filed on Aug. 11, 2004, now abandoned.
Application No. 10/917,254 is a continuation of application No. PCT/US03/04567, filed on Feb. 14, 2003.
Application No. 10/917,254 is a continuation of application No. PCT/US03/18114, filed on Jun. 6, 2003.
Application No. 10/917,254 is a continuation of application No. PCT/US03/27306, filed on Aug. 29, 2003.
(60) Provisional application No. 60/357,441, filed on Feb. 14, 2002.
(60) Provisional application No. 60/387,089, filed on Jun. 6, 2002.
(60) Provisional application No. 60/407,376, filed on Aug. 30, 2002.

(51) Int. Cl. G01N 33/53; G01N 33/537; G01N 33/543; C12N 5/16; C07K 16/40

(52) U.S. Cl. 435/7.92; 530/388.26; 435/338

(57) ABSTRACT

Provided herein is a library of monoclonal antibodies specific for native proteins and native protein complexes of the oxidative phosphorylation (OXPHOS) system (for example, Complex I, II, III, IV, or V, or any protein subunit of any of such complexes). Hybridomas expressing such antibodies and antibodies that competitively inhibit the binding of any such antibody (e.g., antibodies that bind the same or a sterically overlapping epitope) are also contemplated. Methods of using, and kits including, the disclosed antibodies are also provided. Antibodies, methods and kits described herein address a need in the art by providing immunological reagents and assays useful, at least, for detecting mitochondrial diseases associated with deficiencies or alterations in OXPHOS Complexes I, II, III, IV and/or V.

### REFERENCE TO RELATED APPLICATIONS

This is a continuation in part of U.S. patent application Ser. No. 10/917,254, filed Aug. 11, 2004, which is a continuation of International Application No. PCT/US03/04567, filed Feb. 14, 2003, which claims the benefit of U.S. Provisional Application No. 60/357,441, filed Feb. 14, 2002; International Application No. PCT/US03/18114, filed Jun. 2, 2003, which claims the benefit of U.S. Provisional Application No. 60/387,089, filed Jun. 6, 2002; and International Application No. PCT/US03/27306, filed Aug. 29, 2003, which claims the benefit of U.S. Provisional Application No. 60/407,376, filed Aug. 30, 2002. Each of the foregoing applications is incorporated herein in its entirety.

### FIELD OF THE DISCLOSURE

This disclosure relates to antibodies specific for native proteins and/or protein complexes of the electron transport chain (OXPHOS system) and to methods of use thereof. Immunoassays and compositions useful for performing immunoassays (such as, dipsticks and kits) are also disclosed; in particular, provided are immunoassays for detecting native OXPHOS protein subunits and/or OXPHOS enzyme complexes and alterations therein (such as, assembly defects or atypical post-translational modifications).

### BACKGROUND

Mitochondria: The Cellular Powerhouse and More.

Mammalian mitochondria are organelles that produce more than 90% of cellular ATP under aerobic conditions through a process called oxidative phosphorylation. Mitochondria are also involved in fatty acid metabolism, hormone production, ketone body production, apoptosis, and Ca²⁺ homeostasis. Mitochondria house, inter alia, the TCA cycle (also known as the Kreb cycle), enzymes involved in heme biosynthesis and the electron transport chain (OXPHOS system). Due to the large flux of redox reactions necessary to maintain oxidative phosphorylation, the organelle is the site of production of reactive oxygen species (ROS), which in controlled production have a signaling function, but in overproduction are toxic and are believed to be the cause of many human diseases including, for example, Parkinson's disease and other neurodegenerative conditions, diabetes, and the aging process itself.

The OXPHOS system is composed of five large multiprotein enzyme complexes, which collectively transform the reducing energy of NADH and FADH₂ to ATP. NADH ubiquinone oxidoreductase (Complex I) contains 45 different subunits, and succinate ubiquinone reductase (Complex II), ubiquinone-cytochrome c oxidoreductase (Complex III), cytochrome c oxidase (Complex IV) and the ATP synthase (Complex V) have 4, 11, 13 and 16 subunits respectively. Although composed of five individual enzyme complexes (each, an “OXPHOS complex” or “OXPHOS enzyme”) and containing a total of approximately 89 subunit proteins (each, an “OXPHOS protein”), the OXPHOS system has traditionally been considered to function as a single unit. Recent work supports this single-unit concept with evidence of structural associations between complexes, which are believed to enhance overall functional efficiency (Chen et al., J. Biol. Chem., 279: 31761-31768, 2004; Ko et al., J. Biol. Chem., 278: 12305-12309, 2003).

Four of the OXPHOS enzyme complexes (Complexes I, III, IV and V) have a dual genetic origin. That is, they are composed of both nuclear DNA-encoded proteins and mtDNA-encoded proteins. Thus, 7 subunits of Complex I, 1 subunit of Complex III, 3 subunits of Complex IV and 2 subunits of Complex V are encoded by mtDNA.

Mitochondria contain their own DNA (mtDNA) which is prokaryote-like. In mammals, this DNA is a 16 kb double-stranded circular DNA encoding 13 different polypeptides, all involved in oxidative phosphorylation, along with 2 rRNAs and 22 tRNAs. mtDNA lacks protective histones and has minimal repair mechanisms, which leads to a relatively high mutation rate that is further enhanced by the proximity of the DNA to the OXPHOS system, the site of production of ROS. Accumulation of mutations and deletions in mtDNA occurs throughout life in humans and becomes physiologically relevant where they affect sufficient number of copies of the mtDNA to alter oxidative phosphorylation.

Unlike the nuclear genome, which is present in two copies, mtDNA is present in thousands of copies in mammalian cells, all of which are used in translation of gene products made within the organelle on bacterial-like ribosomes. Thus, inheritance and penetrance of mtDNA mutations is not Mendelian, but rather depends on the relative amount (%) of wild-type and mutant mtDNA molecules per cell. The normal state is 100% wild-type mtDNA or wild-type homoplasmy. A mutation in mtDNA can also be homoplasmic (present in all mtDNA molecules of a cell) in which case it is likely to have a functional and possibly pathogenic effect. The presence of a mixture of mutant and wild-type mtDNA molecules in an individual cell is referred to as heteroplasmy. Because normal cells have an excess capacity of mtDNA and mtDNA-encoded proteins, heteroplasmic mutant mtDNA are believed to cause an altered functional (or pathogenic) phenotype if the mutant mtDNAs are present at levels exceeding some threshold value, usually 70-90%. An additional consequence of heteroplasmy is the development of altered functions of mitochondria within a single cell, between cells and between tissues (Wallace, Science, 283: 1482-1488, 1999; Chinnery and Turnbull, Mol. Med. Today, 6: 425-432, 2000).

Early Onset Genetic Diseases of Mitochondria.

A large number of genetic disorders involving mitochondrial components have been described. Those in which the defective gene is involved in oxidative phosphorylation are loosely categorized as mitochondrial disorders (Schon, Trends Biochem. Sci., 25: 555-560, 2000). The dual genetic origin (nuclear DNA and mtDNA), non-Mendelian inheritance and penetrance, cell and tissue mosaicism and different energy thresholds of different tissues make mitochondrial defects difficult to diagnose and treat. Indeed, mitochondrial diseases comprise a heterogeneous group of diseases that can give rise to “any symptom in any organ or tissue with any mode of inheritance” (Munnich et al., Int. Pediatr., 7: 28-33, 1992). At the molecular level they have diverse causes and can arise by mutations in any of the 90 structural genes for the OXPHOS complexes. Mitochondrial disorders can also arise from mutations in assembly factors required for prosthetic group insertion and/or stabilization of partially assembled complexes (Capkova et al., Cas. Lek. Cesk., 141: 636-41, 2002). Finally, they are hard to distinguish from pyruvate dehydrogenase (PDH) defects, which have a similar phenotype (Jordens et al., Ann. Neurol., 52: 95-99, 2002).

OXPHOS protein analysis provides some indication of whether the defect is an assembly problem affecting one or more complexes and/or is a catalytic defect. This information helps narrow a search for a corresponding gene mutation.

Late-Onset Mitochondrial Disease.

In addition to genetic disorders, mitochondrial dysfunction can be late onset with a consequent effect on cell function and viability that leads to a wide range of diseases depending on the tissues involved and the degree of the dysfunction. Several disorders are believed to have mitochondrial involvement, including Parkinson's disease (Greenamyre et al., IUBMB Life., 52: 135-141, 2001; Orth and Schapira, Neurochem. Int., 40: 533-541, 2002; Sherer et al., Neuroscientist, 8: 192-197, 2002; Sherer et al., J. Neurosci., 22: 7006-7015, 2002), Alzheimer's disease (Swerdlow and Kish, Int. Rev. Neurobiol., 53: 341-385, 2002; Mattson, Int. Rev. Neurobiol., 53: 387-409, 2002), Huntington's disease (Arenas et al., Ann. Neurol., 43: 397-400, 1998; Schapira, Biochim. Biophys. Acta, 1410: 99-102, 1999), schizophrenia (Ben-Shachar, J. Neurochem., 83: 1241-1251, 2002), early-onset and late-onset diabetes (Maassen, Am. J. Med. Genet., 115: 66-70, 2002; Kelley et al., Diabetes, 51: 2944-2950, 2002), cardiovascular disease (Marin-Garcia and Goldenthal, J. Card. Fail., 8: 347-361, 2002), and the aging process (Genova et al., FEBS Lett., 505: 364-368, 2001).

Current theories suggest that late onset mitochondrial diseases occur as a result of accumulated mitochondrial damage. Through a combination of inherited genetic defects and the accumulation of spontaneous, unrepaired mutations to mtDNA (some caused by the ROS produced at low levels as a by-product of normal OXPHOS system function) and environmental damage to both mitochondrial proteins and mtDNA, mitochondrial function is progressively degraded. Altered functioning of the OXPHOS system is particularly damaging in this context as it can result in the formation of by-product ROS at higher than normal levels, which in turn causes more OXPHOS system damage, inducing more ROS, etc., in a damaging feed-forward loop.

Ischemia and Reperfusion

Transient ischemia (anoxia) results in the local production of extremely high levels of ROS which can cause long term damage to mitochondria. Ironically, it is the sudden re-supply of oxygen to the ischemic tissue during reperfusion that is believed to be the proximate cause of elevated ROS production. In the initial phase of transient ischemia, oxygen is scarce but tissue demands for ATP remain high, resulting in continued functioning of the electron transport chain except for the terminal reduction of oxygen to water by Complex IV. Therefore, reduced electron acceptors “upstream” of Complex IV accumulate to abnormally high levels. Upon resupply of oxygen, these excess reduced carriers react directly (inappropriately) with oxygen to generate highly toxic partially reduced oxygen species (Pitkanen and Robinson, J. Clin. Invest., 98: 345-351, 1996; Genova et al., FEBS Lett., 505: 364-368, 2001), which are capable of protein, lipid and DNA modifying reactions. The resulting oxidative damage would be expected to occur mainly inside the mitochondrion, because such radicals are so reactive that they are short lived and cannot diffuse far before finding a target for reaction. Accordingly, OXPHOS proteins and mtDNA are likely to be the cellular molecules most affected by such oxidative stress. The resulting defects in mtDNA and OXPHOS proteins may result in continued increased production of ROS, which may also lead to a damaging positive feedback loop.

Mitochondrial Toxicity as a Side Effect of Drug Therapy

Therapeutic drugs can have unintended side-effects on mitochondrial function. These include the popular statins, which act not only as desired to lower levels of serum cholesterol (synthesized in the mitochondrion), but also to lower levels of mitochondrial co-Q₁₀, an essential co-factor in the flow of electrons through the OXPHOS system (De Pinieux et al., Br. J. Clin. Pharmacol., 42: 333-337, 1996). Statins may also react with Complex IV directly (Arenas et al., Neurology, 14: 124-126, 2003). These drug side-effects can damage various tissues, resulting most notably in severe toxic myopathy (Hamilton-Craig, Med. J. Aust., 175: 486-489, 2001). This has led to the withdrawal of at least one particularly harmful statin, cerivasatatin, from the market (Thompson et al., J. Am. Med. Assoc., 289: 1681-1690, 2003). Less serious side-effects such as muscle pain and weakness are relatively common with all statins, affecting up to 5% of patients (Thompson et al., J. Am. Med. Assoc., 289: 1681-1690, 2003).

Certain antibiotics can have disastrous effects on the auditory system of individuals with particular mtDNA genotypes, resulting in permanent deafness (Prezant et al., Nature Genet., 4: 289-294, 1993; Pandya et al., J. Med. Genet., 34: 169-172, 1997). This is an example of a deleterious interaction between the environment and an individual's genetic background. The commonly used non-steroidal anti-inflammatory drugs (NSAIDs) can cause uncoupling of mitochondrial electron transport, resulting in lower mitochondrial energy efficiency and induce gastrointestinal ulcer formation (Fosslien, Ann. Clin. Lab. Sci., 31: 25-67, 2001).

It is likely that the mitotoxic effects of many other prescription drugs have gone unidentified (although not necessarily unnoticed by patients) due to the variable presentation and penetration of mitochondrial defects in a manner analogous to the variable penetration and difficult diagnosis of the inherited mitochondrial disorders described above. Although minor drug-induced effects on mitochondrial efficiency might be non-pathogenic in otherwise healthy individuals, they could tip the balance if they occur in the context of an independent sub-clinical mitochondrial disease, i.e., the effects could combine to push a particular tissue below the mitochondrial functional threshold discussed above. New assays and diagnostic tests are needed that will allow more sensitive, practical ways to characterize, understand and manage these side effects and to help provide personalized molecular medicine.

Nucleotide Reverse Transcriptase Inhibitor (NRTI)-Induced Mitochondrial Toxicity.

Nucleotide reverse transcriptase inhibitor (NRTI)-induced mitochondrial toxicity is in a class of its own, owing to the number of individuals affected and the severity of the toxicity. NTRIs (such as zidovudine (AZT), stavudine (D4T), zalcitabine (DDC), didanosine (DDI), lamivudine (3TC) and abacavir (ABV)) are a mainstay in the treatment of HIV infection. NRTIs are now used world-wide by millions of individuals in combination with protease inhibitors and/or non-nucleoside inhibitors as part of so-called “highly active anti-retroviral therapy” (HAART). NRTIs competitively inhibit HIV reverse transcriptase by incorporating into the newly synthesized DNA strand. Because NRTIs lack the 3′ hydroxyl group needed for chain elongation the growing viral DNA chain is terminated.

The improved survival of patients infected with HIV by the use of anti-retroviral therapies is a major accomplishment of medicine in the 1990s. However, with extensive use of the NRTIs has come greater awareness of long-term toxicities of these therapies. Thus, lactic acidosis (Brinkman, Clin. Infect. Dis., 31: 167-169, 2000; Moyle, Clin. Ther., 22: 911-936, 2000; Carr and Cooper, Lancet, 356: 1423-1430, 2000; John et al., J. AIDS, 15: 717-723, 2001), myopathy, cardiomyopathy, hepatic steatosis, neuropathy, and lipodystrophy (Gan et al., Diabetes Obes. Metab., 3: 67-71, 2001; Moyle, Clin. Ther., 22: 911-936, 2000) are all associated in significant numbers of patients with NRTI therapy. Importantly, the clinical and morphological manifestations of these pathologies are very similar to a set of disorders seen with genetic defects in the enzymes of oxidative phosphorylation, a key function of mitochondria in the cell. It is now believed that altered mitochondrial functioning as a result of altered mitochondrial DNA replication is at the heart of the toxicity associated with NRTI therapy.

Liver and muscle biopsies from an HIV patient, who had been taking anti-retroviral drugs for three years with resulting lactic acidosis, muscular and hepatic disturbances, were shown to have mitochondrial structural abnormalities and mtDNA depletion (Chariot et al., J. Hepatol., 30: 156-160, 1999). Other studies involving electron microscopy of skeletal muscle from patients receiving HAART have identified ragged red fibers and lipid droplet inclusions in mitochondria (Radovanic et al., Ultrastruct. Pathol., 23: 19-24, 1999; Gerschenson et al., AIDS Res. Hum. Retroviruses, 16: 635-644, 2000; Gomez-Zaera et al., J. AIDS, 15: 1643-1651, 2002; van Huyen et al., Am. J. Clin. Pathol., 119: 546-555, 2003). Studies to date have shown that changes in mitochondria structure and/or function as a result of HAART exist in some tissues or cells, but not in others. Most of the recent work is focused on peripheral blood cells and subcutaneous adipose tissues, and examines mtDNA depletion as a consequence of the treatments.

NRTIs compete with nucleotides for incorporation into DNA with much greater selectivity for the HIV reverse transcriptase than the cell nuclear replicative DNA polymerases. However, the mtDNA polymerase γ, the sole polymerase responsible for replicating the mitochondrial genome, is more like a bacterial or viral polymerase than the eukaryotic nuclear polymerase. As a result, mtDNA replication in the presence of NRTIs shows relatively high incorporation of these nucleotide analogues and low excision rates, leading to mtDNA depletion and its pathological consequences. Isolated human mtDNA polymerase γ is inhibited most dramatically by DDC and with the following hierarchy: DDC≧DDI≧D4T≧3TC≧AZT≧ABV. In cells treated with AZT, 3TC, DDC, DDI and D4T there is clear evidence of mtDNA depletion (Lewis et al., J. Clin. Invest., 89: 1354-1360, 1992; Bridges et al., Biochem. Pharmacol., 51: 731-736, 1996; Gerschenson et al., AIDS Res. Hum. Retroviruses, 16: 635-644, 2000; Kakuda, Clin. Ther., 22: 685-708, 2000).

Animal studies also indicate that NRTIs induce mtDNA depletion. Thus, rats treated with AZT show lowered levels of mtDNA in skeletal muscle (Lewis et al., J. Clin. Invest., 89: 1354-1360, 1992), while in monkeys, AZT has been shown to reduce mtDNA copy number and cause abnormal OXPHOS enzyme activity (Gerschenson and Poirier, Ann. N.Y. Acad. Sci., 918: 269-281, 2000; Gerschenson et al., AIDS Res. Hum. Retroviruses, 16: 635-644, 2000). Finally, there are numerous studies reporting reduced mtDNA copy number in patients with HIV infection as a result of NRTI therapy (see, for example, Barile et al., Eur. J. Biochem., 249: 777-785, 1997; Barile et al., Biochem. Pharmacol., 53: 913-920, 1997; Helbert et al., Lancet, 1: 689-690, 1988; Casademont et al., Brain, 119: 1357-1364, 1996; Miura et al., J. Med. Virol., 70: 497-505, 2003; Martin et al., Am. J. Hum. Genet., 72: 549-560, 2003; Gahan et al., J. Clin. Virol., 22: 241-247, 2001; Shikuma et al., J. AIDS, 15: 1801-1809, 2001; Cherry et al., J. AIDS, 30: 271-277, 2002; Gourlain et al., HIV Med., 4: 287-292, 2003; Nolan et al., J. AIDS, 17: 1329-1338, 2003; Pace et al., Antivir. Ther., 8: 323-331, 2003; Hellerstein, Clin. Infect. Dis., 37(Suppl. 2): S52-61, 2003; and reviewed in Cossarizza, Curr. Opin. Infect. Dis., 16: 5-10, 2003, and Cossarizza et al., Antivir. Ther., 8: 315-321, 2003).

Study and Diagnosis of Mitochondrial Disorders

Studies on mitochondrial disorders and molecular diagnosis of patients with such disorders typically involve analysis of the expression and/or function of oxidative enzymes (such as Complexes I, II, III, IV, and V) using isolated mitochondria (Janssen et al., Ann. Clin. Biochem., 40: 3-8, 2003; Bauer et al., Clin. Chem. Lab. Med., 37: 855-876, 1999; Chretien et al., Biochem. Biophys. Res. Commun., 301: 222-224, 2003). Using traditional methods, this analysis is tedious and difficult and can only be done in a limited number of specialized centers. Some of the activity-based tests require the use of freshly prepared mitochondria, which further limits analysis. Because different specialized centers have different expertise, several samples are taken from the same patient and sent to different sites to obtain a full activity analysis and protein profile. As a result, diagnosis of mitochondrial disorders requires significant amounts of biopsy material and often takes months to complete. In some cases, cell culture material, most often fibroblasts, can be used in diagnosis but mtDNA defects with their tissue-variant heteroplasmy do not always present in cell culture material (Singhal et al., J. Postgrad. Med., 46: 224-230, 2000; Janssen et al., Ann. Clin. Biochem., 40: 3-8, 2003).

Hence, there is a need for rapid, preferably high-throughput, methods for qualitative and/or quantitative analysis of Complexes I, II, III, IV and V, including analysis of expression levels, activity levels, physiological and pathological modifications, and/or complex structure and assembly. In some cases, it would be useful if such methods could be performed using very few cells and/or small amounts of biopsy material.

### SUMMARY OF THE DISCLOSURE

Provided herein is a collection of monoclonal antibodies useful, alone or in various combinations, in immune capturing proteins and protein complexes of the OXPHOS system. The OXPHOS system is a single, well-defined metabolic unit consisting of functionally and structurally interrelated proteins and enzyme complexes. The disclosed anti-OXPHOS capture monoclonal antibodies operationally define those surface domains (epitopes) of the OXPHOS system that serve as useful immunocapture binding sites.

Immunocapture is the use of antibodies to specifically bind an OXPHOS protein or protein complex in its native state. Although immunocapture can be used to purify one or more proteins of interest and therefore facilitate many types of subsequent analysis, purification is not necessary for some types of analysis and/or quantification. For example, these antibodies enable the multiplex analysis of protein complexes involved in the OXPHOS system, including protein quantitation, enzyme activity analysis (including quantification), analysis of protein complex assembly status, and focused proteomic analysis of mitochondrial proteins, such as analysis of structural changes due to genetic defects and posttranslational modifications such as those involved in both physiologic and pathogenic regulation of protein function and complex assembly.

The disclosed antibodies, methods and kits overcome problems in the art by providing immunological reagents and assays useful for detecting mitochondrial diseases associated with deficiencies or alterations in OXPHOS enzyme complexes I, II, III, IV and/or V. These immunological reagents and methods, at least, (i) enable personalized mitochondrial medicine, such as measurement of an individual's mitotoxic burden (i.e., his/her current accumulated mitochondrial damage), assessment of an individual's sensitivity to mitotoxic agents (including therapeutic drugs and environmental toxins), and the ability to monitor adverse mitochondrial (mitotoxic) effects of therapeutic drugs taken to manage other non-mitochondrial diseases; (ii) provide tools and tests to monitor the progression of mitochondrial diseases, and to guide therapy for these disorders; (iii) enable high-throughput screening of mitotoxic environmental substances and side-effects of therapeutic drugs; and (iv) enable high-throughput screens to identify new therapeutic drugs that can protect mitochondria from toxic agents and, thus, prevent or treat mitochondrial disorders.

The foregoing and other features and advantages will become more apparent from the following detailed description of several embodiments, which proceeds with reference to the accompanying figures.

### BRIEF DESCRIPTION OF THE FIGURES

FIG. 1 is a bar graph showing isolated Complex I deficiency in patient fibroblast cell lines as revealed by Western blots using monoclonal antibodies specific for Complex I 39-kDa subunit (CI-39), Complex II 70-kDa subunit (CII-70), Complex III core 2 subunit (CIII-core 2), Complex IV subunit II (CIV-II), Complex IV subunit IV (CIV-IV), and Complex V subunit α (CV-alpha). Western blot signals were quantified, and the levels of the indicated subunits in normal fibroblasts (MRC), Rho0 fibroblasts, and 11 patient cell lines (numbered 1-11) were plotted. The signal for each antibody was normalized to the value obtained for that antibody in control skin (C) fibroblasts. Porin was used as the loading control for all gels.

FIG. 2 is a bar graph showing variations in the assembly of Complex I as identified by Western blot using monoclonal antibodies specific for Complex I 39-kDa subunit (CI-39), 30-kDa subunit (CI-30), 20-kDa subunit (CI-20), 18-kDa subunit (CI-18), 15-kDa subunit (CI-15) and 8-kDa subunit (CI-8). Western blot signals were quantified, and the levels of the indicated Complex I subunits in normal fibroblasts (MRC5) and Rho0 fibroblasts and 11 patient cell lines (numbered 1-11) were plotted. The signal for each antibody was normalized to the value obtained for that antibody in control skin (C) fibroblasts. Porin was used as the loading control for all gels.

FIG. 3 shows a series of graphs demonstrating the relationship between the expression level of various Complex I subunits and Complex I enzymatic activity. Each point represents a single patient having a mitochondrial disorder. Panels A-F show the results for the Complex I 8-kDa subunit (A), 15-kDa subunit (B), 18-kDa subunit (C), 20-kDa subunit (D), 30-kDa subunit (E), and 39-kDa subunit (F). In each panel, the dashed line represents what would be expected if there were a perfect correlation between level of subunit expression and Complex I activity.

FIG. 4 shows a series of graphs demonstrating where the Va subunit of Complex IV (Complex IV-Va; panel A), the 39-kDa subunit of Complex I (Complex I-39 kD; panel B), and the 20-kDa subunit of Complex I (Complex I-20 kD; panel C) from three patients with mitochondrial dysfunction elute in a sucrose gradient as compared to control cells. Patient 1 (open diamond), patient 7 (open square), patient 11 (x), and control MRC5 fibroblasts (open circle). In each case the darkest intensity band for each antibody and each sample was set to 100%.

FIG. 5 is a graph showing that human F₁/F₀ ATPase immunocaptured by the anti-F₁/F₀ ATPase mAb 12F4AD8AF8 is capable of oligomycin-sensitive ATP hydrolysis. All measurements were done in triplicate and the data points show the specific signal (ATPase activity) captured by the anti-F₁/F₀ ATPase mAb minus the background signal captured by a null antibody. Activities were measured in the absence (closed circles) and presence of 1.5 μg/ml oligomycin (open squares).

FIG. 6 shows graphs of the ATP hydrolysis activity of immunocaptured human heart mitochondrial F₁/F₀ ATPase at pH 6.5 (FIG. 6A) and pH 7.5 (FIG. 6B) in the presence and absence of IF₁. IF₁, which is a native inhibitor of the F₁/F₀ ATPase, remains bound to the F₁/F₀ ATPase at pH 6.5, but can be stripped (strip IF₁) from the enzyme by treatment at pH 8.0. Bars show the average with standard deviations of samples tested in triplicate.

FIG. 7 shows graphs of the ATPase activities of detergent-solubilized cells (panel A) and protein captured by anti-F₁/F₀ ATPase mAb (panel B), each measured in the absence (open circle) and presence of 1.5 μg/ml oligomycin (closed square). Each data point represents a duplicate measurement. This result demonstrates that immunocapture using anti-F₁/F₀ ATPase mAbs can be used to purify the oligomycin-sensitive, human, mitochondrial F₁/F₀ ATPase.

FIG. 8 is a graph showing that immunocapture of Complex V can reveal defects in fibroblast mitochondrial F₁/F₀ ATPase (F₁/F₀ ATPase). F₁/F₀ ATPase was immunocaptured from human heart muscle (HHM), normal fibroblasts (MRC5), Rho0 fibroblasts (ρ⁰) and fibroblasts from a patient with Luft's disease (GM28) and assayed in the presence and absence of 1.5 μg/ml oligomycin. Bars show the average (±S.D.) of triplicate samples tested at 20 μg mitochondrial protein per well.

FIG. 9 shows a graph of the ATPase activity of the F1/F0 ATPase immunocaptured at pH 6.5 from mitochondria protein of control (MRC5) and GM28 fibroblast cells. The immunocaptured F₁/F₀ ATPase complex was subsequently “stripped” of endogenous IF₁ at pH 8.0 (strip IF₁), and then reconstituted with recombinant IF₁ (add IF₁). GM28 and MRC5 F₁/F₀ ATPase activities have similar profiles, which indicated that GM28 F₁/F₀ ATPase was associated under native conditions with functional IF₁. Bars show the average (±S.D.) of triplicate samples tested at 20 μg mitochondrial protein per well.

FIG. 10 is graph of total maleimide fluorophore-labeled mitochondrial protein versus fluorescence signal measured following immunocapture by a mAb specific for Complex V. Labeling of the mitochondrial protein was stopped by saturating the reaction with BSA, and there was no separation of unreacted dye.

FIG. 11 is a bar graph showing that measurement of the 2-dye immunocapture ratios (Cy3:Cy5) are highly reproducible from experiment to experiment. Single samples of mitochondria were split into two equal aliquots, which were then labeled with either maleimide Cy3 or maleimide Cy5. The labeled mitochondria were then re-combined in equal portions and the mixed samples captured simultaneously in microwells coated with each of the listed capture mAbs. Background was subtracted from each Cy3 and Cy5 reading and raw ratios of Cy3:Cy5 fluorescence calculated for each complex. The raw ratios were normalized by dividing all raw ratios by the raw ratio calculated for Complex V (which sets the normalized Complex V ratio to 1). The results of five trials are shown (two labelings, measured 2 and 3 times respectively).

FIG. 12 is a schematic representation of a two antibody sandwich-type immunodetection assay. One target-antigen-specific antibody (Anti-Complex I) is immobilized in a discrete area on a solid support. Preferably, a negative control antibody (Normal IgG Negative Control) and a positive control antibody (goat-anti-mouse (GAM) Positive Control) are also immobilized in separate zones of the solid support. The antigen (open square) is mixed with a second, detectable target-antigen-specific antibody (black oval), and this mixture traverses the solid support and interacts with the antibodies immobilized thereon.

FIG. 13 shows a series of Complex I-specific dipsticks, which were contacted with a series of samples (67 μl total volume) containing 50 μl human heart mitochondria extracts having differing amounts of mitochondrial protein (0-62.5 μg mitochondrial protein) and 17 μl colloidal-gold-conjugated mAb. The samples were incubated at room temperature for 10 minutes prior to addition of the dipstick. The inset shows a graph of signal density versus μg of mitochondrial protein for the anti-Complex I zone marked by an asterisk. Null mAb was non-specific IgG. “GAM” refers to goat-anti-mouse antibody, which will bind any mouse antibody, including the gold-conjugated anti-Complex I antibody mixed with each sample.

FIG. 14 shows two series of Complex I-specific dipsticks, which were contacted with a series of samples (67 μl total volume) containing 50 μl of normal (MRC5) or mtDNA-defective (Rho0) mitochondria extracts having the indicated amounts of mitochondrial protein and 17 μl colloidal-gold-conjugated mAb. The samples were incubated at room temperature for 10 minutes prior to addition of the dipstick. The inset shows a graph of signal density versus μg of mitochondrial protein for the anti-Complex I zone marked by an asterisk. Null mAb was non-specific IgG. “GAM” refers to goat-anti-mouse antibody, which will bind any mouse antibody, including the gold-conjugated anti-Complex I antibody mixed with each sample.

FIG. 15 shows a series of Complex I-specific dipsticks, which were contacted with a series of samples (67 μl total volume) containing 50 μl of an cellular extract prepared from the indicate number of peripheral blood mononuclear cells and 17 μl colloidal-gold-conjugated mAb. The samples were incubated at room temperature for 10 minutes prior to addition of the dipstick. Null mAb was non-specific IgG. “GAM” refers to goat-anti-mouse antibody, which will bind any mouse antibody, including the gold-conjugated anti-Complex I antibody mixed with each sample.

FIG. 16 is a schematic representation of one embodiment of a quantitative, instrument-free lateral flow device (such as a dipstick).

FIG. 17 illustrates the immunocapture of the five OXPHOS complexes from bovine heart mitochondria with monoclonal capture antibodies. Broad range markers from BioRad (SDS-PAGE Molecular Weight Standards) were applied in the left and right lanes. Filled circles indicate the presence of the heavy chain of IgG co-eluted from the protein G beads.

FIG. 18 demonstrates the purity of immunocaptured Complexes I-V. Three cm wide lanes on 10-22% gels were loaded with samples containing complexes I (A), II (B), III (C), IV (D) and (E). As Molecular weight marker, SeeBlue Plus2™ Pre-Stained Standard from Invitrogen was used. Filled circles indicate the presence of contaminating antibody heavy chain, released from the protein G beads during the elution procedure of Complex V.

FIG. 19 demonstrates phosphoprotein detection with Pro-Q Diamond phosphoprotein gel stain. Pro-Q Diamond stained lanes are indicated with D and SYPRO Ruby with R. As reference the ovalbumin containing broad range marker from BioRad was used (A). Complex I is shown in panel (B), II in (C), III in (D), IV in (E) and ATP synthase in (F). Known phosphoproteins are named. Arrows indicate the positions of novel phosphoproteins. The filled circle indicates the position of the heavy chain of an immunocapture antibody.

FIG. 20 demonstrates the isolation of Complex IV from human and other species using a monoclonal capture antibody. Immunocapture of Complex IV from only 1 mg of human or bovine heart mitochondria (HHM and BHM respectively). Bovine subunits were identified by MALDI-TOF MS and ESI-MS/MS after trypsin and/or chymotrypsin proteolysis. High MS sensitivity allowed the detection of non-Complex IV proteins in gel segments of little or no protein staining.

FIG. 21 shows a spectrum with high quality MALDI-TOF MS data from a single SDS-PAGE gel band. The MALDI mass spectrum displays molecular ions of peptides obtained from in-gel tryptic digestion of a selected protein gel slice. Thirteen peptide mass fingerprints were identified as peptides resulting from tryptic digestion of the following protein, “COX subunit IV” (IV-IV) isolated from bovine heart. Observed masses are labeled and annotated with starting and ending amino acids. Overall, a protein sequence coverage of 41% was observed for this 19.6 kDa protein/theoretical pI of 9.3, where T is a trypsin autolysis product.

FIG. 22 demonstrates LC-MS/MS identification of tissue specific Complex IV peptides. ESI-MS/MS spectra of homologous peptides from Complex IV subunits VIIa-H and VIIa-L. Panel A shows tryptic peptide GGATDNILYR (residues G-45 to R-54) obtained from protein isoform IV-VIIa-H. The molecular ion, [M+2H]2+ at m/z 540.262+ (M=1078.51) was selected for collision induced dissociation (CID). Panel B shows tryptic peptide GGIADALLYR (residues G-47 to R-56) obtained from protein isoform IV-VIIa-L. The molecular ion, [M+2H]2+ at m/z 524.782+ (M=1047.55) was selected for collision induced dissociation (CED). For both tandem mass spectra nearly complete series of y-fragment ions and several b-fragment ions were observed.

FIG. 23 demonstrates Fe²⁺/ascorbate/O₂-generated carbonyl modification of Complex IV. Panel A shows the effect of metal catalyzed oxidation over time upon Complex IV activity in the mitochondrial membranes. The same effect upon activity is measured after immunocapturing the enzyme. In panel B the same immunocaptured Complex IV sample is resolved by a SDS-PAGE showing that all Complex IV bands appear present suggesting assembly is unaffected. Furthermore Complex IV subunit IV can be derivatized by DNPH indicating the creation of oxidation dependant carbonyls. Additionally core 1 and core 2 subunits from the OXPHOS Complex III (bc1) co-purify with Complex IV. One or both of these subunits contains carbonyls at detectable levels even before in vitro oxidation.

FIG. 24 demonstrates peroxynitrite-induced formation of 3-nitrotyrosine modifications of Complex IV. Panel A shows the effect of increasing concentration of peroxynitrite upon Complex IV activity in the mitochondrial membranes. This effect upon activity is also seen when Complex IV is immunocaptured. In panel B the immunocaptured Complex IV sample exposed to 800 μM peroxynitrite is resolved by a SDS-PAGE showing that all Complex IV bands appear present suggesting assembly is unaffected. Furthermore Complex IV subunit Vb contains 3-nitrotyrosine modification(s).

FIG. 25 shows the rate of substrate (ferrocytochrome c) consumption by Complex IV enzyme in samples containing differing amounts of immunocaptured enzyme (from 0.5 pmol to 2.5 pmol). KCN inhibits Complex IV activity; thus, negligible ferrocytochrome c is consumed in the presence of 2.5 pmol Complex IV with added KCN.

FIG. 26 shows a time course of Complex I activity measured by microplate assay in samples containing differing amounts (from 0.016 mg to 0 mg) mitochondria.

FIG. 27 shows the time course of the activity of immunocaptured NADH:UQ1 oxidoreductase (Complex I) on 96-well plates. Samples containing 16 μg heart mitochondria per well were immunocaptured and Complex I activity measured as described in Example 9. The oxidation of NADH was measured by following the decrease of the absorbance at 340 nm either in the absence (♦) or presence of 240 ng rotenone (▪). A control well, to which no mAb was added, is shown (▴).

FIG. 28 shows a schematic representation of an oxidation/reduction reaction taking place on a Complex I dipstick (left). In addition, three Complex I dipsticks having the indicated zones are shown (right). The dipsticks were exposed to the indicated mitochondrial protein samples and, as applicable, Complex I inhibitor.

FIG. 29 shows two Complex IV dipsticks having the indicated zones. The dipsticks were exposed to the indicated mitochondrial protein samples and, as applicable, Complex IV inhibitor.

FIG. 30 shows three Complex V dipsticks having the indicated zones. The dipsticks were exposed to the indicated mitochondrial protein samples and, as applicable, Complex V inhibitor.

FIG. 31 is a graph showing the relationship between the amount of fluorescently labeled protein bound by an anti-Complex V capture mAb tethered to a microtitre plate versus the concentration of total fluorescently labeled human heart mitochondrial protein added to the well.

### Sequence Listing

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:
SEQ ID NO: 1 shows a Complex I NDUFA9 subunit forward primer.
SEQ ID NO: 2 shows a Complex I NDUFA9 subunit reverse primer.
SEQ ID NO: 3 shows a Complex I NDUFS3 subunit forward primer.
SEQ ID NO: 4 shows a Complex I NDUFS3 subunit reverse primer.

### DETAILED DESCRIPTION

I. Overview of Several Embodiments

Disclosed herein are monoclonal antibodies or fragments thereof that competitively inhibits the specific binding of any one of the following monoclonal antibodies: RAC#24-20D1AB7, RAC#24-18G12BC2AA10, RAC#24-17C8E4E11, RAC#24-17G3D9E12, RAC#29-1D4, RAC#29-4G6BB9, RAC#29-6E1BH7, RAC#24A-20E9DH10C12, RAC#23C-4H12BG12AG2, RAC#23B-1A11BC12AB9, RAC#23C-4H12BC11BC5, RAC#23B-10D2, RAC#23C-11A51H12, RAC#23C-12G8, RAC#23C-17A81A8, RAC#23C-29C2, RAC#11B-7E5BA4, RAC#23C-21H10, RAC#23C-22D5, RAC#23C-22H11G43E1, RAC#23C-28G7, RAC#23C-31E91B82G9, MM#1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, and RAC#29-10C6AC9.

Also disclosed are monoclonal antibodies or antibody fragments selected from the group consisting of: RAC#24-20D1AB7, RAC#24-18G12BC2AA10, RAC#24-17C8E4E11, RAC#24-17G3D9E12, RAC#29-1D4, RAC#29-4G6BB9, RAC#29-6E1BH7, RAC#24A-20E9DH10C12, RAC#23C-4H12BG12AG2, RAC#23B-1A11BC12AB9, RAC#23C-4H12BC11BC5, RAC#23B-10D2, RAC#23C-11A51H12, RAC#23C-12G8, RAC#23C-17A81A8, RAC#23C-29C2, RAC#11B-7E5BA4, RAC#23C-21H10, RAC#23C-22D5, RAC#23C-22H11G43E1, RAC#23C-28G7, RAC#23C-31E91B82G9, MM#1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9, and antigen-binding fragments of each thereof. Other embodiments disclose hybridomas expressing any of the foregoing antibodies.

This disclosure envisions methods of detecting the presence of all or part of an OXPHOS enzyme complex (such as, Complex I, Complex II, Complex, III, Complex IV, Complex V, or a combination thereof) in a biological sample, involving (a) contacting a monoclonal antibody specific for a native OXPHOS enzyme complex with a biological sample, wherein all or part of an OXPHOS enzyme complex present in the biological sample and the monoclonal antibody form an immunocomplex, comprising immunocaptured OXPHOS enzyme complex; and (b) detecting the formation of the immunocomplex. Some particular method embodiments further include quantifying the immunocaptured OXPHOS enzyme complex, and/or assaying an enzymatic function of the immunocaptured OXPHOS enzyme complex; and/or detecting a posttranslational modification in the immunocaptured OXPHOS enzyme complex. In more particular embodiments, a detected posttranslational modification includes phosphorylation, oxidative damage, or carbonyl formation.

Other methods of detecting the presence of all or part of an OXPHOS enzyme complex in a biological sample further involve separating the immunocomplex from components of the biological sample that are not substantially bound by the antibody; and/or releasing the immunocaptured OXPHOS enzyme complex from the immunocomplex, and separating subunits of the OXPHOS enzyme complex (such as, by gel electrophoresis). In other embodiments, an immunocaptured OXPHOS enzyme complex is released from the immunocomplex, and the released OXPHOS enzyme complex is isolated.

In some methods of detecting the presence of all or part of an OXPHOS enzyme complex in a biological sample, the biological sample is from a human and/or is a cell lysate, mitochondrial extract, or tissue extract. In specific examples, the cell lysate or mitochondrial extract is from a fibroblast, peripheral blood mononuclear cell (PBMC), needle biopsy, or mucosal epithelial cell. In other specific method embodiments, the biological sample comprises less than about 50 mg total protein or less than about 1×10⁷ cells.

In particular method embodiments, detection of the formation of the immunocomplex involves (a) contacting the immunocomplex with a detectable marker that binds specifically to the immunocomplex; (b) assaying an activity of the immunocaptured OXPHOS enzyme complex; or (c) a combination of (a) and (b). In other examples, high-throughput screening is used to detect the formation of the immunocomplex comprises high-throughput screening. In still other examples, the antibody is attached to a solid support (such as, a bead, a microtiter plate, or a dipstick).

Specific method embodiments involve detecting the presence of all or part of Complex I in a biological sample. In these methods, the antibody is a monoclonal antibody that specifically binds to at least one subunit of Complex I (such as, RAC#24-20D1AB7, RAC#24-18G12BC2AA10, RAC#24-17C8E4E11, RAC#24-17G3D9E12, RAC#29-1D4, RAC#29-4G6BB9, RAC#29-6E1BH7, RAC#24A-20E9DH10C12, or combinations of any thereof).

Other specific method embodiments involve detecting the presence of all or part of Complex II in a biological sample. In these methods, the antibody is a monoclonal antibody that specifically binds to at least one subunit of Complex II (such as, RAC#23C-4H12BG12AG2).

Still other specific method embodiments involve detecting the presence of all or part of Complex III in a biological sample. In these methods, the antibody is a monoclonal antibody that specifically binds to at least one subunit of Complex II (such as, RAC#23B-1A11BC12AB9; RAC#23C-4H12BC11BC5; RAC#23B-10D2; RAC#23C-11A51H12; RAC#23C-12G8; RAC#23C-17A81A8; RAC#23C-29C2, or combinations of any thereof).

Yet other specific method embodiments involve detecting the presence of all or part of Complex IV in a biological sample. In these methods, the antibody is a monoclonal antibody that specifically binds to at least one subunit of Complex IV (such as, RAC#11B-7E5BA4; RAC#23C-21H10; RAC#23C-22D5; RAC#23C-22H11G43E1; RAC#23C-28G7; RAC#23C-31E91B82G9, or combinations of any thereof).

Still other specific method embodiments involve detecting the presence of all or part of Complex V in a biological sample. In these methods, the antibody is a monoclonal antibody that specifically binds to at least one subunit of Complex V (such as, MM# 1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9, or a combination of any thereof).

Also disclosed herein are methods for identifying an agent with potential to cause mitochondrial damage, including the steps of (a) contacting an immunocaptured OXPHOS enzyme complex with a test agent; and (b) assaying the activity of the immunocaptured OXPHOS enzyme complex in the presence and absence of the test agent, wherein a decrease in the activity of the OXPHOS enzyme complex in the presence of the test agent as compared to in the absence of the test agent indicates that the test agent is an agent with potential to cause mitochondrial damage. In particular examples of these methods, the immunocaptured OXPHOS enzyme complex is Complex I, Complex II, Complex, III, Complex IV, Complex V, or a combination thereof (such as, Complex I, Complex IV or a combination thereof). In other examples, the agent is an environmental toxin and/or a drug (such as a drug that is used, or is being tested for use, in highly active anti-retroviral therapy).

In some method for identifying an agent with potential to cause mitochondrial damage, the immunocaptured OXPHOS enzyme complex is from a human subject and the method assesses mitochondrial damage in the human subject. Particular examples involve repeating the method at spaced intervals to assess progressive mitochondrial damage in the human subject. In specific examples, assessing progressive mitochondrial damage detects the onset or stage of a mitochondrial disorder.

Further methods for identifying an agent with potential to cause mitochondrial damage are disclosed. Such methods include (a) contacting a biological system, comprising at least one OXPHOS enzyme complex (such as, Complex I, Complex II, Complex, III, Complex IV, Complex V, or a combination thereof), with a test agent; (b) immunocapturing at least one OXPHOS enzyme complex from the biological system; and (c) determining whether there is a relative change in a level, an activity, the number of subunits, or a post-translational modification of the OXPHOS enzyme complex as compared to a control biological system that is not contacted with the agent, wherein a relative change in the level, the activity, the number of subunits, or the post-translational modification of the OXPHOS enzyme complex identifies the test agent as an agent with potential to cause mitochondrial damage. In some such methods, the biological system comprises a cell (such as, a cell contained within an organism or tissue sample). In other such methods, the level of the OXPHOS enzyme complex is decreased relative to the control biological system, and/or the activity of the OXPHOS enzyme complex is decreased relative to the control biological system. In specific examples, post-translation modification of an OXPHOS enzyme complex includes phosphorylation, oxidative damage, or carbonyl formation, which is not present in the control biological system. In other specific examples, the agent is an environmental toxin and/or a drug (such as a drug used, or is being tested for use, in highly active anti-retroviral therapy).

Methods for detecting a deficiency of an OXPHOS enzyme complex (such as, Complex I, Complex II, Complex, III, Complex IV, Complex V or a combination thereof) in a subject are also disclosed. Such methods involve (a) contacting a biological sample from a subject with a plurality of monoclonal antibodies, each of which is specific for a subunit of an OXPHOS enzyme complex, wherein the plurality of monoclonal antibodies form a plurality of immunocomplexes, each immunocomplex comprising a monoclonal antibody and a specifically bound OXPHOS subunit; (b) detecting the amount of specifically bound OXPHOS subunit for each of the plurality of monoclonal antibodies; and (c) comparing the amount of each specifically bound OXPHOS subunit with an amount of the same OXPHOS subunit in a corresponding control sample of the OXPHOS enzyme complex, wherein a decrease in the amount of any OXPHOS subunit(s) of the OXPHOS enzyme complex in the subject sample as compared to the control sample indicates the presence of a deficiency of the OXPHOS enzyme complex in the subject.

Specific method embodiments involve detecting a deficiency of Complex I. In some of these methods, the plurality of antibodies is a combination of at least two monoclonal antibodies that specifically bind to the 30 kDa, 20 kDa, 15 kDa, or 8 kDa subunits of Complex I. For example, the plurality of antibodies can include RAC#24-20D1AB7, RAC#24-18G12BC2AA10, RAC#24-17C8E4E11, RAC#24-17G3D9E12, RAC#29-1D4, RAC#29-4G6BB9, RAC#29-6E1BH7, RAC#24A-20E9DH10C12, or a combination thereof. In some examples a method for detecting a deficiency of Complex I further involves determining a failure of the Complex I subunits to assemble to form a fully assembled Complex I, thereby determining that the deficiency comprises a failure in Complex I assembly.

Other specific method embodiments involve detecting a deficiency of Complex II. Still other specific method embodiments involve detecting a deficiency of Complex m. In some of these methods the plurality of antibodies comprises RAC#23B-1A11BC12AB9; RAC#23C-4H12BC11BC5; RAC#23B-10D2; RAC#23C-11A51H12; RAC#23C-12G8; RAC#23C-17A81A8; RAC#23C-29C2, or combinations of any thereof.

Yet other specific method embodiments involve detecting a deficiency of Complex IV. In some of these embodiments, the plurality of antibodies is a combination of at least two monoclonal antibodies that specifically bind to the core 1, core 2, I, II, III, IV, Vb, Va, VIaH, VIb, Vic, VIIaH, VIIb, VIIc or VIII subunit of Complex IV. In other examples, the plurality of antibodies includes RAC#11B-7E5BA4; RAC#23C-21H10; RAC#23C-22D5; RAC#23C-22H11G43E1; RAC#23C-28G7; RAC#23C-31E91B82G9, or a combination of any thereof.

Still other specific method embodiments involve detecting a deficiency of Complex V. In some of these methods, the plurality of antibodies is a combination of at least two monoclonal antibodies that specifically bind to the α, β, d, OSCP, or IF₁ subunit of Complex V. In other examples, the plurality of antibodies comprises MM#1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9, or a combination of any thereof.

Contemplated herein are methods for diagnosing late onset mitochondrial disorder (such as, late onset diabetes, Huntington's disease, Parkinson's disease, Alzheimer's diseases, amyotrophic lateral sclerosis, or schizophrenia) in a subject. Such methods involve (a) contacting an antibody specific for Complex I with a biological sample, wherein Complex I present in the biological sample and the antibody form an immunocomplex, comprising immunocaptured Complex I; (b) separating the immunocaptured Complex I from components of the biological sample that are not substantially bound by the antibody; and (c) detecting the presence of a posttranslational modification in one or more subunits of the immunocaptured Complex I, wherein the presence of a posttranslational modification indicates that the subject has late onset mitochondrial disorder. In specific method embodiments, separating the immunocaptured Complex I involves (i) releasing the immunocaptured Complex I from the immunocomplex; and (ii) separating the Complex I subunits from one another by weight difference. In other more particular method embodiments, detecting the presence of a posttranslational modification involves detecting a difference in an immunocaptured Complex I subunit molecular weight as compared to a control Complex I subunit molecular weight.

This disclosure also envisions immunoassay devices for determining presence and/or amount of an OXPHOS enzyme complex (such as, Complex I, Complex II, Complex, III, Complex IV, Complex V, or a combination of any thereof) in a sample. Such devices include a sample contact area; and a respiratory enzyme capture area comprising an immobilized antibody having a binding affinity for an OXPHOS enzyme complex; wherein a sample applied in the sample contact area flows in a direction of flow from the sample contact area to the respiratory enzyme capture area, and formation of a complex between the immobilized antibody and an OXPHOS enzyme complex is detectable to determine the presence and/or amount of the OXPHOS enzyme complex in the sample. In particular device embodiments, the immobilized antibody includes RAC#24-20D1AB7, RAC#24-18G12BC2AA10, RAC#24-17C8E4E11, RAC#24-17G3D9E12, RAC#29-1D4, RAC#29-4G6BB9, RAC#29-6E1 BH7, RAC#24A-20E9DH10C12, RAC#23C-4H 12BG12AG2, RAC#23B-1A11BC12AB9, RAC#23C-4H12BC11BC5, RAC#23B-10D2, RAC#23C-11A51H12, RAC#23C-12G8, RAC#23C-17A81A8, RAC#23C-29C2, RAC#11B-7E5BA4, RAC#23C-21H10, RAC#23C-22D5, RAC#23C-22H11G43E1, RAC#23C-28G7, RAC#23C-31E91B82G9, MM#1-12F4AD8AF8, MM#7-3D5AB 1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9, an antigen-binding fragment of any thereof, or a combination of any thereof.

Further disclosed are immunoassay devices including a solid support (such as, a microtitre plate), which involve a plurality of discrete capture areas, each discrete capture area containing an immobilized monoclonal antibody specific for an OXPHOS enzyme complex. In specific device embodiments, the immobilized monoclonal antibodies are RAC#24-20D1AB7, RAC#24-18G12BC2AA10, RAC#24-17C8E4E11, RAC#24-17G3D9E12, RAC#29-1D4, RAC#29-4G6BB9, RAC#29-6E1BH7, RAC#24A-20E9DH10C12, RAC#23C-4H12BG12AG2, RAC#23B-1A11BC12AB9, RAC#23C-4H12BC11BC5, RAC#23B-10D2, RAC#23C-11A51H12, RAC#23C-12G8, RAC#23C-17A81A8, RAC#23C-29C2, RAC#11B-7E5BA4, RAC#23C-21H10, RAC#23C-22D5, RAC#23C-22H11G43E1, RAC#23C-28G7, RAC#23C-31E91B82G9, MM#1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9, an antigen-binding fragment of any thereof, or a combination of any thereof.

Also disclosed herein are kits including any of the foregoing immunoassay devices. Some kit embodiments also include a standard curve showing a correlation of the activity of the OXPHOS enzyme complex with expression level of the respiratory enzyme in subjects having normal activity of the OXPHOS enzyme complex.

II. Abbreviations and Terms

ANT ADP/ATP translocase
BHM bovine heart mitochondrial membranes
CMF-PBS Dulbecco's phosphate-buffered saline
CoA Coenzyme A
Complex I NADH ubiquinone oxidoreductase
Complex II succinate ubiquinone reductase
Complex III ubiquinone-cytochrome c oxidoreductase
Complex IV (or COX) cytochrome c oxidase
Complex V (or F₁/F₀ ATPase) ATP synthase
DNP 2,4-dinitrophenylhydrazone
DNPH 2,4-dinitrophenylhydrazine
DTT 1,4-dithio-threitol
FP Flavoprotein
GAM goat-anti-mouse (antibody)
HAART highly active anti-retroviral therapy
HGDMEM Dulbecco's modified Eagle's Medium
HHM human heart mitochondrial membranes
IFA incomplete Freund's adjuvant
IF₁ Inhibitor of F₁/F₀ ATPase
LC-MS/MS liquid chromatography mass spectrometry/mass spectrometry
M F₁F₀ mitochondrial F₁/F₀ ATPase
mAb monoclonal antibody
MALDI-TOF matrix assisted laser desorption/ionization time-of-flight
MOPS n-dodecyl-β-D-maltoside
mtDNA mitochondrial DNA
NADH nicotinamide adenine dinucleotide
NRTI nucleotide reverse transcriptase inhibitor
NSA nonspecific antibody
OD optical density
OSCP oligomycin sensitivity-conferring protein
OXPHOS oxidative phosphorylation
PBS phosphate buffered saline
PD population doubling
PDH pyruvate dehydrogenase complex
PMF Peptide Mass Fingerprints
PMSF phenylmethylsulfonyl fluoride
ROS reactive oxygen species

Unless otherwise explained, all technical and scientific terms used herein have the same meaning as commonly understood by one of ordinary skill in the art to which the disclosed subject matter belongs. Definitions of common terms relating to antibodies may be found in Harlow and Lane, Antibodies, A Laboratory Manual, CSHP, New York (1988).

As used herein, the singular terms “a,”, “an,” and “the” include plural referents unless context clearly indicates otherwise. Similarly, the word “or” is intended to include “and” unless the context clearly indicates otherwise. “Comprising” means “including.” Hence “comprising A or B” means “including A or B”, or “including A and B.” All molecular weights, molecular mass values, or lengths given for nucleic acids or polypeptides are approximate, and are provided for description.

In order to facilitate review of the various embodiments of the invention, the following explanations of specific terms are provided:

Analyte: An atom, molecule, group of molecules or compound of natural or synthetic origin (e.g., drug, hormone, enzyme, protein, peptide, protein complex, antigen, antibody, hapten, lectin, apoprotein, cofactor) sought to be detected or measured that is capable of binding specifically to at least one binding partner (e.g., drug, hormone, antigen, antibody, hapten, lectin, apoprotein, cofactor).

Analytes vary in size. Merely by way of example, small molecule analytes may be, for instance, <0.1 nm. However, analytes may be larger than this, including for instance immunoglobulin analytes (such as IgG, which is about 8 nm in length and about 160,000 Daltons) or other protein complexes, such as the mitochondrial protein complexes described herein.

Antibody or Immunoglobin: A polypeptide substantially encoded by an immunoglobulin gene or immunoglobulin genes, or fragments thereof, which specifically binds and recognizes an antigen. Immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

The term “antibody,” as used herein, also includes antibody fragments either produced by the modification of whole antibodies or those synthesized using recombinant DNA methodologies. Antibodies exist, e.g., as intact immunoglobulins or as a number of well-characterized antigen-binding fragments defined as follows: (1) Fab, the fragment which contains a monovalent antigen-binding fragment of an antibody molecule produced by digestion of whole antibody with the enzyme papain to yield an intact light chain and a portion of one heavy chain; two Fab fragments are obtained per antibody molecule; (2) Fab′, the fragment of an antibody molecule obtained by treating whole antibody with pepsin, followed by reduction, to yield an intact light chain and a portion of the heavy chain; two Fab′ fragments are obtained per antibody molecule; Fab′ fragments contain additional heavy chain residues that are not contain by Fab fragments; (3) (Fab′)₂, the fragment of the antibody obtained by treating whole antibody with the enzyme pepsin without subsequent reduction; (4) Fv, a genetically engineered fragment containing the variable region of the heavy chain (V_{H}) and the variable region of the light chain (V_{L}) expressed as two chains; the unmodified Fv fragment is unstable because there are no disulfide bonds connecting the heavy chain and light chain constituents; (5) single chain antibody (scFv), a genetically engineered molecule containing the V_{H} and V_{L} domains linked by a suitable polypeptide linker as a genetically fused single chain molecule; and (6) disulfide-stabilized Fv (dsFv), a genetically engineered heterodimer containing the V_{H} and V_{L} domains, which are linked by disulfide bonds between residues engineered into each domain. Methods of making these fragments are known in the art.

Antigenic: A chemical or biochemical structure, determinant, antigen or portion thereof that is capable of inducing the formation of an antibody.

Array: An arrangement of molecules, particularly biological macromolecules (such as polypeptides or nucleic acids) or biological samples (such as tissue sections) in addressable locations on a substrate, usually a flat substrate such as a membrane, plate or slide.

Binding affinity: A term that refers to the strength of binding of one molecule to another. If a particular molecule will bind to or specifically associate with another particular molecule, these two molecules are said to exhibit binding affinity for each other. Binding affinity is related to the association constant and dissociation constant for a pair of molecules, but it is not critical to the invention that these constants be measured or determined. Rather, affinities as used herein to describe interactions between molecules of the described methods and devices are generally apparent affinities (unless otherwise specified) observed in empirical studies, which can be used to compare the relative strength with which one molecule (e.g., an antibody or other specific binding partner) will bind two other molecules (e.g., an analyte or antigen, such as a protein). The concepts of binding affinity, association constant, and dissociation constant are well known.

Binding partner: Any molecule or composition capable of recognizing and binding to a specific structural aspect of another molecule or composition. Examples of such binding partners and corresponding molecule or composition include antigen/antibody, hapten/antibody, lectin/carbohydrate, apoprotein/cofactor and biotin/(strept)avidin.

Biological Sample: Any sample that may be obtained directly or indirectly from an organism, including whole blood, plasma, serum, tears, mucus, saliva, urine, pleural fluid, spinal fluid, gastric fluid, sweat, semen, vaginal secretion, sputum, fluid from ulcers and/or other surface eruptions, blisters, abscesses, tissues, cells (such as, fibroblasts, peripheral blood mononuclear cells, or muscle cells), organelles (such as mitochondria), organs, and/or extracts of tissues, cells (such as, fibroblasts, peripheral blood mononuclear cells, or muscle cells), organelles (such as mitochondria) or organs. An “organism” includes, without limitation, plants, animals, or microbes. The term “animal” includes vertebrate or invertebrate animals, such as mammals (for example, humans), insects (for example, Drosophila melanogaster), nematodes (for example, Caenorhabditis elegans), and fish (for example, Danio rerio, aka, zebrafish). A biological sample may also be a laboratory research sample such as a cell culture supernatant. The sample is collected or obtained using methods well known to those skilled in the art.

Capture Antibody: An antibody (usually a monoclonal antibody or engineered antibody molecule that has monoclonal specificity) that can specifically bind an epitope present on one or more native, target OXPHOS protein(s). Typically, a capture antibody epitope is contained within one OXPHOS protein; however, a single epitope may span two or more native OXPHOS proteins when such proteins are associated in a partially or fully assembled OXPHOS complex. A target OXPHOS protein(s) may be substantially free of associated proteins or may be incorporated into a completely or partially assembled OXPHOS enzyme complex (for additional description of capture antibodies, see section herein entitled “‘Capture’ Antibodies Specific for Native OXPHOS Complexes and Native OXPHOS Proteins”).

Complementarity Determining Regions or CDRs: Amino acid sequences which together define the binding affinity and specificity of the natural variable binding region of a native immunoglobulin binding site (such as Fv), a T cell receptor (such as V_{α} and V_{β}), or a synthetic polypeptide which mimics this function.

Conjugate: When used in the verb form, the term “conjugate” means the covalent coupling of one molecule (e.g., a mAb specific for an OXPHOS enzyme complex, such as Complex I, II, III, IV, or V, or subunits thereof) to another molecule (e.g., a fluorochrome) or a small particle (e.g., a colloidal gold particle). Such coupling may be achieved by chemical means, either with or without the use of a linking group. When used in the noun form, the term “conjugate” means a coupled molecular complex formed by conjugation.

Detecting or Detection: Refers to quantitatively or quantitatively determining the presence of an analyte(s) under investigation (e.g., an OXPHOS enzyme complex or subunit(s) thereof). “Detecting Formation of a Complex” refers to detecting a complex comprising a detector reagent by any method suitable for observing the particular label associated with the detector reagent; for instance, visual observation of a colored (or otherwise visible) label, measurement or visual detection of a fluorescent, chemiluminescent or radioactive label.

ELISA: Enzyme-linked immunosorbent assay. A form of quantitative immunoassay based on the use of antibodies (or antigens) that are linked to an insoluble carrier surface, which is then used to capture the relevant antigen (or antibody) in the test solution. The antigen-antibody complex is then detected by measuring, for example, the activity of a captured antigen or an enzyme that is directly or indirectly attached to the antigen (or antibody).

Enzymatic Activity: A detectable (and usually quantifiable) characteristic of at least one function of an enzyme (such as, an OXPHOS enzyme), often monitored over time or in comparison to a standard curve. Methods are well known to those of ordinary skill in the art, for detecting, determining, monitoring, and/or quantifying various enzymatic activities. Also well known are ways of using enzymatic activity assays to assess the ability of compounds (for instance, test compounds) to affect the function of the enzyme, for instance, as an inhibitor or enhancer.

For instance, “ATPase activity” is usually contemplated as the ability to detectably hydrolyze ATP. ATPase activity can be measured using various assays known to those of ordinary skill in the art, including those assays provided herein, for instance, in Example 2. In some examples, ATPase activity is measured in solution by detecting (quantitatively or qualitatively) free phosphate released by enzyme activity (such as, Complex V activity). Methods of detecting free phosphate are known and include, for example, both calorimetric and fluorescent techniques (see, e.g., Aggeler et al., J. Biol. Chem., 277: 33906-33912, 2002). In other examples, ATPase activity of an immobilized enzyme (for instance, Complex V immunocaptured on a dipstick) is detected, for example, by fluorescent techniques (such as, PᵢPer™ Phosphate Assay Kit or EnzChek® Phosphate Assay Kit available from Molecular Probes), or modification of tissue-based histochemical techniques (see, e.g., Bancroft and Stevens, Theory and Practice of Histological Techniques, 4th edition, London: Churchill-Livinstone, 1996).

“Oxidoreductase activity” is the ability of an enzyme to reversibly oxidize (remove protons and electrons, or reducing equivalents from) a first substrate molecule and contemporaneously reduce (add protons and electrons, or reducing equivalents to) a second substrate molecule. First and second substrate molecules typically are, but need not be, proteins, carbohydrates, lipids, or small co-factors.

Oxidation and/or reduction can be detected by any method known in the art. In some examples, a detectable change in a physical property of the oxidized and/or reduced substrate molecule(s) is measured; for example, a change in optical density (OD) at some defined wavelength. In particular examples, OD₃₄₀ can be used to monitor the ratio of NAD/NADH redox (such as, in assays of Complex I activity), or OD₆₀₀ can be used to monitor reduction of 2,6-dichlorophenolindophenol (such as, in assays for Complex II activity), or OD₅₅₀ can be used to monitor oxidation of cytochrome c (II) (such as, in assays for Complex IV activity) (see, e.g., Birch-Machin and Turnbull, Meth. Cell Biol., 65: 97-117, 2001). In other examples, oxidation and/or reduction can be detected by monitoring a change in the properties of a prosthetic group in the oxidoreductase enzyme; for example, the ratio of OD₆₀₅/OD₆₃₀ can be used to monitor heme aa3 of Complex IV (see, e.g., Rickwood et al., in Mitochondria. A Practical Approach, ed. by Darley-Usmar et al., Oxford: IRL Press, 1987). In still other examples, oxidation and/or reduction can be detected by coupling the oxidation or reduction reaction of interest to another more easily monitored redox reaction, such as oxidation or reduction of a chromogenic or fluorogenic substrate (see, e.g., Birch-Machin and Turnbull, Meth. Cell. Biol., 65: 97-117, 2001; Amplex Red reagent (10-acetyl-3,7-dihydroxyphenoxazine available from Molecular Probes)). Further examples of oxidoreductase activity assays are provided, at least, in Examples 1, 7, 8, 9, and 10.

“Reductase activity” is the ability of an enzyme to reduce (add electrons or reducing equivalents to) a substrate molecule, which typically is, but need not be, a protein, a carbohydrate, a lipid or a small co-factor. The reducing equivalents are obtained by the enzyme from some other molecule which is thereby oxidized either contemporaneously with, or at some time prior to, the reductase enzyme/substrate reaction. Reductase activity can be measured using various assays known to those of ordinary skill in the art. For example, assays for activity of Complex II can follow reduction of the oxidized substrate 2,6-dichlorophenolindophenol by monitoring changes in OD₆₀₀ (Birch-Machin and Turnbull, Meth. Cell Biol., 65: 97-117, 2001). Additional reductase activity assays including those assays provided herein in Examples 1, 7, 8, 9, and 10.

“Oxidase activity” is the ability of an enzyme to oxidize (remove protons and electrons or reducing equivalents from) a substrate molecule, which typically is, but need not be, a carbohydrate, a lipid or a small co-factor. The reducing equivalents are typically transferred by the enzyme to some other molecule which is thereby reduced either contemporaneously with, or at some time after, the oxidase enzyme/substrate reaction. Oxidase activity can be measured using various assays known to those of ordinary skill in the art, including those assays provided in Examples 1, 7, 8, 9 and 10. In one particular example, Complex IV oxidase activity can be detected by observing the oxidation of cytochrome c by measuring OD₅₅₀ (Birch-Machin and Turnbull, Meth. Cell Biol., 65: 97-117, 2001).

Epitope (or antigenic determinant): A site on the surface of an antigen molecule to which a single antibody molecule binds; generally an antigen has several or many different antigenic determinants and reacts with antibodies of many different specificities.

Fluorescence in situ hybridization (FISH): In this technique, fluorescent molecules are used to label a DNA probe, which can then hybridize to a specific DNA sequence in a chromosome spread so that the site becomes visible through a microscope. FISH has been used to highlight the locations of genes, sub-chromosome regions, entire chromosomes, or specific DNA sequences. It has been used for mapping and the detection of genomic rearrangements, as well as studies on DNA replication.

Highly Active Anti-retroviral Therapy (HAART): A combination therapy, composed of multiple anti-HIV drugs, which is prescribed to many HIV-positive subjects, in some instances even before AIDS symptoms are apparent. The therapy usually includes one nucleoside analog, one protease inhibitor and either a second nucleoside analog or a non-nucleoside inhibitor of reverse transcription.

Immunocapture: A method of isolating a protein or protein complex (such as a native OXPHOS protein or native OXPHOS complex), using the specific binding of that protein/complex to an antibody (such as, a monoclonal antibody). In particular examples, immunocapture refers to a method of using anti-OXPHOS protein or anti-OXPHOS complex antibodies to specifically bind an OXPHOS protein or OXPHOS complex, respectively, in its native state. An immunocapture antibody may, but need not, be immobilized to a surface, such as a bead, a microtitre plate, or nitrocellulose. Under those circumstances, immunocapture can be used to purify a protein or protein complex of interest (such as a native OXPHOS protein or native OXPHOS complex). In other examples, immunocapture of a protein or protein complex (such as a native OXPHOS protein or native OXPHOS complex) may occur in solution (wherein the antibody is not substantially immobilized).

Label: Any molecule or composition bound to an analyte, analyte, detector reagent, analog or binding partner that is detectable by spectroscopic, photochemical, biochemical, immunochemical, electrical, optical or chemical means. Non-limiting examples of labels include enzymes, colloidal gold particles, colored latex particles, radioactive isotopes, enzyme substrates, co-factors, ligands, chemiluminescent or fluorescent agents, haptens, protein-adsorbed silver particles, protein-adsorbed iron particles, protein-adsorbed copper particles, protein-adsorbed selenium particles, protein-adsorbed sulphur particles, protein-adsorbed tellurium particles, protein-adsorbed carbon particles, and protein-coupled dye sacs. Methods for labeling and guidance in the choice of labels appropriate for various purposes are discussed, e.g., in Sambrook et al., Molecular Cloning: A Laboratory Manual, CSHL, New York, 1989 and Ausubel et al., Current Protocols in Molecular Biology, Greene Publ. Assoc. and Wiley-Intersciences, 1998. The attachment of a compound (e.g., an antibody) to a label can be through covalent bonds, adsorption processes, hydrophobic and/or electrostatic bonds, as in chelates and the like, or combinations of these bonds and interactions and/or may involve a linking group.

Specific example detectable labels suitable for conjugating to antibodies used in the methods, including high throughput screening formats, include radiolabels linked to the antibodies using various chemical linking groups or bifunctional peptide linkers. A terminal hydroxyl can be esterified with inorganic acids, e.g., ³²P phosphate, or ¹⁴C organic acids, or else esterified to provide linking groups to the label. Enzymes of interest as detectable labels will primarily be hydrolases, particularly esterases and glycosidases, or oxidoreductases, particularly peroxidases. Fluorescent compounds include fluorescein and its derivatives, rhodamine and its derivatives, dansyl, umbelliferone, and so forth. Chemiluminescers include luciferin, and 2,3-dihydrophthalazinediones (e.g., luminol), and the like.

Lateral flow device: An analytical device in the form of a test strip used in lateral flow chromatography, in which a test sample fluid, suspected of containing an analyte, flows (for example by capillary action) through the strip (which is frequently made of bibulous materials such as paper, nitrocellulose, and cellulose). The test fluid and any suspended analyte can flow along the strip to a detection zone in which the analyte (if present) interacts with a detection agent to indicate a presence, absence and/or quantity of the analyte.

Numerous lateral flow analytical devices have been disclosed, and include (without limitation) those shown in U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; and 6,368,876; EP 0810436; and WO 92/12428; WO 94/01775; WO 95/16207; and WO 97/06439.

Many lateral flow devices are one-step lateral flow assays in which a biological fluid is placed in a sample area on a bibulous strip (though, non-bibulous materials can be used, and rendered bibulous by applying a surfactant to the material), and allowed to migrate along the strip until the liquid comes into contact with a specific binding partner that interacts with an analyte in the liquid. Once the analyte interacts with the binding partner, a signal (such as a fluorescent or otherwise visible dye) indicates that the interaction has occurred. Multiple discrete binding partners can be placed on the strip (for example in parallel lines) to detect multiple analytes in the liquid. The test strips can also incorporate control indicators, which provide a signal that the test has adequately been performed, even if a positive signal indicating the presence (or absence) of an analyte is not seen on the strip.

Lateral flow chromatography strip: A test strip used in lateral flow chromatography, in which a test sample fluid, suspected of containing an analyte, flows (for example by capillary action) through the strip (which is frequently made of materials such as paper or nitrocellulose). The test fluid and any suspended analyte can flow along the strip to a detection zone in which the analyte (if present) interacts with a detection agent to indicate a presence, absence and/or quantity of the analyte. A “dipstick” is a lateral flow chromatography strip that is (or, a portion of which is) directly immersed into a sample-containing solution. Often, a dipstick is not incorporated within a housing, as is a customary lateral flow device.

Linking group: A chemical arm between two compounds, for instance a compound and a label (e.g., an analyte and a label). To accomplish the requisite chemical structure, each of the reactants must contain a reactive group. Representative combinations of such groups are amino with carboxyl to form amide linkages; carboxy with hydroxy to form ester linkages or amino with alkyl halides to form alkylamino linkages; thiols with thiols to form disulfides; or thiols with maleimides or alkylhalides to form thioethers. Hydroxyl, carboxyl, amino and other functionalities, where not present in the native compound may be introduced by known methods.

Likewise, a wide variety of linking groups may be employed. The structure of the linkage should be a stable covalent linkage formed to attach two compounds to each other (e.g., the label to the analyte). In some cases the linking group may be designed to be either hydrophilic or hydrophobic in order to enhance the desired binding characteristics, for instance of the modified ligand and its cognate receptor. The covalent linkages should be stable relative to the solution conditions to which linked compounds are subjected. Examples of linking groups will be from 1-20 carbons and 0-10 heteroatoms (NH, O, S) and may be branched or straight chain. Without limiting the foregoing, it should be obvious that only combinations of atoms that are chemically compatible comprise the linking group. For example, amide, ester, thioether, thiol ester, keto, hydroxyl, carboxyl, ether groups in combinations with carbon-carbon bonds are particular examples of chemically compatible linking groups.

Mitochondrial Damage: any physical alteration in mitochondrial components, including mtDNA, proteins (such as, one or more OXPHOS proteins), or lipids, that alters mitochondrial function in a way that is detrimental to cell physiology, growth or faithful replication.

Mitochondrial Disorder: A disease resulting from altered mitochondrial function, caused by any alteration or combination of alterations of mitochondrial components (for instance, mitochondrial protein (such as, one or more OXPHOS proteins), mtDNA, or lipid) caused by genetic and/or environmental factors, including autotoxicity caused by normal cellular metabolic processes. “Late onset mitochondrial disorder” or “late onset disease” means such diseases as late onset diabetes (Diabetes Type I), Huntington's, Parkinson's and Alzheimer's diseases, ALS (amyotrophic lateral sclerosis), Schizophrenia and the like, wherein the subject is free of the disease in early life, but develops the disease during puberty or thereafter, sometimes as late as age 70 or 80.

Native: The native form of a biological molecule is generally considered to be the conformation the molecule takes in the biological milieu in which it normally functions (e.g., under conditions of physiologically normal pH, osmolarity, and/or redox state). This is contrasted with the “denatured” form in which the biological molecule has been altered in some way, generally as a result of exposure to an extreme environmental condition (e.g., heat, pH, salt concentration, redox, or radiation) that either irreversibly or reversibly modifies the molecule, causing the molecule to change its conformation and aggregate, unfold or be altered in some way, generally with detrimental effects on function. Because denaturation results in changes in shape of the molecule, and in particular changes in the surface features of the molecule, it can alter or even eliminate surface epitopes. Therefore, it is common to observe that antibodies that bind to such conformational-sensitive epitopes will either bind only to the native molecule or to the denatured molecule.

Solid Support (or substrate): Any material which is insoluble, or can be made insoluble by a subsequent reaction. Numerous and varied solid supports are known to those in the art and include, without limitation, nitrocellulose, the walls of wells of a reaction tray, test tubes, polystyrene beads, magnetic beads, membranes, microparticles (such as latex particles), and sheep (or other animal) red blood cells. Any suitable porous material with sufficient porosity to allow access by detector reagents and a suitable surface affinity to immobilize capture reagents (e.g., monoclonal antibodies) is contemplated by this term. For example, the porous structure of nitrocellulose has excellent absorption and adsorption qualities for a wide variety of reagents, for instance, capture reagents. Nylon possesses similar characteristics and is also suitable. Microporous structures are useful, as are materials with gel structure in the hydrated state.

Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

It is contemplated that porous solid supports, such as nitrocellulose, described hereinabove are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

The surface of a solid support may be activated by chemical processes that cause covalent linkage of an agent (e.g., a capture reagent) to the support. However, any other suitable method may be used for immobilizing an agent (e.g., a capture reagent) to a solid support including, without limitation, ionic interactions, hydrophobic interactions, covalent interactions and the like. The particular forces that result in immobilization of an agent on a solid phase are not important for the methods and devices described herein.

A solid phase can be chosen for its intrinsic ability to attract and immobilize an agent, such as a capture reagent. Alternatively, the solid phase can possess a factor that has the ability to attract and immobilize an agent, such as a capture reagent. The factor can include a charged substance that is oppositely charged with respect to, for example, the capture reagent itself or to a charged substance conjugated to the capture reagent. In another embodiment, a specific binding member may be immobilized upon the solid phase to immobilize its binding partner (e.g., a capture reagent). In this example, therefore, the specific binding member enables the indirect binding of the capture reagent to a solid phase material.

Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

A “lateral flow substrate” is any solid support or substrate that is useful in a lateral flow device, including for instance a dipstick.

Specific binding partner: A member of a pair of molecules that interact by means of specific, noncovalent interactions that depend on the three-dimensional structures of the molecules involved. Typical pairs of specific binding partners include antigen/antibody, hapten/antibody, hormone/receptor, nucleic acid strand/complementary nucleic acid strand, substrate/enzyme, inhibitor/enzyme, carbohydrate/lectin, biotin/(strept)avidin, and virus/cellular receptor.

The phrase “specifically binds to an analyte” or “specifically immunoreactive with”, when referring to an antibody, refers to a binding reaction which is determinative of the presence of the analyte in the presence of a heterogeneous population of molecules such as proteins and other biologic molecules. Thus, under designated immunoassay conditions, the specified antibodies bind to a particular analyte and do not bind in a significant amount to other analytes present in the sample. A variety of immunoassay formats may be used to select antibodies specifically immunoreactive with a particular analyte. For example, solid-phase ELISA immunoassays are routinely used to select monoclonal antibodies specifically immunoreactive with a protein. See Harlow and Lane, Antibodies, A Laboratory Manual, CSHP, New York (1988), for a description of immunoassay formats and conditions that can be used to determine specific immunoreactivity.

Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Except as otherwise noted, the methods and techniques of the present invention are generally performed according to conventional methods well known in the art and as described in various general and more specific references that are cited and discussed throughout the present specification. See, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

III “Capture” Antibodies Specific for Native OXPHOS Complexes and Native OXPHOS Proteins

Disclosed herein are antibodies (such as, monoclonal antibodies) that are specific for native OXPHOS enzyme complexes (such as, Complex I, II, III, IV or V) and native OXPHOS protein subunits. Prior to the present disclosure, anti-OXPHOS monoclonal antibodies were of limited use and only applicable in techniques that did not require the target OXPHOS protein or OXPHOS complex to maintain its native protein/complex conformation (such as, Western blot or immunohistochemistry). However, because OXPHOS protein/complex conformation appears to be easily altered (and, therefore, mAb recognition completely abrogated), such antibodies did not bind native OXPHOS proteins/complexes, for example, in tissue, cell or mitochondrial extracts. This important limitation rendered earlier anti-OXPHOS antibodies useless for isolating native OXPHOS proteins and/or the native OXPHOS complexes of which such proteins are part (see, for example, Haab et al., Genome Biol., 2: 1-13, 2001; Michaud and Snyder, BioTechniques, 33: 1308-1316, 2002; Kingsmore and Patel, Curr. Opin. Biotechnol., 14: 74-81, 2003). Thus, the disclosed capture antibodies satisfy a need in the art for immunological reagents that can detect deficiencies or alterations in the structure and/or function of one or more native OXPHOS proteins or native OXPHOS enzyme complexes. For example, the disclosed antibodies are of sufficient specificity and affinity that they can be used to capture detectable amounts of native target protein even if the target protein is present at low concentration and even if the sample contains large amounts of other non-target proteins. Such antibody reagents enable, at least, the detection and diagnosis of OXPHOS-related diseases.

The target antigen of a capture antibody disclosed herein is a native OXPHOS protein(s) (typically, but not always, a single OXPHOS protein) to which the antibody binds directly via a specific interaction between the antibody's binding site and a small region (epitope) on the surface of the target antigen(s). Non-limiting examples of OXPHOS proteins to which a capture antibody can bind include Complex I (CI)-39 kDa subunit, CI-30 kDa subunit, CI-20 kDa subunit, CI-17 kDa subunit, CI-15 kDa subunit, CI-14 kDa subunit, CI-8 kDa subunit, Complex II (CII)-70 kDa subunit, CII-30 kDa subunit, Complex III (CIII)-Core 1 subunit, CIII-Core 2 subunit, CIII-iron-sulfur subunit, CIII-10 kDa, Complex IV (CIV)-subunit I, CIV-subunit II, CIV-subunit III, CIV-subunit IV, CIV-subunit Va, CIV-subunit Vb, CIV-subunit VIa-H, CIV-subunit VIa-L, CIV-subunit VIb, CIV-subunit VIc, CIV-subunit VIIa-H/L, CIV-subunit VIIb, CIV-subunit SURF-1, Complex V (CV)-α subunit, CV-β subunit, CV-OSCP subunit, CV-d subunit, CV-IP subunit, CV-β subunit, CV-β subunit, or CV-β subunit). In particular examples, a target antigen of an anti-OXPHOS capture antibody is CI-8 kDa subunit, CI-15 kDa subunit (NDUFA6), CI-subunit GRIM 19, CI-20 kDa subunit (ND6), CII-70 kDa subunit, CIV-15 kDa subunit (likely subunit IV), CV-α subunit, CV-β subunit, CV-IP subunit, or CV-subunit ATP5J.

In specific embodiments, a capture antibody “captures” not only the target OXPHOS protein(s), but also any additional proteins specifically associated with the target antigen; for example, the target antigen assembled into all of part of a OXPHOS enzyme complex (such as, a partially assembled or disassembled OXPHOS complex, or an improperly assembled OXPHOS complex). The co-capture of target-antigen-associated proteins along with the specifically targeted OXPHOS protein can reveal structural and functional relationships between the target OXPHOS protein antigen and associated proteins.

A capture antibody, in some instances, recognizes an epitope contained within a single OXPHOS protein in its native conformation. In this case, the epitope may be preferentially (or only) available when the OXPHOS protein is substantially free of other associated proteins, such as other proteins of an OXPHOS complex; for example, the epitope may be masked when the single OXPHOS protein is incorporated into an OXPHOS complex. In other instances, an epitope contained within a single OXPHOS protein may be preferentially available whether or not the protein is incorporated into a fully or partially assembled OXPHOS complex (or otherwise specifically associated with other cellular components). In other instances, it is contemplated that the capture antibody recognizes an epitope available only (or preferentially available) on an assembled or partially assembled OXPHOS complex. For example, a capture antibody epitope might be formed only when two or more subunits are associated normally, in which case, the epitope can (but need not) consist of juxtaposed structural aspects provided by two or more subunits. In other embodiments, the epitope is preferentially (or only) formed in an assembled or partially assembled OXPHOS complex, at least in part, due to conformational changes caused by the association of subunits into the complex. Thus, it is contemplated that some capture antibodies will be specific for assembled (or partially assembled) OXPHOS complexes, and will be unable to specifically bind one isolated OXPHOS subunit. In other instances, the binding affinity of a capture antibody is measurably dissimilar for association to an isolated OXPHOS subunit and that subunit (or combination of subunits) when it (they) is (are) assembled into an OXPHOS complex (or part thereof). In some instances, the binding affinity of the capture antibody will be substantially similar for the isolated OXPHOS subunit and that subunit as incorporated into an OXPHOS complex.

In more particular embodiments a fully assembled immunocaptured OXPHOS enzyme complex has enzymatic activity, such as ATPase activity (e.g., Complex V), oxidoreductase activity (e.g., Complex I or III), reductase activity (e.g., Complex II), or oxidase activity (e.g., Complex IV).

Several representative OXPHOS capture monoclonal antibodies are shown in Table 1. Among the exemplar anti-OXPHOS capture antibodies are eight mAbs specific for native Complex I, one mAb specific for native Complex II, seven mAbs specific for native Complex III, six mAbs specific for native Complex IV, and thirteen mAbs specific for native Complex V. Table 1 indicates to which mitochondrial enzyme complex the mAb binds (“Antigen”) and, more specifically in some examples, to which subunit of the complex the mAb binds on a Western blot (“WB MW”). The isotypes of indicated mAbs are shown, as are the species specificities of the listed mAbs. Each of these antibodies is commercially available, for example, from MitoScience (Eugene, Oreg.).

The disclosed library of anti-OXPHOS capture antibodies operationally define those surface domains (epitopes) of the OXPHOS system that serve as useful binding sites for immunocapture techniques. It is probable that the epitopes bound by the disclosed capture antibodies encompass the most immunogenic and available sites present on the target OXPHOS complex. It is known that certain regions of proteins, when injected as antigens to stimulate antibody production, tend to dominate the immune response, because they are the most highly immunogenic (Berzofsky and Berkower, Immunogenicity and Antigen Structure, in Fundamental Immunology, 4th ed., ed. by Paul, Lippincott-Raven Publishers, 1999, pages 651-699). Conversely, some regions never provoke a significant immune response (Berzofsky and Berkower, Immunogenicity and Antigen Structure, in Fundamental Immunology, 4th ed., ed. by Paul, Lippincott-Raven Publishers, 1999, pages 651-699). Therefore, first-identified epitopes (such as those represented by the mAb library disclosed herein) are most likely to dominate repeated independent immunizations. Resultantly, the disclosed capture antibody library likely represents most of the species that make up a genus of anti-OXPHOS capture antibodies. Moreover, each genus of capture antibodies that specifically binds a particular native OXPHOS complex (such as, Complex I, II, III, IV, or V) is represented by the species identified, for example, in Table 1.

This disclosure also relates to hybridoma cell lines that produce monoclonal antibodies having the specificity of the capture antibodies described herein.

TABLE 1
OXPHOS Immunocapture Monoclonal Antibodies

| | WB | | | WB | | | | | | |
|---|---|---|---|---|---|---|---|---|---|---|
| Antigen | MW | MAb | Isotype | Conc | IC Conc | Capture | Human | Mouse | Rat | Bovine |
| Complex I | | | | | | | | | | |
| C-I-Capture 1 | ? | RAC#24-20D1AB7 | IgG2b, k | − | ? | + | + | | | + |
| C-I-Capture 2 | 08 kD | RAC#24-18G12BC2AA10 | IgG2b, k | + | + | + | | | | + |
| C-I-08 | 08 kD | RAC#24-17C8E4E11 | IgG1, k | 1 ug/ml | HH | + | +(HB) | + | + | + |
| | | | | | 5 ug/ml | | | | | |
| C-I-15 | 15 kD | RAC#24-17G3D9E12 | IgG1, k | 0.25 ug/ml | HH | + | + | −(+100 kd) | + | + |
| NDUFA6 | | | | | 5 ug/ml | | | | | |
| C-I-GRIM 19 | 19 kD | RAC#29-1D4 | nd | + | − | + | + | nd | nd | + |
| C-I-GRIM 19 | 19 kD | RAC#29-4G6BB9 | IgG | + | − | + | + | nd | nd | + |
| | | | | | (cyto+)HH | | | | | |
| C-I-GRIM 19 | 19 kD | RAC#29-6E1BH7 | IgG2b, k | + | +HH | + | + | nd | nd | + |
| C-I-20 | 20 kD | RAC#24A-20E9DH10C12 | IgG1, k | 0.5 ug/ml | − | + | + | + | nd | + |
| (likely ND6) | | | | | | | | | | |
| Complex II | | | | | | | | | | |
| C-II-Capture | 70 kD? | RAC#23C-4H12BG12AG2 | IgG1, k | +? | | + | + | nd | nd | + |
| Complex III | | | | | | | | | | |
| C-III-Capture | 70 kD | RAC#23B-1A11BC12AB9 | IgG2a, k | 5 ug/ml | nd | + | + | nd | nd | + |
| C-III-Capture | 70? | RAC#23C-4H12BC11BC5 | IgG1, k | +? | ? | + | + | nd | nd | + |
| C-III-Capture | ? | RAC#23B-10D2 | nd | nd | nd | + | + | nd | nd | + |
| C-III-Capture | neg | RAC#23C-11A51H12 | IgG1, k | − | nd | + | + | nd | nd | + |
| C-III-Capture | 70 kd | RAC#23C-12G8 | nd | + | | + | + | nd | nd | + |
| C-III-Capture | 70? | RAC#23C-17A81A8 | nd | +? | | + | + | nd | nd | + |
| C-III-Capture | neg | RAC#23C-29C2 | nd | − | | + | + | nd | nd | + |
| Complex IV | | | | | | | | | | |
| C-IV capture | ? | RAC#11B-7E5BA4 | IgG1, k | − | ? | + | + | nd | nd | + |
| C-IV capture | 15 kD | RAC#23C-21H10 | nd | + | | + | + | nd | nd | + |
| C-IV capture | ?30/70 | RAC#23C-22D5 | nd | +? | | + | + | nd | nd | + |
| C-IV capture | ?15/10 | RAC#23C-22H11G43E1 | IgG1, k | +? | | + | + | nd | nd | + |
| C-IV capture | 15 kD | RAC#23C-28G7 | nd | + | | + | + | nd | nd | + |
| C-IV capture | 15 kD | RAC#23C-31E91B82G9 | IgG1, k | + | | + | + | nd | nd | + |
| Complex V | | | | | | | | | | |
| C-V-F1 capture | ? | MM#1-12F4AD8AF8 | IgG2b, k | − | − | + | + | nd | nd | + |
| C-V-Beta | 52 kD | MM#7-3D5AB1 | IgG1, k | <0.5 ug/ml | + | + | + | + | nd | + |
| capture | | | | | | | | | | |
| C-V-Alpha | 53 kD | MM#1-7H10BD4F9 | IgG2b, k | <0.5 ug/ml | 5 ug/ml | +/− | + | + | + | + |
| C-V-Capture | nd | RAC#23C-1G1 | nd | nd | | + | + | nd | nd | + |
| C-V-Capture | 70? | RAC#23C-24C9 | nd | ? | | + | + | nd | nd | + |
| C-V-Capture | ˜20 kD | MM#1-8E12 | nd | + | − | + | + | nd | nd | + |
| C-V-IP | 10 kD | RAC#25A-5E2D7 | IgG1, k | <0.5 ug/ml | 5 ug/ml | + | + | + | +/− | + |
| | | | | | | | (10/18) | (10/18) | (18 += | (10/18) |
| | | | | | | | | | COX4?) | |
| C-V-ATP5J | 8 kD | RAC#29-2A5 | nd | + | + | + | + | nd | nd | + |
| C-V-ATP5J | 8 kD | RAC#29-6G5 | nd | + | +HH | +/− | + | nd | nd | + |
| C-V-ATP5J | 8 kD | RAC#29-8C7CC4 | IgG. | + | +HH | + | + | nd | nd | + |
| C-V-ATP5J | 8 kD | RAC#29-9G3 | nd | + | + | + | + | nd | nd | + |
| C-V-ATP5J | 8 kD | RAC#29-10A3 | nd | + | + | +/− | + | nd | nd | + |
| C-V-ATP5J | 8 kD | RAC#29-10C6AC9 | IgG1, k | + | + | + | + | nd | nd | + |

HH = Heat-Induced-Antigen-Retrieval (20 min incubation at 90-100 C in 0.1 M Tris/HCl pH 9.5, with 5% urea (wt/vol)) preferred for optimal reactivity to antigens fixed with aldehydes.
nd = not determined

A. Monoclonal Antibody Production

Monoclonal antibodies can be prepared from murine hybridomas according to the classical method of Kohler and Milstein (Nature, 256: 495-497, 1975) or derivative methods thereof (for example, Marusich, J. Immunol. Meth., 114: 155-159, 1988). In one specific, non-limiting embodiment, a mouse is repetitively inoculated with a few micrograms of the selected protein over a period of a few weeks to several months. Concentration of protein in the final preparation can be adjusted, for example, by concentration on an Amicon filter device, to the level of a few micrograms per milliliter.

Various immunogens containing OXPHOS enzyme complexes, subunits (OXPHOS proteins) or fragments thereof may be used to produce antibodies in mice. For example, whole mitochondria may be used as an immunogen. In other examples, one or more substantially pure OXPHOS enzyme complex(es) or subunit(s) thereof are suitable immunogens. Substantially pure enzyme complexes or their subunits can be isolated, for example, from wild-type cells or cells transfected with nucleic acid sequences encoding one or more subunits of one or more OXPHOS enzyme complexes. In addition, recombinant nucleic acids encoding one or more subunits of one or more OXPHOS enzyme complexes (such as, naked DNA or mammalian expression vectors) can be directly injected into mice so that the subsequently expressed protein serves as an immunogen.

Alternatively, peptide fragments from one or more subunit(s) of an OXPHOS enzyme complex may be utilized as immunogens. Such fragments may be synthesized chemically using standard methods, or may be obtained by cleavage of an isolated OXPHOS enzyme complex or its individual subunit(s) followed by purification of the desired peptide fragments. Peptides as short as three or four amino acids in length are immunogenic when presented to an immune system in the context of a major histocompatibility complex (MHC) molecule, such as MHC class I or MHC class II. Accordingly, peptides comprising at least 3 and preferably at least 4, 5, 6 or more consecutive amino acids may be employed as immunogens for producing antibodies. Because naturally occurring epitopes on proteins frequently comprise amino acid residues that are not adjacently arranged in the peptide when the peptide sequence is viewed as a linear molecule, it may be advantageous to utilize longer peptide fragments from an OXPHOS enzyme complex or its individual subunit(s) for producing antibodies, for example at least 10, 15, 20, 25, or 30 consecutive amino acid residues may be employed.

In some examples, it is advantageous to select peptides from the C- or N-terminus of the target subunit or from another region of the target subunit that is likely to be exposed on the surface of the full-length protein. Selecting peptide immunogens from hydrophilic protein regions or regions of high mobility may also be advantageous. Structural analyses of an OXPHOS enzyme complex and/or its subunits are useful for such determinations and computer programs are also available to predict structural elements from the known sequences of the proteins. Complex II (Xia et al., Science, 277: 60-66, 1997; Iwata et al., Science, 281: 64-71, 1998), Complex IV (Tsukihara et al., Science, 272: 1136-1144, 1996) and Complex V (Abrahams et al., Nature, 370: 621-628, 1994) have each been crystallized and, therefore, their molecular structures are known in great detail. Complex I structural information is available to about 20 angstrom resolution (Grigorieff, J. Mol. Biol., 277: 1033-1046, 1998); moreover, water-soluble subcomplexes of Complex I, which are likely to be more immunogenic than membrane-embedded domains of the complex, have been characterized (Sazanov and Walker, J. Mol. Biol., 302: 455-464, 2000).

After a period of immunization, the inoculated mouse is sacrificed, and the antibody-producing cells of the spleen isolated. The spleen cells are fused with mouse myeloma cells using polyethylene glycol, and the excess, non-fused, cells destroyed by growth of the system on selective media comprising aminopterin (HAT media). Successfully fused cells are diluted and aliquots of the dilution placed in wells of a microtiter plate, where growth of the culture is continued. Antibody-producing clones are identified by detection of antibody in the supernatant fluid of the wells by any suitable immunoassay procedures, including without limitation those methods described below. Selected positive clones can be expanded and their monoclonal antibody product harvested for use. Detailed procedures for monoclonal antibody production are described in Harlow and Lane (Antibodies, A Laboratory Manual, Cold Spring Harbor Laboratory Press, New York, 1988).

B. Detecting mAbs Specific for OXPHOS Enzyme Complexes

The determination that an antibody specifically binds to an antigen is made by any one of a number of standard immunoassay methods; for instance, Western blotting (see, Sambrook et al. (eds.), Molecular Cloning: A Laboratory Manual, 2nd ed., vols. 1-3, Cold Spring Harbor Laboratory Press, Cold Spring Harbor, N.Y., 1989). To determine that a given antibody preparation specifically detects an OXPHOS enzyme complex (or a subunit thereof) by Western blotting, total cellular protein is extracted from cells or mitochondria and electrophoresed on an SDS-polyacrylamide gel. The proteins are electrophoretically transferred to a membrane (for example, nitrocellulose), and the antibody preparation is incubated with the membrane. After washing the membrane to remove non-specifically bound antibodies, the presence of specifically bound antibodies is detected by the use of a detector molecule (such as, an anti-mouse antibody conjugated to an enzyme such as alkaline phosphatase). Antibodies that specifically detect a subunit of an OXPHOS complex will be shown, by this technique, to bind substantially only a single band corresponding to the molecular weight of the particular subunit to which the antibody binds.

In some circumstances, it is advantageous to determine if an antibody specific for a particular enzyme subunit can also immunocapture all or part of the OXPHOS enzyme complex of which the subunit is part. For this purpose, the antibody to be tested can be bound to a solid support; for example, by adsorbing the antibody to the solid support, or by pre-coating the solid support with a goat anti-mouse antibody, which binds the test antibody (for instance, by its Fc region so as not to interfere with the antigen binding sites of the test antibody). The immobilized test antibody is then incubated with a mitochondrial protein preparation, which is prepared so as to preserve intact (native) OXPHOS enzyme complexes. For example, solubilizing mitochondria in a gentle detergent, such as n-dodecyl-β-D-maltoside, is known to preserve intact OXPHOS enzyme complexes. After an incubation period, non-bound contaminants can be removed and protein(s) bound to the immobilized antibody are released (for example, using SDS) and characterized by gel electrophoresis.

Purified Complex I, II, III, IV, or V each have a characteristic subunit protein pattern on an SDS polyacrylamide gel (see, for example, Murray et al., Electrophoresis, 25: 2520-2525, 2004). Thus, an antibody capable of immunocapturing an intact (fully assembled) OXPHOS enzyme complex can be identified, for example, by the banding pattern of the protein that it captures in the foregoing assay. In some examples, a partially assembled OXPHOS complex is immunocaptured. In this case, fewer than all of the subunits of an OXPHOS enzyme complex would be observed by gel electrophoresis, but the partial pattern would still be recognizable.

In more specific examples, it is of interest to determine whether an immunocaptured OXPHOS enzyme complex has functional activity. Functional assays for each of the OXPHOS enzyme complexes (such as, Complex I, II, III, IV or V) are known in the art (see also, section entitled “OXPHOS Enzyme Activity Assays” herein). Such assays may be performed directly in a 96-well plate. A mAb is identified as specific for a functional OXPHOS enzyme complex when protein it binds exhibits an enzymatic activity characteristic of Complex I, II, III, IV, or V.

An anti-OXPHOS capture antibody can also be identified by performing a mass spectrometric analysis on the immunocaptured antigen. Complex I, II, III, IV and V (and their respective subunits) exhibit characteristic peptide mass fingerprints (see, for instance, Examples 1 and 7). Hence, it is possible to identify an antigen bound by an anti-OXPHOS capture antibody (and, thereby, determine the specificity of the antibody) with this technique.

C. High-Throughput Screening of Hybridomas

Antibodies capable of capturing all or part of an OXPHOS complex are relatively rare. To identify such antibodies, it is advantageous to screen large numbers of hybridomas. Any high-throughput system for hybridoma screening known in the art may be performed. In one example, hybridoma supernatants are placed in discrete locations on a solid support, such as in individual wells of a multi-well (such as, a 96-well) tissue culture plate. Proteins present in the hybridoma supernatant, such as monoclonal antibodies, are directly (for instance, by adsorption) or indirectly (for instance, by pre-adsorbing goat anti-mouse antibody (to Fc region) to the solid support) immobilized on the solid support. A sample containing intact OXPHOS enzymes (such as cells or mitochondrial solubilized in a gentle detergent, such as n-dodecyl-β-D-maltoside) is incubated with the immobilized hybridoma proteins (or antibodies). In some examples, proteins in the sample are labeled, for example, with a sulfhydryl-reactive or amine-reactive dye. After sufficient time to permit binding of mitochondrial proteins to mAbs (for example, from about 1 to about 2 hours), unbound contaminants are removed using any solution that will not disrupt antibody/antigen interactions (such as, phosphate-buffered saline containing a gentle, non-ionic detergent at low concentration (for instance, 0.05% Tween-20)).

Any method of detecting binding of OXPHOS enzyme complexes (or one or more subunits thereof) to immobilized hybridoma mAbs may be used. When using a pre-labeled mitochondrial protein sample, bound proteins can be directly detected. Because each OXPHOS enzyme complex (such as, Complex I, II, III, IV or V) has intrinsic activity, it is possible to detect the binding of one or more such complexes by performing any of several functional assays known in the art for each complex (see also, section entitled “OXPHOS Enzyme Activity Assays” herein). Such assays may be performed directly in a 96-well plate used to screen a number of hybridoma supernatants. In addition, detectable binding agents (such as, enzyme-conjugated secondary antibodies) specific for an OXPHOS complex of interest (or subunit thereof) may be used to detect the presence of an immunocomplex between the OXPHOS complex (or subunit thereof) and an immobilized hybridoma mAb.

Proteins detectably bound to an immobilized hybridoma mAb (such as, one or more bound OXPHOS enzyme complexes), optionally, can be released from the mAb (and other detection agents) and thereby isolated. In some instances, isolated OXPHOS enzyme complexes may be further characterized, for example, by determining a function activity thereof, by separating subunits thereof by gel electrophoresis to determine the number of subunits present (or, to confirm the presence the expected number of subunits) and/or to examine post-translational modifications of the subunits.

D. Competitive Inhibitors of Anti-OXPHOS Capture Antibodies

As discussed above, the disclosed library of anti-OXPHOS capture antibodies operationally define those surface domains (epitopes) of the OXPHOS system that serve as useful binding sites for immunocapture techniques. It is probable that the epitopes bound by the disclosed capture antibodies encompass the most immunogenic and available sites present on the target OXPHOS complex. Accordingly, also disclosed herein are antibodies that bind to the same or a sterically overlapping epitope of a disclosed anti-OXPHOS capture antibody (such as those listed in Table 1).

Methods of epitope mapping are common in the art (see, for example, Harlow and Lane, Using Antibodies: A Laboratory Manual, Cold Spring Harbor, N.Y.: Cold Spring Harbor Press, 1999, Chapter 11; Harlow and Lane, Antibodies: A Laboratory Manual, Cold Spring Harbor, N.Y.: Cold Spring Harbor Press, 1988, p. 590; Jia et al., J. Immunol. Meth., 288(1-2): 91-98, 2004). The simplest way to determine whether two monoclonal antibodies bind to the same (or overlapping) epitope(s) on a protein antigen (such as, an OXPHOS protein or complex) is to carry out a simple competition binding assay. If there is competition (that is, the two antibodies interfere with each other's ability to bind the antigen), then the two antibodies recognize the identical or sterically overlapping epitopes. If the antibodies do not affect each other's binding to the same antigen, then they likely recognize distinct epitopes.

There are many assays known in the art that measure the competition of antibodies for binding to an antigen (see, for example, Wagener et al., J. Immunol., 130: 2308-2315, 1983; Ransom, Practical Competitive Binding Assay Methods, Philadelphia: Elsevier (C.V. Mosby), 1976; Harlow et al., Antibodies: A Laboratory Manual, Cold Spring Harbor Laboratory, Cold Spring Harbor, N.Y., 1988, page 590).

In one representative competition assay, a first antibody (such as a first anti-OXPHOS capture mAb) is immobilized to a solid support, such as to one or more wells of a 96-well microplate. A labeled target antigen (for example fluorochrome-labeled OXPHOS complex) is then applied in a solution that either contains or lacks a soluble second antibody (such as a second anti-OXPHOS mAb). After sufficient time for antibody-antigen interaction (for example, 30-60 minutes), the unbound target antigen and unbound second antibody are both washed away. Binding of the labeled target antigen by the immobilized first antibody can be determined by measuring the amount of label (e.g., fluorescence) bound to the well. If the second antibody binds to the same epitope or an overlapping epitope as the immobilized first antibody, then the second antibody will compete with the first antibody for this binding site, thereby reducing the amount of labeled target antigen bound per well. The amount of such reduction will vary as a function of second antibody concentration.

IV. OXPHOS Protein Immunocapture and Related Methods

The disclosed antibodies, methods and kits permit detection of deficiencies or alterations in one or more OXPHOS proteins or enzyme complexes to enable, for example, the detection and diagnosis of OXPHOS-related diseases. In particular examples, it is now possible (i) to screen subjects to identify those having (suspected of having) an inherited mitochondrial disease, a late onset mitochondrial disorder, or an environmental toxin-induced mitochondrial dysfunction; (ii) monitor and manage mitochondrial diseases and to guide therapy for mitochondrial disease; (iii) monitor the course of mitochondrial disease in trials of potential therapeutic drugs to help assess efficacy of these drugs in treating/preventing mitochondrial disease; (iv) screen drugs for unintended mitotoxic effects, and other substances, including potential environmental toxins, for mitotoxic effects when delivered either in vivo (and the OXPHOS complexes captured and analyzed post-exposure) or in vitro (applied directly to OXPHOS complexes captured before exposure); (v) screen at-risk populations, e.g., farm workers that handle pesticides, for evidence of exposure to mitotoxic agents, (vi) screen populations to identify individuals susceptible to mitotoxic drugs and other substances, including potential environmental toxins (by immunocapturing OXPHOS complexes directly from each individual and then determining the sensitivity of their personal OXPHOS enzymes. In addition, the disclosed antibodies, methods and kits are useful to screen for protective drugs that can prevent or treat mitochondrial disorders.

The following description is equally applicable to each OXPHOS complex (such as, Complex I, II, III, IV, or V) or individual OXPHOS protein subunit or any combination thereof for which a capture antibody is described herein.

A. Qualitative and Quantitative Detection of Native OXPHOS Complexes or Proteins

Provided herein are methods for determining the amount of one or more OXPHOS complexes (e.g., Complex I, II, III, IV and/or V) or OXPHOS proteins in a biological sample of a subject by contacting isolated “capture” antibodies that specifically bind (immunocapture) the native target OXPHOS complex(es) or protein(s) with a sample comprising solubilized mitochondrial proteins and protein complexes, so that the antibodies bind to target OXPHOS complex/protein present in the sample to form an antibody/target assemblage. In some embodiments, remaining sample contents are separated from the antibody/target assemblage. The amount of captured OXPHOS protein or captured OXPHOS complex in the antibody/target assemblage is determined.

Any methods known in the art can be used to measure the amount of OXPHOS protein and/or OXPHOS complex present in the antibody/target assemblage. In one embodiment, substantially all proteins in the sample can be pre-labeled with one or more commercially available protein-reactive fluorescent dyes (for example, amine-reactive succinimidyl esters of fluorochromes or sulfhydryl-reactive maleamide fluorochrome derivatives; exemplar fluorochromes include fluorescein isothiocyanate (FITC), rhodamine isothiocyanate, Texas Red™, Oregon Green™, any of the Alexa™ dyes, any of the Cy-dyes). Then, following immunocapture, the amount of OXPHOS protein (or OXPHOS complex) is determined by measuring the amount of fluorescence in the antibody/target assemblage.

In another embodiment, the amount of captured OXPHOS protein or OXPHOS complex in an antibody/target assemblage can also be measured by contacting the assemblage with a second anti-OXPHOS antibody. A second anti-OXPHOS antibody preferably does not bind to the same epitope as the original capture antibody. The second anti-OXPHOS antibody can be directly detectable (for example, by incorporation of a fluorescent or radioactive tag or by linkage to an enzyme or enzymatic domain that produces a detectable product). Alternatively, the second anti-OXPHOS antibody can be detected indirectly by subsequent contact with another detectable, specific reagent. A detectable specific reagent specifically binds the second anti-OXPHOS antibody and, preferably, does not bind the original capture antibody. Appropriate detectable, specific reagents are well known in the art and include, for example, labeled (or enzyme-linked) antibody specific for the second anti-OXPHOS antibody (such as, labeled (or enzyme-linked) goat anti-mouse antibody). In particular examples, an isotype-specific detectable specific reagent can be used where the first and second anti-OXPHOS antibodies are differing isotypes.

A second anti-OXPHOS antibody used to detect a capture antibody/target assemblage need not be another “capture” antibody. However, in some circumstances, the use of a second capture mAb can simplify the detection step because, for example, additional steps are typically needed to expose epitopes for non-capture antibodies (such as, fixation or SDS denaturation). When using a non-capture second anti-OXPHOS antibody for detection purposes, the capture antibody/target OXPHOS protein (or complex) assemblage can be stabilized in any number of ways, including fixation or covalent cross-linking to the original capture antibody and, then, treated as needed to render the epitope of the second anti-OXPHOS antibody accessible.

Optionally, the analysis of more than one OXPHOS complex can be examined using a set of monoclonal capture antibodies that are specific for the respective target complexes.

In some of the disclosed methods, it can be advantageous to separate the capture antibody/target assemblage from free antigen and other soluble contaminants in the sample. In some examples, such separation can be accomplished by immobilizing the capture antibody. Methods of immobilizing antibodies are well known in the art and include, for example, covalent or non-covalent attachment of the antibody to solid supports, such as glass, cellulose, nitrocellulose, polyacrylamide, nylon, polystyrene, polyvinyl chloride, polypropylene, or polymerized dextrans. Such solid supports can take any number of forms, such as microtitre plates, beads, or resins (which may be packed into columns).

When the target antigen (such as, a native OXPHOS protein or complex) is bound to an immobilized capture antibody, the immobilized antibody/target assemblage can be physically separated from non-immobilized components of the sample, for example, by removing non-immobilized components (such as, by washing a microtitre plate to which the antibody/target assemblage is immobilized) and/or by selectively collecting the immobilized antibody/target assemblage (such as, by centrifugation of beads to which the antibody/target assemblage is immobilized).

Certain disclosed methods involve single step, or homogeneous, non-separation assays, which do not entail capture antibody immobilization or separation of antibody-bound OXPHOS protein from non-bound sample components. One example involves the spontaneous formation of an insoluble lattice of interconnected antigens (e.g., OXPHOS protein or complex) and antibodies (e.g., capture antibodies). This type of assay involves contacting a sample containing an OXPHOS antigen that is at least bivalent (that is, has at least two copies of at least one epitope present in two locations on the antigen) with an antibody that is at least bivalent (that is, has at least two epitope binding sites in a single molecule). Non-limiting examples of bivalent OXPHOS antigens include Complex IV and Complex V. For example, Complex IV is normally present as a dimer; therefore, all Complex IV epitopes are present in two copies per complex. As another example, Complex V includes two of each of the α and β subunits and approximately ten of the c subunit; therefore all epitopes present on Complex V subunits α, β, and c would be suitable bivalent target epitopes. IgG mAbs (and certain fragments thereof) are bivalent and are suitable for such assays. At certain concentrations of antibody and antigen, where neither the antigen nor the antibody is in excess and both reactants are present at sufficient concentration, most antibodies will bind two separate antigens and most antigens will be bound by two separate antibodies to form insoluble aggregates. An ordinarily skilled artisan can empirically determine the ratios and absolute concentrations for a particular antibody/antigen pair.

Insoluble antibody/antigen aggregates can be observed qualitatively and/or quantified without removal of other soluble components. Aggregation can be measured, for example, as an increased turbidity of a sample or, in another example, as a zone of aggregation “precipitin lines” in a gel of agarose. In the latter example, antibody and antigen are applied in adjacent wells of an agarose gel and allowed to diffuse toward each other. The precipitin lines form spontaneously in zones where the antibody/antigen ratios and absolute concentrations of each are favorable for the required aggregation interaction. This format also enables quantitative measurement of the relative amount of antigen in different samples as the precipitin zone will be localized closer to antigen wells of low concentration and farther from antigen wells of high concentration. The precipitin lines can be visualized directly without further manipulation as opaque lines and sensitivity can be improved if the gels are first washed, and the precipitates then stained with any of a number of common protein reactive dyes such as Coomassie brilliant blue or amido black.

Optionally and beneficially, an antibody/antigen aggregation assay test can be made more sensitive if (i) the insoluble aggregates are washed free of soluble components and stained with a protein-reactive dye prior to quantification of the amount of dye bound to the aggregate; or (ii) the target antigen is labeled (for example, with a fluorescent or radioactive tag) prior to mixing the antibody with the antigen-containing sample).

Another example of a homogeneous, non-separation based assay useful for detection and quantification of immunocaptured OXPHOS protein(s) or complex(es) is measurement of target antigen concentration by antigen-induced inhibition of fluorescence polarization (see, for example, Dandliker et al., Immunochemistry, 10: 219-227, 1973; Dandliker and De Saussure, Immunochemistry, 7: 799-828, 1970; High-Throughput Screening: The Discovery of Bioactive Substances, ed. by J. P. Devlin, published by M. Dekker, 1997). In this assay, a trace amount of fluorescently labeled, freely soluble small target antigen shows no fluorescence polarization when excited by a polarized light source, because the molecules are free to tumble randomly in the time period between excitation and emission. By comparison, the same labeled soluble target antigens show strong fluorescence polarization if bound (still in solution) by soluble antibodies because the antibody-antigen complex tumbles much more slowly. Addition of unlabeled antigen competes for antibody binding sites, leaving the labeled antigen free and unpolarized. The inhibition of fluorescence polarization is directly proportional to the concentration of unlabeled antigen (such as OXPHOS protein or complex). In particular examples, the labeled tracer antigen is a small fluorescently labeled peptide that contains the target OXPHOS protein epitope, which eliminates the need to use a native OXPHOS protein antigen or an OXPHOS complex containing such antigen as the tracer.

The amount of immunocaptured OXPHOS enzyme complex can also be measured by using an intrinsic property of the immunocaptured OXPHOS enzyme. For example, immunocaptured Complex V can be quantified by measuring ATP hydrolysis activity of the immunocaptured material (Aggeler et al., J. Biol. Chem., 277: 33906-33912, 2002). Analogous enzyme assays can be performed for each of the remaining OXPHOS enzyme complexes (see, for example, Examples 1, 7, 8, 9 and 10, and section herein entitled “OXPHOS Enzyme Activity Assays”).

Any biochemical activity of an immunocaptured OXPHOS enzyme (e.g., Complex I, II, III, IV, or V) can be used as a qualitative or quantitative marker of activity function, because the antibody/antigen capture of the target OXPHOS complex provides specificity to the assay. This specificity ensures, for example, that substantially all of the “captured” enzyme activity is attributable to the targeted OXPHOS enzyme complex. This feature advantageously simplifies the type and number of enzyme activity assays that need to be run because, for example, crude cell extracts (or even extracts of purified mitochondria) contain enzymes with biochemical activities similar to each of the OXPHOS complex and these non-OXPHOS enzymes contribute to the measured signal. Therefore, in non-immunocapture assays it is necessary to run each sample in a set of parallel assays with and without a variety of specific enzyme inhibitors and, thereafter, calculate the amount enzyme activity that is due to only the OXPHOS enzyme of interest.

Another benefit of using intrinsic enzyme activity of the immunocaptured OXPHOS enzyme complex is that such an assay can detect a wide variety of defects in OXPHOS complexes including, for example, the presence or absence of one or more target subunits; misassembly of the OXPHOS complex, catalytic defects, post-translational modifications that inactivate or activate the OXPHOS complex, or lack of a normally expressed post-translational modification.

If quantification of captured OXPHOS complex(es) is combined with quantification of enzyme activity, the specific activity of the captured enzyme can be determined. Specific activity can be used to distinguish between alterations in enzyme turnover rates from alterations in enzyme amounts.

Assays provided herein can be used to determine whether a particular OXPHOS complex or protein subunit is produced in low quantity as compared to control values obtained from corresponding control samples. For example, “corresponding samples” would be mitochondria isolated from a test subject (e.g., patient) fibroblast cell line compared with mitochondria isolated from a control skin fibroblast cell line (for instance, isolated from skin fibroblasts of a normal individual or a group of normal individuals). In addition to fibroblasts, mitochondria protein-containing samples for use in the described methods can be obtained from whole cell extracts or from mitochondria isolated from such cells. Many cell and tissue sources are suitable for analysis as mitochondria are found in almost all cells and tissues. These include tissues commonly used for routine diagnostic analysis, such as peripheral blood cells, shed epithelial cells from various mucosa, and tissue biopsies, in particular muscle biopsies.

Although fibroblast cells are particularly convenient as a source of patient samples for diagnostic assays, it should be understood that OXPHOS complexes and OXPHOS proteins subunits can be isolated from any mammalian cell, including human cells, with cells having high energy requirements having the largest supply of mitochondrial proteins. For example, cells that can be used in the provided methods include (but are not limited to) skeletal muscle cells, skin cells, adipose cells, neural cells, cardiomyocytes, pancreatic islet cells, hematopoietic cells, liver cells, kidney cells, T cells, B cells, mucosal epithelial cells and other cell types. Examples of tissue samples that can be utilized to obtain cells for use in the methods include saliva, mucosal cells and semen, for example. Alternatively, the assay can be performed utilizing OXPHOS complex(es) or mitochondria that have been immunopurified from patient cells or experimental cells by any method known in the art, such as the methods described in the Examples herein.

In specific embodiments, methods of detecting OXPHOS protein(s) or complex(es) are used to identify OXPHOS deficiency in a subject by contacting one or more monoclonal antibodies specific for each of a plurality of subunits of one or more OXPHOS enzyme complexes with a subject sample so that the antibodies immunocapture subunits present in the sample. The antibodies are each conjugated to a detectable label. The amount of each subunit immunocaptured by a respective antibody is then determined and the amount of each of the subunits is compared with an amount thereof present in a corresponding normal (e.g., non-disease, pre-treatment, etc.) sample. A decrease in the amount of any of the subunits in the subject's test sample as compared to the normal (or control) sample indicates the presence of an OXPHOS deficiency in the subject.

B. OXPHOS Complex Assembly Profile

Certain defects of the OXPHOS system can be traced to failure of one or more OXPHOS complexes to properly assemble. For example, Complex I fails to properly assemble when one or more of its subunits contain particular mutations (Triepels et al., J. Biol. Chem., 276: 8892-8897, 2001). The disclosed immunocapture methods can also be used to determine the assembly status of OXPHOS enzyme complexes. The immunocaptured material can be analyzed in any number of ways to determine the subunit composition, and hence assembly status, of the immunocaptured OXPHOS complex. These methods include, without limitation (i) one-dimensional SDS-PAGE followed by protein staining or mass spectrometry (see, for instance, various Examples herein, and Aggeler et al., J. Biol. Chem., 277: 33906-33912, 2002; Murray et al., J. Biol. Chem., 278(39): 37223-37230, 2003; Murray et al., Electrophoresis, 25: 2520-2525, 2004); (ii) two-dimension gels (isoelectric focusing and SDS-PAGE) followed by protein staining or mass spectrometry (see, for instance, various Examples herein and Aggeler et al., J. Biol. Chem., 277: 33906-33912, 2002; Murray et al., J. Biol. Chem., 278(39): 37223-37230, 2003; Murray et al., Electrophoresis, 25: 2520-2525, 2004); (iii) one-dimension SDS-PAGE followed by western blotting with appropriate subunit-specific antibodies (see, for instance, Aggeler et al., J. Biol. Chem., 277: 33906-33912, 2002; Murray et al., Electrophoresis, 25: 2520-2525, 2004; (iv) MudPIT (Multidimensional Protein Identification Technology) mass spectrometry (see, for instance, Eng et al., J. Am. Soc. Mass Spectrom., 5: 976-989, 1994; Link et al., Nat. Biotechnol., 17(7): 676-82, 1999; Washburn et al., Nat. Biotechnol., 19(3): 242-7, 2001; Lin et al., American Genomic/Proteomic Technology, 1(1): 38-46, 2001; Tabb et al., J. Proteome Res., 1: 21-26, 2002), and (v) direct detection of individual OXPHOS proteins using a second anti-OXPHOS antibody (see, for instance, Aggeler et al., J. Biol. Chem., 277: 33906-33912, 2002; Murray et al., J. Biol. Chem., 278(39): 37223-37230, 2003; Murray et al., Electrophoresis, 25: 2520-2525, 2004). In addition to antibodies that bind to a fully assembled OXPHOS enzyme complex, capture antibodies that are known to bind specifically to a subcomplex (a partially assembled complex lacking one or more subunit proteins) or to a single particular subunit can be used to determine the amount of the respective subcomplex or subunit being produced by the subject whose sample is being analyzed.

C. Detection of Post-Translational Modifications

Post-translational modifications of one or more OXPHOS protein subunits (such as, a Complex I subunit) are believed to occur in the pathogenesis of late-onset mitochondrial disorders such as, Parkinson's disease, late onset diabetes (NIDDM), Huntington's and Alzheimer's diseases, amyotrophic lateral sclerosis, schizophrenia, and the like. The presence of one or more post-translational modifications of one or more OXPHOS proteins that is different from those post-translational modifications seen in normal samples identifies a subject as having (or as a candidate for having) a late-onset mitochondrial disorder. Thus, disclosed herein are methods of identifying post-translational modifications of immunocaptured OXPHOS protein or complexes by immunocapture of one or more OXPHOS proteins or complexes followed by analysis of immunocaptured material by mass spectrometry (see, for instance, Examples 1 and 7, and Murray et al., Electrophoresis, 25: 2520-2525, 2004), or additional antibodies specific for the post-translational modification.

Consistent correlation of a particular OXPHOS protein post-translational modification (or set of post-translational modifications) with a particular late-onset mitochondrial disease identifies the post-translational modification(s) as surrogate markers (disease biomarkers) for that disease. Disease biomarkers can be used to diagnose disease, monitor disease progression and monitor the efficacy of therapeutic or preventative treatments. The described antibody reagents and assays can be used to identify and characterize mitochondrial disease biomarkers.

Pathogenic post-translational modifications to OXPHOS proteins (such as, Complex I subunits) are thought to be caused by oxidative damage and result in one or more subunits having oxidized amino acids with characteristic chemical, structural, antigenic modifications and/or molecular weight differences. Oxidative damage to OXPHOS proteins can occur under physiological conditions through the action of reactive oxygen species, including those containing nitrogen such as peroxynitrite (ONOA-). Peroxynitrite has been shown in vitro to target tyrosine residues in proteins through free radical addition to produce 3-nitrotyrosine. Mass spectral patterns associated with 3-nitrotyrosine containing peptides allow identification of peptides containing this modification. For example, matrix-assisted laser desorption/ionization (MALDI) mass spectrometry has previously been used to characterize peptides containing 3-nitrotyrosine (Sarver et al., J. Am Soc Mass Spectrom 12: 439-448, 2001). In this study, a unique series of ions were found for these peptides in addition to the mass shift of +45 Da corresponding to the addition of the nitro group. Specifically, two additional ions were observed at roughly equal abundance that correspond to the loss of one and two oxygens, and at lower abundances, two ions are seen that suggest the formation of hydroxylamine and amine derivatives. Post-translational modifications of cysteine and tryptophan residues are, in addition to tyrosine, major sites of reaction by free radicals.

Several known mass spectrometry protocols (e.g., Wells et al., Mol. Cell Proteomics 1: 791-804, 2002; and Wirth et al., Proteomics 2: 445-1451, 2002) can be used to detect these sites of damage to an OXPHOS protein or OXPHOS complex (such as Complex I, II, III, IV, or V). In such protocols, mass spectrometry is used to detect post-translational modifications of various subunits by comparing the molecular weight of particular subunits obtained from the patient with those of a corresponding normal subunit. A difference in molecular weight between the two indicates that the patient's target OXPHOS protein or complex activity is impaired and that the patient should be more thoroughly screened for aberrant post-translational modification of subunits in that complex, suggesting late-onset mitochondrial disease(s).

Alternatively, OXPHOS subunits obtained from a patient sample as described herein can be separated by gel electrophoresis and post-translational modifications identified by Western blotting with antiphosphotyrosine or antinitrotyrosine antibodies followed by mass spectrometry of the identified subunits, for example using LC/MS/MS. Yet another method for determining the presence of post-translational modifications of subunits involves an immunoassay wherein the subunits obtained from a patient sample are separated (for instance, as described herein in Example 1) and are contacted with an antibody that binds specifically to nitrotyrosine, such as the anti-nitrotyrosine antibody, rabbit IgG fraction commercially available from Molecular Probes (Eugene, Oreg., Cat # A-21285). Binding of the anti-nitrotyrosine antibody to a subunit of the target OXPHOS complex indicates that the patient's OXPHOS complex activity may be impaired and that the patient should be more thoroughly screened for late-onset mitochondrial disease(s).

D. Methods for Identifying Compounds That Affect OXPHOS Structure and/or Function

OXPHOS complexes (such as, Complex I) are permanently and adversely affected by a variety of substances (referred to as “mitotoxins”) including, without limitation, environmental toxins (such as, pesticides), drugs used to treat non-mitochondrial disease (such as, reverse transcriptase inhibitors and antibiotics), and drug impurities in narcotic drugs. Still another embodiment, therefore, provides screening methods for identifying an agent that causes a mitochondrial disorder.

In one such screening method, samples consisting of live animals, isolated cells or cell extracts, or isolated mitochondria are treated with a test agent. Preferably, a control sample is also prepared which does not receive the test agent, but is otherwise treated the same as a test sample. The solvent in which the test sample is dissolved, or a substance known to be harmless to the sample, may be administered to the control sample instead of the test agent. Following treatment with a test agent, one or more OXPHOS complexes are isolated from the test (and control) samples by immunocapture. In another approach, one or more OXPHOS complexes are immunocaptured, purified and, then, treated with a test agent. Following treatment with a test agent, the activity, assembly status, subunit composition, total mass, and/or biomarker burden of the treated OXPHOS complex(es) can be determined as previously described. A lower level of activity (or activities), or an altered assembly, or an altered subunit composition, or reduced total mass, or increased biomarker burden (e.g., quality or quantity of abnormal post-translational modifications) in the sample exposed to the agent indicates that the agent has the potential to cause a mitochondrial disorder. Such assays can be done in high-throughput format (for example, in a 96-well plate).

The provided methods can also be used to assess the extent of toxin damage to OXPHOS complex activity in the cells of individual subjects. To assess progressive damage to OXPHOS complex activity in the cells of the subject caused by any agent or disease, the method can be repeated at suitably spaced intervals, with decreased activity over time indicating increased and/or continuing damage. Similarly, the biomarker burden in such samples can be assessed by immunocapture analysis. As used herein, the term “suitably spaced intervals” will vary according to the type of toxin, or drug or the type of disorder the progress of which is being monitored, as well as according to the general health of the subject. For example, a suitably spaced interval may range from one day to 1 year, or 10 days to six months, or 30 days to 3 months, depending upon the disorder being monitored, the magnitude of the toxicity suspected, the circumstances of the subject's exposure to the toxin or drug, frequency of exposure or administration, and the like.

This disclosure further contemplates that immunocaptured OXPHOS proteins or complexes can be used to screen for compounds that protect OXPHOS proteins from damage induced by mitotoxins (such as, environmental toxins and therapeutic drugs). Such protective agents could be used to prevent or inhibit the advancement of damage to the OXPHOS system and its components. Also contemplated are methods of screening for therapeutic drugs effective for the treatment of early-onset and late-onset mitochondrial disorders. Assays for such protective and/or therapeutic drugs would evaluate the effects of known toxic agents alone or in the presence of potential protective/therapeutic drugs. The assays could be performed either in vivo (e.g., with immunocapture and analysis post-treatment) or in vitro (e.g., with immunocapture prior to treatment).

E. Detecting Alterations in mtDNA

Alteration of OXPHOS complex functioning due to reduced synthesis and/or alteration of mtDNA can be detected and/or monitored (e.g., for onset or stage of the disorder) using the disclosed methods. mtDNA depletion can be the result of inherited genetic defects, and can also accompany highly active anti-retroviral therapy (HAART) for HIV infection with nucleoside reverse transcriptase inhibitors, such as AZT and DDC, and in anti-cancer chemotherapy using similar nucleoside analogs. The methods described here can be used, for instance, to help guide HAART or chemotherapy to minimize mitotoxic side-effects.

Detection of one or more of OXPHOS Complex I, III, IV or V can serve as a surrogate for direct measurement of mtDNA because these four enzymes contain mtDNA-encoded proteins as structural and/or functional components. The activity and stability of each of these complexes is dependent on the presence of their mtDNA-encoded subunits (Marusich et al., Biochem. Biophys. Acta., 1362: 145-159, 1997; Janes et al., J. Histochem. Cytochem., 52: 1011-1018, 2004). The correlation between mtDNA levels and the quantity(ies) or activity(ies) of one or more of OXPHOS Complex I, III, IV or V is high; therefore measurement of either the level(s) of one or more of these complexes or the corresponding enzyme activity(ies) can give an good measurement of the levels of mtDNA in the sample. Standard curves of this relationship are known (or can be easily generated by one of ordinary skill in the art based on this disclosure) to permit quantification of mtDNA (see, e.g., Janes et al., J. Histochem. Cytochem., 52: 1011-1018, 2004).

Traditional methods of measuring mtDNA-dependent enzyme activity involve whole cell or tissue extracts or purified subcellular preparations (e.g., Birch-Machin and Turnbull, Meth. Cell Biol., 65: 97-117, 2001). However, these measurements are difficult and require relatively large amounts of sample. In addition, because many enzymes in a particular sample have related biochemical and enzymatic properties, if crude extracts are used, it is necessary to use a series of appropriate specific inhibitors to determine the fraction of the total activity that is due to the OXPHOS complex of interest. There is also little standardization among traditional techniques of measuring mtDNA-dependent enzyme activity. For example, Gellerich et al. (Mitochondrion, in press) sent bovine skeletal muscle homogenate on dry ice (as a test case) to 14 different laboratories in 8 countries, where each group measured the activities of Complexes I, I+III, II, II+III, IV and V, as well as citrate synthase. The activity measurements for each complex, or set of complexes, in the different laboratories varied by more than one order of magnitude. Immunocapture of Complex I, II, III, IV or V, or combinations thereof as described herein provides a convenient, simple, sensitive, accurate and/or standardized alternative method to detecting and/or quantifying the activities of OXPHOS complexes.

As demonstrated herein, the disclosed antibodies can be used to immunocapture all five OXPHOS complexes. As further demonstrated herein, quantitative measurement of these OXPHOS enzyme levels and/or activities can be made, for instance, in microplate- and bead-based assays. Moreover, other examples herein show that enzyme activities of immunocaptured OXPHOS complexes (such as, Complexes I and V) can be detected using a simple dipstick-based format that is instrument free.

F. Additional Monoclonal Antibodies Useful in the Disclosed Methods

Following the immunocapture of a native OXPHOS protein or OXPHOS complex, it may be useful to perform one or more subsequent assays (or method steps) that either denature the OXPHOS protein/complex (for example, Western blots using SDS-PAGE) or for which the native conformation of the OXPHOS protein/complex is unimportant (for example, use as a “second” anti-OXPHOS detector antibody (as discussed above). In these circumstances, other “non-capture” anti-OXPHOS antibodies may be useful. Some examples of additional non-capture (ancillary) antibodies that may be useful in the disclosed methods are shown in Table 2. The “non-capture” antibodies in Table 2 are commercially available from Molecular Probes and MitoScience (both of Eugene, Oreg.).

TABLE 2
Non-capture OXPHOS and Other MAbs

| | | | | | | Hu- | | | Bo- |
|---|---|---|---|---|---|---|---|---|---|
| Antigen | MW | MAb | Isotype | WB Conc | IC Conc | man | Mouse | Rat | vine |
| Complex I | | | | | | | | | |
| C-I-08 | 08 kD | RAC#24-18G7AC5 | nd | 100% CM | ? | + | nd | nd | nd |
| C-I-17 NDUFB6 | 17 kD | RAC#24A-22B8BE8H5 | IgG1, k | 0.25 ug/ml | − | + | + | nd | + |
| C-I-22 NDUFS4 | 22 kD | MM#10-2C7CD4AG3 | IgG1, k | <0.5 ug/ml | − | + | nd | nd | nd |
| C-I-30 NDUFS3 | 30 kD | RAC#24A-17D950C9H11 | IgG2a, k | 1 ug/ml | HIAR 5 ug/ml | + | + | + | + |
| C-I-30 NDUFS3 | 30 kD | RAC#24A-17G8BC3 | G2a/M? | 10F CM 1:4 | 10F CM 1:64 | + | + | nd | + |
| C-I-30 NDUFS3 | 30 kD | MM#7-3F9DD2 | IgG1, k | 0.5 ug/ml | 5 ug/ml | + | + | nd | +(?) |
| C-I-39 NDUFA9 | 39 kD | RAC#24-20C11B11B11 | IgG1, k | 1 ug/ml | HIAR 10 ug/ml | + | + | + | + |
| Complex II | | | | | | | | | |
| C-II-30 (FeS) | 30 kD | RAC#23-21A11AE7 | IgG2a, k | 5 ug/ml | HIAR 5 ug/ml | + | + | + | + |
| C-II-70 (FL) | 70 kD | RAC-#23-2E3GC12FB2AE2 | IgG1, k | 0.02 ug/ml | HIAR 0.2 ug/ml | + | + | + | + |
| Complex III | | | | | | | | | |
| C-III-Core 1 | 49 kD | MM#2-16D10AD9AH5 | IgG1, k | 0.2 ug/ml | 5 ug/ml | + | + | + | + |
| C-III-Core 1 | 49 kD | MM#2-13C11AF11 | IgG1, k | +++ | negative | + | +/− | nd | nd |
| C-III-Core 2 | 49.5 kD | RAC-#23-13G12AF12BB11 | IgG1, k | 0.5 ug/ml | HIAR 1 ug/ml | + | + | + | + |
| C-III-FeS | 25 kD | MM#2-5A5AC8 | IgG2b, k | 0.5 ug/ml | HIAR 5 ug/ml | + | + | + | + |
| C-III-10 kD | 10 kD | MM#2-1H9DE5DG5BC8 | IgG2a, k | <0.5 ug/ml | nd | + | + | nd | + |
| Complex IV | | | | | | | | | |
| C-IV-1 | 40 kD | RAC#18-1D6E1A8 | IgG2a, k | 0.5 ug/ml | 5 ug/ml | + | + | + | + |
| C-IV-2 (Human) | 24 kD | RAC#21-12C4F12 | IgG2a, k | 1 ug/ml | 5 ug/ml | + | − | − | +/− |
| C-IV-2 (Bovine) | 24 kD | RAC#21-15B4C1 | IgG | CM | | +/− | nd | − | + |
| C-IV-3 | 30 kD | RAC#14-DA5BC4 | IgG2a, k | 2 ug/ml | ? | + | − | nd | nd |
| C-IV-4 | 17 kD | RAC#11A-20E8C12 | IgG2a, k | 0.5 ug/ml | 5 ug/ml | + | + | + | + |
| C-IV-4 | 17 kD | RAC#4-10G8D12C12 | IgG2a, k | 0.5 ug/ml | HIAR 5 ug/ml | + | − | − | + |
| C-IV-5a | 12 kD | RAC#1-6E9B12D5 | IgG2a, k | 2 ug/ml | HIAR 5 ug/ml | + | + | +weak | + |
| C-IV-5b | 11 kD | RAC#7-16H12H9 | IgG2b, k | 2 ug/ml | neg | + | +(&40 kd) | + | + |
| C-IV-6aH | 09 kD | RAC#7-4H2A5 | IgG2a, k | 25 ug/ml | | +/− | ? | − | + |
| C-IV-6aL | 09 kD | RAC#15-14A3AD2BH4 | IgG1, k | 5 ug/ml | neg | +/− | + | +/− | + |
| C-IV-6b | 10 kD | RAC#7-3F9D3D11AF6 | IgG1, k | 1 ug/ml | | + | + | + | + |
| C-IV-6c | 08 kD | RAC#10-3G5F7G3 | IgG2b, k | 2 ug/ml | 5 ug/ml | + | − | + | + |
| C-IV-7aHL | 06 kD | RAC#10-6D7G8E5BH11 | IgG2a | 10 ug/ml | | + | +(&10 kd) | + | + |
| C-IV-7b-VIIb | 06 kD | RAC#3-1F2H9 | | neat CM | | nd | − | nd | + |
| C-IV-7b-VIIb | 06 kD | RAC#3-2G7H8AD9 | IgG1, k | 25 ug/ml | | +/− | − | − | + |
| Complex V | | | | | | | | | |
| C-V-Alpha | 53 kD | MM#1B-15H4C4 | IgG2b, k | 0.2 ug/ml | 2 ug/ml | + | + | + | + |
| (wide XR) | | | | | | | | | |
| C-V-Beta | 52 kD | RAC#5-7E3F2 | IgG2a, k | 4 ug/ml | nd | + | +/− | + | + |
| C-V-d | 21 kD | MM#1-7F9BG1 | IgG2b, k | <1 ug/ml | 1 ug/ml | + | +/− | nd | + |
| C-V-d | 21 kD | MM#1-12F4BB2 | IgG2b, k | positive | negative | + | + | nd | + |
| C-V-OSCP | 20 kD | MM#5-4C11C10D12 | IgG1, k | 0.1 ug/ml | 1 ug/ml (HIAR) | + | − | nd | nd |
| PDH | | | | | | | | | |
| PDH-E1-alpha | 42 kD | MM#5-8D10E6 | IgG1, k | 0.01-0.1 ug/ml | OK 0.1 ug/ml | + | + | nd | + |
| PDH-E1-alpha | 42 kD | MM#5-9H9AF5 | IgG1, k | 0.04 ug/ml | 5 ug/ml | + | + | nd | + |
| PDH-E1-beta | 35 kD | MM#3-17A5E2H8 | IgG1, k | 5 ug/ml | neg | + | + | + | + |
| PDH-E2 | 72 kD | MM#3-15D3G9C11 | IgG1, k | 0.01 ug/ml | 0.1 ug/ml??? | + | − | − | + |
| PDH-E2/E3bp | 72/55 | MM#3-13G2AE2BH5 | IgG2a, k | 0.5 ug/ml | 1-2 ug/ml | + | + | nd | + |
| Other | | | | | | | | | |
| TIM-22 | 21 kD | MM#9-2A9BG3 | IgG1, k | 8 ug/ml | − | + | − | nd | nd |
| SMAC/Diablo | 21 kD | MM#9-3A11AD3 | IgG2a, k | 50 ug/ml | positive | + | + | nd | nd |
| Cyclophilin D | 21 kD | RAC#27C-E11AE12BD4 | IgG1, k | 1 ug/ml | positive | + | nd | nd | |
| Cyclophilin D | 21 kD | RAC#27C-B7AG4AC8 | IgG1, k | 1 ug/ml | positive | + | nd | nd | |
| SURF-1 | 29 kD | RAC#26B-21H2BG4 | IgG1, k | 1 ug/ml | nd | + | + | nd | + |
| ANT | ˜32 kD | MM#10B-5F51BB5AG7 | IgG1, k | 1 ug/mlor | 5 ug/ml(HIAR) | + | nd | nd | + |
| | | | | less | | | | | |
| ANT | ˜32 kD | MM#10B-6E55H11 | IgG . . . | +CM | bkgrdCM | + | nd | nd | nd |
| Porin | 39 kD | MM#4A-20B12AF2 | IgG2b, k | 1 ug/ml | HIAR 0.2 ug/ml | + | + | nd | + |

HIAR = Heat-Induced-Antigen-Retrieval (20 min incubation at 90-100 C. in 0.1 M Tris/HCl pH 9.5, with 5% urea (wt/vol)) preferred for optimal reactivity to antigens fixed with aldehydes.
CM = hybridoma culture medium
nd = not determined

V. Mitochondrial Enzyme Function and Disease

Numerous medical disorders have been linked to mitochondrial dysfunction. For example, alteration of OXPHOS functioning due to reduced synthesis and/or post-translational modification of component proteins (and mtDNA) are believed to contribute to Parkinson's disease, Huntington's disease, Alzheimer's disease, Downs Syndrome, schizophrenia, late-onset type II diabetes (also called NIDDM), and even in the aging process itself. Moreover, altered OXPHOS can also be an unintended consequence and complication of the treatment of human diseases; for example, reperfusion injury is a problem for heart attack victims and a critical issue in all organ transplants. Re-oxygenation of anaerobic tissue produces high concentrations of toxic free radicals, which react with the highly reduced OXPHOS proteins, and it is this process that is thought to kill cells. Therapy for HIV infection with nucleoside reverse transcriptase inhibitors, such as AZT and DDC, causes myopathy and lipidopathy in many patients and is believed to be due to a loss of oxidative OXPHOS function resulting from the reduction of mitochondrial protein synthesis. The myopathy that is an occasional side effect of statin use to treat hypercholesterolemia has also been attributed to mitochondrial toxicity of these drugs. Reviews of the molecular bases of mitochondrially related health disorders are found in Lib et al. (J. Histochem. Cytochem., 50: 877-884, 2002) and Hanson et al. (J. Histochem. Cytochem., 50: 1281-1288, 2002).

As described below, certain diseases have been linked to the function (or dysfunction) of specific OXPHOS enzyme complexes. As described throughout this specification, the study, diagnosis, and evaluation of these diseases will be facilitated using the disclosed antibodies, methods and kits.

A. Parkinson's Disease.

Complex I dysfunction has been associated with the late onset mitochondrial disease, Parkinson's disease. Reagents, which selectively inhibit Complex I function, including MPTP and several plant toxins (such as, rotenone) produce a syndrome very like ideopathic Parkinson's disease (Betarbet et al., Nat. Neurosci., 3: 1301-1306, 2000; Dauer et al., Proc. Natl. Acad. Sci. USA, 99: 13972-13974, 2002; Sherer et al., Neuroscientist, 8: 192-197, 2002). Epidemiological studies show that workers exposed to high levels of such herbicides have a higher incidence of Parkinson's disease (Butterfield et al., Neurology, 43: 1150-1158, 1993; Przedborski et al., Restor. Neurol. Neurosci., 16: 135-142, 2000; Jenner, Trends Neurosci., 24: 245-247, 2001). In addition, a relationship between Parkinson's disease onset and severity and polymorphisms in the Complex I genes on mtDNA has been reported (Tanaka, J. Neurol., 249(Suppl. 2): I111-118, 2002; van der Walt et al., Am. J. Hum. Genet., 72: 804-811, 2003). Lowered Complex I activity has also been reported in the substantia nigra and other parts of the brain, skeletal muscle, platelets and lymphocytes of Parkinson's disease patients (Bindoff et al., J. Neurol. Sci., 104: 203-208, 1991; Swerdlow et al., Ann. Neurol., 40: 663-671, 1996). In most studies, the levels of Complex I deficit are not large, which has led to the suggestion that there is an amplification effect in that reduced Complex I activity increases generation of oxidative free radicals (Greenamyre et al., IUBMB Life., 52: 135-141, 2001; Orth and Schapira, Am. J. Med. Genet., 106: 27-36, 2001) which react within the mitochondrion and with nearby cytosolic proteins such as parkin and synuclein to induce cell death (Sherer et al., J. Neurosci., 22: 7006-7015, 2002; Greene et al., Proc. Natl. Acad. Sci. USA., 100: 4078-4083, 2003). The substantia nigra is already under oxidative stress because of H₂O₂ production associated with dopamine synthesis (Greenamyre et al., IUBMB Life., 52: 135-141, 2001; Antunes et al., Biochim. Biophys. Acta, 1556: 233-238, 2002; Sherer et al., J. Neurosci., 22: 7006-7015, 2002), so even a small increase in levels of free radical production in this tissue could tip the balance towards the selective degeneration of the domaninergic neurons. Increased generation of peroxides and superoxides because of altered Complex I has been reported by several investigators (Bharath et al., Biochem. Pharmacol., 64: 1037-1048, 2002; Jenner, Ann. Neurol., 53 (Suppl. 3): S26-36, 2003; Kalivendi et al., Biochem. J., 371: 151-164, 2003). Both hydroxyl radical and peroxynitrite-induced reduction of Complex I activity has been reported recently in in vitro studies (Bautista et al., Biochem. Biophys. Res. Commun., 275: 890-894, 2000; Riobo et al., Biochem. J., 359: 139-145, 2001; Murray et al., J. Biol. Chem., 278(39): 37223-37230, 2003). Accumulation of this damage will be determined by the rate of free radical reaction in relation to the rates of degradation of the proteins versus new synthesis, and as a function of the lifetime of a cell and its replacement.

Disclosed herein are antibodies specific for native Complex I (or its native subunit(s), as indicated in Table 1), such as RAC#24-20D1AB7, RAC#24-18G12BC2AA10, RAC#24-17C8E4E11, RAC#24-17G3D9E12, RAC#29-1D4, RAC#29-4G6BB9, RAC#29-6E1BH7, or RAC#24A-20E9DH10C12 (see also, Table 1). Such antibodies can immunocapture all or part of Complex I. Accordingly, these antibodies are useful, for example, to detect and quantify the expression of partially or fully assembled Complex I (or its subunits), to determine the assembly state of Complex I, to detect post-translational modifications in one or more subunits of Complex I, to capture Complex I for activity measurements, and/or for inclusion in immunoassay devices. Given the above-described associations between Parkinson's disease and Complex I expression and activity, it is expected that the disclosed Complex I-specific antibodies, at least, will be readily adapted to the study, diagnosis, and evaluation of Parkinson's disease.

B. Alzheimer's Disease

Mitochondrial dysfunction is believed to play a role in Alzheimer's disease (Mattson, Int. Rev. Neurobiol., 53: 387-409, 2002; Swerdlow and Kish, Int. Rev. Neurobiol., 53: 341-385, 2002; Castellani et al., J. Neurosci. Res., 70: 357-360, 2002). The levels and activity of cytochrome c oxidase (Complex IV or COX) in the brains of Alzheimer's patients has received particular interest. Levels of Complex IV subunits I, II, IV and VIc were 42-47% lower in the temporal and parietal cortices of Alzheimer's patients, but normal levels of the enzyme complex were measured in the patients' cerebral cortices (Kish et al., J. Neurochem., 72: 700-707, 1999). Activity and protein profile studies on hippocampal tissue also showed selective loss of Complex IV in Alzheimer's patients (Verwer et al., Exp. Neurol., 163: 440-451, 2000; Bosetti et al., Neurobiol. Aging, 23: 371-376, 2002). Levels of mtDNA and Complex IV were found to increase in neurons while neuronal activity was down and there was considerable oxidative damage as reported by nitrotyrosine formation (Castegna et al., J. Neurochem., 85: 1394-1401, 2003).

Antibodies specific for native Complex IV (or its native subunit(s)), such as RAC#11B-7E5BA4, RAC#23C-21H10, RAC#23C-22D5, RAC#23C-22H11G43E1, RAC#23C-28G7, and RAC#23C-31E91B82G9 are disclosed herein (see Table 1). Such antibodies can immunocapture all or part of Complex IV. Accordingly, these antibodies are useful, for example, to detect and quantify the expression of partially or fully assembled Complex IV (and its subunits), to determine the assembly state of Complex IV, to detect post-translational modifications in one or more subunits of Complex IV, to capture Complex I for activity measurements, and/or for inclusion in immunoassay devices. Given the above-described associations between Alzheimer's disease and Complex IV expression and activity, it is expected that the disclosed Complex IV-specific antibodies, at least, will be readily adapted to the study, diagnosis, and evaluation of Alzheimer's disease.

C. Schizophrenia.

Altered mitochondrial function has been described in schizophrenia with various reports that Complex I and cytochrome c oxidase (Complex IV) levels and/or activity are reduced in the brains of patients (Ben-Shachar et al., Int. J. Neuropsychopharmacol., 2: 245-253, 1999; Maurer et al., Schizophr. Res., 48: 125-136, 2001; Ben-Shachar, J. Neurochem., 83: 1241-1251, 2002; Blass, Int. Rev. Neurobiol., 51: 325-376, 2002). For example, lowered Complex I activity in platelets of schizophrenics was correlated with the severity of psychotic symptoms in these patients (Dror et al., Mol. Psychology, 7: 995-1001, 2002; Elkashef et al., Prog. Neuropsychopharmacol. Biol. Psychiatry, 26: 145-148, 2002). Reduced expression of two Complex I subunits in schizophrenic subjects have also been reported (Dror et al., Mol. Psychology, 7: 995-1001, 2002).

Given the above-described associations between schizophrenia and the expression and activities of Complexes I and IV, it is expected that the disclosed Complex I- and/or Complex IV-specific antibodies (see examples listed above), at least, will be readily adapted to the study, diagnosis, and evaluation of schizophrenia.

D. Diabetes

Genetic evidence suggests a link between OXPHOS deficiency and early- and late-onset diabetes. In around 1% of cases of insulin-dependent diabetes, the cause appears to be a point mutation (T3243G) in mtDNA which leads to reduced energy production in islet cells and decreased insulin secretion (Choo-Kang et al., Diabetes, 51: 2317-2320, 2002; Maassen et al., J. Endocrinol. Invest., 25: 477-484, 2002). Consistently, animal models in which levels of mtDNA have been reduced by drugs replicate the symptoms of diabetes (Wallace, Methods Mol. Biol., 197: 3-54, 2002). It is thought that reduced OXPHOS activity increases oxidative stress which alters calcium regulation to lower insulin secretion (Wollheim, Diabetologia, 43: 265-277, 2000; Silva et al., Nat. Genet., 26: 336-340, 2000; Maechler and Wollheim, Nature, 414: 807-812, 2001; Sakai et al., Biochem. Biophys. Res. Commun., 300: 216-222, 2003).

Accumulation of oxidative damage resulting in environmental and diet-induced OXPHOS defects may account for late-onset, type 2 (also called non-insulin-dependent) diabetes (Santos et al., Diabetes Metab. Res. Rev., 17: 223-230, 2001; Turko et al., J. Biol. Chem., 278(36): 33972-33977, 2003). In particular, Complex I deficiency has been reported in skeletal muscle of type 2 diabetics in a study of 10 patients and 10 controls (Kelley et al., Diabetes, 51: 2944-2950, 2002). Also altered OXPHOS functioning in peripheral blood cells has been reported to be a potential marker of type 2 diabetes by several groups (Pugnaloni et al., Eur. J. Histochem., 45: 85-94, 2001; Song et al., Diabetes Care, 24: 865-869, 2001).

The disclosed library of antibodies (see, for instance, Table 1) contains mAbs specific for all five OXPHOS enzyme complexes (e.g., Complexes I, II, III, IV and V). These antibodies are useful, for example, to detect and quantify the expression of partially or fully assembled OXPHOS enzyme complexes (and their subunits), to determine the assembly state of one or more OXPHOS enzyme complexes, to detect post-translational modifications in one or more subunits of one or more OXPHOS enzyme complexes, to capture one or more OXPHOS enzyme complexes for activity measurements, and/or for inclusion in immunoassay devices. Given the above-described associations between diabetes and OXPHOS defects, including for example Complex I deficiency, it is expected that antibodies specific for particular OXPHOS enzyme complexes (such as anti-Complex I mAbs) or combinations of antibodies specific for two or more of the OXPHOS enzyme complexes, at least, will be readily adapted to the study, diagnosis, and evaluation of diabetes.

E. Cardiac Disease

Mitochondrial defects can present as cardiac disease in several ways (Casademont and Miro, Heart Fail. Rev., 7: 131-137, 2002; Marin-Garcia and Goldenthal, J. Card. Fail., 8: 347-361, 2002). For example, there are genetic links. Homoplasmic mtDNA mutations have been found to give rise to inherited hypertrophic cardiomyopathy (Taylor et al., J. Am. Coll. Cardiol., 41: 1786-1796, 2003). Mutations in an assembly factor for cytochrome c oxidase (Complex IV) also cause hypertrophic cardiomyopathy (Antonicka et al., Am. J. Hum. Genet., 72: 101-114, 2003). ADP/ATP translocase (ANT), which is closely associated with Complex V (the F₁/F₀ ATPase), is a specific target for the autoimmune form of idiopathic dilated cardiomyopathy (Manchado et al., J. Mol. Cell. Cardiol., 34: 571-582, 2002). It has been claimed that the link between these genetic alterations and disease is free radical damage leading to apoptosis of heart cells.

Another mitochondrial link to cardiac disease is so-called “anoxia/reperfusion” injury. When heart attack victims or cardiac surgery patients are reperfused with oxygenated blood after tissue has become anoxic and the respiratory chain reduced, there is a burst of free radical production. The resultant free radicals are thought to lead to oxidative damage of the tissue (Monteiro et al., Rev. Port. Cardiol., 22: 233-254, 2003). Many ongoing studies are trying to identify the sites and extent of this damage to OXPHOS components (Petrosillo et al., FASEB. J, 17: 714-716, 2003) and to find therapies that will prevent the heart damage.

The antibody reagents disclosed herein can be used, for example, to detect and quantify the expression of partially or fully assembled OXPHOS enzyme complexes (and their subunits), to determine the assembly state of one or more OXPHOS enzyme complexes, to detect post-translational modifications in one or more subunits of one or more OXPHOS enzyme complexes, to capture one or more OXPHOS enzyme complexes for activity measurements, and/or for inclusion in immunoassay devices. Given the above-described associations between cardiac disease and function of the OXPHOS system, including for example Complex V, it is expected that antibodies specific for particular OXPHOS enzyme complexes (such as anti-Complex V mAbs) or combinations of antibodies specific for two or more of the OXPHOS enzyme complexes, at least, will be readily adapted to the study, diagnosis, and evaluation of cardiac disease. Immunocapture and activity analysis of Complex V can be used to monitor the endogenous levels and activated IF₁, a regulatory protein suggested to play an important role in conserving ATP (cellular energy) during ischemic stress (see, e.g., Aggeler et al., J. Biol. Chem., 277: 33906-33912, 2002). Representative examples of mAbs specific for native Complex V (or its native subunit(s)) are shown in Table 1.

F. Nucleotide Reverse Transcriptase Inhibitor (NRTI) Toxicity

It is believed that much pathology that arises secondary to NRTI treatment is the result of mitochondrial dysfunction. NRTI-related pathology includes, for example, lactic acidosis, lipodystrophy, neuropathy and cardiovascular dysfunction.

Lactic acidosis occurs in as many as 35% of HIV patients receiving single NRTI or combination therapy for more than 6 months (Brinkman, Clin. Infect. Dis., 31: 167-169, 2000; Moyle, Clin. Ther., 22: 911-936, 2000; Carr and Cooper, Lancet, 356: 1423-1430, 2000; John et al., J. AIDS, 15: 717-723, 2001). Lactic acidosis is believed to result from reduced activity of the OXPHOS system, which leads to increased glycolysis in skeletal muscle, liver, and other tissues. Deficiencies in the OXPHOS system are thought to arise secondary to loss of mtDNA, which encodes various subunits of the OXPHOS enzyme complexes.

Lipodystrophy occurs in about 50% of patients receiving NRTIs (Gan et al., Diabetes Obes. Metab., 3: 67-71, 2001). This toxic side effect mimics two inherited mitochondrial disorders, Madeling's disease (a multiple symmetrical lipomatosis) and familial lipomas, both of which are believed due to a specific mutation (A8344G) in mtDNA (Moyle, Clin. Ther., 22: 911-936, 2000).

Peripheral neuropathies are reported in up to 50% of HIV-infected patients (Wulff et al., Drugs, 59: 1251-1260, 2000). Such conditions are common with OXPHOS defects (Wallace, Science, 283: 1482-1488, 1999). Similarly, many HIV-infected patients present with heart muscle dysfunction, such as congestive heart failure and dilated cardiomyopathy. As discussed above, cardiac dysfunctions occur in a significant proportion of patients with mtDNA defects, which affect OXPHOS system function.

The disclosed antibodies, which are specific for OXPHOS enzyme complexes, can be used, for example, to detect and/or monitor NRTI-induced mitochondrial toxicity. For example, antibodies specific for particular OXPHOS enzyme complexes (such as Complex I and/or Complex IV) may be incorporated into immunoassay devices (such as a dipstick or other lateral flow device) for rapid, easy point-of-care detection of NRTI-related OXPHOS defects, including mtDNA depletion.

VI. OXPHOS Complexes and Related Diseases

A. Complex I

Disorders of mitochondrial energy metabolism occur in humans with a frequency of 1 in 10,000 live births (Bougeron et al., Nat. Genet., 11: 144-149, 1995). Most are caused by the dysfunction of one or more of the enzyme complexes of oxidative phosphorylation (OXPHOS). Isolated enzymatic deficiency of the first OXPHOS complex, NADH:ubiquinone oxidoreductase (EC 1.6.99.3) or Complex I, is one of the most frequent causes of mitochondrial respiratory chain disorders (Loeffen et al., Hum. Mut., 15: 123-134, 2000). Complex I defects are also strongly implicated in the pathogenesis of degenerative diseases, such as Parkinson's disease. Specific inhibitors of Complex I such as MPP+ can cause Parkinson's-like disease in both animal models and in humans (Nicklas et al., Life Sci., 36: 2503-2508, 1985). In addition, systemic exposure to environmental Complex I-specific toxins (such as rotenone) induce a Parkinson's-like disorder in rats and humans (Betabet et al., Nat. Neurosci. 3: 1301-13306, 2000; Gorell et al., Neurology, 50: 1346-1350, 1998).

Complex I is the first multiprotein complex of the OXPHOS system (Walker, Q. Rev. Biophys., 25: 253-324, 1992) and participates in the formation of a proton gradient across the inner mitochondrial membrane coupled to transfer of electrons from NADH to ubiquinone. This proton gradient provides part of the proton-motive force used for ATP production. Two other sites in the cytochrome chain also couple electron transfer to ATP production in the same way so that for every pair of electrons from NADH that are oxidized by O₂, three ATPs are produced. Alterations in Complex I reduce or eliminate energy production in mitochondria and, therefore, are pathogenic.

Complex I is the largest of the respiratory chain complexes, made up of seven different subunits encoded on mitochondrial DNA (mtDNA; ND1-6 and ND4L) and 38 or more different subunits encoded by nuclear genes (Grigorieff, Curr. Opin. Struct. Biol., 9: 476-483, 1999; Skehel et al., FEBS Lett., 438: 301-305, 1998) Together, these subunits form a complex with an estimated molecular mass of about 900,000 daltons (Walker, Q. Rev. Biophys., 25: 253-324, 1992). Mutations in both the mitochondrial- and nuclear-encoded genes are known to cause Complex I deficiencies (Smeitink et al., Am. J Hum. Genet., 64: 1505-1510, 1999). However, in addition to the structural genes, there may be additional genes encoding proteins required for the assembly of a functional Complex I. So-called “assembly factors” involved in assembly of Complex IV and the ATP synthase have already been reported (Ackerman, et al., Proc. Natl. Acad. Sci. USA, 87: 4986-4990, 1990; Tiranti et al., Am. J. Hum. Genet., 63: 1609-1621, 1998; Papadopoulou et al., Nat. Genet., 23: 333-337, 1999). For example, mutations in SURF1, an assembly factor required for full assembly of Complex IV, has been shown to cause cytochrome c oxidase deficiency in many of reported cases of Leigh's disease (Ackerman, et al., Proc. Natl. Acad. Sci. USA, 87: 4986-4990, 1990; Teraoka et al., Hum. Genet., 105: 560-563, 1999, and Poyau et al., Hum. Genet., 106: 194-205, 2000).

The genes for all of the components of Complex I have now been identified (Walker et al., Methods Enzymol., 260: 14-34, 1995), opening up the possibility of genetic approaches for diagnosis. However, such an analysis would not be able to identify Complex I deficiencies caused by mutations in assembly factors, until these factors are identified and even then would not yield sufficient information to understand the genotype-phenotype relationships of the various mutations that can occur. Therefore, in addition to genetic analysis, new and better protein-based approaches for detecting and characterizing Complex I deficiencies as well as diseases associated with mitochondrial dysfunction are needed. These methods are provided herein.

Example methods described herein use monoclonal antibodies characterized as specifically binding to Complex I of the mitochondrial respiratory chain and immunocapturing Complex I, wherein Complex I retains functional activity. It should be understood that the invention monoclonal antibodies may be able to immunocapture Complex I in the absence of all 45 subunits being present, although antibodies that precipitate the entire 45 subunit complex are preferred. The disclosure also provides isolated monoclonal antibodies that specifically bind to subunits of Complex I, for example the 39 kDa, 30 kDa, 20 kDa, 18 kDa, 15 kDa, and 8 kDa subunits of Complex I. In particular examples, anti-8 kDa subunit (e.g., RAC#24-18G12BC2AA10; RAC#24-17C8E4E11), anti-15 kDa subunit (e.g., RAC#24-17G3D9E12), anti-19 kDa (e.g., RAC#29-1D4; RAC#29-4G6BB9; RAC#29-6E1BH7) and anti-20 kDa (e.g., RAC#24A-20E9DH10C12) subunit monoclonal antibodies can be used to immunocapture native Complex I and the respective native OXPHOS protein subunits (see Table 1).

Non-limiting method embodiments provide for detecting Complex I deficiency in a patient by contacting one or more capture monoclonal antibodies specific for one or more Complex I subunits with a subject sample so that the antibodies immunocapture fully or partially assembled Complex I present in the sample. In some examples, substantially all of the proteins in the sample to be tested are tagged with a detectable label; thus, immunocaptured proteins (such as, OXPHOS proteins or complexes) can be directly detected following removal of unbound material. In another example, a labeled secondary antibody specific for the immunocaptured protein can be added to detect the capture of the target protein (e.g., OXPHOS protein or complex) by the capture antibody. The amount of each Complex I subunit immunocaptured by a respective Complex I subunit antibody is then determined and compared with the amount thereof determined to be present in a corresponding normal sample, wherein a decrease in the amount of any of the Complex I subunits in the sample as compared to the amount in a normal sample indicates the presence of a Complex I deficiency in the subject. Since Complex I enzymatic activity for energy production in a cell primarily depends upon the amount of functioning fully assembled Complex I that is present in the cell, the assay can be used to detect a decrease in Complex I enzymatic activity in the cells of the subject whose sample is tested.

B. Complex II

Complex II (succinnate:ubiquinone oxidoreductase) transfers electrons in the form of reduced FADH₂ from the citric acid cycle intermediate succinate, to the carrier ubiquinone (also known as coenzyme Q). Although no protons are pumped across the membrane by Complex II because there is not a large enough drop in free energy associated with the electron transfer, the subsequent transfer of these electrons to cytochrome c (catalyzed by Complex III) and oxygen (catalyzed by cytochrome c oxidase) are each accompanied by obligatory proton pumping and eventual ATP production. Fewer diseases appear to be associated with defects in Complex II than with defects in other OXPHOS complexes. Presumably this discrepancy arises because all four subunits of Complex II are encoded by nuclear DNA and therefore, the enzyme complex is not affected directly by mutations in mtDNA (unlike Complexes I, III, IV and V). In addition, it is believed that Complex II defects are less likely to be pathogenic because Complex II contributes less to the formation of the inner mitochondrial proton gradient.

Complex II can be immunocaptured intact, for example, using mAb RAC#23C-4H12BG12AG2, and analyzed for subunit composition by one-dimensional SDS-PAGE (as described, for instance, in Example 6 and Murray et al., Electrophoresis, 25: 2520-2525, 2004). As shown in Example 6, immunocapture of Complex II followed by SDS-PAGE visualized with appropriate detection reagents can be used to assess (i) Complex II post-translational modifications and (ii) Complex II subunit composition and supercomplex formation.

C. Complex III

Complex II (ubiquinone-cytochrome c oxidoreductase) accepts electrons from reduced coenzyme-Q and transfers them to oxidized cytochrome c. This redox reaction is accompanied by obligatory pumping of protons across the inner mitochondrial membrane, contributing to formation of the chemiosmotic gradient. In mammals, Complex III is composed of a single mtDNA-encoded protein, cytochrome b, and 10 nuclear-encoded proteins.

By way of example, monoclonal antibodies RAC#23B-1A1BC12AB9; RAC#23C-4H12BC11BC5; RAC#23B-10D2; RAC#23C-11A51H12; RAC#23C-12G8; RAC#23C-17A81A8; and RAC#23C-29C2 are specific for capture of native Complex III (or its native subunit(s), as indicated in Table 1) (see also, Table 1 and Murray et al., Electrophoresis, 25: 2520-2525, 2004). The antibody specifically immunocaptures Complex III from detergent solubilized mitochondria (human heart or bovine heart). The specificity of immunocapture was determined by comparing the protein subunit composition of the immunocaptured target antigen with that of Complex III purified by other means.

Anti-Complex III capture mAbs (see, e.g., Table 1) will extremely useful in biochemical and structural studies of Complex III in both normal cell physiology and in disease. For example, such antibodies enable simple, high-throughput assays for (i) quantitation of Complex III in cell and tissue extracts, (ii) quantitation of Complex III enzymatic specific activities in cell and tissue extracts (if the immunocaptured enzyme retains enzyme activity), and (iii) focused proteomics of Complex III purified from microscale samples of cells and/or tissues subjected to different physiologic, disease or defined chemical treatment (i.e., potential Complex III-reactive toxins) to identify specific molecular sites on Complex III, and the nature of chemical modifications associated with these conditions (the sites so identified are potentially regulative in both normal enzyme function and in altered enzyme function in disease states and therefore will be prime targets for development of drugs that can modify enzyme function and ameliorate or prevent disease-associated changes).

OXPHOS capture antibodies have a broader range of utilities than do non-capture OXPHOS antibodies. Thus, an OXPHOS capture antibody (such as, anti-Complex III capture mAbs and other antibodies listed in Table 1) may substitute for a non-capture antibody in a particular method involving non-native OXPHOS complexes or subunits (such as, Western blot or immunocytochemistry), but a non-capture antibody will not substantially bind a native OXPHOS complex or subunit; therefore, a non-capture antibody is not useful in methods involving native OXPHOS complexes or subunits.

D. Complex IV

Complex IV is the terminal enzyme of the respiratory chain, transferring electrons from reduced cytochrome c to molecular oxygen. This transfer is accompanied by obligatory pumping of protons across the inner membrane, contributing to formation of the electrochemical gradient used by Complex V (ATP synthase) to form ATP. Mammalian Complex IV is composed of thirteen protein subunits, including three encoded by mtDNA and ten encoded by nuclear DNA. Because the three mtDNA-encoded subunits form the functional and structural core of the enzyme, defects in these subunits, or their absence due to depleted mtDNA, usually results in not only functional defects, but also defects in the assembly of the remaining nuclear-DNA-encoded subunits. In addition, a number of nuclear-DNA-encoded Complex IV assembly factors have been identified on the basis of their association with severe inherited disorders (as discussed previously). Therefore, the ability to detect assembly defects in Complex IV, such as by the immunocapture assays described herein, affords the ability to detect most known defects in Complex IV.

By way of example, monoclonal antibodies RAC#11B-7E5BA4; RAC#23C-21H10; RAC#23C-22D5; RAC#23C-22H11G43E1; RAC#23C-28G7; RAC#23C-31E91B82G9 are specific for capture of native Complex IV (or its native subunit(s)) (see Table 1). These antibodies specifically immunocaptures (e.g., immunoprecipitates) Complex IV from detergent solubilized mitochondria (human heart or bovine heart). The specificity of immunocapture was determined by comparing the protein subunit composition of the immunoprecipitate with that of Complex IV purified by other means. Complex IV immunocaptured, at least, by MAb RAC#11B-7E5BA4 retains enzymatic activity (see Example 7 and Murray et al., Electrophoresis, 25: 2520-2525, 2004).

Anti-Complex IV capture mAbs (see, e.g., Table 1) will useful in biochemical and structural studies of Complex IV, in both normal cell physiology and in disease. Such antibodies enable, for instance, simple, high-throughput assays for 1) quantitation of Complex IV in cell and tissue extracts, 2) quantitation of Complex IV enzymatic specific activities in cell and tissue extracts, and 3) focused proteomics of Complex IV purified from microscale samples of cells and/or tissues subjected to different physiologic, disease or defined chemical treatment (i.e., potential Complex IV-reactive toxins) to identify specific molecular sites on Complex IV, and the nature of chemical modifications associated with these conditions (the sites so identified are potentially regulative in both normal enzyme function and in altered enzyme function in disease states and therefore will be prime targets for development of drugs that can modify enzyme function and ameliorate or prevent disease-associated changes).

E. Complex V (F₁/F₀ ATPase)

A typical adult human utilizes approximately 50 kg of ATP per day under normal activity levels, requiring roughly a 1000× turnover of the 50 g of ATP/ADP present in the body. The F₁/F₀-type ATPase, also known as ATP synthase and Complex V of OXPHOS, produces the great majority of this ATP in mitochondria, through the process of oxidative phosphorylation.

F₁/F₀ ATPase is composed of two parts, the F₀, which is an integral membrane complex that functions as a proton pore; and the F₁, which can catalyze ATP hydrolysis. When the relative proton concentration on the outside of the membrane is increased by electron transfer reactions, the F₀ complex is a proton channel and can let the protons run down the concentration gradient, releasing free energy. The close association of F₁ with F₀ causes the released free energy to be used to reverse the hydrolysis of ATP, with the net synthesis of ATP from ADP and Pᵢ. The proton channel can be blocked by the classic inhibitor of the enzyme, oligomycin. F₁/F₀ ATPase functions as a small molecular motor in which catalytic site events in the F₁ part are coupled to proton translocation in the F₀ part by rotation of a mobile domain or “crankshaft” within the protein complex.

In mitochondria, the hydrolytic activity of F₁/F₀ ATPase is regulated by an inhibitor protein, IF₁. Its binding to F₁/F₀ ATPase depends on pH. Below neutrality, IF₁ is dimeric and forms a stable complex with the F₁/F₀ ATPase. At higher pH values, for example pH 8.0 or 8.5, IF₁ forms a tetramer and is inactive. Tetramer formation masks the inhibitory region preventing binding of IF₁ to ATP synthase.

The mammalian form of the F₁/F₀ ATPase has been extensively studied using beef heart and rat liver as a source. It is complex of 16 different subunits with α₃, β₃, γ, δ, and ε comprising the F₁ part and a, b, c, d, e, f, g, A6L, OSCP and coupling factor 6 providing the F₀ and stator (Capaldi and Aggeler, Trends Biochem. Sci., 27: 154-160, 2002; Ko, et al., J. Biol. Chem., 275: 32931-32939, 2000). Also associated in the complex at physiological pH is an intrinsic inhibitor protein IF₁ (Ko, et al., J. Biol. Chem., 275: 32931-32939, 2000; Karrash and Walker, J. Mol. Virol., 290,379-384, 1999).

Not surprising given the key role of F₁/F₀ ATPase in energy metabolism, alterations of the protein complex result in human disease. Mutations in the mitochondrially encoded subunit a of the enzyme have been shown to cause Leigh's disease and other pathological conditions, depending on the specific mutation and the levels of heteroplasmy, i.e., the relative levels of normal and mutated mtDNA in cells. Alterations in F₁/F₀ ATPase as a result of chemical insults can also cause pathology, and may be a predisposing factor for neurodegenerative diseases because of the role that that the enzyme plays in apoptosis (Nijtmans et al., J. Biol. Chem., 276: 6755-6762, 2001; Dimauro and Schon, Am. J. Med. Genet., 106: 18-26, 2001; Schon et al., J. Bioenerg. Biomembr., 29: 131-148, 1997). Also, there is recent evidence of the physiological regulation of F₁/F₀ ATPase activity by phosphorylation-dephosphorylation reactions (Pedersen, J. Bioenerg. Biomembr., 31: 291-304, 1999; Matsuyame et al., Mol. Cell, 1: 327-336, 1998) as well as by the inhibitor protein IF₁.

Investigation of the relationship between alterations in structure and functioning of F₁/F₀ ATPase in various physiological and pathological conditions requires a simple way of isolating the enzyme complex from small amounts of human tissue, mostly needle biopsy or cell culture material. However, the protocols developed for isolating beef heart mitochondrial F₁/F₀ ATPase require too much material. Prior to this disclosure, an assay that isolated the enzyme from small amounts of human tissue has not yet been described. Thus, there was a need in the art for methods for monitoring dysfunction of OXPHOS, and particularly F₁/F₀ ATPase in a format that is simple, reproducible, uses small amounts of tissue that can be obtained from needle biopsy, collected blood. This need is satisfied by methods and compositions provided herein.

The disclosure provides microscale methods for the immunocapture and functional detection of active mitochondrial F₁/F₀ ATPase from solubilized human mitochondria using one or more specific anti-human monoclonal antibodies in an immunocapture format. Non-limiting examples of mAbs that can be used to immunocapture native Complex V (or its native subunits) are shown in Table 1. These antibodies enable, for instance, capture mAb-based assays suitable for high-throughput screening of samples containing mitochondria, for example obtained from human heart, human brain, human cultured fibroblast or bovine heart. Such high-throughput assays can be used to measure both the total amount of Complex V protein in a sample and also the amount of Complex V enzyme activity in a sample. As shown in Example 2, anti-Complex V capture mAbs bind an antigen having oligomycin-sensitive ATPase activity, which is characteristic of the mitochondrial F₁/F₀ ATPase. Negative control antibodies fail to capture detectable activity. The assay is also quantitative, and can be used to measure the amount of solubilized mitochondrial F₁/F₀ ATPase in samples relative to a reference control containing a known amount of F₁/F₀ ATPase, for example. Thus, the described assay can be used, for instance, to detect disorders in production and/or utilization of F₁/F₀ ATPase in patient samples (see, e.g., Aggeler et al., J. Biol. Chem., 277: 33906-33912, 2002).

The described assays are also sensitive, requiring as little as 10 nanograms of mitochondrial protein per test, and have a wide dynamic range of at least 1000-fold. For example, when human heart mitochondria are used as a target, the assay is quantitative over a range from 10 nanograms to 10 micrograms of mitochondrial protein per sample. In light of this, it is believed that the methods are suitable for use in high-throughput screening assay formats.

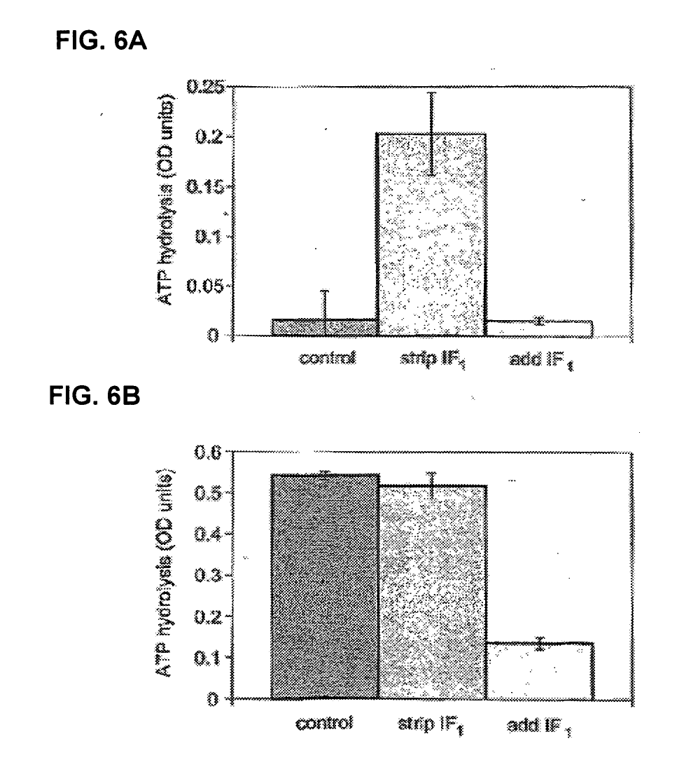

In another embodiment, the F₁/F₀ ATPase functional immunocapture assay is suitable for use as a diagnostic assay to detect any type of activity-affecting defect of mitochondrial F₁/F₀ ATPase in a subject, such as catalytic defects, the presence or absence of target subunit antigen, defects in assembly of the enzyme complex, and the like. In another embodiment, there is provided a F₁/F₀ ATPase functional immunocapture assay for determining interactions between mitochondrial F₁/F₀ ATPase and the inhibitor protein (IF₁), which can be added or removed from the captured enzyme complex in a dose-dependent, and pH-sensitive fashion that mimics normal interactions between F₁/F₀ ATPase and IF₁. For example, ATP hydrolysis activity of mitochondrial F₁/F₀ ATPase solubilized, immunocaptured and assayed at pH in the range from about 6.0 to about neutrality is relatively low, but the enzyme could be greatly activated (>10-fold) by conditions that strip the IF₁ from the protein (30 minutes exposure to pH above 7.0, for example 8.0, 8.2 or about 8.5. The inhibition could be reversed by addition of recombinant IF₁, which reduced the rate of ATP hydrolysis to that before stripping. These results show that enzyme isolated and captured at pH above neutrality was considerably more active when assayed at pH below neutrality (i.e., pH 6.5 than that isolated at the more acidic pH, but could be greatly inhibited by addition of purified IF₁ (FIG. 6).

Recombinant human IF₁ can also be added or removed from the captured enzyme in a dose-dependent, and pH-sensitive fashion that mimics normal interactions between F₁/F₀ ATPase and IF₁. A method is also provided by the invention for obtaining solubilized mitochondrial F₁/F₀ ATPase that is fully saturated with IF, for use in such assays in a patient-specific manner. Therefore, the functionality of endogenous IF₁ or endogenous F₁/F₀ ATPase can be determined or monitored within small samples, e.g. nanosamples. Such an assay is valuable as a research tool for studying the interactions between human mitochondrial F₁/F₀ ATPase and its inhibitor.

Yet another embodiment is a method for screening to detect agents, such as small molecules, drugs, or proteins that modify the inhibitor activity of IF₁ for human mitochondrial F₁/F₀ ATPase, for example by binding to IF₁ so as to prevent its inhibitor activity. Such small molecules, drugs, or proteins are desirable therapeutic agents that could be used to regulate ATPase activity of F₁/F₀ ATPase and thereby the energy balance and efficiency of energy utilization of cells and tissues. Such modulators have utility, for instance, in treatment of disorders of energy production or utilization.

Examples of such screening assays comprise contacting a sample containing F₁/F₀ ATPase in the presence of IF, and a test compound, and determining the degree to which the test compound modifies the inhibitor activity of IF₁ in the sample, wherein a decrease of IF inhibitor activity indicates the test compound inhibits IF₁. Compounds that increase IF inhibitor activity may also be useful in treating disorders of energy production or utilization by inhibiting ATPase activity. The screening assay can also be used to determine the degree to which a test compound increases IF₁ inhibitor activity.

In still another embodiment, the invention provides isolated monoclonal antibodies characterized as specifically binding to mitochondrial F₁/F₀ ATPase and immunocapturing the entire 16 subunit complex, wherein the complex retains functional activity. It should be understood that the monoclonal antibody may be able to immunocapture the functional complex in the absence of all 16 subunits being present, although 16 is preferred. In particular examples, a monoclonal antibody wherein the antibody has the specificity and avidity of any one of MM#1-12F4AD8AF8; MM#7-3D5AB1; MM#1-7H10BD4F9; RAC#23C-1G1; RAC#23C-24C9; MM#1-8E12; RAC#25A-5E2D7; RAC#29-2A5; RAC#29-6G5; RAC#29-8C7CC4; RAC#29-9G3; RAC#29-10A3; and RAC#29-10C6AC9 is included. In another example, an anti-Complex V mAb (see, e.g., Table 1) captures an F₁/F₀ ATPase (such as, human or bovine F₁/F₀ ATPase) that retains enzyme activity.

In another embodiment, anti-Complex V capture antibodies that both immunocapture and inhibit Complex V enzyme activity are contemplated. For example, mAb MM#7-3D5AB1 (which binds to an epitope on Complex V β subunit) and MM#1-7H10BD4F9 (which binds an epitope on the Complex V a subunit) are capable of inhibiting ATPase activity of Complex V either when the enzyme is free in solution or when the enzyme is previously immunocaptured by another anti-Complex V capture mAb.

Alterations in F₁/F₀ ATPase reduce or eliminate energy production in mitochondria and so are pathogenic. Mutations of F₁/F₀ ATPase in patients (genetically derived) have been described and it is well known that severity of clinical symptoms are positively correlated with severity of enzyme dysfunction (Garcia et al., Biol. Chem. 275, 11075-11081, 2000). In addition, it is believed that regulation of Complex V ATPase activity during periods of anoxia, e.g., transient ischemia in stroke or cardiac arrest, is important for preservation of cellular energy stores and cellular survival (Garcia et al., Biol. Chem. 275, 11075-11081, 2000). The present disclosure provides evidence of the utility of antibody analysis in the characterization of F₁/F₀ ATPase deficiencies of all types.

VII. OXPHOS Enzyme Activity Assays

Once an OXPHOS enzyme complex has been immune captured using the methods and compositions described herein, it can optionally be assayed for an enzymatic activity associated with that complex. By way of example, such assays provide information not only on the quantity of enzyme complex that has been captured, but also on the relative or absolute functionality of the captured complex.

Advantageously, any general biochemical activity of a target OXPHOS enzyme complex can be used as a qualitative or quantitative indicator of the OXPHOS complex's presence in the sample and/or its function because the specificity of the capture antibody provides proof of the identity of the captured antigen. To illustrate this advantage, consider that there are many enzymes in the cell capable of hydrolyzing ATP; thus, simply showing that a crude cellular extract containing a mixture of proteins has ATPase activity does not prove that a particular ATPase in question is present in the mixture, nor does it allow measurement of that particular enzyme's activity. However, once a particular ATPase antigen (such as Complex V) is captured by an appropriate capture mAb (such as, MM#1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9, as described in Table 1), then all of the ATPase activity present in the antibody-bound (captured) material can be ascribed to the ATPase antigen known to be captured by the mAb used. This the disclosed immunocapture assays provide simple, rapid assays of OXPHOS enzyme activity and eliminate the traditional need to test parallel samples in the presence and absence of various specific inhibitors of particular enzymes.

There are wide variety of published protocols for colorimetric or absorbance based assays to measure each of the mitochondrial enzyme activities in solution (see, for example, Rickwood et al., in Mitochondria. A Practical Approach, ed. Darley-Usmar et al., Oxford:IRL Press, 1987; Birch-Machin and Turnbull, Meth. Cell Biol., 65: 97-117, 2001; Chretien et al., Biochem. Biophys. Res. Commun., 301: 222-224, 2003; Janssen et al., Ann. Clin. Biochem., 40: 3-8, 2003). Widely known in-solution assays are easily adapted by one of ordinary skill in the art for use on immunocaptured enzymes.

Immunocaptured Complex V ATP hydrolysis activity can be measured with an established end-point assay in which the reaction of molybdenum with phosphate released by ATP cleavage is measured spectrophotometrically (see also, Aggeler et al., J. Biol. Chem., 277: 33906-33912, 2002). Alternatively, by incorporating another well-established method for analysis of real-time kinetic ATPase activity in solution (Rickwood et al., in Mitochondria. A Practical Approach, ed. Darley-Usmar et al., Oxford:IRL Press, 1987), immunocaptured ATPase enzyme kinetics can be measured in real time by following the linked oxidation of NADH to NAD spectrophotometrically by monitoring the rate of change in absorbance at OD₃₄₀. The assays for immunocaptured Complex V activity can also be made more sensitive by using fluorescence-based assays for free inorganic phosphate (such as those commercially available through Molecular Probes, Inc., Eugene, Oreg.). Specific assays for measuring Complex V activity are shown, for instance, in Examples 2 and 10.

Oxidation and/or reduction (for example, catalyzed by Complexes I, II, III, or IV) can be detected by any method known in the art. In some examples, a detectable change in a physical property of the oxidized and/or reduced substrate molecule(s) is measured; for example, a change in optical density (OD) at some defined wavelength. In particular examples, OD₃₄₀ can be used to monitor the ratio of NAD/NADH redox (such as, in assays of Complex I activity), or OD₆₀₀ can be used to monitor reduction of 2,6-dichlorophenolindophenol (such as, in assays for Complex II activity), or OD₅₅₀ can be used to monitor oxidation of cytochrome c (II) (such as, in assays for Complex IV activity) (see, e.g., Birch-Machin and Turnbull, Meth. Cell Biol., 65: 97-117, 2001). In other examples, oxidation and/or reduction can be detected by monitoring a change in the properties of a prosthetic group in the oxidoreductase enzyme; for example, the ratio of OD₆₀₅/OD₆₃₀ can be used to monitor heme aa3 of Complex IV (see, e.g., Rickwood et al., in Mitochondria. A Practical Approach, ed. by Darley-Usmar et al., Oxford:IRL Press, 1987). In still other examples, oxidation and/or reduction can be detected by coupling the oxidation or reduction reaction of interest to another more easily monitored redox reaction, such as oxidation or reduction of a chromogenic or fluorogenic substrate (see, e.g., Birch-Machin and Turnbull, Meth. Cell. Biol., 65: 97-117, 2001; Amplex Red reagent (10-acetyl-3,7-dihydroxyphenoxazine available from Molecular Probes)).

Protocols for measuring Complex I activity in solution traditionally rely on monitoring absorbance changes at around 340 nm as NADH is consumed (see, e.g., Chretien et al., Biochem. Biophys. Res. Commun., 301: 222-224, 2003). The intrinsic diaphorase activity of Complex I can also be used as a reporter of enzyme function using resazurin as the electron donor (see Example 1). Resazurin is not fluorescent but, on reduction, it is converted to highly fluorescent resorufin, which has a high fluorescence intensity coefficient. Complex I activity assays are also described, for instance, in Examples 1 and 8.

Methods for analysis of Complex II and III in solution are well known in the art (see, for example, Birch-Machin and Turnbull, Meth. Cell Biol., 65: 97-117, 2001). There are several dyes with the correct midpoint potentials for electron transfer through Complexes II or III which change color or fluorescence yield in going from oxidized to reduced. For example, Complex II can follow reduction of the oxidized substrate 2,6-dichlorophenolindophenol by monitoring changes in OD₆₀₀ (Birch-Machin and Turnbull, Meth. Cell Biol., 65: 97-117, 2001). These dyes, which are available commercially (e.g., Molecular Probes), may be used in the well-established solution assays for measuring the activities of immunocaptured Complexes II and III (for instance, in 96-well microassays).

The activity of immunocaptured Complex IV can be determined, for example, by measuring the oxidation of ferrocytochrome c at OD₅₅₀ (see, e.g., Birch-Machin and Turnbull, Meth. Cell Biol., 65: 97-117, 2001), and as described in Example 7.

In every case, once an assay protocol is selected for the OXPHOS complex(es) selected for study, it is then possible to determine the relationship between normal (for instance, control) sample concentration and enzyme activity captured using an antibody or set of antibodies described herein. For example, measurement of a simple dilution series of each sample will be used to provide sufficient data to plot curves of captured enzyme activity per well versus sample concentration. A comparison of the slopes generated by each sample will give a measure of the relative concentrations of total enzyme activities per sample (this measurement will not discriminate between a reduction in the amount of a fully active enzyme and a reduction in the specific activity of enzyme present at normal concentration). However, comparison of Vₘₐₓ per well in the plateau region of the curve will provide an accurate measurement of relative specific activities of the two samples. These measurements therefore provide results useful for either diagnostic purposes or experimental analysis of mitochondrial function.

VIII. Immunodetection Assays

Analytical tests have been developed for the routine identification or monitoring of physiological and pathological conditions using different biological samples (e.g., urine, serum, plasma, blood, saliva, and so forth). Many of these tests are based on the highly specific interactions between specific binding pairs. Examples of such binding pairs include antigen/antibody, hapten/antibody, lectin/carbohydrate, apoprotein/cofactor and biotin/(strept)avidin. Furthermore, many of these tests involve devices (e.g., solid phase, lateral-flow test strips, flow-through tests) with one or more of the members of a binding pair attached to a mobile or immobile solid phase material such as latex beads, glass fibers, glass beads, cellulose strips or nitrocellulose membranes (see, for example, U.S. Pat. Nos. 4,703,017; 4,743,560; 5,073,484). Some particular examples are described in further detail below.

A. Immunoassay Techniques

Various immunoassay techniques can utilized the immunocapture monoclonal antibodies disclosed herein, including magnetic separation using antibody-coated magnetic beads, “panning” with antibody attached to a solid matrix (for instance, a bead or microtiter plate), and flow cytometry (see, e.g., U.S. Pat. No. 5,985,660; and Morrison et al., Cell, 96: 737-749, 1999). Particular useful immunoassay methods include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, 1994, Current Protocols in Molecular Biology, Vol. 1, John Wiley & Sons, Inc., New York). Exemplary immunoassays are described briefly below (but are not intended by way of limitation).

Immunoprecipitation protocols generally comprise combining an antibody of interest (such as an anti-OXPHOS capture antibody) with a sample containing an antigen recognized by the antibody (such as an OXPHOS protein or complex) and separating the antibody/antigen complex from other components of the sample. Any sample containing an antigen of interest can be used in an immunoprecipitation assay, including, for example, cell lysates, cellular fractions, or isolated organelles (such as, mitochondria) or extracts thereof. A cell lysate can be prepared by any method known in the art; for example, cells can be placed in lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate). One advantage of the disclosed capture antibodies is the ability to bind native OXPHOS proteins and complexes. Thus, it is beneficial to prepare the sample in a manner that maintains the native structure and/or function of the target OXPHOS protein and/or complex. In some examples, the sample is prepared using a gentle non-ionic detergent, such as n-dodecyl-β-D-maltoside, Cymal-5, n-decyl-β-D-thiomaltopyranoside, Hega-11, and n-tridecyl-β-D-maltopyranoside (Ko et al., J. Biol. Chem., 278: 12305-12309, 2003).

Samples useful in immunoassays are collected or obtained using methods well known to those skilled in the art. Sample containing OXPHOS proteins or complexes may be obtained from any biological source. Examples of biological sources include, but are not limited to, blood serum, blood plasma, urine, spinal fluid, saliva, fermentation fluid, lymph fluid, tissue culture fluid and ascites fluid of a human or animal, or any sample that may be expected to contain mitochondria (e.g., in cells suspended in a fluid sample, or in a tissue sample). The sample may be diluted, purified, concentrated, filtered, dissolved, suspended or otherwise manipulated prior to immunoassay to optimize the immunoassay results.

Following preparation of an appropriate sample, an antibody of interest is added to the sample preparation and incubated under conditions and for a sufficient period of time to allow antibody-antigen binding (e.g., 1-4 hours at 4° C.). The antibody/antigen complex can be separated from non-bound sample components by a variety of methods; for instance, adding protein A and/or protein G sepharose beads to the antibody/sample mixture, incubating for about an hour or more at 4° C., washing the beads in a buffer and resuspending the beads in a solution appropriate for, e.g., storage of the antibody/antigen complex or subsequent analyses of the bound antigen (such as, western blot analysis, or enzyme activity assay, or detection of post-translational modification).

One of ordinary skill in the art would be knowledgeable as to the parameters that can be modified to increase the binding of the antibody to an antigen and decrease the background (e.g., pre-clearing the sample with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

The ability of an antibody of interest to specifically immunoprecipitate a particular antigen (such as an OXPHOS complex or subunit) can be assessed by a variety of methods, including, for example, western blot analysis. Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of ordinary skill in the art would be knowledgeable as to the parameters that can be modified to increase the signal detected and to reduce the background noise. For further discussion regarding western blot protocols see, e.g., Ausubel et al., eds, 1994, Current Protocols in Molecular Biology, Vol. 1, John Wiley & Sons, Inc., New York at 10.8.1.

In some examples, ELISAs involve coating one or more wells of a microtitre plate (such as a 96-well plate) with an antibody, such as an anti-OXPHOS capture antibody and, thereafter, add a sample containing an antigen of interest (such as an OXPHOS protein or complex). Different antibodies (such as, a plurality of anti-OXPHOS capture antibodies) can be place in separate wells to run multiple assays in parallel. Immunocaptured proteins may be detected by any method known in the art. In one example, all proteins in the sample can be labeled, for instance, with an amine-reactive or thiol-reactive dye; then, immunocaptured protein can be directly detected once non-bound sample components are removed. In another example, a second antibody (e.g., specific for the antigen) conjugated to a detectable compound may be added following the addition of the antigen of interest to the coated well.

Another way to perform an ELISA is to prepare an antigen and coat one or more wells of a microtiter plate (such as, a 96-well microtitre plate) with the antigen. Thereafter, an antibody of interest, which can (but need not) be conjugated to a detectable compound such as an enzymatic substrate (e.g., horseradish peroxidase or alkaline phosphatase), is added to the well(s), incubated for a period of time, and detected by appropriate methods. In this embodiment, the antibody of interest does not have to be conjugated to a detectable compound; instead, a second antibody (which recognizes the primary antibody) conjugated to a detectable compound may be added to the well. Alternatively, the primary antibody may be biotinylated, which can be detected with a labeled (or enzyme-conjugated) avidin or streptavidin compound.

One of ordinary skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

The binding affinity of an antibody to an antigen and the off rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by SCATCHARD plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody. Screening protocols for characterizing monoclonal antibodies for antibody specificity are set forth herein.

B. Solid Phase Immunoassay Devices

Solid phase immunoassay devices provide sensitive detection of analytes in biological fluid samples. Solid phase immunoassay devices incorporate a solid support to which one member of a ligand-receptor pair, usually an antibody, antigen, or hapten, is bound. Common early forms of solid supports were plates, tubes, or beads of polystyrene, which were known from the fields of radioimmunoassay and enzyme immunoassay. More recently, a number of porous materials such as nylon, nitrocellulose, cellulose acetate, glass fibers, and other porous polymers have been employed as solid supports.

In the more common forms of dipstick assays, as typified by home pregnancy and ovulation detection kits, immunochemical components such as antibodies are bound to a solid phase. The assay device is “dipped” for incubation into a sample suspected of containing the subject analyte. Enzyme-labeled antibody is then added, either simultaneously or after an incubation period. The device next is washed and then inserted into a second solution containing a substrate for the enzyme. The enzyme-label, if present, interacts with the substrate, causing the formation of colored products, which either deposit as a precipitate onto the solid phase or produce a visible color change in the substrate solution. EP-A 0 125 118 discloses such a sandwich type dipstick immunoassay. EP-A 0 282 192 discloses a dipstick device for use in competition type assays.

Flow-through type immunoassay devices were designed to obviate the need for incubation and washing steps associated with dipstick assays. U.S. Pat. No. 4,632,901 discloses a sandwich immunoassay device wherein antibody (specific to a target antigen analyte) is bound to a porous membrane or filter to which a liquid sample is added. As the liquid flows through the membrane, target analyte binds to the antibody. The addition of sample is followed by addition of labeled antibody. The visual detection of labeled antibody provides an indication of the presence of target antigen analyte in the sample.

Migration assay devices usually incorporate within them reagents that have been attached to colored labels, thereby permitting visible detection of the assay results without addition of further substances. See, for example, U.S. Pat. No. 4,770,853; WO 88/08534; and EP-A 0 299 428.

Examples of devices useful in the methods described herein may include a strip of absorbent material, which can be made of different substances each joined to the other in zones, which may be abutted and/or overlapped. The absorbent strips are fixed on a solid support. Zones within each strip may differentially contain the specific binding partner(s) and/or other reagents required for the detection and/or quantification of the particular analyte being tested for, for example, an OXPHOS protein or complex. Thus these zones can be viewed as functional sectors or functional regions within the test device. A fluid (e.g., a sample) applied to the strip migrates distally through all the functional regions of the strip. The final distribution of the fluid in the individual functional regions depends on the adsorptive capacity and the dimensions of the materials used.

1. Lateral Flow Devices

The construction and design of lateral flow devices is very well known in the art, as described in the immediately preceding section, and see, for example, Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., Keene, N.H. 03431, and Allen and Singh, Instrument-Free Quantitative Test Systems, in Diagnostics in the year 2000: antibody, biosensor, and nucleic acid technologies, ed. by Singh et al., published by Van Nosgtrand Reinhold, 1993, pages 147-176. Lateral flow devices may have a wide variety of physical formats that are equally well known in the art. Any physical format that supports and/or houses the basic components of a lateral flow device in the proper function relationship is contemplated by this disclosure.

Some of the materials that may be useful for the components of a lateral flow device are shown in Table 3. However, one of skill in the art will recognize that the particular materials used in a particular lateral flow device will depend on a number of variables, including, for example, the analyte to be detected, the sample volume, the desired flow rate and others, and can routinely select the useful materials accordingly.

TABLE 3
Exemplar Materials Useful for Components of a Lateral Flow Device

| Component | Useful Material |
|---|---|
| Sample Pad | Glass fiber |
| | Woven fibers |
| | Screen |
| | Non-woven fibers |
| | Cellulosic filters |
| | Paper |
| Conjugate Pad | Glass fiber |
| | Polyester |
| | Paper |
| | Surface modified polypropylene |
| Membrane | Nitrocellulose (including pure nitrocellulose |
| | and modified nitrocellulose) |
| | Nitrocellulose direct cast on polyester support |
| | Polyvinylidene fluoride |
| | Nylon |
| Absorbent Pad | Cellulosic filters |
| | Paper |

a. Sample Pad

The sample pad is an optional component of a lateral flow device that initially receives the sample, and may serve to remove particulates from the sample. Among the various materials that may be used to construct a sample pad (see Table 3), a cellulose sample pad may be beneficial if a large bed volume (e.g., 250 μl/cm²) is a factor in a particular application. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

b. Membrane and Application Solution

The membrane serves to immobilize the capture reagent and to provide a surface across or through which the applied sample will flow. Nitrocellulose (whether pure or modified in any manner known in the art) is a contemplated membrane for a lateral flow device. Nitrocellulose is thought to bind proteins by hydrogen bonding, hydrophobic interactions, and by electrostatic mechanisms (see, e.g., Millipore Corporation, A Short Guide Developing Immunochromatographic Test Strips, 2nd Edition, pp. 1-40, 1999, available by request at (800) 645-5476).

For protein-containing capture reagents, the dipole of the nitrate ester of nitrocellulose interacts with the strong dipole of the peptide bonds of the protein. Salts at high concentrations, detergents, and water in an application solution may weaken and destabilize electrostatic interactions between a nitrocellulose membrane and a protein to be applied to the membrane. Thus, it is preferable, though not required, to use a low molarity buffer, for example, 2-10 mM phosphate, borate or carbonate buffers, to solubilize protein-containing capture reagents for immobilization onto nitrocellulose.

The pH of an application solution may, but need not, be adjusted to increase binding of the capture reagent to a nitrocellulose membrane. For example, the solubility of a protein-containing capture reagent in an application solution is at a minimum when the pH of the application solution is within about ±1 pH unit of the pI of the protein-containing capture reagent.

Optionally, 1 to 5% methanol, ethanol or isopropanol may be added to an application solution. An application solution may be applied to a membrane manually or in an automated manner. For example, a reagent dispensing module (e.g., Matrix 1600, Kinematic Automation, Twain Harte, Calif.) may be used to apply capture reagent to nitrocellulose.

Blocking of a membrane in a lateral flow device before addition of test samples is generally not necessary. For example, proteins that are present in the sample and other blocking agents, which may be added, e.g., to the sample pad or conjugate pad, are generally sufficient to prevent an analyte from being non-specifically adsorbed onto the membrane. If optional blocking a membrane is desired for a particular application, useful blocking agents include, for example, gelatin (0.1%-0.5%), nonfat dry milk (0.5%-2%), casein (1%-2%), BSA (1%-2%), IgG (1%-2%), PVP 8-10 kD (0.5%-1.0%), and PVA 8-10 kD (0.5%-1.0%).

C. Conjugate Pad

The conjugate pad serves to, among other things, hold a detector reagent. In some embodiments, a detector reagent may be applied externally, for example, from a developer bottle, in which case a lateral flow device need not contain a conjugate pad (see, for example, U.S. Pat. No. 4,740,468).

Detector reagent(s) contained in a conjugate pad is released into solution upon application of the test sample. A conjugate pad may be treated with various substances to influence release of the detector reagent into solution. For example, the conjugate pad may be treated with PVA or PVP (0.5% to 2%) and/or Triton X-100 (0.5%). Other release agents include, without limitation, hydroxypropylmethyl cellulose, SDS, Brij and β-lactose. A mixture of two or more release agents may be used in any given application. In the particular disclosed embodiment, the detector reagent in a conjugate pad is a labeled non-capture anti-OXPHOS antibody.
d. Absorbent Pad The use of an absorbent pad in a lateral flow device is optional. The absorbent pad acts to increase the total volume of sample that enters the device. This increased volume can be useful, for example, to wash away unbound analyte from the membrane. Any of a variety of materials is useful to prepare an absorbent pad, see, for example, Table 3. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad.
   C. Detection Methods for Immunoassays

1. Sandwich Assay (“2 Site Assay”)

One principle category of immunochromatographic assay is the “sandwich” assay. In general, sandwich immunochromatographic procedures involves the formation of a complex between a primary antibody (such as an anti-OXPHOS capture antibody), its antigen (such as an OXPHOS protein or complex), and a secondary, detectable (or detector) reagent (such as, a labeled or enzyme-conjugated antibody which also binds the antigen). In this complex, the antigen is “sandwiched” between two molecules that each bind the antigen. Preferably, a primary antibody and a secondary detector reagent do not bind the same (or competing) site on the antigen. In one example, one of the components of the sandwiched complex is immobilized on a chromatographic medium containing a band or zone of the immobilized component. The chromatographic medium often is in the form of a strip that resembles a dipstick. In one embodiment, an anti-OXPHOS capture antibody is immobilized on a chromatographic medium, for example, on a dipstick. In this case, a sample and a detector reagent are mixed and applied in the chromatographic medium so that the sample and the detector reagent come in contact with the zone of the immobilized capture antibody. If the antigen (e.g., an OXPHOS protein or complex) is present in the sample, a sandwich complex will form where an anti-OXPHOS capture antibody is immobilized. As a result, the detector reagent complex will also be localized in this immobilization zone and can be detected. Detection of the detector reagent in the zone of the immobilized component indicates the presence of the analyte (e.g., OXPHOS protein or complex) in the sample. This technique can be used to obtain quantitative or semi-quantitative results. Examples of sandwich immunoassays performed on test strips are described in U.S. Pat. Nos. 4,168,146 and 4,366,241.

“Multiplex” simultaneous analysis of many different analytes within a single sample can be done by using mixtures and arrays of appropriate pairs of primary antibody and secondary detectors. In such cases, the primary antibodies can be arrayed in a set of separate discrete zones and the secondary detectors labeled with different, distinguishable markers, such as a range of different fluorochromes. This allows the simultaneous measurement, in parallel, of many different analytes in a single sample, conserving both time and sample.

2. Labeled Sample

The specificity of a disclosed anti-OXPHOS capture antibody enables the specific immunocapture of a target antigen (such as an OXPHOS protein or complex) from a sample. Thus, it is possible to label substantially all proteins in a sample (for example, with an amine-reactive or thiol-reactive detector molecule), and still specifically detect a particular protein of interest in the sample (such as an OXPHOS protein or complex).

The disclosed methods contemplate labeling substantially all protein components of a sample with a detectable molecule, including, for example, a fluorescent dye, a detectable enzyme or enzyme domain (such as, horseradish peroxidase, β-galactosidase, alkaline phosphatase, chloramphenicol acetyltransferase), a metal colloid (such as gold or silver), biotin, or a short segment of DNA (e.g., an oligonucleotide). Such detectable molecules are well known in the art and any such detectable marker is contemplated for use in the disclosed methods.

After combining the labeled sample with the antibody(ies) of interest (such as, anti-OXPHOS capture antibodies), the non-bound components of the sample can be removed and the specifically bound and labeled antigen(s) can be readily detected.

3. Two-Marker System

In some circumstances, it may be advantageous to add more than one sample (such as a test and a control sample) to the same capture antibody or set of capture antibodies (for example, on an antibody array or a dipstick with multiple antibody zones). For instance, this technique permits detection and comparison of different levels of a protein (such as an OXPHOS protein or complex) in the individual samples. In this case, it is useful to differentially label the samples (for example, with different fluorescent markers) so that antigens from each sample, which are bound by the same antibody (or set of antibodies), can be distinguished.

In one embodiment, a two-dye system involves solubilizing a reference sample (such as, a control mitochondrial or cell extract) and the test sample (such as a mitochondrial or cell extract from a patient) in a gentle, non-ionic detergent to maintain OXPHOS proteins or complexes in their native state. Optionally, a protease inhibitor cocktail can be included to prevent degradation of the OXPHOS targets. The solubilized reference sample is labeled with a first detectable marker (such as, a first fluorescent dye) in a first labeling reaction, and the test sample is labeled with a second detectable marker (such as, a second fluorescent dye) in a second (and separate) labeling reaction. Unreacted dye is inactivated in each sample using methods known in the art. Following labeling, the two samples are mixed in equal amounts, and added to an antibody (or set of antibodies) of interest (such as, an anti-OXPHOS capture antibody). Particular examples involve antibodies (including anti-OXPHOS capture antibodies) located on an array or immobilized on a dipstick. After a sufficient period for antibody/antigen binding and removal of non-bound sample components (for example, by washing an array or dipstick containing the antigen/antibody complexes), specific immunocapture of the various labeled target proteins can be measured by detecting the first and second detectable markers (for example by 2-color fluorescence using a microplate reader).

The ratios of the signals from the first and second detectable markers (e.g., for reference and test samples) provides a qualitative indicator of the relative levels of target antigen in the respective samples. Quantitative values can be calculated by comparing the measured ratio to previously prepared standard curves in which are plotted the relationship between signal ratios and actual antigen concentration ratios in reference:test samples. The data for the standard curves is established using pairs of samples containing a wide range of known amounts of target antigen, e.g., covering the range from (i) no antigen in the reference sample and excess antigen in the test sample to (ii) excess antigen in the reference sample and no antigen in the test sample, with several samples at intermediate ratios (for example, near 1:1).

In some examples, a known concentration of an exogenous control protein is added to each sample (for example, a test and control sample) prior to dye-labeling of the samples. Accordingly, the exogenous control protein in each sample will also be labeled with the respective detectable marker. The samples are then contacted with one or more test antibodies (such as, anti-OXPHOS capture antibodies) and an antibody specific for the exogenous control protein; for example in an array or dipstick format. Thus, for example in a situation where two fluorescent markers are used, the fluorescence ratio (dye 1 dye 2) of the exogenous control protein after antibody binding can be used to verify that the labeling and detection procedures were performed correctly and also to provide a normalization value to apply to all other protein ratios.

IX. Antibody Arrays

A recent trend in biology, biotechnology and medicine is the use of arrays of immobilized biological compounds, such as antibodies, in studies of immunoassays and enzymatic reactions. Antibody arrays are useful in a wide variety of applications, including multi-analyte detection, and diagnosis, prognosis and treatment of human diseases, such as leukemia, breast cancer and, potentially, heart failure (reviewed in Lal et al., Drug Discov. Today, 7(18 Suppl): S143-149, 2002). The disclosed anti-OXPHOS capture antibodies enable antibody arrays capable of detecting one or more native OXPHOS proteins or complexes (such as one or more of Complexes I, II, III, IV, or V). Such OXPHOS arrays Antibody arrays are well known in the art and any known array configuration is contemplated by this disclosure. Non-limiting examples of protein (including antibody) arrays are described in Ivanov et al., Mol. Cell. Proteomics, 3(8): 788-795, 2004; Huang, Comb. Chem. High Throughput Screen, 6(8): 769-775, 2003; Anderson et al., Brain, 126(Pt 9): 2052-2064, 2003; Angenendt et al., Anal. Biochem., 309(2): 253-260, 2002; U.S. Pat. No. 4,591,570; U.S. Pat. No. 4,829,010; U.S. Pat. No. 5,486,452; Eur. Pat. No. EP-A-063810; Haab et al., Genome Biol. 2: 1-13, 2001), Kingsmore and Patel, Curr. Opin. Biotechnol., 14: 74-81, 2003; Michaud and Snyder, BioTechniques, 33: 1308-1316, 2002.

An array may be regular (arranged in uniform rows and columns, for instance) or irregular. The number of addressable locations on the array can vary, for example from a few (such as three) to more than 50, 100, 200, 500, 1000, 10,000, or more. A “microarray” is an array that is miniaturized to such an extent that it benefits from microscopic examination for evaluation.

Within an array, each arrayed molecule (e.g., anti-OXPHOS capture antibody) or sample (more generally, a “feature” of the array) is addressable, in that its location can be reliably and consistently determined within the at least two dimensions on the array surface. Thus, in ordered arrays the location of each feature is usually assigned to a sample at the time when it is spotted onto or otherwise applied to the array surface, and a key may be provided in order to correlate each location with the appropriate feature(s).

Within an array, each arrayed molecule can be located at multiple sites, allowing for simultaneous independent measurements of the same analyte in a single sample in a single experiment. This configuration facilitates, for instance, calculation and statistical analysis of analyte concentrations and activities with greater accuracy and higher confidence levels.

Often, ordered arrays are arranged in a symmetrical grid pattern, but samples could be arranged in other patterns (e.g., in radially distributed lines, spiral lines, or ordered clusters). Arrays are in general computer readable, in that a computer can be programmed to correlate a particular address on the array with information (such as identification of the arrayed sample and binding data, including for instance signal intensity). In some examples of computer readable array formats, the individual spots on the array surface will be arranged regularly, for instance in a Cartesian grid pattern, that can be correlated to address information by a computer.

The sample application spot (or feature) on an array may assume many different shapes. Thus, though the term “spot” is used herein, it refers generally to a localized deposit of nucleic acid or other biomolecule, and is not limited to a round or substantially round region. For instance, substantially square regions of application can be used with arrays, as can be regions that are substantially rectangular (such as a slot blot-type application), or triangular, oval, irregular, and so forth. The shape of the array substrate itself is also immaterial, though it is usually substantially flat and may be rectangular or square in general shape. Also contemplated herein are arrays of containers capable of receiving fluid, for instance, arrays of microwells or other cavities such as those of a 96-well plate or other multi-sample plate(s).

In one embodiment, an anti-OXPHOS capture antibody array (such as a 96-well plate) includes a plurality of anti-OXPHOS capture antibodies, for example, at least 5, at least 7, at least 10, at least 15 different capture antibodies placed in discrete addressable locations. In particular examples, antibodies having differing specificities can be placed on an array, including at least one anti-OXPHOS capture antibody (such as, anti-Complex I, anti-Complex II, anti-Complex III, anti-Complex IV or anti-Complex V or any combination thereof) and one or more of (i) an antibody specific for pyruvate dehydrogenase enzyme complex), (ii) an antibody specific for an endogenous mitochondrial control protein (such as, porin), (iii) an antibody specific for an exogenous non-mitochondrial control protein (such as, plasmodium malaria lactate dehydrogenase), or (iv) a null antibody (such as, pooled normal mouse immunoglobulins known not to bind any mitochondrial proteins).

Anti-OXPHOS capture antibody arrays are particularly advantageous, at least, because (i) they allow simultaneous analysis of a complete bioenergetic system, namely the OXPHOS system, and (ii) they allow analysis of nearly 100 different OXPHOS proteins with the use of as few as 5 arrayed OXPHOS capture mAbs. This enhanced analytic power as compared to, for example, other 5 mAb arrays for individual proteins, results from the fact that the OXPHOS capture mAbs can capture fully or partially assembled OXPHOS complexes; thus, by capturing up to all five OXPHOS complexes in a single array, it is possible to monitor each of the subunits of the captured OXPHOS complex(es) (in some cases nearly 100 different OXPHOS protein subunits).

Optionally, one or more control antibodies are included in a disclosed antibody array. For example, it can be helpful to include an antibody specific for an exogenous (non-human, non-mitochondrial) control protein, such as plasmodium malaria lactate dehydrogenase. As described previously, an exogenous control protein can be used to can be used to verify that the labeling and detection procedures were performed correctly and also to provide a normalization value to apply to all other protein ratios (see section entitled “Two Marker System”). Another useful control antibody to include in a disclosed array binds a non-OXPHOS mitochondrial target protein that has relatively constant expression under many biological conditions, such as antibodies specific for porin or citrate synthase. These controls can serve as optional benchmarks for comparative longitudinal studies. In other examples, an antibody array can include a null antibody (such as, pooled normal mouse IgG, pre-screened to verify that it does not bind any mitochondrial proteins) to establish the levels of background noise.

X. Kits for Detection and/or Quantification of Mitochondrial Protein(s)

Assay devices as described herein can be provided in the form of kits. Such kits will include one or more assay devices and instructions for the use of the device(s). The instructions may provide direction on how to apply sample to the test device, the amount of time necessary or advisable to wait for results to develop, and details on how to read and interpret the results of the test. Such instructions may also include standards, such as standard tables, graphs, or pictures for comparison of the results of a test. These standards may optionally include the information necessary to quantify analyte using the test device, such as a standard curve relating intensity of signal or number of signal lines to an amount of analyte therefore present in the sample.

In still another embodiment, the disclosure provides a kit for determining the quantity and/or activity of one or more OXPHOS complexes. The kit comprises one or more capture antibodies. The kit may contain a detectable label, such as a fluorescent label or an enzymatic label, with which the antibody can be tagged for detection of formation of a complex between the capture antibody and the OXPHOS complex when the antibody is contacted with a complex containing sample. Alternatively, the kit may contain any of the monoclonal antibodies contained in Tables 1 and 2, or any set of those antibodies. The antibodies can be bound to a solid support, such as a 96-well microtiter plate or beads. The kit may further comprise instructions for performing immunoassay of a sample containing an OXPHOS protein or OXPHOS complex (such as, Complex I, II, III, IV, or V) or combination thereof using the contents of the kit.

In specific examples, a kit is designed to measure the relative concentrations of OXPHOS complexes and associated proteins in a variety of paired samples (e.g., normal versus test samples), including isolated mitochondria, whole cell detergent extracts, and tissue extracts. One non-limiting representative kit includes (i) one or more 96-well microtitre plates including an immobilized set of monoclonal antibodies each specific for a different OXPHOS complex/protein (for example, one or more antibodies provided in Table 1) or one or more control proteins (such as porin); (ii) an exogenous control target protein; (iii) detergent and protease inhibitor cocktail for mitochondrial solubilization; (iv) two spectrally distinct protein-reactive fluorescent dyes; (v) a blocking protein, such as bovine serum albumin; and, optionally, (vi) a reference normal mitochondrion total protein sample.

The following examples are provided to illustrate certain particular features and/or embodiments. These examples should not be construed to limit the invention to the particular features or embodiments described.

### EXAMPLES

### Example 1

### Proteomic Analysis of Complex I is Useful in the Detection of Human Complex I Defects

Complex I defects are one of the most frequent causes of mitochondrial respiratory chain disorders. This Example demonstrates that there is a correlation between OXPHOS Complex I levels and/or assembly patterns and Complex I disorders; thus, methods of detecting Complex I can facilitate the diagnosis of Complex I deficiencies.

A. Materials and Methods

1. Purification of Complex I From Bovine Heart

Biochemically purified bovine heart Complex I as well as the flavoprotein, iron-sulfur protein, and hydrophobic protein subfractions of Complex I isolated as described previously (Hatefi, Meth. Enzymol., 53: 11-14, 1978; Galante et al., Meth. Enzymol., 53: 15-21, 1978; Galante et al., Arch. Biochem. Biophys., 192: 559-568, 1979) were kindly supplied by Dr. Youssef Hatefi (The Scripps Institute, La Jolla, Calif.). Immunopurified bovine heart Complex I was generated by solubilizing bovine heart mitochondria in 1% n-dodecyl-β-D-maltoside (LM; Calbiochem, S. Dak., CA), centrifuging twice (10,000×g, 12 minutes) to remove insoluble material, passing the supernatant over an immunoaffinity column generated as described previously (Harlow et al., Antibodies: A Laboratory Manual, Cold Spring Harbor Laboratory, Cold Spring Harbor, N.Y., 1988) using the 15-kDa Complex I mAb 17G3D9E12 created as described below, washing with phosphate-buffered saline (PBS) containing 0.05% LM, and eluting with 100 mM glycine, pH2.5.

2. Cell Lines

MRC5 fibroblasts were obtained from the American Type Culture Collection (ATCC), and MRC5-RHOO fibroblasts were derived from the MRC5 fibroblasts by culturing the cells in permissive medium supplemented with 50 ng/ml ethidium bromide as described previously (Marusich et al., Biochim. Biophys. Acta, 1362: 145-159, 1997). Patient fibroblasts were obtained from skin biopsies of young children in whom an isolated Complex I deficiency has been confirmed in muscle tissue as well as in cultured fibroblasts, using the slightly modified method of Fischer et al. (Fischer et al., Clin. Chim. Acta, 155: 263-273, 1986). The phenotypes and genotypes of the patients included in this study have been extensively described by Loeffen et al. (Loeffen et al., Hum. Mut., 15: 123-134, 2000). Control fibroblasts were obtained from post-circumcision tissue from a child in the same age range in whom biochemical enzyme analyses revealed normal results.

3. Monoclonal Antibodies

The monoclonal antibodies used in this Example were developed at the University of Oregon (Eugene, Oreg.) by immunizing mice with purified bovine Complex I as described previously (Marusich., J. Immunol. Meth., 114: 155-159, 1988). The antigen used to generate monoclonal antibodies was beef heart Complex I purified according to Hatefi (Meth. Enzymol., 53: 11-14, 1978). Virgin female BALB-C mice, 6-8 weeks old were given a primary intraperitoneal immunization of an emulsion consisting of one part aqueous antigen solution and three parts complete Freund's adjuvant. Subsequent boosts were given at 3-4 week intervals, and were also delivered intraperitoneally, but consisted of antigen emulsified in incomplete Freund's adjuvant (IFA). The final boosts were given 3 and 4 days before the splenocytes were harvested, and were delivered intraperitoneally, in either IFA or saline.

4. Splenocyte Preparation

Animals were euthanized by cervical dislocation, their spleens removed aseptically, and then teased apart with forceps to prepare a splenocyte suspension in 10 ml of high glucose Dulbecco's modified Eagle's Medium (HGDMEM). Undissociated tissue fragments were allowed to sediment for 1-2 minutes at one gravity, and the supernatant was saved. The splenocytes were then collected by centrifugation at 300×g for 5 minutes, and resuspended in 10 ml HGDMEM. A 10 μl aliquot of this suspension was then combined with 490 μl of 3% acetic acid and the splenocytes counted. Splenocytes were then mixed with the appropriate number of myeloma cells and immediately processed for cell fusion. The remaining splenocytes were collected by centrifugation, resuspended at a concentration of 4.5×10⁷ cells/ml in freezing medium (one part DMSO, nine parts 20F-HgDMEM) and 1.5 ml aliquots sealed in cryotubes. The cryotubes were then placed in a Styrofoam box, frozen at −80° C. for one day, and then transferred to a liquid nitrogen freezer. Cell fusions were conducted as known in the art except that macrophage feeder cells were replaced in cell culture media by P388D1 cells (ATCC) producing plasmacytoma-growth factor.

5. Establishing Antibody Specificity

The newly generated monoclonal antibodies (mAbs) were sequentially screened for (1) binding to purified bovine Complex I adsorbed to polystyrene; (2) binding specifically to a single subunit in denaturing Western blots of bovine Complex I; (3) binding to a single subunit in denaturing Western blots of the flavoprotein, iron-sulfur protein, or hydrophobic protein subfractions of bovine Complex I; (4) binding to a single subunit in denaturing Western blots of immunopurified bovine Complex I; (5) binding to a single subunit in denaturing Western blots of human mitochondria; and (6) reactivity and mitochondrial localization in immunohistochemistry of human mitochondria. Immunohistochemistry was carried out as described previously (Marusich et al., Biochim. Biophys. Acta, 1362: 145-159, 1997).

The monoclonal antibody concentrations used in these studies were: anti-Complex-I-39 kDa, anti-Complex-I-15 kDa, anti-Complex-I-8 kDa, anti-Complex-IV Va, and anti-Complex V-α at 2.0 μg/ml, anti-Complex II-70 kDa at 0.15 μg/ml (Marusich et al., Biochim. Biophys. Acta, 1362: 145-159, 1997), anti-Complex III-core Complex at 0.3 μg/ml, anti-Complex II at 3.0 μg/ml, anti-Complex IV at 0.5 μg/ml (Capaldi et al., Meth. Enzymol., 260: 117-132, 1995), and anti-Complex I-20 kDa and anti-Complex 1-25 kDa as twice-diluted hybridoma cell culture supernatants. The commercially obtained anti-porin antibody (Calbiochem) used in the assay as the control for equal loading of Western blots was diluted 1:120,000.

6. Overexpression of Complex I Subunits in Escherichia coli

On the basis of estimated molecular weights and by determining with which Complex I subfraction (flavoprotein, iron-sulfur protein, or hydrophobic protein) each antibody reacted, a list of possible Complex I antigens was compiled for each antibody. The cDNA of two selected Complex I subunits (NDUFA9 and NDUFS3) was then amplified by PCR from a commercially available human heart cDNA library (Life Technologies, Inc., St. Paul, Minn.) using the following forward and reverse primers:

Forward Primers:

| | |
|---|---|
| NDUFA9: | |
| 5′-TAT ATC ATG AGC CAT CAT CAT CAT | (SEQ ID NO: 1) |
| CAT CAC ATG GCG GCT GCC GCA CAA | |
| TCC-3′; | |
| and | |
| NDUFS3: | |
| 5′-CAG CCG GAT CCT CGA GCA TAT GGC | (SEQ ID NO: 3) |
| TCT AAA TGT TGA CGG TCT TGG CC-3′. | |

Reverse Primers:

| | |
|---|---|
| NDUFA9: | |
| 5′-TAT ATA CCA TGG GCC ATC ATC ATC | (SEQ ID NO: 2) |
| ATC ATC ATG AGA GCG CCG GGG CCG ACA | |
| CGC-3′, | |
| and | |
| NDUFS3: | |
| 5′-GCG CGC GCC ATA TGC TAC TTG GCA | (SEQ ID NO: 4) |
| TCA GGC TTC TTG TCT-3′ | |

The resultant PCR products were subcloned into the pET15B vector (Novagen) using BspHI-NdeI and NcoI-NdeI (New England Biolabs, Beverly, Mass.) restriction sites, respectively. BL21-DE3 cells (Novagen) were transformed with the plasmids, and when the cells reached an absorbance of 0.6, they were induced for 3 hours with 1 mM isopropyl-1-thio-β-D-galactopyranoside. Cells before and after induction were analyzed by Western blot. Antigens with which antibodies reacted after, but not before, induction are listed in Table 4.

7. Antibody Characterization

Screening involved an assay for binding to native Complex I, Western blotting, and/or immunohistochemistry. Biochemically purified and immunopurified bovine heart Complex I, biochemically purified iron-sulfur protein, flavoprotein, and hydrophobic protein subfractions of bovine Complex I and human fibroblast cell lines from controls and patients were used in the screening. Unequivocal identification of two of the antibodies was also made by Western blotting after overexpressing the candidate human subunit antigens subunits (NDUFA9 and NDUFS3) in E. coli as described above. Seven monoclonal antibodies screened in this way met the following standards for long-term culturing: They react with biochemically purified bovine Complex I, immunopurified bovine Complex I, solubilized human mitochondria, and whole-cell extracts to generate a single band in Western blot. Where two or more antibodies were obtained to a particular subunit, the antibody that worked best in immunohistochemistry or effectively immunoprecipitated Complex I, or both, was chosen. Table 4 summarizes the information on the antibodies prepared and screened as described above.

TABLE 4
Selected Capture and Non-Capture Complex I Antibodies.

| | | | | | Capture | |
|---|---|---|---|---|---|---|
| Antigen | MW | MAb | WB Conc | IC Conc | Bead | Plate |
| C-I-08 | 08 kD | RAC#24-17C8E4E11 | 1 μg/ml | HH 5 μg/ml | + | − |
| C-I-08 | 08 kD | RAC#24-18G7AC5 | 100% CM | nd | | − |
| C-I-14 | 15 kD | RAC#24-21A6BE1BA3AD1 | 0.1 μg/ml | nd | | + |
| NDUFS5 | | | | | | |
| C-I-15 | 15 kD | RAC#24-17G3D9E12 | 0.25 μg/ml | HH 5 μg/ml | + | − |
| NDUFA6 | | | | | | |
| C-I-17 | 17 kD | RAC#24A-22B8BE8H5 | 0.25 μg/ml | − | | − |
| NDUFB6 | | | | | | |
| C-I-17 | 17 kD | RAC#24A-21C11BC11AA1 | <0.5 μg/ml | − | | +/− |
| NDUFB6 | | | | | | |
| C-I-20 ND6 | 20 kD | RAC#24A-20E9DH10C12 | 0.5 μg/ml | − | + | − |
| C-I-20 | 20 kD | RAC#24A-20E7CD3 | +CM | | | + |
| C-I-30 | 30 kD | RAC#24A-17D950C9H11 | 0.5 μg/ml | HH 5 μg/ml | | − |
| NDUFS3 | | | | | | |
| C-I-30 | 30 kD | RAC#24A-17G8BC3 | 10F CM | 10F CM | | − |
| NDUFS3 | | | 1:4 | 1:64 | | |
| C-I-30 | 30 kD | MM#7-3F9DD2 | 0.5 μg/ml | 5 μg/ml | | − |
| NDUFS3 | | | | | | |
| C-I-39 | 39 kD | RAC#24-20C11B11B11 | 0.1-2.0 μg/ml | HH 10 μg/ml | | − |
| NDUFA9 | | | | | | |
| | nd | RAC#24-20D1AB7 | negative | nd | ++ | ++ |
| | 39 kD | RAC#24-22C2 | +CM | nd | nd | + |
| | 18 kD | RAC#24-22H12 | +CM | nd | nd | + |
| | 8 kD | RAC#24-18G12 | +CM | nd | nd | ++ |
| C-I- | 22 kD | MM#10-1G11 | +CM | nd | nd | + |
| NDUFS4 | | | | | | |
| C-I- | 22 kD | MM#-5H5 | +CM | nd | nd | + |
| NDUFS4 | | | | | | |
| C-I- | 22 kD | MM#-6C6 | +CM | nd | nd | + |
| NDUFS4 | | | | | | |
| C-I- | 22 kD | MM#-7F10 | +CM | nd | nd | + |
| NDUFS4 | | | | | | |
| C-I- | 22 kD | MM#-7H10 | +CM | nd | nd | + |
| NDUFS4 | | | | | | |
| C-I- | 22 kD | MM#-9F9 | +CM | nd | nd | + |
| NDUFS4 | | | | | | |
| C-I- | 22 kD | MM#-10D4 | +CM | nd | nd | + |
| NDUFS4 | | | | | | |
| C-I- | 22 kD | MM#-10G10 | +CM | nd | nd | + |
| NDUFS4 | | | | | | |
| | 75 kD | RAC#24A-20C6 | +CM | nd | nd | + |
| | 75 kD | RAC#24-15A5 | +CM | − | nd | + |
| | 50 kD | RAC#24-15B10 | +CM | nd | nd | + |
| | 50 kD | RAC#24-16G12 | +CM | nd | nd | + |

An antibody described in Table 4 was considered to “capture” native Complex I if (i) after mixing a Complex I-containing sample and the antibody in solution, Complex I was bound (via the antibody) to Protein-G-coated beads, and/or (ii) fluorescently labeled protein from a Complex I-containing sample was bound (after washing) to a microtitre plate coated (directly or indirectly) with the anti-Complex I capture antibody.

B. Proteomic Characterization of Human Complex I Disorders

A group of human patients were prescreened to identify the presence of DNA alterations in each of the known nuclear-encoded “structural” genes of Complex I as described previously (Triepels et al., Hum. Genet., 106: 385-391, 2000; Smeitink et al., Hum. Mol. Genet., 7: 1573-1579, 1998). Fibroblasts were cultured from 11 patients in whom an isolated enzymatic Complex I deficiency had been confirmed in muscle tissue as well as cultured fibroblasts.

In seven of the patients, the pathogenic mutation was identified genetically, and for four patients the genetic defect was not identified. The residual Complex I activities of the 11 patient fibroblast cell lines ranged from 35 to 85%. Specifics results for each patient are provided in Table 5 below.

TABLE 5
Genetic and Biochemical Characteristics of Patients' Cell Lines

| Patient | | | Residual CI |
|---|---|---|---|
| Number | Mutation | Gene | activity (%)^{a} |
| 1 | 364C | NDUFS7 | 68 |
| | A(V122M) | | |
| 2 | 236C | NDUFS8 | 69 |
| | T(P79L)^{b} | | |
| | 305G | | |
| | A(R102H)^{b} | | |
| 3 | Insert 471 AAGTC | NDUFS4 | 75 |
| 4 | Delete G290 (stop) | NDUFS4 | 62 |
| 5 | 1237T C(S413P) | NDUFS2 | 41 |
| 6 | 683G | NDUFS2 | 55 |
| | A(R228Q) | | |
| 7 | 1268C T(T423M) | NDUFV1 | 73 |
| 8 | None | | 35 |
| 9 | None | | 67 |
| 10 | None | | 85 |
| 11 | None | | |

^{a}Complex I (CI) activity (mU)/Complex IV activity (mU) in patients expressed as a percentage of the lowest activity ratio measured in control cells (n = 14).
^{b}Both mutations were compound heterozygous.

1. Fibroblast Culture and Mitochondrial Protein Isolation

Control and patient fibroblasts were grown in M199 (Life Technologies), 5 mg/liter TWEEN 20™ medium with 10% fetal calf serum, 100 IU/ml penicillin, and 100 IU/ml streptomycin. Approximately 30×10⁶ cells were harvested at 95% confluence after mild trypsinization (3-5 minutes) with 2-3 ml 0.25% trypsin solution/175-cm² (5×10⁶ cells) cell culture. Cells were resuspended in 50 ml 10% fetal calf serum-phosphate buffered saline (PBS). Cells were rinsed three times with 1% fetal calf serum-PBS as well as with PBS and finally frozen at 80° C. To obtain mitochondrial pellets, cells were solubilized in 5 ml homogenization buffer (1 mM EDTA, 0.25 M sucrose, 10 mM Tris, pH 7.4) containing protease inhibitors (0.5 μg/ml leupeptin, 0.5 μg/ml pepstatin, and 1 mM phenylmethylsulfonyl fluoride). Cells were repeatedly (three times) homogenized with a motorized pestle (15-20 strokes), and the postnuclear supernatants were pooled after centrifugation (10 minutes, 1500×g). Mitochondrial pellets were obtained by centrifugation of the collected postnuclear supernatants (15 minutes, 10,000×g). The mitochondrial pellets were washed twice (15 minutes, 10,000×g) with 2 ml washing buffer (1 mM EDTA, 0.25 M sucrose, 10 mM Tris/HCl, pH 7.5) including protease inhibitors (0.5 μg/ml leupeptin, 0.5 μg/ml pepstatin, and 1 mM phenylmethylsulfonyl fluoride). Finally, pellets were saved in 200 μl protease inhibitors/washing buffer and stored frozen at 80° C. Protein amounts were estimated A_{λ280} determination.

2. Western Blot Analysis of Mitochondrial Proteins

Approximately 5 μg/lane mitochondrial proteins prepared as described above, dissolved in SDS-polyacrylamide gel electrophoresis-Tricine sample buffer (BioRad) containing 2% β-mercaptoethanol (30 minutes, 37° C.), were separated on 10-20% gradient polyacrylamide gels in a Mini-Protein II™ Apparatus (Bio-Rad, Mahwah, N.J.). After electrophoresis (100-V stacking gel and 150-V running gel), proteins were transferred electrophoretically (2 hours, 0.10 A) to 0.45-μm polyvinylidine difluoride membranes in transfer buffer (10% methanol in 10 mM 3-[cyclohexylamino]-1-propanesulfonic acid, pH 11) on ice. The polyvinylidine difluoride membranes were blocked overnight with 5% nonfat dried milk powder in Dulbecco's phosphate-buffered saline (CMF-PBS). Afterward the blots were treated with primary, non-capture anti-Complex I monoclonal antibodies (see, e.g., Table 4) diluted in 5% milk CMF-PBS for 2 hours. After rinsing the blot three times with CMF-PBS and 0.05% TWEEN 20™, the blots were incubated for 2 hours with horseradish peroxidase-conjugated goat anti mouse IgG+IgM (heavy and light chain) at 0.2 μg/ml (Jackson Immunoresearch, Westgrove, Pa.) in CMF-PBS. Specific detection of the secondary antibody was obtained with ECL Plus™ chemiluminescent Western blotting detection reagent (Amersham Pharmacia Biotech, Piscataway, N.J.) after rinsing the blots with CMF-PBS three times.

Fluorescence was quantified using a Storm 860™ chemifluorescence imager (Molecular Dynamics, Sunnyvale, Calif.) and the accompanying Molecular Dynamics Imagequant™ software. Ratios of individual proteins detected in the immunoassay were calculated in relation to the porin signal. The ratios of the control fibroblast cell line were set to 100%, and the ratios of other cell lines were reported in comparison with this. The results obtained represent average values of two to five independent experiments for each subunit.

3. Confirmation of Isolated Complex I Deficiency Using Western Blot Data

Mitochondria were isolated from each of the patient fibroblast cell lines, a control skin fibroblast cell line, and normal and Rho0 MRC5 fibroblasts (a lung fibroblast cell line). Samples of each were examined by Western blotting with mixtures of antibodies, including ones specific to the 39-kDa subunit of Complex I, the 70-kDa subunit of succinate dehydrogenase (Complex II), core II protein of Complex III, subunit II of cytochrome c oxidase (Complex IV), subunit IV of Complex IV, the α subunit of F₁F₀ (Complex V), and porin (as a control for equal loading of lanes).

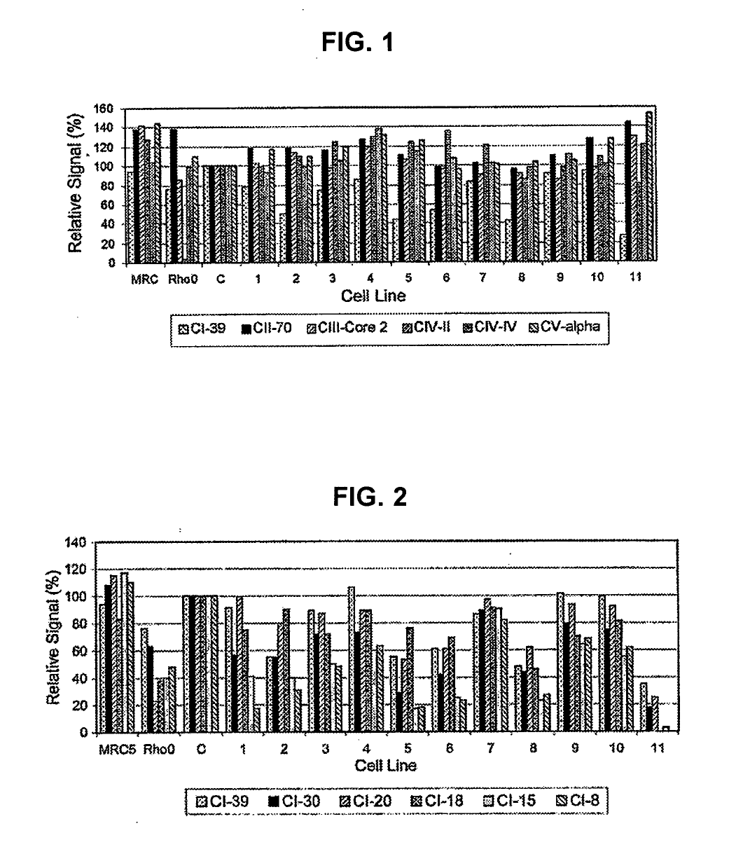

FIG. 1 summarizes the data with a bar graph in which the levels of each complex are quantified by determining the amount of the component subunit in each patient cell line in relation to that found in control skin fibroblasts. These data demonstrate that the levels of the 39-kDa subunit of Complex I, but not that of any of the other OXPHOS subunit probed, are diminished in most of the patient cell lines. This result is different than the result in MRC5-Rho0 mitochondria, in which loss of mtDNA leads to an absence of Complex IV subunit II and to lower levels of the core II protein of Complex III.

Rho0 cells are a model for cellular mtDNA defects because Rho0 cells completely lack mtDNA-encoded proteins (such as mtDNA-encoded subunit II of Complex IV). In Rho0 cells, OXPHOS complexes that normally contain mtDNA-encoded proteins at their core (CI, CIII, CIV, and to a lesser extent, CV) do not assemble properly and contain lower levels of many nuclear-DNA-encoded proteins (such as core II of Complex III). Some nuclear-DNA-encoded proteins do persist in Rho0 cells as components of “partially assembled” subcomplexes (such as subunits IV and Va of Complex IV; see, e.g., Marusich et al., Biochim. Biophys. Acta, 1362: 145-159, 1997).

4. Variations in Complex I Assembly Identified by Western Blotting

The mitochondrial samples of the 11 patient fibroblasts were examined for levels of six different subunits of Complex I (referred to by their apparent molecular weights as listed in Table 4. Usually, mAbs to the 30-, 20-, 15-, and 8-kDa subunits were used as an antibody mixture along with porin, and the amounts of the 39- and the 18-kDa subunits were quantified relative to porin separately. A bar graph of the levels of the six components of Complex I in the different samples is shown in FIG. 2. A significant reduction in the levels of one or more components of Complex I was seen in the patient samples, except for patient 7, who had a mutation in NDUFV1. The patterns of subunit loss were similar in patients 3 and 4, each of which has a different mutation in NDUFS4. Similarly, the pattern of subunit loss was the same in patients 5 and 6, each with a different mutation in the same subunit, NDUFS2. Patients 9 and 10, both of which have unidentified mutations, show remarkable similarity in the pattern of subunit loss. This pattern most closely resembles that of patients 3 and 4.

In Rho0 cells, where there is an absence of the mitochondrially encoded subunits of Complex I, a different pattern from any of the patient samples was observed. In this case, the levels of the 20- and 18-kDa subunits were as low as, or lower than, those of the 15- and 8-kDa subunits. Subunit amounts were lowest in patient 11, which identified this patient as a likely candidate for a mutation in an assembly factor (see below).

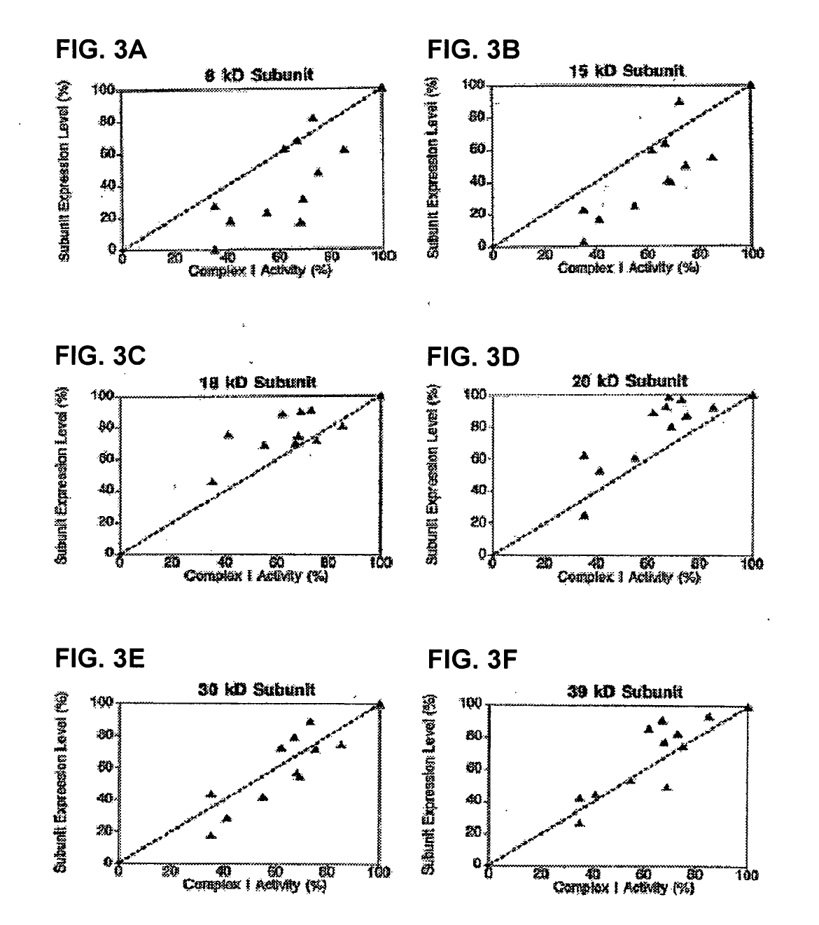

The relationship between the loss of each subunit as detected by Western blot analysis and the residual Complex I activity is shown in FIGS. 3A-F. In each panel, the dashed line represents what would be expected if there were a perfect correlation between loss of subunit and loss of activity. The levels of the 39- and 30-kDa subunits most closely track the loss of activity. However, in most cases the levels of the 20- and 18-kDa subunits are higher than predicted from the activity effects, whereas the levels of the 15- and 8-kDa subunits are lower than the residual levels of enzymatic activity.

5. Sucrose Gradient Analysis of Patient Cell Lines

Mitochondria isolated from cells lines of patient 7 (with a mutation in NDUFV1), patient 1 (with a mutation in NDUFS7), and patient 11 (with an unknown mutation) were each dissolved in 1% LM and subjected to sucrose gradient centrifugation using a discontinuous gradient as follows:

Mitochondria (1 mg) from control MRC5 fibroblasts and three patient cell lines (patients 1, 7 and 11) were pelleted (10,000×g, 10 minutes, 4° C.) and resuspended at a protein concentration of 5 mg/ml in 100 mM Tris/HCl, 1 mM EDTA, pH 7.5, 1 μg/ml pepstatin, 1 μg/ml leupeptin, 1 mM phenylmethylsulfonyl fluoride, 1% LM. The mitochondria were incubated in this solution for 20 minutes on ice with stirring before any insoluble membranes were pelleted again by centrifugation (10,000×g, 10 minutes, 4° C.). The supernatant was layered on top of a discontinuous sucrose gradient composed of 250 μl of 35% sucrose, 500 μl of 30% sucrose, 750 μl of 27.5% sucrose, 1 ml of 25% sucrose, 1 ml of 20% sucrose, and 1 ml of 15% sucrose. All sucrose solutions contained 100 mM Tris/HCl, pH 8.0, 0.05% LM, 1 μg/ml pepstatin, 1 μg/ml leupeptin, 1 mM phenylmethylsulfonyl fluoride. The gradient was then centrifuged overnight at 4° C. (128,000×g, 16.5 hours, SW 50.1). The sucrose gradient was fractionated from the bottom of the tube into 500 μl fractions, which were frozen at 80° C. For Western blotting, 20 μl of each fraction was loaded per lane.

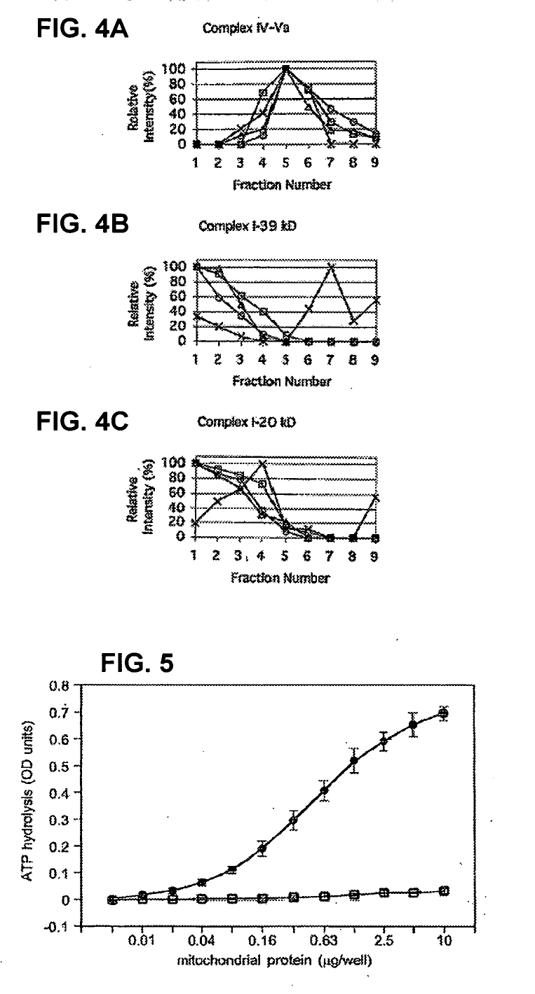

In this gradient, the five complexes of OXPHOS were separated by size, and each was identified by Western blotting of the fractions with monoclonal antibodies as described above. Densitometric scans of the Western blots were then made, and, for convenience, the relative expression levels of each subunit in the various fractions were expressed as a percentage of the highest intensity band in the gradient. For example, FIGS. 4A-C show the distribution in the gradient of the Va subunit of cytochrome c oxidase as well as the 39- and 20-kDa subunits of Complex I for the three patient cell lines and control MRC5 fibroblasts. Complex I, with a molecular weight of close to 900,000 daltons, elutes before the other respiratory chain complexes after gradient separation. The 39- and 20-kDa subunits of patients 1 and 7 eluted at a position similar to that of control Complex I, which indicates complete or near complete assembly of Complex I in these patients. In comparison, the 39- and 20-kDa subunits from patient 11 migrated in subcomplexes of approximately 200 and 500 kDa, respectively, and there was also a free subunit (eluting in fraction 9). Thus, assembly of Complex I in patient 11 is poor.

6. MALDI-TOF and LC-MS/MS Analysis of Complex I Tryptic Peptides

Matrix Assisted Laser Desorption/Ionization Time-of-Flight (MALDI-TOF) is a commonly known technique that permits mass analysis using a minimal amount of sample. This technique was used to identify the individual polypeptides of Complex I, which were immunocaptured from human heart with mAbs made against bovine Complex I.

Immunocapture: Eighty (80) μg of monoclonal antibody 17G3D9E12 (Molecular Probes, Eugene, Oreg.) was bound to 20 μg of swollen protein G-agarose beads (Sigma). The antibody was cross-linked to the beads with 25 mM dimethylpimelimidate (Sigma) for 30 min at room temperature in 0.2 M sodium borate, pH 9.0. Cross-linking was terminated with 0.2 M ethanolamine solution, pH 8.0, for 3 hours at room temperature. Antibody cross-linked beads were collected by gentle centrifugation at 3000 rpm in a microfuge and resuspended in phosphate-buffered saline. This conjugate was incubated overnight at 4° C. with the supernatant from 10 mg of solubilized mitochondria. Beads were washed six times with phosphate-buffered saline supplemented with 0.05% n-dodecyl-β-D-maltoside. Immunocaptured Complex I was eluted twice with 60 μl of 0.1 M glycine, pH 2.5, supplemented with 0.05% n-dodecyl-β-D-maltoside. The sample was then dialyzed in phosphate-buffered saline, 0.05% n-dodecyl-β-D-maltoside to neutralize the pH of the solution. MAb 17G3D9E12 typically immunoprecipitated 1 μg of enzyme/250 μg of heart mitochondria, a yield of purified organelle membranes that could be obtained routinely from as little as 10 mg of heart tissue.

Electrophoresis of NADH Dehydrogenase Complex: For one-dimensional electrophoresis samples separated on a 10-22% acrylamide gels containing 0.05% SDS, 0.375 M Tris-HCl pH 8.6. For two-dimensional electrophoresis, 100-μl samples were denatured in 350 μl of rehydration buffer (7 M urea, 2 M thiourea, 65 mM dithiothreitol, 0.8% pH 3-10 carrier ampholyte (Fluka), 2% CHAPS (Sigma), 1% Zwittergen 310 (Sigma), 0.1% SDS) for 15 minutes at room temperature. Each sample was used to hydrate 18-cm immobiline pH gradient strips pH 3-10 (Amersham Biosciences) for 12 hours. Then isoelectric focusing was performed in three stages of applied potential difference: 500 V for 1 hour, 1000 V for 1 hour, and 8000 V for up to 10 hours, until 60,000 Vh were achieved. Focused strips were then soaked in SDS-PAGE buffer (50 mM Tris-HCl, pH 8.8, 6 M urea, 30% glycerol, 2% SDS, 0.01% bromphenol blue, 100 mM dithiothreitol) for 15 minutes at room temperature. Strips were applied to 15% acrylamide gels for SDS-PAGE. Gels were stained with Coomassie Brilliant Blue (Sigma) or Sypro Ruby (Molecular Probes) gel stains. Silver staining was performed according to Shevchenko et al. (Anal. Chem., 68: 850-858, 1996).

Sample Preparation for Analysis by Mass Spectrometry: Sypro Ruby and silver-stained two-dimensional gel spots were excised using a ProteomeWorks™ Robotic Imager and Spot Cutter (Bio-Rad) and processed for mass spectrometric analysis as described previously (Taylor et al., J. Proteome Res., 1: 451-458, 2002). Silver-stained two-dimensional samples were manually destained in 5 mM potassium ferricyanide and 1 mM sodium thiosulfate, while Sypro Ruby-stained gel spots were destained in a ProGest™ digestion robot (Genomic Solutions Inc.). Reduction, alkylation, and digestion of both Sypro Ruby- and silver-stained two-dimensional gel spots were performed using the ProGest. Silver-stained one-dimensional gel bands (and destained as previously described) and Coomassie-stained one-dimensional gel bands were manually processed as described previously (Aggeler et al., J. Biol. Chem., 277: 33906-33912, 2002). Half of the final volume of digests from silver- and Sypro Ruby-stained gels (5-8 μl) was further subjected to strong cation exchange ZipTip (Millipore) clean up and concentration.

Peptides were eluted directly onto the MALDI targets, and spectra were automatically acquired on a Voyager DE-STR™ as reported previously (Taylor et al., J. Proteome Res., 1: 451-458, 2002). Peptide mass fingerprints from base-line-corrected, noise-filtered, and de-isotoped peaks were obtained and then analyzed using the program Protein Prospector, MS-Fit (Clauser et al., Anal. Chem., 71: 2871-2882, 1999) with and without the Intellical algorithm from Applied Biosystems as described previously (Taylor et al., J. Proteome Res., 1: 451-458, 2002). Since many of the NADH dehydrogenase subunits are small, predicted peptide mass fingerprints often contained only a limited number of peptides to match to peaks in the MALDI spectra. In these instances manual inspection of the MALDI data was performed as described by Aggeler et al. (J. Biol. Chem., 277: 33906-33912, 2002).

As shown in Table 6, MALDI-TOF spectrometry identified 23 different Complex I polypeptides and LC-MS/MS identified 40 subunits. The subunits A1 and B2 were identified by MALDI-TOF but went undetected by LC-MS/MS; therefore, combining the data from these methods results in identification 42 of the 45 putative Complex I subunits. In Table 4, Complex I proteins (bold) are represented where possible by their gene names (Human Genome Nomenclature Committee prefix NDUFx) and the theoretical mass of the mature human polypeptide. Minor contaminating proteins are shown in parentheses. A minimum of four tryptic peptides was used for all MALDI-TOF MS identifications. For LC-MS/MS, the number of peptide fragments determined is shown.

TABLE 6
Detection of Trypic Peptides from Complex I Subunits

| | | | | Predicted |
|---|---|---|---|---|
| | MALDI-TOF | LC-MS/MS | LC-MS/MS | mature subunit |
| Band | identification | identification | peptides | mass (kDa) |
| 1 | (IgG) | (IgG) | | |
| 2 | S1 | S1 | 30 | 77.0 |
| 3 | (F₁α/β) | (F₁α/β) | | (55.2/51.8) |
| 4 | V1 | V1 | 10 | 48.6 |
| 5 | | ND5 | 5 | 67.0 |
| 6 | S2 | S2 | 13 | 49.1 |
| 7 | | A10 | 4 | 37.1 |
| 8 | | ND4 | 1 | 51.6 |
| 9 | A9 | A9 | 16 | 38.9 |
| 10 | (ANT1) | (ANT1)/ND2 | 1 | (33.0)/39.0 |
| 11 | S3 | S3/ND1 | 10/3 | 26.4/35.7 |
| 12 | V2 | V2 | 7 | 23.7 |
| 13 | B9/B10 | B9/B10 | 8/5 | 21.7/20.7 |
| 14 | S7/S8 | S7/S8 | 4/7 | 19.8/20.3 |
| 15 | | NP17.3¹ | 3 | 17.3 |
| 16 | B8/A8 | B8/A8 | 5/6 | 18.8/20.0 |
| 17 | B7 | B7 | 8 | 16.3 |
| 18 | B6 | B6/S4 | 5/4 | 15.4/15.4 |
| 19 | B5 | B5 | 4 | 17.0 |
| 20 | 17.2 kDa² | 17.2 kDa² | 18 | 17.1 |
| 21 | GRIM-19 | GRIM-19 | 10 | 16.6 |
| 22 | B4 | B4 | 5 | 15.1 |
| 23 | A6/C2 | A6/C2 | 5/5 | 15.0/14.2 |
| | | A7/ND3 | 5/1 | 12.4/13.2 |
| 24 | | A11/A5/S5 | 5/7/6 | 14.9/13.3/12.4 |
| 25 | | S6/B3 | 4/4 | 10.7/11.3 |
| 26 | A2/B2 | | | 10.8/8.6 |
| | | A2/V3 | 4/1 | 10.8/8.4 |
| 27 | | A3/A4 | 2/1 | 9.3/9.4 |
| 28 | A1 | | | 8.0 |
| 29 | B1 | B1/A B1 | 1/3 | 7.0/10.2 |

¹Neuronal protein, GenBank Accession No. AAH07362
²17.2 kDa protein related to the 13 kDa Differentiation Association Protein

As demonstrated by analysis of Complex I tryptic peptides, Complex I subunits are greatly concentrated by immunocapture. Thus, immunocapture permits the identification of Complex I subunits in small isolates, such as biopsy material, blood cells, or cell cultured material.

C. Activity Assays for Immunopurified Complex I from Human and Bovine Heart Mitochondria

Three different enzymatic assays useful for assessing the functionality of the first mitochondrial respiratory chain complex, Complex I, are described below.

1. NADH-Ubiquinone-1 Reductase Assay

Complex I function is assayed in small amounts of mitochondria with the substitution of a hydrophilic quinone, ubiquinone-1, for the hydrophobic endogenous substrate, ubiquinone-10. In one well of a 96-well plate, 1 μg of immunopurified Complex I or 10 μg of isolated frozen-thawed mitochondrial membranes were added to phosphate buffer containing antimycin A, potassium cyanide, and ubiquinone-1. The initial rate of nicotinamide adenine dinucleotide (NADH) oxidation at 37° C. is followed by reading the absorbance of the sample at 340 nm with a plate spectrophotometer for 2-5 minutes after the addition of NADH to the mixture. Complex I-specific rates were calculated by subtracting the residual rate observed after the addition of a potent Complex I inhibitor, rotenone, to a reference sample containing the identical conditions and solutions outlined above.

2. NADH-Hexamineruthinium (III) Chloride Reductase Assay

As an indicator of potential Complex I flavoprotein (FP) content, the artificial electron acceptor, hexamineruthinium (III) chloride, is commonly used. This oxidant has the ability to accept electrons from NADH through the FP fraction of Complex I. It is an important and useful indicator for general Complex I function. Assay conditions are analogous to those described above for ubiquinone-1. In one well of a 96-well plate, 1 μg of immunopurified Complex 1 or 10 μg of isolated frozen-thawed mitochondrial membranes are added to phosphate buffer containing antimycin A, potassium cyanide, and hexamineruthinium (III) chloride. The initial rate of NADH oxidation at 37° C. is followed by reading the absorbance of the sample at 340 μm with a spectrophotometer plate reader for 2-5 minutes after the addition of NADH to the mixture. A reference sample without proteins is used to the record the background NADH oxidation activity of hexamineruthinium chloride (III). This reaction is not sensitive to the potent Complex I inhibitor, rotenone.

3. NADH-Resazurin Diaphorase Assay

A sensitive fluorescent assay for Complex I function uses a non-fluorescent compound, resazurin, which is reduced by NADH and Complex I to produce the fluorescent product, resorufin. The assay conditions are analogous to those described above for ubiquinone-1 and hexamineruthinium (III) chloride. In one well of a 96-well plate, 1 μg of immunopurified Complex 1 or 10 μg of isolated frozen-thawed mitochondrial membranes are added to phosphate buffer containing antimycin A, potassium cyanide, and resazurin. The initial rate of resazurin reduction at 37° C. is followed by reading the fluorescence (ex. 530 nm, em. 590 nm) of the sample with a fluorescence plate reader for 2-5 minutes after the addition of NADH. Complex I-specific rates are calculated by subtracting the residual rate observed after the addition of a potent Complex I inhibitor, rotenone, to a reference sample containing the identical conditions and solutions outlined above. The diaphorase-resazurin method is more sensitive by approximately 20-fold when compared to the other standard assays described here.

This Example demonstrates that the expression of Complex I and its subunits can be used to identify or diagnose human Complex I deficiency disorders. Eleven different patients, in which OXPHOS enzyme activity measurements had identified an isolated Complex I deficiency, were examined. In seven of these patients, the mutations had been determined by extensive gene sequencing. In the other four patients, a mutation has not yet been identified. Screening with an antibody mixture containing a mAb against at least one subunit of each of the five OXPHOS complexes supports the conclusion from enzymatic data that all of the patients were deficient in Complex I alone.

Several different patterns of steady-state levels of Complex I subunits were found in this example. For two of the subunits, NDUFS4 and NDUFS2, two patient cell lines were available with different mutations in the same gene. In both cases, the subunit profiles resulting from the two different mutations were essentially the same. In general, the subunits behaved as three classes. The levels of the 39- and 30-kDa subunits varied in the same way, as did the 20- and 18-kDa subunits, whereas the 15- and 8-kDa subunits are a third class. In most patients, the levels of the 20- and 18-kDa subunits were higher than the levels of functional complex as measured by enzymatic activity, whereas the levels of the 15- and 8-kDa subunits were lower.

As shown by the data in FIGS. 4A-C, the steady-state levels of fully assembled Complex I depend on expression levels of all of the subunits examined in this example. When any one subunit is mutated, the levels of assembled Complex I were reduced. The lower levels of the 15- and 8-kDa subunits in relation to activity could indicate more lability of these subunits after assembly of the complex. One of the patient cell lines, from patient 11, had very low levels of all of the subunits examined and values significantly lower than expected based on activity measurements. It could be that enzymatic activity was overestimated or that the complex is more labile to detergent solubilization with a resulting proteolysis of polypeptides because of the mutation. The defect in Patient 11 most likely involves a mutation in an assembly factor for Complex I. The levels of subunits are low, and these subunits are not in a fully assembled complex based on the sucrose gradient data. The comparison of OXPHOS subunit profiles enables patients to be sorted, as in genetic complementation studies, so that with wider screening of patients, a group of possible Complex I assembly factor mutants can be collected for chromosomal analysis and gene identification, as was done for the SURFI mutations of cytochrome c oxidase.

### Example 2

### Purification and Characterization of Functionally Active Human F₁/F₀ ATPase by Immunocapture

This Example demonstrates that human mitochondrial F₁/F₀ ATP synthase (also known as F₁/F₀ ATPase or Complex V) can be isolated in one-step immunological approach, which uses a monoclonal antibody specific for F₁. The immunocaptured Complex V displayed ATP hydrolysis activity that was fully oligomycin and IF₁ sensitive. The disclosed ATP hydrolysis assay of Complex V can be carried out with as little as 10 ng of heart mitochondria/well and as few as 3×10⁴ cultured fibroblast cells/well. IF₁ was co-isolated with F₁/F₀ ATPase when the immunocapture procedure was carried out at pH 6.5 but was absent when the ATP synthase was isolated at pH 8.0. The system described in this Example can be used, for example, to screen patient-derived samples for alterations in the amount and/or functionality of the F₁/F₀ ATPase and/or IF₁.

A. Material and Methods

1. Monoclonal Antibodies.

Monoclonal antibodies described in this Example were produced in the Monoclonal Antibody Facility at the University of Oregon (Eugene, Oreg.) using standard protocols for hybridoma development and antibody purification. Representative mAbs and their respective Complex V subunit specificities include: CV-α (7H10BD4F9), CV-β (3D5AB1), CV-d (7F9BG1), CV-OSCP (4C11C10D12), CV-IF₁ (5E2D7), CI-39 (20C11B11B11), CII-30 (21A11AE7), CIII-core 2 (13G12AF12BB11), and CIV-II (12C4F12). Additional Complex V-specific antibodies are shown in Tables 1 and 7.

To make anti-F₁/F₀ ATPase capture mAb, mice were immunized with purified bovine F₁/F₀ ATPase, and the resulting immune splenocytes used to construct mouse-mouse hybridomas. MAbs were then screened for ability to bind immobilized F₁/F₀ ATPase in ELISA assays and the positives were re-screened to identify mAbs that could capture active enzyme.

TABLE 7
Non-limiting Exemplar Complex V Antibodies

| Complex V | | | | | |
|---|---|---|---|---|---|
| Antigen | | WB | | | Cap- |
| (MW) | MAb | Conc | IC Conc | IP | ture |
| C-V-F1 | MM#1-12F4AD8AF8 | negative | negative | + | ++ |
| C-V-Alpha | MM#1-7H10BD4F9 | <0.5 μg/ml | 5 μg/ml | +/− | + |
| (53 kD) | | | | | |
| C-V-Beta | MM#7-3D5AB1 | <0.5 μg/ml | positive | nd | 2nd |
| (52 kD) | | | | | ab¹ |
| C-V-F1F0 | MM#1C-1A08 | nd | nd | nd | ++ |
| C-V-F1F0 | MM#1C-1H04 | nd | nd | nd | ++ |
| C-V-F1F0 | MM#1C-2A11 | nd | nd | nd | ++ |
| C-V-F1F0 | MM#1C-2B04 | nd | nd | nd | ++ |
| C-V-F1F0 | MM#1C-2F02 | nd | nd | nd | ++ |
| C-V-F1F0 | MM#1C-2F05 | nd | nd | nd | ++ |
| C-V-F1F0 | MM#1C-2F08 | nd | nd | nd | ++ |
| C-V-F1F0 | MM#1C-2G08 | nd | nd | nd | ++ |
| C-V-F1F0 | MM#1C-2G09 | nd | nd | nd | ++ |
| C-V-F1F0 | MM#1C-2H07 | nd | nd | nd | ++ |
| C-V-F1F0 | MM#1C-2H10 | nd | nd | nd | ++ |
| C-V-F1F0 | MM#1C-3A05 | nd | nd | nd | ++ |
| C-V-F1F0 | MM#1C-3A08 | nd | nd | nd | ++ |
| C-V-F1F0 | MM#1C-3H01 | nd | nd | nd | ++ |
| C-V-F1F0 | MM#1C-4C07 | nd | nd | nd | ++ |
| C-V-F1F0 | MM#1C-4F05 | nd | nd | nd | ++ |
| C-V-F1F0 | MM#1C-4H06 | nd | nd | nd | ++ |
| C-V-F1F0 | MM#1C-5F08 | nd | nd | nd | ++ |
| C-V-F1F0 | MM#1C-6D08 | nd | nd | nd | ++ |
| C-V-F1F0 | MM#1C-6E01 | nd | nd | nd | ++ |
| C-V-F1F0 | MM#1C-6E02 | nd | nd | nd | ++ |
| C-V-F1F0 | MM#1C-6G09 | nd | nd | nd | ++ |
| C-V-F1F0 | MM#1C-7B04 | nd | nd | nd | ++ |
| C-V-F1F0 | MM#1C-7B12 | nd | nd | nd | ++ |
| C-V-F1F0 | MM#1C-8B08 | nd | nd | nd | ++ |
| C-V-F1F0 | MM#1C-8D11 | nd | nd | nd | ++ |
| C-V-F1F0 | MM#1C-8E08 | nd | nd | nd | ++ |
| C-V-F1F0 | MM#1C-8F01 | nd | nd | nd | ++ |
| C-V-F1F0 | MM#1C-8G01 | nd | nd | nd | ++ |
| C-V-F1F0 | MM#1C-8G08 | nd | nd | nd | ++ |
| C-V-F1F0 | MM#1C-8H05 | nd | nd | nd | ++ |
| C-V-F1F0 | MM#1C-9C12 | nd | nd | nd | ++ |
| C-V-F1F0 | MM#1C-9E02 | nd | nd | nd | ++ |
| C-V-F1F0 | MM#1C-9F06 | nd | nd | nd | ++ |
| C-V-F1F0 | MM#1C-10A-H09 | nd | nd | nd | ++ |
| C-V-F1F0 | MM#1C-10A-H11 | nd | nd | nd | ++ |
| C-V-F1F0 | MM#1C-10B03 | nd | nd | nd | ++ |
| C-V-F1F0 | MM#1C-10C03 | nd | nd | nd | ++ |
| C-V-F1F0 | MM#1C-10C10 | nd | nd | nd | ++ |
| C-V-F1F0 | MM#1C-10C11 | nd | nd | nd | ++ |
| C-V-F1F0 | MM#1C-10F07 | nd | nd | nd | ++ |
| C-V-F1F0 | MM#1C-10H01 | nd | nd | nd | ++ |

¹MM#7-3D5AB1 binds native Complex V at a site different from that recognized by MM#1-12F4AD8F8 and, therefore, MM#7-3D5AB1 can be used as a second detection mAb in 2-site assays, at least, in combination with MM#1-12F4AD8F8.

2. Tissue and Cell Preparation.

Mitochondria from normal, human heart ventricle muscle (removed post-mortem from a 41 year old male who had died of brain cancer) were obtained from Analytical Biological Services, Inc, who prepared the mitochondria by tissue homogenization and differential centrifugation essentially as previously described by Marusich et al. (Biochim. Biophys. Acta, 1362: 145-159, 1997). Isolated mitochondria were suspended in final wash buffer (10 mM Tris, pH 7.5, 1 mM EDTA, 0.25 M Sucrose) with protease inhibitors (0.5 μg/ml leupeptin, 0.5 μg/ml pepstatin, and 1 mM PMSF) at 40 mg/ml mitochondrial protein and stored frozen until use. Protein concentrations were determined by the Bradford assay using gamma globulin as a standard.

Normal human fibroblasts (MRC5, a diploid strain derived from fetal human lung) were obtained from the American Type Culture collection and used between population doubling (PD) 30 and 45. Rho0-MRC5 cells (lacking mtDNA) were derived by culturing normal MRC5 cells for 12-14 population doublings in ethidium bromide (50 ng/ml). Patient-derived GM00028 fibroblasts were obtained from the HIGMS Human Genetic Mutant Cell Repository at the Coriell Institute for Medical Research and used at early passage levels.

All cell cultures were maintained and mitochondria isolated by cell homogenization and differential centrifugation as previously described (Marusich et al., Biochim. Biophys. Acta, 1362: 145-159., 1997). The mitochondria were suspended in final wash buffer with inhibitors and protein concentrations were determined by measuring the OD₂₈₀ of 0.01 ml of mitochondria diluted in 1 ml of 0.6% SDS and heated to 94-100° C. for 4 minutes. Pierce BCA protein assay (Pierce Chemical) was used to determine that 1 mg/ml of mitochondrial protein corresponds to an OD₂₈₀ of 2.1. To prepare whole cell extracts, cultured cells were grown to confluence, dissociated by trypsinization, the trypsin inactivated with fetal calf serum, the cells washed twice with PBS, cells counted, stored frozen as cell pellets and then used as described below.

3. Immunocapture and Measurement of ATPase Activity

Solubilization. Samples were thawed and placed in 25 mM HEPES pH 7.5 (to dissociate F₁F₀/IF₁ interactions) or 10 mM n-dodecyl-β-D-maltoside (MOPS) (Anatrace) pH 6.5 (to retain F₁F₀/IF₁ interactions), containing 0.4% (w/v) MOPS, and the protease inhibitors leupeptin (0.5 μg/ml), pepstatin (0.5 μg/ml) and PMSF (1 mM). Mitochondria were solubilized at 5 mg/ml and whole cells were solubilized at 2×10⁷ cells/ml. Samples were mixed well and then kept on ice for 30 minutes with occasional agitation. Insoluble material was then removed by centrifugation at 16,000×g for 20 minutes at 4° C.

Immunocapture. Assays were run in 96-well plates (Falcon Probind™) using 100 μl/well. The wells were prepared for immunocapture by first adsorbing goat anti-mouse IgG-Fe (IgG₁+IgG₂ₐ+IgG_{2b}+IgG₃) at 5 μg/ml in TBS (50 mM Tris pH 7.5, 150 mM NaCl) with 0.02% azide overnight at 4° C., washing 3 times with TBS alone, incubating for 1 hour at room temperature with the anti-ATPase capture mAb 12F4AD8AF8 at 5 μg/ml in TBS with 2.5% BSA, and then washing 4 times with TBS. To control for non-specific adsorption of ATPase, a non-specific mouse monoclonal antibody was used as a null capture mAb. Wells were then loaded with solubilized test samples diluted in TBS with 2.5% BSA, incubated for 2 hours at room temperature and then washed 4 times with TBS.

Certain antibody orientations and/or certain distances of the captured F₁F₀ from the polystyrene plate may be advantageous for retaining full biological properties of the enzyme. Enzyme captured as described above (using a layer of goat-anti-mouse IgG to capture and orient mAb 12F4AD8AF8) could be completely inhibited by oligomycin (see below). However, enzyme captured by mAb 12F4AD8AF8 directly adsorbed to the plate had high ATPase activity but could not be inhibited by oligomycin.

ATPase activity. ATPase reaction buffer (25 mM HEPES pH 7.5 or 10 mM MOPS pH 6.5, with 25 mM KCl, 5 mM MgCl₂, 5 mM KCN and 2 mM ATP) was added at 100 μl/well and incubated for 3 hours at 37° C. The reaction was stopped and free Pᵢ generated by ATP hydrolysis was detected essentially as described by Walker et al. (Meth. Enzymol., 260: 163-190, 1995) by addition of 0.5% ammonium molybendate in 0.7 M H₂SO₄ (42 μl/well) followed by 10% ascorbic acid (4 μl/well). After incubation for 30 minutes at room temperature to allow color development, absorbance was measured at 690 nm.

IF₁ Stripping Conditions. IF₁ was dissociated from previously immunocaptured IF₁/F₁F₀ complexes by exposure to IF₁ stripping buffer (30 mM Tris-sulfate pH 8, 250 mM KCl, 2 mM EDTA, 75 mM sucrose) for 30 minutes at 37° C. (Van Raaij et al., Biochem. 35: 15618-15625, 1996). The wells were then washed with either a pH 7.5 wash buffer (TBS) or a pH 6.5 wash buffer (10 mM MOPS pH 6.5, 150 mM NaCl and 1 mM MgCl₂), and ATPase activity measured at either pH 7.5 or pH 6.5 as described above.

IF₁ Addition Conditions. Recombinant human IF₁ was obtained by PCR-based amplification of the appropriate gene from a human heart CDNA library (Clontech), cloning into pET15b (Novagen), transformation of E. coli and overexpression and purification of the His-tagged protein on a Ni column by standard protocols (Novagen). Isolated protein was diluted in pH 6.5 wash buffer (as described above) containing 1 mM ATP and incubated with previously immunocaptured F₁F₀. The wells were then washed with pH 6.5 wash buffer and ATPase activity measured at pH 6.5 as described above.

Western Blot. Samples were dissolved in Tris-Glycine sample buffer containing 4% SDS and 2% (β-mercaptoethanol, warmed to 37° C. for 30 minutes, cooled to room temperature and then applied to Tris-Glycine SDS-PAGE 10-22% gradient minigels.

Proteins were separated by electrophoresis at 100 V for 2 hours at room temperature, after which the gels were soaked with gentle mixing for 30 minutes in cold CAPS transfer buffer (10% methanol in 10 mM 3-[cyclohexylamino]-1-propanesulfonic acid, pH 11). The proteins were then electrophoretically transferred (2 hours at 100 mA) to 0.45 μm PVDF membranes (Millipore) in ice-cold 3-[cyclohexylamino]-1-propanesulfonic acid (CAPS) transfer buffer. Mitochondrial protein-loaded membranes were blocked overnight in 5% non-fat dry milk in PBS at 4° C. then incubated overnight at 4° C. in mixtures of subunit-specific monoclonal antibodies as desired (see Table 7) in 5% milk/CMF-PBS with 0.02% azide, washed four times with PBS combining 0.05% TWEEN 20™, incubated 2 hours at room temperature in 5% milk/PBS containing HW-GAM-IgG-Fe (Jackson ImmunoResearch) at 0.08 μg/ml, washed as above, rinsed with PBS and the bound antibodies visualized with ECL-Plus™ (Amersham) on a STORM imaging system (Molecular Dynamics), reading blue fluorescence.

Immunoprecipitation of Mitochondrial F₁/F₀ ATPase (M F₁F₀). Monoclonal antibody 12F4AD8 (2 mg) was bound to 250 mg Protein G Agarose (Sigma) essentially as described by Schneider et al. (J. Biol. Chem., 257: 10766-10769, 1982) in 3 ml PBS for 1 hour at room temperature. After washing the beads twice with 10 ml 0.2 M NaBorate (pH 9.0), dimethylpimelimidate was added to a final concentration of 20 mM. The reaction was stopped after 30 minutes by washing the beads with 0.2 M ethanolamine (pH 8.0) and incubation for 2 hours at room temperature. The coupled antibody was kept in PBS at 4° C. in the presence of 1 mM NaN₃.

For immunoprecipitation of M F₁F₀, mitochondria from human heart were washed with 10 mM Tris, 250 mM sucrose, 0.5 mM EDTA, pH 7.5 and resuspended at 3 mg/ml in the presence or absence of 1 mM ADP in 20 mM Bis-Tris, 150 mM sucrose, 0.5 mM EDTA, pH 6.5, supplemented with protease inhibitors leupeptin (2 μg/ml), pepstatin (2 μg/ml) and PMSF (2 mM). The mitochondria were solubilized by addition of 1 volume of the same buffer (without inhibitors) containing 0.45% n-dodecyl-β-D-maltoside and incubated for 20 minutes at 4° C. The extract was centrifuged in a TLA100.2 centrifuge at 65,000 rpm for 30 minutes at 4° C. Protein G agarose beads with bound nonspecific antibody (NSA) were added at 40 μl/1 ml supernatant and shaken at room temperature for 1 hour. After centrifugation for one minute at 10,000×g, the supernatant was exposed a second time to 40 μl NSA beads, followed by immunoprecipitation with 40 μl 12F4AD8 beads for 2 hours at room temperature. The beads were then washed 6 times with the same buffer, containing 0.05% n-dodecyl-β-D-maltoside and the ATP synthase eluted twice with 70 μl 0.1 M glycine, 0.05% n-dodecyl-β-D-maltoside, pH 2.5. Na-phosphate, pH 8.0 was added to a final concentration of 0.1 M to adjust the pH to about 7.5.

SDS-PAGE Analysis of Subunit Profile. Mitochondria (2.3 mg) were solubilized at 1.5 mg/mil in 0.225% n-dodecyl-β-D-maltoside in the absence or presence of 1 mM ADP and F₁/F₀ ATPase was immunoprecipitated with the F₁-specific monoclonal antibody 12F4AD8AF8. The subunit composition was analyzed by 14-24% SDS-PAGE, followed by mass spectrometry with MALDI-TOF and LC-MS/MS of trypsinized Coomassie Brilliant Blue stained brands. Subunits were identified by the number of matched fragments (x), and the coverage of the full sequence of mature protein (y %): subunit α (32, 55%), subunit β (28, 50%), subunit γ (12, 34%), subunit b (18, 57%), OSCP (13, 71%), subunit d (12, 56%), subunit a (5, 8%), subunit δ (2, 16%), subunit f STR and LC-MS/MS with a C-18 reversed phase capillary column and a LCQ Ion Trap Mass spectrometer (Finnigan) equipped with a dynamic nanospray source (Finnigan) as described by Aggeler et al. (J. Biol. Chem., 277(37): 33906-33912, 2002).

4. Other Methods.

Protein concentrations were determined with BCA (Pierce). Polyacrylamide gels were stained with Coomassie Brilliant Blue (Serva) or for higher sensitivity with Sypro-Ruby (Molecular Probes, Inc., Eugene, Oreg.) as described in Marusich, J. Immunol. Meth., 114: 155-159, 1988).

B. Results

1. Human Mitochondrial F₁/F₀ ATPase can be Isolated by Immunoprecipitation as a Complex of 16 Different Subunits.

When immobilized on beads or 96-well plates, anti-F₁/F₀ ATPase “capture” mAb (12F4AD8AF8) immunocaptured F₁/F₀ ATPase from detergent-solubilized extracts of human tissues or cell culture material as described above.

The subunit profile of enzyme isolated in this straightforward one-step procedure from human heart mitochondria showed that all 16 different polypeptide subunits previously reported to be present in bovine F₁/F₀ ATPase were all present in the human form of the enzyme. Proteins were immunoprecipitated from human heart mitochondria that had been solubilized in 0.225% n-dodecyl-β-D-maltoside in the absence or presence of ADP (1 mM). The various polypeptides were identified by a combination of reactivity with (4, 48%), inhibitor protein IF₁, (3, 22%), subunit g (6, 43%), Coupling factor 6 (4, 38%), subunit e (4, 62%), subunit A6L (5, 53%), subunit ε (3, 30%). Assignment was supported with LC-MS/MS for subunits with fewer than 10 matched peptide fragments. When peptides of a subunit were detected in more than one band, number of fragments and signal intensities were used for selection.

In a second experiment, mitochondrial F₁/F₀ ATPase from 1.4 mg mitochondria was applied on a 4 cm wide lane for SDS-PAGE and blotted on IMMOBILON-P™. Lanes 4-mm wide were cut and analyzed with monoclonal antibodies against subunit α (MM#1-7H10BD4F9), β (MM#7-3D5AB1), OSCP (MM#5-4C11C10D12), and d (MM#1-7F9BG1), which are described in Table 2 (non-capture ancillary mAbs) and are commercially available from Molecular Probes, Inc. (Eugene, Oreg.) and MitoScience (Eugene, Oreg.). Alkaline phosphatase-conjugated goat anti-mouse antibodies were used for color development.

2. Identification of ATP Synthase Subunits by MALDI-TOF and LC-MS/MS.

Samples from immunoprecipitation were supplemented with dissociation buffer with 50 mM 1,4-dithio-threitol (DTT) for SDS-PAGE, which was performed according to Laemmli (Nature, 227: 680-685, 1970) on 14-24% acrylamide gradient gels. Bands were cut out after staining with Coomassie Brilliant Blue and analyzed with mass spectrometry after trypsinization. Gel pieces were destained by two washes in 0.2 ml 50% acetonitrile in 0.1 M NH₄HCO₃ for 45 minutes at 37° C. After dehydration with 0.1 ml acetonitrile, the gel pieces were dried in a Speed-Vac, 100 μl of 0.1 M DTT in 0.1 M NH₄HCO₃ was added for 1 hour at 56° C. followed by one wash with water and addition of 0.1 M iodoacetamide for 30 minutes in the dark. The gel pieces were then washed twice with 0.1 ml NH₄HCO₃ for 15 minutes and once with 50% acetonitrile in 0.1 M NH₄HCO₃. After treatment with acetonitrile, gel pieces were dried in a 3 Speed-Vac and 20 μl freshly prepared modified trypsin at 25 μg/ml (Promega) in 10% acetonitrile and 50 mM NH₄HCO₃ was added. After 10 minutes, the gel pieces were overlaid with 50 μl buffer and incubated at 37° C. overnight. The digestion was terminated by adding 2 μl trifluoroacetic acid. After addition of 0.1 ml water, the supernatant was placed in a siliconized pre-washed eppendorff tube. The gel pieces were extracted twice for 30 minutes with 100 μl 50% acetonitrile and 0.2% trifluoroacetic acid with occasional shaking. The supernatants were pooled and the samples dried in a Speed-Vac and re-dissolved with 20 μl 5% acetonitrile, 0.1% acetic acid for MALDI-TOF analysis with Voyager DE-subunit-specific mAbs and mass spectrometry using both MALDI-TOF and LC-MS/MS procedures as described above.

The α, β, γ, b, OSCP, d, a, δ and f subunits were each detected as a single band. Subunits g, F6, e, A6L and ε were observed in more than one closely spaced band, based on the detection of fragments by mass spectrometry. Also present near the bottom of the gel was what was interpreted to be subunit c. This subunit could not be identified by the mass spectrometry analysis because it generated too few tryptic digest fragments of appropriate molecular weights. However, subunit c must be present as the enzyme was active and inhibitor (oligomycin) sensitive as isolated. In support of this conclusion, the presence of subunit c was recently confirmed in human F₁/F₀ ATPase labeled with ¹⁴C-DCCD, solubilized in the same detergent as used here, but affinity purified with a Sepharose-EAH column (Garcia et al., J. Biol. Chem., 275: 11075-11081, 2000). Finally, immunoprecipitated F₁/F₀ ATPase also was shown to contain IF₁ when solubilization and capture conditions allow F₁/F₀ ATPase/IF₁ interactions.

The protocol described above was chosen to isolate the active enzyme without significant loss of subunits. Lower concentrations of detergent gave a less pure preparation. At higher levels, there was loss of activity. Evident in electrophoresis gels and confirmed by mass spectrometry analysis, were minor amounts of other mitochondrial respiratory chain proteins of Complexes I and IV (including COX IV). The presence of these proteins may indicate that, at 0.225% n-dodecyl-β-D-maltoside, a small amount of the F₁/F₀ ATPase was retained in supercomplexes of the oxidative phosphorylation machinery. Similar supercomplexes of Complexes I, III and IV have been reported before for yeast and mammalian mitochondria.

One unexpected protein consistently appeared in the immunoprecipitate in large amounts and in a nucleotide-dependent manner. This polypeptide, with an apparent MW on the SDS polyacrylamide gels of around 30,000 daltons, was identified by mass spectrometry as the ADP/ATP translocase (ANT). ANT was associated with F₁/F₀ ATPase in the absence of ADP, but was not observed when nucleotide was added before and during the immunoprecipitation step. Although not recognized as such, ANT was also present in human F₁/F₀ ATPase isolated earlier by Sepharose-EAH chromatography. This result indicates a direct physical interaction of ANT and F₁/F₀ ATPase under some conditions. Such an interaction between ANT and ATP synthase had been suggested earlier based on fluorescence quenching experiments by Ziegler and Penefsky (J. Biol. Chem., 268: 25320-25328, 1993).

Human F₁/F₀ ATPase captured by the mAb was active as an ATPase. Using the procedures described above, a microscale 96-well format was used to facilitate measurements of ATPase activity of immunoprecipitated material. Goat anti-mouse IgG specific for the Fe portion of mouse IgG was absorbed to 96-well plates and used to capture mouse anti-F₁/F₀ ATPase mAb in favorable orientations. The wells were then incubated with solubilized tissue or cell samples, washed, and any immunocaptured F₁/F₀ ATPase was detected by colorimetric ATP hydrolysis measurements.

As shown in FIG. 5, this assay of ATPase activity of F₁/F₀ ATPase is sensitive, quantitative, and has a large dynamic range. Positive signals were obtained from as little as 10 ng/well of solubilized human heart mitochondrial protein, and the assay only saturated at 10 μg/well, i.e., a range of three orders of magnitude. FIG. 5 shows the results of activity measurement of the enzyme in the presence and absence of oligomycin. When isolated by the disclosed one-step immunoprecipitation method, ATPase activity of human F₁/F₀ ATPase could be inhibited more than 90% by oligomycin. This is a higher level of inhibition than reported for enzyme isolated by fractionation or column chromatography methods.

It is now well established that an endogenous inhibitor protein IF₁ controls the activity of mammalian F₁/F₀ ATPase and that the effects of this inhibitor are pH dependent. To determine whether these results could be replicated with immunoprecipitated F₁/F₀ ATPase, human heart mitochondria were solubilized and F₁/F₀ ATPase immunocaptured (all at pH 6.5 or pH 8) as described herein. Immunocaptured material was then subjected to SDS-PAGE, transferred to a PVDF membrane and probed with mAbs specific for the α (MM#1-7H10BD4) and d (MM#1-7F9BG1) subunits of F₁/F₀ ATPase as well as a mAb specific for IF₁ (RAC#25A-5E2D7) (antibodies commercially available from Molecular Probes (Eugene, Oreg.) and MitoScience (Eugene, Oreg.)). These Western blots showed that IF₁ was associated with F₁/F₀ ATPase when solubilized and immunoprecipitated at pH 6.5. However, IF₁ was not associated with F₁/F₀ ATPase if these procedures were carried out at about pH 8.0.

Consistent with the above observations, the ATP hydrolysis activity of F₁/F₀ ATPase solubilized, immunocaptured and assayed at pH 6.5 was relatively low (FIG. 6A), but the enzyme could be greatly activated (>10-fold) by conditions that strip the IF₁ from the protein (e.g., 30 minutes exposure to pH 8.0). The inhibition could be reversed by addition of recombinant IF₁, which reduced the rate of ATP hydrolysis to that before stripping. These results show that enzyme isolated and captured at pH 8.0 was considerably more active when assayed at pH 6.5 than that isolated at the more acidic pH, but could be greatly inhibited by addition of purified IF₁ (FIG. 6B).

Antibody capture can be used to isolate F₁/F₀ ATPase from human fibroblast cell lines, including Rho0 cells with partly assembled enzyme.

The polypeptide composition of F₁/F₀ ATPase immunoprecipitated from normal and Rho0 human fibroblasts was assayed as described herein. For the experiments to examine subunit profiles, mitochondria were isolated from cells prior to immunoprecipitation. For activity studies, the enzyme could be obtained in sufficient quantities directly from cell extracts. The subunit profiles of the enzyme from fibroblast cells showed more impurities as a result of the much lower concentrations of enzyme per mg mitochondrial protein in these cell lines than in heart and because the number of cells used for this study was low, being in the range that can be readily generated from patient fibroblast specimens for detection of mitochondrial diseases. For subunit detection of small amounts of protein, Sypro-Ruby (Molecular Probes, Eugene, Oreg.) was used as stain rather than Coomassie Brilliant Blue and the greater sensitivity of Sypro-Ruby revealed minor impurities readily, as was seen comparing the gel subunit profile of F₁/F₀ ATPase from human heart mitochondria in gels. All of the subunits of the enzyme seen in human heart F₁/F₀ ATPase could also be resolved in the fibroblast immunoprecipitate.

Comparison of enzyme from normal and Rho0 cells showed that the α, β, γ, δ and ε subunits of the F₁ part were present in equal amounts in the two cell lines. In addition, there were close to equal amounts of b, OSCP and d in the two. Subunits f, g and/or F6 appeared diminished in enzyme from Rho0 cells and, as expected, subunits a and A6L, the two subunits encoded on mitochondrial DNA, were missing in the F₁/F₀ ATPase from Rho0.

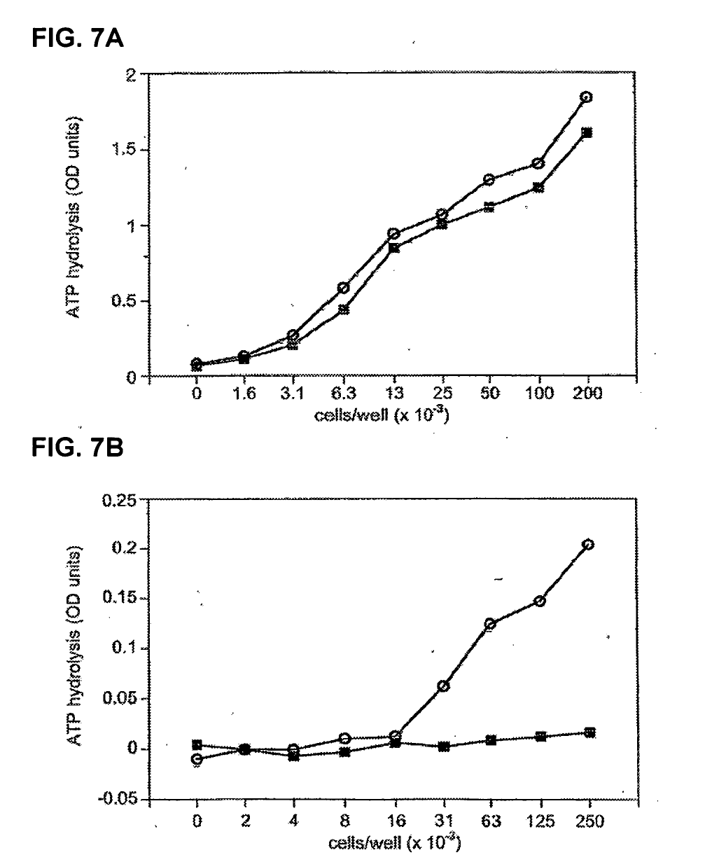

As with enzyme purified from human heart, F₁/F₀ ATPase immunocaptured from fibroblasts retained ATPase activity in the 96-well plate assay. A full activity analysis could be completed on isolated mitochondria, or more simply with unfractionated extract, from a small number of cells (FIG. 7). For example the data in FIG. 7B was generated with the material extracted from 2×10⁶ cultured fibroblasts. This figure shows a complete dilution series with and without oligomycin; all points measured in duplicate. FIGS. 7A and 7B also show the advantage of the immunocapture approach for analyzing F₁/F₀ ATPase activity of fibroblasts (or other cultured cells). As shown in FIG. 7A, only a small portion of the total ATP hydrolysis activity in such cell extracts was oligomycin sensitive, because of the presence of many other different ATPase and a relatively low proportion of mitochondrial F₁/F₀ ATPase synthase. However, as shown in FIG. 7B, immunocaptured enzyme was more than 90% oligomycin sensitive, which permits a clear analysis of the functionality of this key enzyme of oxidative phosphorylation.

To determine if the immunocapture assay described here can be used to detect functional defects in human fibroblasts, normal human fibroblasts were compared with Rho0 fibroblasts and GM00028 fibroblasts obtained from a patient with Luft's disease (which is human myopathy characterized by abnormally large mitochondria and defective respiratory control with normal phosphorylation; Dimauro et al., J. Neurol. Sci., 27: 217-232, 1976). GM0028 fibroblasts have been reported to have an IF₁ defect (Yamada and Huzel, Biochim. Biophys. Acta, 1139: 143-147, 1992). Rho0 cells have a complete absence of mtDNA-encoded proteins and have previously been shown to contain defective F₁/F₀ ATPase. The defective F₁/F₀ ATPase in Rho0 cells lacks mtDNA-encoded subunits 6 (a) and 8 (A6L) and is oligomycin insensitive (Garcia et al., J. Biol. Chem., 275: 11075-11081, 2000).

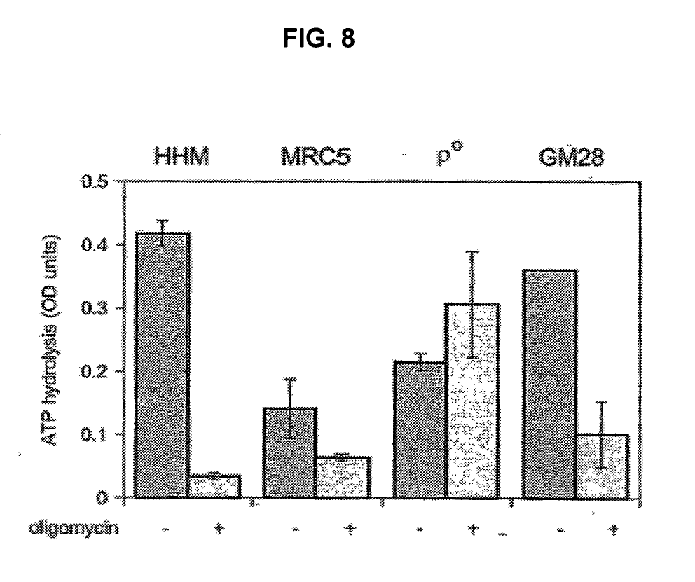

As shown in FIG. 8, active F₁/F₀ ATPase could be immunocaptured from mitochondria of all three cell lines. Human heart mitochondria (HHM) served as a positive control. Rho0 and GM00028 (GM28) fibroblasts displayed higher F₁/F₀ ATPase activities than the control MRC5 fibroblasts (about 1.5× and 2.5× of control values, respectively). As expected, the F₁/F₀ ATPase immunocaptured from Rho0 cell mitochondria were oligomycin insensitive. In contrast, F₁/F₀ ATPase from MRC5 and GM00028 cell lines showed comparable oligomycin sensitivity.

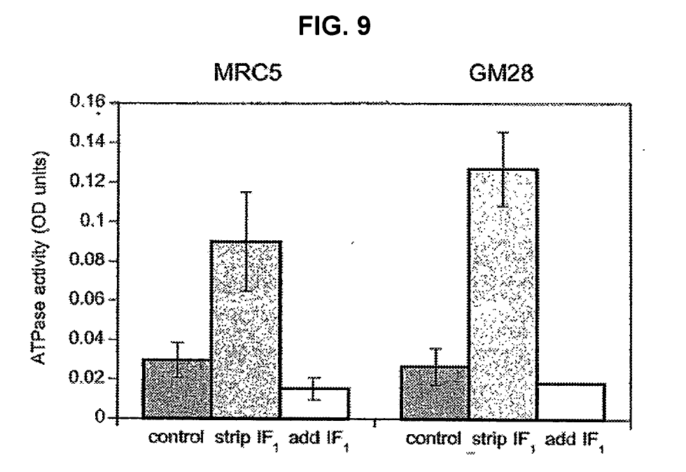

As shown in FIG. 9, F₁/F₀ ATPase immunocaptured from GM00028 and MRC5 cells at pH 6.5 had approximately the same ATPase activity. ATPase activity was increased was increase in both sample by treatment in high pH after capture. These results indicate that F₁/F₀ ATPase immunocaptured from GM00028 and MRC5 cells was associated with, and inhibited by, endogenous IF₁. High pH treatment stripped the endogenous IF₁ and ATP hydrolysis activity was increased. The “stripped” F₁/F₀ ATPase in both cell types was inhibited by added recombinant IF₁ (5 μg/ml), which indicates that IF₁ functionally reassociates with the stripped, immunocaptured enzyme.

Because these results were not consistent with the earlier report that GM00028 cells lacked IF₁ (Yamada and Huzel, Biochim. Biophys. Acta, 1139: 143-147, 1992), a second independent assay of IF₁ function was performed. Western blots of mitochondria from Luft's patient MRC5 cells and GM00028 were probed with an IF₁-specific monoclonal antibody, together with a cocktail of mAbs specific for subunits of the 5 OXPHOS complexes as previously reported (Marusich, Biochim. Biophys. Acta, 1362: 145-159, 1997; (J. Biol. Chem., 268: 25320-25328, 1993) this very sensitive method can reveal specific defects in the enzymes of the OXPHOS system.

Fibroblast mitochondria (5 μg per lane) were analyzed by probing with a cocktail of mAbs (anti-IF₁ at 0.5 μg/ml; anti-CI-39 at 2 μg/ml; anti-CII-30 at 5 μg/ml; anti-CIII-core 2 at 0.4 μg/ml; anti-CIV-II at 2 μg/ml; and anti-CV-α at 4 μg/ml), and visualizing with chemiluminescence using HRP-conjugated goat anti-mouse antibodies and ECL-Plus™. Analysis of the Western blots showed that GM28 fibroblast cells mitochondria contain normal levels of IF₁ as well as the OXPHOS Complexes I, II, III, IV and V. It was noted that Rho0 cells lack mtDNA-encoded subunit II of Complex IV, indicating full depletion of mtDNA. Thus, the assay showed the expected absence of mtDNA-encoded subunits in Rho0 cells, but also revealed clearly that GM00028 mitochondria exhibited a normal OXPHOS subunit profile, indistinguishable from control MRC5 cells. Moreover GM00028 cells contained normal levels of IF₁, which is consistent with the activity measurements described above.

Example 2 describes (among other things) quantitative microscale immunocapture of Complex V (F1/F0 ATPase) (see also, Aggeler et al., J. Biol. Chem., 277: 33906-33912, 2002) using enzymatic activity of the captured enzyme complex as the reporter. Example 2 demonstrates, for example, that the disclosed methods are capable of measuring the relative levels of enzyme from a variety of samples over a wide dynamic range. For example, Complex V ATPase activity could be measured quantitatively using from 10 ng to 10 μg of human heart mitochondria per well, or with as few as 3,000 cells per well from fibroblast cultures. Because the activity of the immunocaptured enzyme itself provided the reporter function, the disclose methods also permit the monitoring of samples for the presence or absence of various normal regulatory molecules, such as the Complex V inhibitor protein IF₁.

### Example 3

### Microscale Immunocapture Assays

This Example describes methods for simplifying quantitation of target proteins and for facilitating the simultaneous processing of large sample numbers probed with large numbers of capture antibodies; in particular, by labeling target proteins with fluorescent dyes and formatting the capture mAbs in a microarray. This method also has the advantage of permitting detection of target proteins that do not have enzymatic activity.

Commercially available protein reactive fluorescent dyes (amine-reactive succinimidyl esters and thiol-reactive maleimides) were used to label solubilized human and bovine heart mitochondrial proteins. After labeling the mitochondrial proteins with fluorescent dye, unreacted dye was removed to prevent the dye from subsequently reacting directly with capture antibodies and/or blocking agents which would give unacceptable background fluorescence. Unreacted dye may be removed by, for example, quenching, gel filtration, or dialysis. In this Example, unreacted dye was removed at the end of the labeling period by addition of excess bovine serum albumin (BSA). An additional benefit of removing unbound dye is that the degree of dye:protein conjugation (the F:P ratio, reported either on a molar or weight basis) can be determined. This value can serve as a quality control check of the labeling procedure, and can also enable determination of the absolute amount of protein immunocaptured per well.

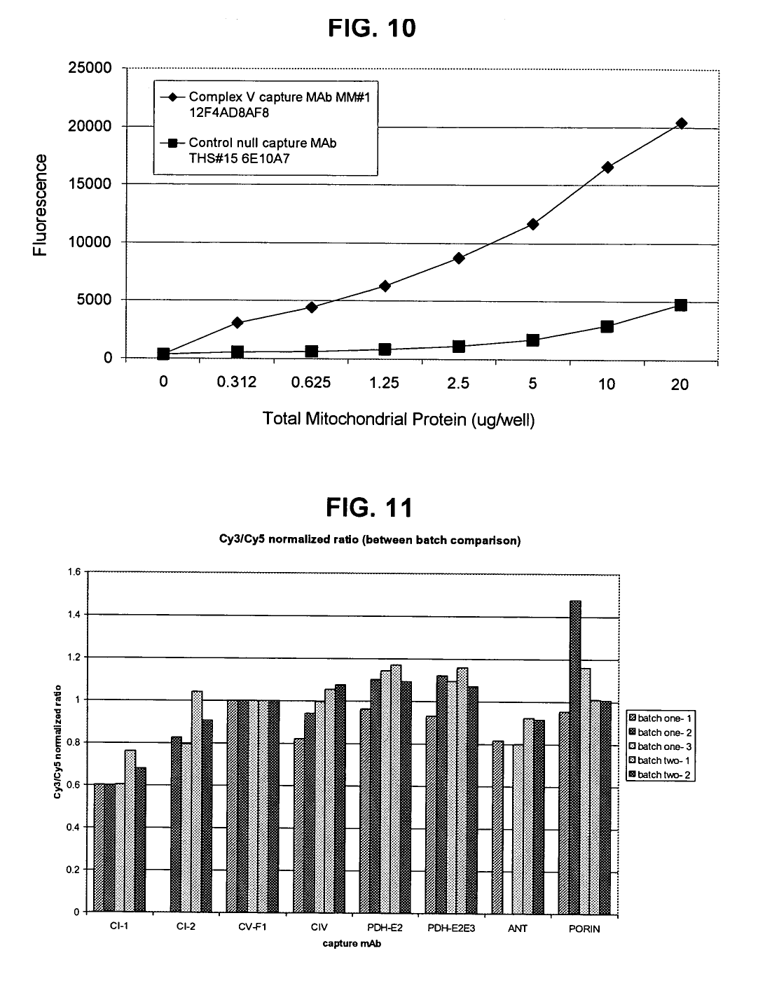

FIG. 10 shows a dilution series for the immunocapture of Complex V from fluorescently labeled solubilized bovine heart mitochondria. This result demonstrates that the fluorescence signal measured in this assay is proportional to target antigen concentration. It will be possible to use such binding curves as standard curves to which the fluorescence values obtained using unknown samples can be referred, allowing calculation of target antigen concentration in the unknown sample. Standards can include, but are not limited to, purified target antigens, in which case the results would be reported in terms of grams of target antigen or moles of target antigen per sample. Standards can also include unpurified samples prepared from some reference sample, e.g., normal heart and the target antigen concentration reported in “normal units” of target antigen per mg sample protein.

Quantitative and comparative data can also be obtained by 2-dye ratio measurement as previously described. FIG. 11 shows the pooled results of repeated experiments in which a single sample of heart mitochondria was solubilized, divided in two, and the two portions labeled with two different thiol-reactive maleimide dyes (Cy3 and Cy5) and unreacted dye removed/blocked as described above. The labeled mitochondrial were then mixed at 1:1 ratios (Cy3:Cy5) and applied to 96-well antibody-containing microtitre plates. Individual wells of the plate were pre-coated in triplicate with 8 different capture mAbs, namely, 2 mAbs specific for Complex I (CI-1: 20D1AB7 and CI-2: 18G12BC2), a mAb specific for Complex V (CV-F1: 12F4AD8AF8), a mAb specific for Complex IV (CIV: 7E5BA4), 2 mAbs specific for PDH (PDH-E2: 15D3G9C11 and PDH-E2/E3: 13G2AE2BH5), a mAb specific for ANT (ANT: 5F51BB5AG7) or a mAb specific for porin (PORIN: 20B12AF2). Background capture was determined by measuring the amount of material captured in wells coated by pooled normal mouse antibody known not to bind mitochondrial proteins.

Following a period sufficient to permit the bound mAbs to capture their particular targets (such as, about 1 hour, 6 hours or 24 hours), the plates were washed to remove any non-bound protein, the bound fluorescence signals were measured, and the ratio of Cy3:Cy5 fluorescence was calculated for each well. For each experiment, the Cy3:Cy5 ratios were normalized by multiplying all measured Cy3:Cy5 ratios by the measured Cy5:Cy3 ratio of Complex V. This normalization has the effect of setting normalized Complex V ratio at 1:1 and makes comparison of results with other complexes and experiment to experiment variation more clear and eliminates any procedural differences resulting from differences in Cy3 and Cy5 labeling efficiencies. As shown in FIG. 11, each mAb captured both Cy3 and Cy5 labeled proteins from the mixed mitochondrial protein samples. The ratios were very constant from experiment to experiment over the course of up to five trials indicating that the method is highly reproducible. Similar 2-dye analyses using “capture” of labeled nucleotides by immobilized complementary nucleotides (gene chips) have been widely used to monitor the relative expression levels of mRNAs. If an unknown sample is labeled with one dye, e.g., Cy3, while a reference sample is labeled with the second dye, e.g., Cy5, then any difference in measured Cy3:Cy5 ratio of unknown:reference as compared to the ratio of reference:reference is indicative and directly proportional to the quantitative difference in relative concentrations of target mRNA in the unknown sample compared to the reference sample.

This Example demonstrates, inter alia, methods (and corresponding kits) for (i) determining a comprehensive snapshot of the status of many mitochondrial proteins at a single time, e.g., a microarray approach, and (ii) detailed proteomic analysis of individual multiprotein enzyme complexes and their post-translational modifications. Such assays and kits have many applications, such as to monitor normal physiologic variations in expression of the OXPHOS system proteins and complexes in cells or tissues exposed to variations in normal physiologic conditions, to monitor expression of OXPHOS proteins in various disease states and to serve as diagnostic tests for such diseases, and to monitor the mitochondrial health of individuals, to identify mitotoxic compounds, to identify therapeutic drugs that can protect mitochondria from mitotoxic compounds, and to monitor mitochondrial status in subjects undergoing HAART. Typically, such assays will be performed in a setting containing useful ancillary instrumentation, such as fluorescent microplate or microarray readers and mass spectrometers.

### Example 4

### Point-of-Care Immunoassays Useful for the Detection of Mitochondrial Dysfunction

This Example demonstrates a quantitative simple, point-of-care diagnostic test for mitochondrial dysfunctions, such as mtDNA depletion. The standard protocols currently used for measuring mtDNA depletion are Southern blotting and real time polymerase chain reaction. Both methods provide quantitative results, but neither is suitable for point-of-care testing, at least, because these methods require (i) trained technicians, (ii) expensive equipment, and (iii) isolation of mtDNA and nuclear DNA, which is technically demanding and may entail the handling of HIV-infective material. Moreover, these commonly used methods are time consuming and are not conducive to high throughput analysis.

A. OXPHOS Protein Expression is an Accurate Marker of mtDNA Depletion

Rather than measure mtDNA levels directly, the method described in this Example measures the initial consequences of mtDNA depletion, namely, the altered assembly of OXPHOS system complexes. As described previously, mtDNA-encoded proteins form the core structures of four of the five OXPHOS enzyme complexes, namely Complex I, Complex III, Complex IV and Complex V. Assembly of these complexes is therefore strictly dependent on the presence of the mtDNA-encoded subunits, and these subunits are made in progressively reduced amount as the mitochondrial genome is depleted. Advantageously, it is the copy number of assembled and functional OXPHOS enzymes rather than the copy number of the mitochondrial genome that most accurately reflects OXPHOS dysfunction and, therefore, the pathology resulting from mtDNA depletion.

Previous studies have shown that OXPHOS protein levels can be used as a surrogate marker for mtDNA levels (Marusich et al., Biochim. Biophys. Acta, 1362: 145-159, 1997). For example, Marusich et al. (Biochim. Biophys. Acta, 1362: 145-159, 1997) showed that mitochondrially encoded Complex IV subunits I and II were fully lost during mtDNA depletion (which depletion was secondary to an inherited disorders or caused by the mitotoxin, ethidium bromide) and that only a partially assembled, non-functional subcomplex composed of a subset of nuclear encoded subunits persisted.

In particular, this Example demonstrates that DDC-dependent depletion of mtDNA from fibroblasts is correlated with the loss of Complex IV subunit I (Janes et al., J. Histochem. Cytochem., 52: 1011-1018, 2004). Normal, human fibroblasts (cell strain MRC5) were treated with DCC for five to seven population doublings. Complex IV subunit I in treated fibroblasts was detected immunocytochemically with mAb RAC#18-1D6E1A8 using standard protocols (Janes et al., J. Histochem. Cytochem., 52: 1011-1018, 2004; RAC#18-1D6E1A8 is commercially available from Molecular Probes, MitoScience, and Abcam). Subunit I levels were determined relative to mitochondrial mass measured by porin content (a mitochondrial outer membrane protein encoded by nuclear DNA). Porin content was also determined immunocytochemically using porin-specific mAb 31HL (available from Calbiochem). mtDNA depletion in DCC-treated fibroblasts was detected using real time PCR and FISH with a mtDNA probe in accordance with commonly used methods (see, Janes et al., J. Histochem. Cytochem., 52: 1011-1018, 2004).

DDC depletes fibroblasts of mtDNA within three population doublings (PD). The disappearance of Complex IV subunit I protein lags, but full depletion occurs by six PDs. Accordingly, a standard curve, which relates the time course of subunit disappearance with the quantitative determination of mtDNA depletion, can be readily produced (see, Janes et al., J. Histochem. Cytochem., 52: 1011-1018, 2004).

Although direct measurement of any OXPHOS protein that is encoded by mtDNA can be used to report mtDNA depletion, certain of the OXPHOS complexes can provide stronger signals and/or more consistent results. It is believed that Complexes I and IV are particularly advantageous for reporting mtDNA levels, for example, because Complex I requires seven mtDNA-encoded subunits for assembly and Complex IV requires three. These mtDNA-encoded subunits form the catalytic core of each complex. Accordingly, both Complex I and Complex IV fail to assemble properly in Rho0 cells (which lack mtDNA altogether), in patients with inherited mtDNA depletion disorder (Marusich et al., Biochim. Biophys. Acta, 1362: 145-159, 1997), and in mitochondrial diseases involving point mutations in certain mitochondrially encoded subunits (Triepels et al., J. Biol. Chem., 276: 8892-8897, 2001; Hanson et al., J. Biol. Chem., 276: 16296-16301, 2001).

B. Lateral Flow Dipstick Devices: Simple, Quantitative Assays.

Lateral flow dipstick devices present an attractive simple solution to the problem of making an effective point-of-care diagnostic for mitochondrial dysfunction. These assays are simple, rapid, and can be self-reporting with qualitative or quantitative visual results. In some examples, such assays can provide relatively precise quantitative data if used in conjunction with (i) reusable dipstick readers (such as, the Quantum Designs reader (San Diego, Calif.), or any of a variety of readers available from Arista Biological (Allentown, Pa.)), or (ii) a hand-held disposable reader/dipstick dedicated for use with a single target antigen (such as the commercially available reader provided by Metrika, Inc.).

As described throughout this specification, mAbs are available that can specifically immunocapture partially or fully assembled OXPHOS enzyme complexes, and thereby detect mitochondrial dysfunction (including, for example, mtDNA depletion). Non-limiting representative capture mAbs are listed in Table 1.

Lateral flow devices (including, for example, dipsticks) are well known in the art, and any such device employing the disclosed mAbs and methods is contemplated herein. This Example describes one specific, non-limiting lateral flow format referred to as a two-antibody sandwich dipstick. In this format, one target antigen-specific mAb (such as, anti-Complex I or anti-Complex IV) is immobilized to a solid support (such as nitrocellulose) while another target antigen-specific mAb (such as, anti-Complex I or anti-Complex IV) is labeled with some marker, such as colloidal gold. Preferably, the two mAbs are specific for non-overlapping epitopes of the target antigen so that the mAbs can bind simultaneously to a single target antigen molecule. In the presence of the target antigen, a sandwich is formed, with an accumulation of the antigen and labeled mAb at the site of the immobilized capture mAb. A visible marker, such as colloidal gold, can be seen by the naked eye, which allows instrument-free readout. One of ordinary skill in the art will appreciate that a positive signal is generated only if an intact target antigen (that has both target epitopes) is captured between the two mAbs. Many known OXPHOS complex defects (for example, in Complex I or Complex IV) affect assembly of the enzyme complex and, therefore, can be expected to result in loss of signal in the dipstick test. mtDNA depletion also is known to result in the loss of assembled OXPHOS complexes, such as Complex I and Complex IV, when mtDNA-encoded protein subunits constitute the functional core of the enzyme complex.

1. Anti-Complex I-Specific Dipstick

A Complex I-specific dipstick was constructed with the first capture mAb (CI-1; RAC#24-20D1AB7, commercially available from MitoScience (Eugene, Oreg.)) immobilized in one or more zones drawn perpendicularly across the narrow width of a 0.5×4 cm rectangular nitrocellulose membrane. The membrane was laminated to a plastic backing to provide strength and support, and an absorbent pad was affixed to the top of the membrane at one end of the long side.

Test samples can be prepared from a variety of biological materials, including, for example, mitochondrial or whole cell extracts. Mitochondrial extracts were prepared by mixing mitochondria (5 mg protein per ml) with 1% (w/v) lauryl maltoside (a gentle non-ionic detergent), 100 mM NaCl, 25 mM HEPES pH 7.5, and the protease inhibitors, pepstatin (0.5 μg/ml), leupeptin (0.5 μg/ml) and PMSF (1 mM). The mixture was incubated at 4° C. for 30 minutes and then insoluble material was removed by centrifugation at 16,000×g for 20 minutes. As demonstrated in previous Examples, these conditions solubilize the OXPHOS enzymes in a fully-assembled state. In other examples, intact cell pellets can be solubilized in 1.5% lauryl maltoside at cell concentrations of approximately 1-2×10⁷ cells/ml. In some examples, it can be advantageous to dilute solubilized mitochondrial or whole-cell samples in a blocking buffer (such as, 0.2% lauryl maltoside, 5% (w/v) nonfat dry milk, 150 mM NaCl, 50 mM Tris-HCl, pH 7.5).

Test samples (e.g., containing Complex I target antigen) were pre-mixed with an excess of a second Complex I-specific capture mAb (CI-2; RAC#24-18G12BC2AA10, commercially available from MitoScience (Eugene, Oreg.)). This second capture mAb was conjugated to a particle with high light scattering properties, such as colloidal gold (gold-CI-2). The mAb-gold conjugates were prepared using standard protocols (Hughes, Preparation of Colloidal Gold Probes, in Immunochemical Protocols, 3rd ed., Methods in Molecular Biology, Vol. 295, pages 155-172, 2004).

When the free long end of the membrane was dipped in a liquid test sample (0.025-0.1 ml), the sample was wicked up along the membrane and passed through the narrow zone of immobilized CI-1 mAb. After about 10 minutes, the dipstick was transferred to a new well containing 50 μl of wash buffer (150 mM NaCl, 50 mM Tris-HCl, pH 7.5.). The dipstick can be read immediately, or can be air dried and stored for later densitometric analysis or as archival data. The developed dipsticks were stable indefinitely and the immunocaptured gold did not fade.

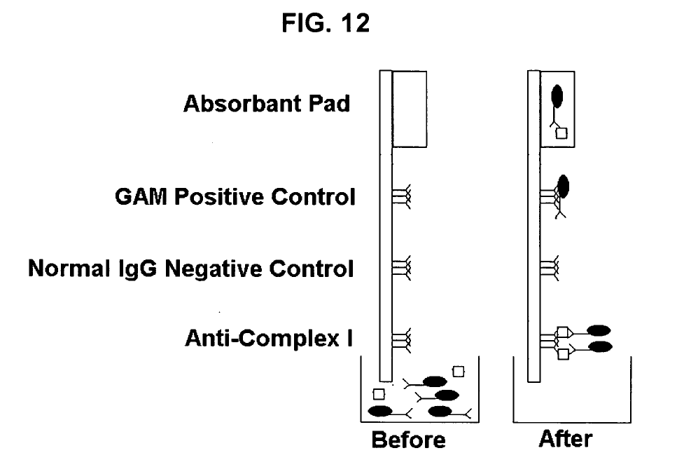

If Complex I was present in a particular sample, it was immunocaptured and concentrated along the line of immobilized CI-1 mAb. Successful immunocapture of Complex I from the sample was self-reporting and visually apparent because of the concomitant capture of antigen-bound gold-CI-2. The intensity of the CI-1 line is proportional to the concentration of Complex I in the sample. A schematic representation of this exemplar device is shown in FIG. 12.

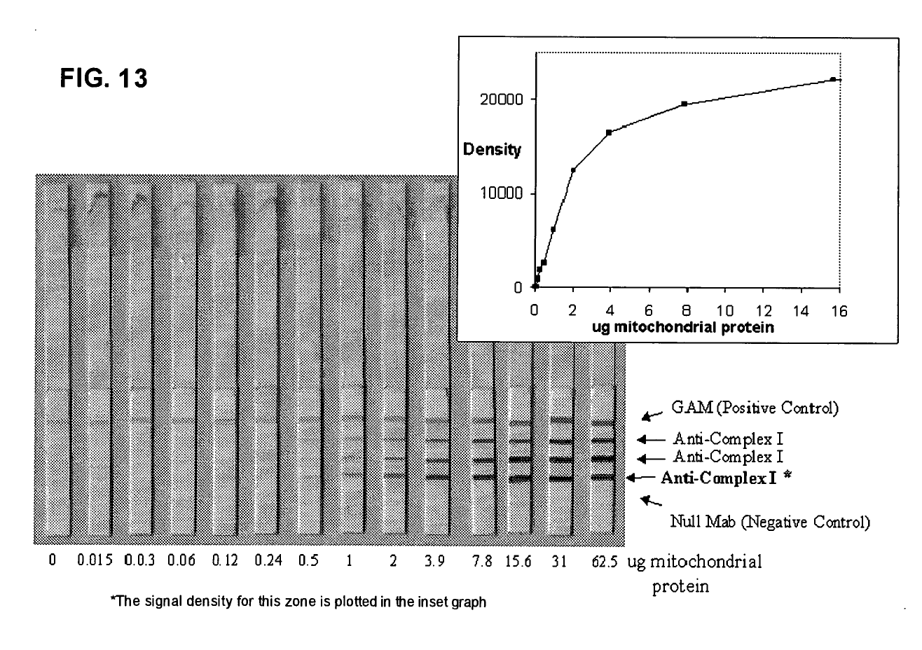

FIG. 13 shows the immunocapture of human Complex I from solubilized human heart mitochondria using a prototype dipstick. FIG. 13 also shows several controls, including (i) a dipstick run with sample buffer but no added mitochondria (see, dipstick at “0” μg mitochondrial protein) to show background immunocapture (background was undetectable by eye or by densitometric scanning), (ii) an internal negative control on each dipstick consisting of a zone of null or normal mouse antibody (this line revealed no detectable non-specific immunocapture), and (iii) an internal positive control on each dipstick consisting of a zone of goat-anti-mouse IgG (GAM) that directly immobilizes the gold-conjugated target-antigen-specific mAb (gold-CI-2) even in the absence of a Complex I sandwich. This positive control GAM line ensures that the reagents were added properly and that the sample passed through all antibody capture zones.

FIG. 13 shows that prototype Complex I-specific dipsticks are sensitive and have a wide dynamic range. A positive signal was detected both visually and by densitometry in samples containing as little as 0.12 μg human heart mitochondrial protein, and the signal increased in relationship to added mitochondria over at least a 100-fold range (0.12 μg/dipstick-15.8 μg/dipstick) (see, e.g., FIG. 13 inset). Background capture on the negative control mAb zone was undetectable at all sample concentrations, while the specific anti-Complex I zones showed signal only in the presence of added mitochondria. The positive control GAM line was positive at all sample concentrations.

2. Detection of Mitochondrial Dysfunction from mtDNA Depletion with a Prototype Complex I Dipstick

This subsection demonstrates that a Complex I specific dipstick can be used to detect the effects of mtDNA depletion. Human fibroblasts lacking mtDNA (Rho0 cells) were prepared in vitro by growing isolated fibroblasts for several cell generations in the presence of ethidium bromide at low concentration, as previously described (Marusich et al., Biochim. Biophys. Acta, 1362: 145-159, 1997). Ethidium bromide is specifically mitotoxic at these concentrations and exerts its toxic effect primarily by depleting mtDNA. Mitochondrial extracts were prepared from wild type and Rho0 cells, as described by Marusich et al. (Biochim. Biophys. Acta., 1362: 145-159, 1997), and intact enzyme complexes were solubilized with mild non-ionic detergent. Solubilization conditions were: 5 mg/ml mitochondrial protein, 1% wt/vol lauryl maltoside (a gentle non-ionic detergent), 100 mM NaCl, 25 mM HEPES pH 7.5, and protease inhibitors pepstatin (0.5 μg/ml), leupeptin (0.5 μg/ml) and PMSF (1 mM). The samples were incubated at 4° C. for 30 minutes and then insoluble material removed by centrifugation at 16,000×g for 20 minutes. Such extracts were tested using a prototype Complex I-specific dipstick as described in the preceding subsection.

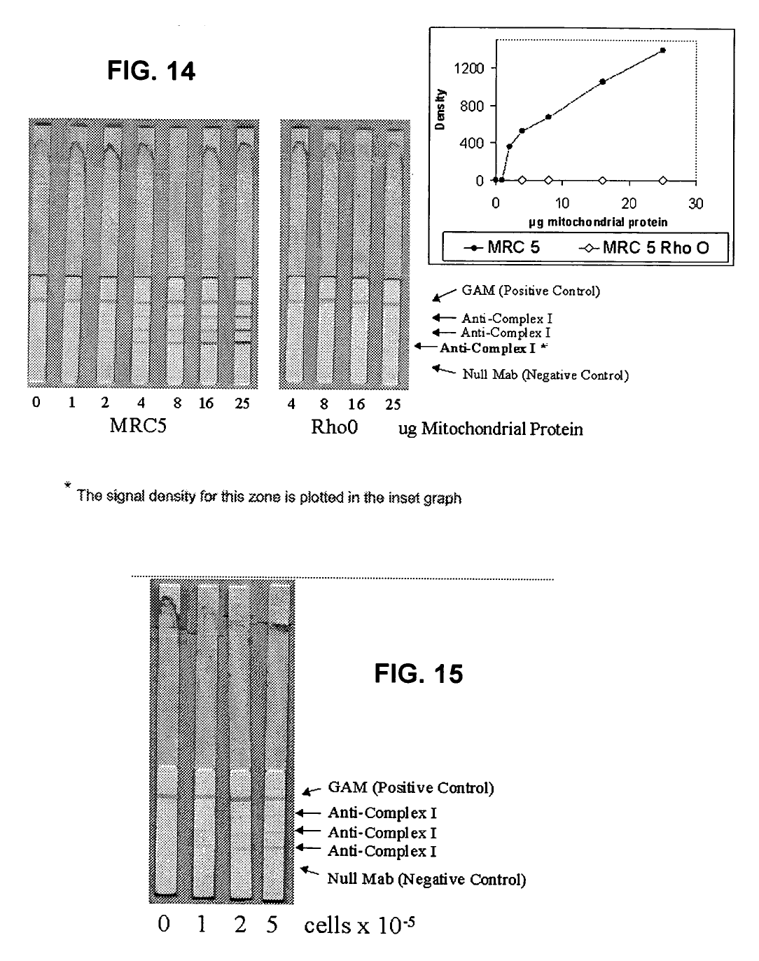

As shown in FIG. 14, Complex I was not detected in the Rho0 cells with even with sample containing as much as 25 μg Rho0 mitochondrial protein. Without being bound by theory, it is believed that Complex I fails to assemble in Rho0 cells because the catalytic core of Complex I is composed of mtDNA-encoded protein subunits. In comparison, Complex I was detected with as little as 2 μg of a protein extract from normal (MRC5) fibroblast mitochondria.

These results permit quantitative comparison of the amount of Complex I contained in normal and defective mitochondria. For example, Rho0 mitochondria showed no detectable signal at any concentration tested (up to 25 μg/test) while normal mitochondria showed detectable signals in samples containing as little as 2 μg/test; therefore, Rho0 mitochondria must contain Complex I at levels less than 8% of those found in normal cells.

3. Detection of Complex I in Peripheral Blood Mononuclear Cells (PBMCs)

This subsection demonstrates that Complex I can be detected by the prototype dipsticks using peripheral blood mononuclear cells (PBMCs). PBMCs are a sample that is useful in a clinical setting, at least, because PBMCs are readily available, easily collected, and exhibit metabolic dysfunction in HAART. One such tissue is peripheral blood, in particular the PBMCs. As shown in FIG. 15, as few as 1.25×10⁵ solubilized PBMCs provided a detectable signal using prototype Complex I dipsticks, and 5×10⁵ cells provided a robust signal. PBMCs can be prepared by any of a number of commercially available kits or by using a Ficoll-hypaque gradient solution (Amersham-Pharmacia Ficoll-Paque, Cat. No. 17-1440-02; Sigma Histopaque-1077, Cat. No. H8889) as described in the National Institute for Allergy and Infectious Disease, Adult Clinical Trials Group (ACTG) Specimen Procession Guide Index. A typical blood sample provides up to 1×10⁷ PBMCs, which is more than sufficient material for performing the disclosed dipstick test.

This Example shows, inter alia, that dipsticks specific for OXPHOS enzymes (such as, Complex I) can provide both qualitative and quantitative results, and can be used to detect mitochondrial dysfunction due to mtDNA depletion in fibroblasts and PBMCs.

### Example 5

### Quantitative Instrument-Free Dipsticks

Instrument-free dipsticks permit the quantification of target OXPHOS antigen levels without the need for a separate dipstick reader. As previously described, an OXPHOS-antigen-specific mAb-colloidal gold conjugate and an OXPHOS-antigen-specific mAb immobilized to a solid support (such as a dipstick) will be used.

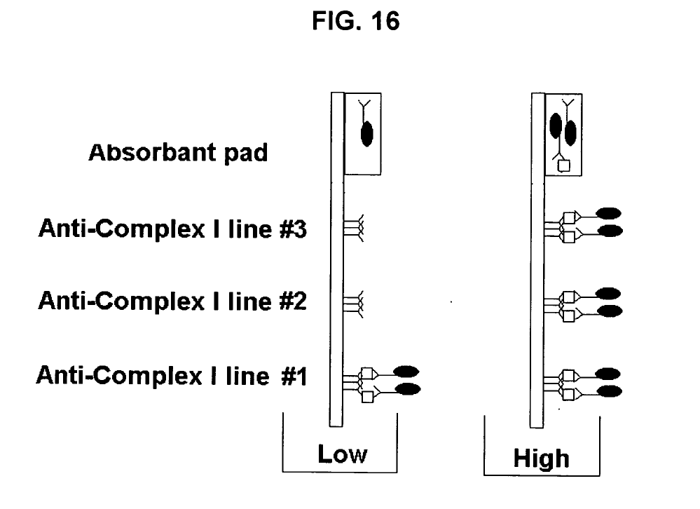

In its simplest format, a quantitative instrument-free device will be a dipstick with a single antigen-specific zone, and readout will be done visually by comparing the intensity of the signal to a calibrated reference card. In another example, the immobilized antigen-specific mAb will be immobilized either in a long continuous uniform zone or as series of multiple repeated zones (lines) rather than a single line. Such a configuration will provide a thermometer like readout of antigen concentration (see, e.g., FIG. 16).

In these examples, the target antigen (and its associated gold-conjugate mAb, which will be present in excess) will pass though this long zone (or series of lines) of immobilized capture mAb, and will be progressively immunocaptured. If there is only a small amount of target antigen in the sample, it will all be captured in the initial zone, making a single line. In contrast, if the antigen is present in large amount, it will saturate binding by immobilized capture mAb in the initial zone(s) and excess free antigen will move on to be captured by additional capture mAb immobilized in the next zone, and so on.

Optionally, the gradient of signal will be calibrated with a reference card, or by the simultaneous running of a reference sample on a separate dipstick. The reference sample can be one provided in a kit, or a normal reference control sample generated by the end user.

### Example 6

### Immunocapture of OXPHOS Complexes and Detection of Post-Translational Modifications, as Exemplified by Phosphoprotein Detection

As discussed in detail throughout this specification, there is considerable interest in OXPHOS enzymes because of the variety of diseases which are caused by defective functioning of mitochondria. However, proteomic analysis of mitochondria is not simple, for example, because of the large number of proteins involved (1,500 by some estimates; Lopez et al., Electrophoresis, 21: 3427-3440, 2000). These mitochondrial proteins are present in a wide range of copy number with many showing a variety of post-translationally modified forms. Adding to the complications of analysis, the majority of mitochondrial proteins are in a narrow molecular mass range (between 10,000 and 30,000 Da) and a significant proportion is hydrophobic.

2D-electrophoretic separation of detergent-solubilized proteins is often used to analyze mitochondrial proteins. However, even at the highest resolution, not all proteins are resolved by this method; for example, hydrophobic proteins do not routinely enter such gels, it is difficult to resolve reproducibly basic proteins, and limitations on the amounts of total protein that can be loaded preclude identification of the low copy number proteins (Santoni et al., Electrophoresis, 21: 1054-1070, 2000). More complicated methods have evolved to circumvent such problems, including sucrose density gradient subfractionation followed by SDS solubilization and single dimension electrophoresis (Hanson et al., Electrophoresis, 22: 950-959, 2001; Taylor et al., Nat. Biotechnol., 21: 281-286, 2003). Nevertheless, sucrose gradient fractions are still complex mixtures of proteins, which present problems for analyzing post-translational modifications of OXPHOS complexes.

This Example demonstrates that immunocapture of OXPHOS enzyme complexes overcomes the problems of the prior methods. In particular, this Example demonstrates monoclonal antibodies capable of immunocapturing all five OXPHOS complexes and evaluation of post-translational modifications present in such complexes. Using immunocapture, Complexes I, II, III, IV and V were obtained in good yield from small amounts of tissue in more than 90% purity in one step.

A. Materials and Methods

1. Preparation of Mitochondria

Bovine heart mitochondria were prepared according to Smith (Meth. Enzymol., 10: 81-86, 1967). Briefly, ventricles were homogenized and particulate material was removed by centrifugation at 1000×g. Mitochondria were collected from the supernatant by spinning down at 12000×g and resuspended in the isotonic buffer, 10 mM Tris-HCl pH 7.8, 0.25 M sucrose, 0.2 mM EDTA, 0.5 mM PMSF. Protein concentration was determined by the BCA method (Pierce).

2. Isolation of Mitochondrial OXPHOS Complexes I-V

All mitochondrial samples were washed with 20 mM Tris-HCl pH 7.5, 1 mM EDTA. The mitochondria were resuspended at a concentration of 10 mg/ml in the same buffer supplemented with protease inhibitors (leupeptin at 2 μg/ml, pepstatin at 2 μg/ml and 2 mM PMSF). Next, 0.01 volumes each of phosphatase inhibitor cocktails I and II (Sigma) were added. An equal volume of 2% n-dodecyl-β-D-maltoside (Anatrace) was added to a final concentration of 1% detergent at 5 mg/ml protein, and incubated for 30 min on ice. Insoluble material was removed from the samples by centrifugation in a TLA100.2 (Beckman) at RCF max 89,000×g for 30 minutes at 4° C.

Monoclonal antibodies used in this Example are shown below in Table 8. The approximate molecular mass of monomeric enzyme is given in the table; however, Complex II, III and IV exist as tightly bound homodimers within the inner mitochondrial membrane (Yu et al., Biochim. Biophys. Acta, 1275: 47-53, 1996; Lancaster et al., Nature, 402: 377-385, 1999; Tsukihara et al., Science, 272: 1136-1144, 1996). In addition, Complexes I, II, III and IV are believe to exist as a loosely bound functional units and Complex V is thought to exist as a loosely associated dimer (Schagger and Pfeiffer, Embo J., 19: 1777-1783, 2000).

| OXPHOS | | | Approx. | Number of | mAb |
|---|---|---|---|---|---|
| Enzyme | | E.C. # | MW | Subunits | Clone |
| I | NADH:ubiquinone | 1.6.5.3 | 1000 kDa | 45 | 18G12 |
| | oxidoreductase | | | | |
| II | Succinate:ubiquinone | 1.3.5.1 | 130 kDa | 4 | 4H12 |
| | oxidoreductase | | | | |
| III | ubiquinol cytochrome | 1.10.2.2 | 250 kDa | 11 | 1A11 |
| | c oxidoreductase | | | | |
| IV | cytochrome c oxygen | 1.9.3.1 | 200 kDa | 13 | 7E5 |
| | oxidoreductase | | | | |
| V | F₁F₀ ATP synthase | 3.6.3.14 | 650 kDa | 17 | 12F4 |

For immunoisolation, 100 μg of monoclonal antibody were bound to 10 μl of swollen protein G agarose beads (Sigma). The antibody was crosslinked to the beads with 25 mM dimethylpimelimidate (Sigma) for 30 minutes at room temperature in 0.2 M sodium borate, pH 9.0. Cross-linking was terminated with 0.1 M ethanolamine solution, pH 8.0, for 3 hours at room temperature. Antibody cross-linked beads were collected by gentle centrifugation at 3000 rpm in a microfuge and resuspended in phosphate buffered saline.

The antibody cross-linked beads were incubated for 3 hours at room temperature with the supernatant from 5 mg of solubilized mitochondria with rotation. Beads were washed 3 times with PBS supplemented with 0.05% n-dodecyl-β-D-maltoside. Immunocaptured proteins were eluted with 50 μl SDS-PAGE loading buffer without reducing agent and heated at 95° C. for 5 minutes.

3. Electrophoresis and Detection of OXPHOS Phosphoproteins

Electrophoresis was performed according to Laemmli (Nature, 227: 680-685, 1970) using a Tris-HCl 10-22% acrylamide gel. Fluorescent staining and subsequent imaging of the gel with Pro-Q Diamond phosphoprotein gel stain and then SYPRO Ruby protein gel stain (Molecular Probes) were performed as described by Schulenberg et al. (J. Biol. Chem., 278: 27251-27255, 2003). Gels were also stained for total protein with the commonly used Coomassie stain (Brilliant Blue R from Sigma).

4. Western Blotting Detection of OXPHOS Subunits

One-dimensional gels (three cm wide lanes on 10-22% gels) were transferred in CAPS buffer from gels to PVDF membranes (0.45 μm pore size) according to Triepels (J. Biol. Chem., 276: 8892-8897, 2001). After transfer, 4 mm wide PVDF membrane strips were cut and exposed to antibodies against individual subunits. OXPHOS protein subunits were detected by the monoclonal antibodies listed in Table 8, followed by a secondary goat anti-mouse polyclonal antibody conjugated to alkaline phosphatase then visualized by the NBT/BCIP method (Biorad). Monoclonal antibodies used in FIG. 18 strips 1-6 were detected with Trueblot™ (eBiosciences) an HRP conjugated goat antibody specific against a native disulfide bond of mouse antibodies and visualized by diaminobenzidene in a colorimetric assay (Sambrook et al., Molecular Cloning: A Laboratory Manual, Cold Spring Harbor Press, vol. 3, section 18.75, 1989).

TABLE 8
Antibodies Used to Confirm the Subunit Composition
and Purity of Immunocaptured OXPHOS Complexes.

| | | Subunit | MW | mAb | μg/ml |
|---|---|---|---|---|---|
| I | 1 | I - NDUFA9 | 39 kDa | 20C11 | 1 |
| | 2 | I - NDUFS3 | 26 kDa | 17D9 | 1 |
| | 3 | I - NDUFS4 | 15 kDa | 2C7 | 1 |
| | 4 | I - ND6 | 19 kDa | 20E9 | 1 |
| | 5 | IV - COXVIb | 10 kDa | 3F9 | 1 |
| II | 1 | IV - COXVIc | 8 kDa | 3G5 | 2 |
| | 2 | II - SDHA | 68 kDa | 2E3 | 0.1 |
| | 3 | II - SDHB | 29 kDa^{a} | 21A11 | 5 |
| | 4 | III - core 1 | 49 kDa | 16D10 | 1 |
| III | 1 | III - core 1 | 49 kDa | 16D10 | 1 |
| | 2 | III - core 2 | 47 kDa | 13G12 | 0.5 |
| | 3 | III - Rieske FeS | 22 kDa | 5A5 | 2 |
| | 4 | III - 10 kDa | 10 kDa | 1H9 | 2 |
| | 5 | V - Subunit d | 19 kDa | 7F9 | 0.5 |
| IV | 1 | IV - COXI | 57 kDa | 1D6 | 1 |
| | 2 | IV - COXIV | 17 kDa | 20E8 | 1 |
| | 3 | IV - COXVb | 11 kDa | 16H12 | 5 |
| | 4 | IV - COXVIb | 10 kDa | 3F9 | 1 |
| | 5 | II - SDHB | 29 kDa^{a} | 21A11 | 5 |
| V | 1 | V - α | 55 kDa | 15H4 | 1:1000^{b} |
| | 2 | V - β | 52 kDa | 3D5 | 1 |
| | 3 | V - Subunit d | 19 kDa | 7F9 | 1 |
| | 4 | V - OSCP | 21 kDa | 4C11 | 1 |
| | 5 | V - IF1 | 10 kDa | 5E2 | 1 |
| | 6 | I - ND6 | 19 kDa | 20E9 | 1 |
| | 7 | V - Subunit d | 19 kDa | 7F9 | 1 |

^{a}The estimate of molecular mass of SDHB is based on the human homologue.
^{b}1:1000 dilution from cell culture condition media.

5. Detection of OXPHOS Phosphoproteins

Complexes I-V were immunocaptured with monoclonal antibodies and applied on a 10-22% SDS polyacrylamide gel. The gel was stained with Pro-Q Diamond phosphoprotein gel stain according to the protocol of the manufacturer except that the destaining was carried out for 3×30 minutes (as described by Schulenberg et al., J. Biol. Chem., 278: 27251-27255, 2003). After imaging the gel using 532 nm excitation and a 580 nm long pass emission filter the gel was stained with SYPRO Ruby and imaged using 473 nm excitation and a 580 nm long pass emission filter.

B. Identification of Immunocaptured OXPHOS Complexes and Phosphoproteins

OXPHOS enzymes are the major protein components of mitochondrial membranes. Until now the isolation of any of the OXPHOS complexes has been a lengthy procedure requiring large quantities of tissue. However, as described in this Example, mAbs described in this Example (and elsewhere in the specification) are capable of immunocapturing the five native OXPHOS complexes using a simple, rapid protocol. Phosphorylation of immunocaptured OXPHOS complexes were analyzed using the dye Pro-Q Diamond (Molecular Probes). This protocol is equally efficient for isolation of OXPHOS complexes from bovine heart mitochondrial and from human tissues.

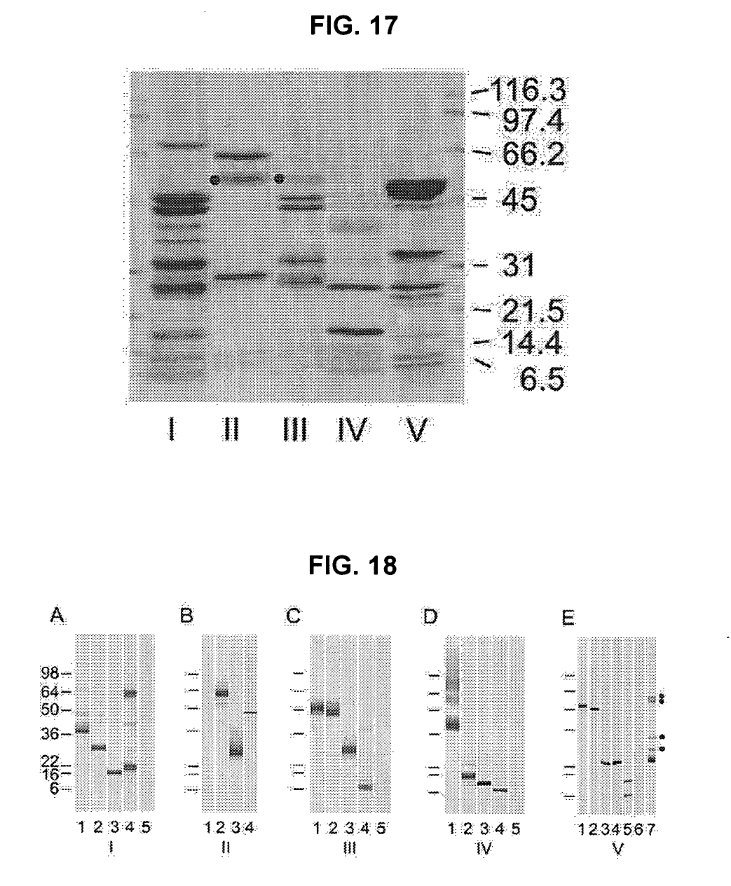

The subunit composition of the five OXPHOS complexes obtained by immunocapture is shown in FIG. 17, based on SDS-PAGE using a 10-22% gradient gel and staining with Coomassie brilliant blue. On this gel system, Complex I was resolved into 27 bands. Mass spectrometry of these 27 bands identified 44 of the 45 subunits that are known components of the complex (Murray et al., J. Biol. Chem., 278: 13619-13622, 2003). The only subunit not identified in this way was ND4L, which is small, very hydrophobic, and does not generate any peptides, by trypsin treatment, that are detectable by mass spectrometry. Complex II was resolved into 4 subunit bands on this gel. The 70 kDa and 30 kDa bands were the flavoprotein (Fp) and iron-sulfur protein (Ip) subunits of succinate dehydrogenase respectively, while the 15 and 13 kDa protein bands were the membrane-anchoring cytochrome b associated subunits often termed cybL and cybS. The band running immediately below the 70 kDa succinate dehydrogenase flavoprotein was a small amount of antibody heavy chain released from the beads. Complex III was resolved into 8 bands, which when analyzed by mass spectrometry, include 10 of the 11 subunits known to comprise this complex. The one Complex III subunit not identified by mass spectrometry was subunit XI, an extremely small protein. Complex V, as purified by immunocapture, was resolved by this gel into 8 bands. The largest band included both the α and β subunit. Mass spectrometry of this complex identified the presence of 15 of the 17 known subunits of the complex with only the highly hydrophobic proteolipid and coupling factor B undetected (Aggeler et al., J. Biol. Chem., 277: 33906-33912, 2002). Mass spectrometry also identified the adenine nucleotide translocase as a major component, consistent with another report of the association of this protein with the ATP synthase (Ko et al., J. Biol. Chem., 278: 12305-12309, 2003). Complex IV, the cytochrome c oxidase, was resolved on this gel system into 6 bands. The band with mass around 40 kDa was subunit I, which stained poorly with Coomassie blue. The band at approximately 25 kDa contained both subunits II and III, and the lower bands contained 9 of the 10 remaining subunits known to be a part of this complex. The only subunit not identified by mass spectrometry was subunit VIII, a small hydrophobic polypeptide which went undetected for the same reasons given above for Complex I subunit ND4L. Immunocaptured Complex IV also contained a small proportion, probably less than 10%, of Complex III. This is most likely due to the existence of a supercomplex between complexes III and IV (Cruciat et al., J. Biol. Chem., 275: 18093-18098, 2000; Schagger and Pfeiffer, Embo J., 19: 1777-1783, 2000). Thus, the detergent solubilization conditions described in this Example, which maintain OXPHOS complexes in a structural and functional state, do not disrupt every III/IV supercomplex.

In addition to antibodies capable of isolating structurally and functionally intact OXPHOS complexes from solubilized mitochondria, this Example also includes “non-capture” monoclonal antibodies specific to 31 of the 89 OXPHOS proteins for use in Western blotting applications (mAbs against 7 subunits of Complex I, 2 of Complex II, 4 of Complex III, 12 of Complex IV and 6 of Complex V). FIG. 18 shows Western blots using a subset of these non-capture mAbs (see also, Table 2), which were used solely to confirm the presence of selected subunits in an immunocaptured complex and the absence of contamination from other OXPHOS complexes. For the mitochondrially encoded subunits ND6 of Complex I and subunit I of Complex IV more than one band was observed. The higher molecular mass bands represented aggregates of these subunits after heating the samples. In the case of the ATP synthase, but not with the other complexes, there was cross reactivity of the secondary antibody with both the heavy and light chain of the immunocapture antibody (FIG. 18E, strip 7), which had been released as Complex V was eluted from the protein G beads. This cross reactivity was eliminated by using a recently available secondary antibody that recognizes only native mAb structure (FIG. 18E, strips 1-6). FIG. 18 further emphasizes the purity of the 5 OXPHOS complexes when immunocaptured. Note the absence of cytochrome c oxidase in Complex I (FIG. 18A, strip 5) of succinate dehydrogenase in Complex IV (FIG. 18D, strip 5) and NADH dehydrogenase in Complex V (FIG. 18E, strip 6), or ATP synthase in Complex III (FIG. 18C, strip 5). Immunocaptured Complex II contains a small amount of Complex III core 1 protein (FIG. 18B, strip 4).

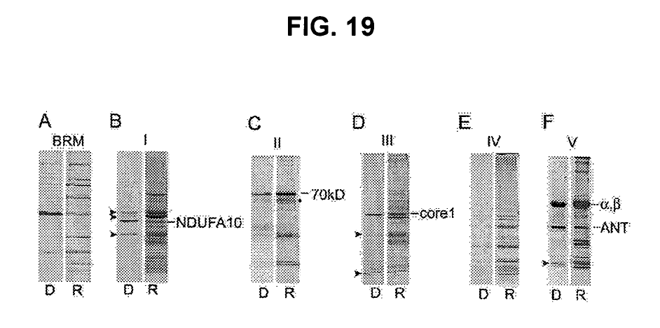

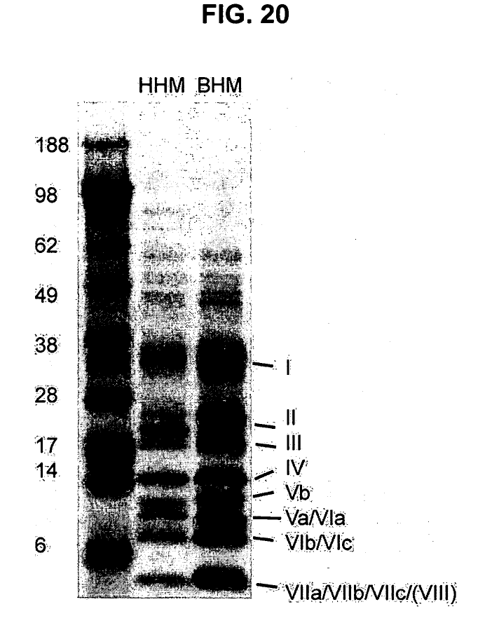

FIG. 19A shows a broad range marker (BioRad) serving as a control in which a set of non-mitochondrial proteins of known phosphorylation state were examined by SDS-PAGE followed by Pro-Q Diamond staining to identify phosphorylation and SYPRO Ruby staining to detect protein levels. Ovalbumin, the only phosphoprotein in this mixture was heavily stained with Pro-Q Diamond. This dye also stains non-phosphorylated proteins at a low but detectable level which background is preferably subtracted during analysis (FIG. 19A). FIG. 19 demonstrates that the known set of OXPHOS phosphoproteins (Schulenberg et al., J. Biol. Chem., 278: 27251-27255, 2003) were identified by this method, i.e., NDUFA10 of Complex I (FIG. 19B), the 70 kDa succinate dehydrogenase flavoprotein of Complex II (FIG. 19C), and the α and β subunits of Complex V, along with the adenine nucleotide translocase (ANT) (FIG. 19F). In addition, the increased purity of immunocaptured Complex III demonstrates a much greater phosphoprotein staining intensity of core 1 than core 2 (FIG. 19D) as compared to Schulenberg et al. (J. Biol. Chem., 278: 27251-27255, 2003).

Several other potential phosphoproteins of the OXPHOS complexes are indicated by arrows in FIG. 19. These included NDUFS2 and NDUFV1 of Complex I, cytochrome b and one of the smaller subunits of Complex III (IX, X or XI), as well as an approximately 8 kDa ATP synthase subunit which could be the proteolipid subunit c or the ε subunit of Complex V.

In summary, this Example demonstrates that immunocapture using anti-OXPHOS capture antibodies is superior to sucrose gradient separation and other more traditional methods of OXPHOS enzyme isolation, at least, because (i) it is rapid to perform and is a one-step procedure which minimizes degradation of proteins and loss of modification; (ii) only small amounts of tissue or cell culture material are needed; (iii) the purity of OXPHOS enzymes is considerably greater, reducing the potential for contamination of subunit bands by non-related co-eluting proteins which, at least, facilitates mass spectrometric identification and detection of post-translational modifications. This Example further shows the utility of the immunocapture approach to resolve and analyze phosphorylated OXPHOS subunits.

### Example 7

### Immunocapture of OXPHOS Complexes and Detection of Post-Translational Modifications, as Exemplified by Detection of Oxidative Damage

Defects in cytochrome c oxidase (Complex IV or COX), the terminal component of the mitochondrial electron transport chain, are a common cause of so-called mitochondrial diseases such as Leigh's disease. A Complex IV activity deficit has also been detected in Alzheimer's disease. This example demonstrates an immunocapture approach for isolating Complex IV from very small amounts of tissue and cell culture material. This purification facilitates Complex IV subunit analysis by SDS-PAGE and mass spectrometry. Activity measurements show that the enzyme bound to capture beads retains cytochrome c oxidase (Complex IV) activity while being completely cyanide sensitive. This antibody capture approach was used to isolate Complex IV from mitochondria that had been treated in vitro with either peroxynitrite or hydroxyl radicals, two oxidants believed to contribute to cellular oxidative stress found in neurodegenerative diseases such as Alzheimer's. The reactions of both oxidants were relatively specific and confined to subunit Vb, in the case of peroxynitrite damage, and subunit IV, in the case of hydroxyl radicals. The reaction of both oxidants caused a reduction of Complex IV (cytochrome c oxidase) activity which could be measured in mitochondria and after isolation of the enzyme.

A. Materials and Methods

1. Preparation of Bovine and Human Mitochondria. Bovine heart mitochondria were prepared according to Smith (Meth. Enzymol., 10: 81-86, 1967). Briefly, ventricles were homogenized and particulate material was removed by centrifugation at 1000×g. Mitochondria were collected from the supernatant by spinning down at 12000×g and resuspended in the isotonic buffer, 10 mM Tris-HCl pH 7.8, 0.25 M sucrose, 0.2 mM EDTA, 0.5 mM PMSF. Kidney mitochondria were prepared in a similar fashion from bovine kidney cortex. Human heart mitochondria, prepared from the heart of a 47-year-old man who died of brain cancer, were obtained from Analytical Biological Services (Wilmington, Del.). Protein concentration was determined by the BCA method (Pierce).

2. Immunocapture of Complex IV. Five (5) mg of mouse anti-Complex IV monoclonal antibody 7E5BA4 (MitoScience LLC) was incubated by constant turning overnight at 4° C. with 1 ml 1 mL of swollen protein G agarose beads (Sigma). Beads were washed in phosphate buffered saline (PBS), 1.4 mM KH₂PO₄, 8 mM NaH₂PO₄, 140 mM NaCl, 2.7 mM KCl, pH 7.3. The antibody/bead conjugates were cross-linked for 30 min with 20 mM dimethylpimelimidate (Sigma) in 0.2 M sodium borate pH 9.0. The reaction was stopped by incubation with 0.1 M ethanolamine pH 8.0 for 3 hours. Beads were resuspended in PBS. For the immunocapture of Complex IV, 5 mg mitochondria in 1 mL PBS were solubilized with 20 mM dodecyl-β-D-maltoside (Calbiochem) lauryl maltoside for 30 min on ice. Insoluble material was removed by centrifugation at 70 70,000×g for 30 min. The recovered supernatant was incubated with 10 μl antibody-conjugated beads overnight at 4° C. while turning. Beads were washed 3 times in 1 mL PBS, 0.1 mM dodecyl-β-D-maltoside. Immunocaptured protein concentration was established by elution in 1% SDS and assay followed by the BCA method (Pierce). It was established that beads, prepared in this way, captured about 1 μg Complex IV per 1L beads.

3. Electrophoresis of Bovine Heart and Kidney Complex IV. Ten (10) μg of Complex IV from bovine heart and kidney mitochondria were resolved by 4-12% Bis-Tris NuPAGE gels using the MES electrophoresis buffer (Invitrogen). Gels were stained with Coomassie brilliant blue (Sigma).

4. Fe³Fe²⁺ Catalyzed Oxidation of Mitochondria. Mitochondria at 5 mg/mL in 25 mM KH₂PO₄ pH 7.2, 5 mM MgCl₂ were incubated for 0, 0.5, 1, 2, or 4 hours with 1 mM FeCl₃/25 mM sodium ascorbate. Incubation could not be performed for less than 0.5 h because iron could only be removed by rapid dialysis, which was performed for 30 min against 25 mM potassium phosphate pH 7.2, 5 mM MgCl₂ using 12000 Da MWCO dialysis membrane (Spectrapor). After oxidation, mitochondrial membranes were subjected to the Complex IV activity assay described below. The Complex IV enzyme was then isolated by the immunocapture extraction method above.

5. Peroxynitrite Catalyzed Oxidation of Mitochondria. Mitochondrial membranes at 5 mg/mL protein in 25 mM potassium phosphate, pH 7.2, 5 mM MgCl₂ were placed on ice. Peroxynitrite 88 mM (Upstate) was diluted in 0.3 M NaOH and distributed on the side of microcentrifuge tubes containing mitochondria to a final concentration of 0, 50, 100, 200, 400, 800, or 1600 μM. The reaction was initiated by vigorous mixing and incubation 30 min on ice. Following this oxidative treatment, Complex IV activity was measured before and after Complex IV immunocapture.

6. Complex IV Activity in Mitochondrial Membranes and After Immunocapture. Potassium cyanide-sensitive Complex IV activity was measured in bovine heart mitochondria or with immunopurified isolated enzyme. Ferrocytochrome c was generated by dissolving bovine heart cytochrome c (Sigma) into assay buffer and reducing it with a saturating amount of L-(+)-ascorbic acid (MCB Reagents). Reduced bovine heart cytochrome c was purified over a Sephadex G-15 (Pharmacia) column equilibrated with assay buffer, collected in 1 mL fractions, and the concentration measured at 550 nm in a Beckman DU7. The oxidation of ferrocytochrome c was followed at 550 nm in a 1 mL reaction vessel in the DU7 or in a 96 well plate (Corning) using a Victor2 1460 multi-plate reader (Perkin-Elmer) with a narrow band pass 550 nm filter. Reaction vessels contained 30 μM ferrocytochrome c and assays were carried out at 22° C. in Complex IV assay buffer (25 mM KH₂PO₄, pH 7.2, 2 mM dodecyl-β-D-maltoside). The reaction was initiated by the addition of either 2 μg mitochondria or immunocaptured Complex IV attached to beads. The initial rate of ferrocytochrome c oxidation was followed for 2 min. An absorption coefficient of 19 mM⁻¹cm⁻¹ for ferrocytochrome c and a light path length of 1 cm (Beckman DU7). Alternatively a Victor 2 96 well plate reader was used when analyzing multiple samples.

7. Western Blot Detection and Isolation of Oxidized Proteins. For carbonyl detection in Complex IV the Oxyblot system was used (Chemicon). Briefly, Complex IV was eluted from beads in 6% SDS and oxidized carbonyls derivatized by DNPH. Samples were resolved by electrophoresis on two NuPAGE 4-12% Bis Tris gels with MES running buffer. One gel was stained with Coomassie brilliant blue before protein band isolation for mass spectrometry analysis. The other gel was Western blotted according to Murray et al. (J. Biol. Chem., 278: 37223-37230, 2003) before being probed with anti-DNP rabbit polyclonal antibody using the ECL+ (Amersham) detection method. For the detection of peroxynitrite modified proteins, Complex IV was eluted from beads in 6% SDS, diluted in protein electrophoresis buffer without SDS (Tris-HCl pH 6.8, 5% glycerol, 0.02 mg/mL bromophenol blue). Proteins were resolved by NuPAGE 4-12% Bis Tris gels with MES running buffer. As above, one gel was stained with Coomassie brilliant blue while the other was transferred to PVDF before being Western blotted with an anti-3nitrotyrosine polyclonal antibody (Molecular probes) as described in Murray et al. (J. Biol. Chem., 278: 37223-37230, 2003).

8. In-Gel Proteolytic Digestion of Proteins. For proteolysis, sequencing grade modified porcine trypsin (Biorad) and bovine chymotrypsin (Roche) were used. HPLC solvents such as acetonitrile and water were obtained from Burdick & Jackson. Protein spots of interest were manually excised out of the gel and processed with the automatic in-gel digester robot ProGest (Genomic Solutions). The gel spots were destained and dehydrated with acetonitrile. Subsequently, they were reduced with 10 mM DTT at 60° C. for 30 min and alkylated with 100 mM iodoacetamide (37° C., 45 min). All samples were then incubated with 125 ng sequencing grade trypsin at 37° C. for 4 hours. The resultant tryptic peptides were extracted from gel slices by 10% formic acid extraction and analyzed by mass spectrometry. In addition, gel bands from bovine heart Complex IV were digested with chymotrypsin as well. After reduction and alkylation as described above, samples were incubated with chymotrypsin at room temperature for 4 hours. The resultant chymotryptic peptides were extracted with 50% acetonitrile/5% formic acid, concentrated and analyzed.

9. MALDI-TOF MS. Mass spectra of digested gel spots were obtained by MALDI-TOF MS on a Voyager DE-STR plus (Applied Biosystems). All mass spectra were acquired in positive-ionization mode with reflectron optics. The instrument was equipped with a 337 nm nitrogen laser and operated under delayed extraction conditions; delay time 190 nsec, grid voltage 66-70% of full acceleration voltage (20-25 kV). All peptide samples were prepared using a matrix solution consisting of 33 mM α-cyano-4-hydroxycinnamic acid (Agilent Technologies) in acetonitrile/methanol (1/1; v/v); 1 μL of analyte (0.1-1 pmol of material) was mixed with 1 μL of matrix solution, and then air-dried at room temperature on a stainless steel target. Typically, 50-100 laser shots were used to record each spectrum. The mass spectra obtained were externally calibrated with an equimolar mixture of angiotensin I, ACTH 1-17, ACTH 18-39, and ACTH 7-38.

10. ESI-MS, MS/MS. In all cases, the proteolytic peptide mixtures were analyzed by reverse-phase nano-HPLC-MS/MS. Briefly, peptides were separated on an Ultimate nanocapillary HPLC system equipped with a PepMap™ C18 nano-column (75 μm I.D.×15 cm) (Dionex) and CapTrap Micro guard column of 0.5 μl bed volume (Michrom). Peptide mixtures were loaded onto the guard column and washed with the loading solvent (0.05% formic acid, flow rate: 20 μL/min) for 5 min, then transferred onto the analytical C18-nanocapillary HPLC column and eluted at a flow rate of 300 nL/min using the following gradient: 2% solvent B in A (from 0-5 min), and 2-70% solvent B in A (from 5-55 min). Solvent A consisted of 0.05% formic acid in 98% H₂O/2% ACN and solvent B consisted of 0.05% formic acid in 98% ACN/2% H₂O. The column eluant was directly coupled to a ‘QSTAR Pulsar i’ quadrupole orthogonal TOF mass spectrometer (MDS SCIEX) equipped with a Protana nanospray ion source (ProXeon Biosystems). The nanospray needle voltage was typically 2300 V in the HPLC-MS mode. Mass spectra (ESI-MS) and tandem mass spectra (ESI-MS/MS) were recorded in positive-ion mode with a resolution of 12000-15000 full-width half-maximum. For collision induced dissociation tandem mass spectrometry (CID-MS/MS), the mass window for precursor ion selection of the quadrupole mass analyzer was set to ±1 m/z. The precursor ions were fragmented in a collision cell using nitrogen as the collision gas. Spectra were calibrated in static nanospray mode using MS/MS fragment-ions of a renin peptide standard (His immonium-ion with m/z at 110.0713, and b₈-ion with m/z at 1028.5312) providing a mass accuracy of ≦50 ppm.

11. Database Searches for Protein Identification. Mass spectrometric data were analyzed with the bioinformatics database system RADARS (Genomic Solutions) (Field et al., Proteomics, 2: 36-47, 2002) and Mascot (Matrix Sciences) (Perkins et al., Electrophoresis, 20: 3551-3567, 1999). Routinely, MALDI-MS data were analyzed with RADARS using the search engine ProFound for Peptide Mass Fingerprints (PMF) matching against peptides from known protein sequences entered in publicly available protein databases (e.g. NCBI) using the following parameters: internal calibration using trypsin autolysis masses (m/z 842.5100 and 2211.1046), 100 ppm mass accuracy, 2 missed proteolytic cleavages allowed. In all cases, tryptic and chymotrypsin digestion extracts of proteins were analyzed by HPLC-ESI-MS and MS/MS, these data were then submitted to the search engine Mascot that analyzes peptide sequence information from tandem mass spectra. For this example, a custom-designed database was incorporated into in-house licensed search engine Mascot, such as a generated “bovine Complex IV” database that allowed for more specific searches. Both search engines applied, provide a statistical scoring parameter, for example, Profound searching peptide mass fingerprint data uses a so-called ‘expectation value’ for data quality control that becomes smaller as the probability of a nonrandom (real) protein hit increases, e.g. 1×10⁻² is a 1 in 100 chance of being a random hit (confidence>99.0%); protein matches are considered significant for scores with expectation value<5×10⁻² (confidence>95%) (Field et al., Proteomics, 2: 36-47, 2002). The search engine Mascot uses a probability based ‘Mowse Score’ to evaluate data obtained from tandem mass spectra, e.g. for a score>37, protein matches are considered significant (Perkins et al., Electrophoresis, 20: 3551-3567, 1999).

B. Quantitative Immunocapture of Complex IV

The mouse monoclonal antibody 7E5BA4 is known to immunocapture Complex IV from human tissues. The conserved specific antigen to which it binds is undetermined at this time since it does not react in a Western blotting protocol, presumably because the antigen is denatured by SDS prior to electrophoresis. A typical immunocapture uses 10 μl antibody-bead conjugate and yields 10 μg of Complex IV from as little as 250 μg of mitochondria, amounts that are easily obtained from platelets, needle biopsy, or cultured cells. Two consecutive immunocapture steps with 10 μl beads effectively bound all available Complex IV enzyme from the sample, a total yield of 20 μg from 250 μg mitochondria. Simple calculation indicate that 1 mg of beef heart mitochondrial membranes contains a total of about 80 μg Complex IV, i.e. 0.4 nmol/mg. These data agree with results from previous studies quantifying the levels of OXPHOS components within mitochondria (Murray et al., FEBS Lett., 529: 173-178, 2002).

C. Electrophoresis and Peptide Mass Fingerprinting Analysis of Complex IV

FIG. 20 shows a typical SDS gel profile of Complex IV immunocaptured from bovine heart mitochondria when resolved on a 4-12% Bis-Tris NuPAGE gel system (Invitrogen). This gel system is advantageous because the Complex IV subunit profile was more easily reproduced from one experiment to another, preventing the need for repeated mass spectrometry of protein bands. Individual subunits of Complex IV were identified by peptide mass fingerprinting (PMF) and tandem mass spectrometry (nano-HPLC-MS MS/MS). Protein bands were excised from the gel, digested with trypsin and/or chymotrypsin, and analyzed by MALDI-TOF mass spectrometry prior to subjecting the extracts to nano-HPLC-MS MS/MS to obtain peptide sequence data.

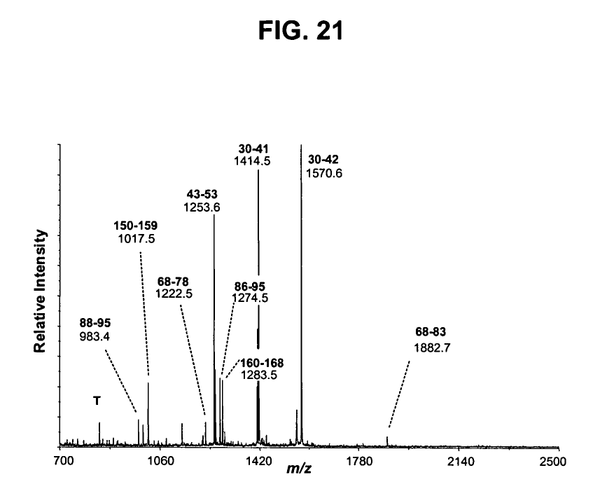

As shown in FIG. 21, MALDI-MS peptide mass fingerprint data were often sufficient to rapidly identify proteins from gel bands. As an example, when the band annotated as Complex IV subunit IV (see FIG. 20) was excised and fully analyzed, thirteen subunit IV tryptic peptides were observed by MALDI-MS. These peptide mass fingerprints provided coverage of 41% of the protein sequence and yielded a significant RADARS search engine score of 5.7×10⁻⁵. Similarly, other Complex IV subunits could be identified by MALDI-MS with most yielding good sequence coverage, e.g. 67% for subunit Vb (score: 2.0×10⁻³), and 74% for subunit VIb (score: 3.1×10⁻⁵). Thus by using a highly purified system coupled to high resolution 1D electrophoresis, it proved, in the majority of cases, possible to produce a clean mass fingerprint and therefore to obtain a conclusive protein assignment for each gel band.

D. Tandem Mass Spectrometry Analysis of Complex IV Subunits

Nano-HPLC-MS MS/MS analysis of Complex IV subunits was also undertaken to maximize the peptide coverage of each protein. All subunits were identified by LC-MS/MS and searched with Mascot search engine (Perkins et al., Electrophoresis, 20: 3551-3567, 1999). By analyzing both trypsin and chymotrypsin-generated peptides, it was possible to obtain good coverage of 12 of the 13 Complex IV subunits (see FIG. 20 and Table 9). Table 9 details the number of proteolytic peptides, their coverage of the protein (%) and the Mascot score for these peptides when identifying proteins. From the data, it is clear that trypsinization is complemented by chymotrypsinization, yielding greater combined peptide coverage. This complementation is especially useful when analyzing hydrophobic proteins such as the mtDNA encoded Complex IV subunits I, II and III. However, neither proteolytic digest was able to generate detectable peptides from the smallest Complex IV subunit, the nuclear encoded subunit, VIII.

TABLE 9
Complex IV Subunits Identified after Immunocapture
from Bovine Heart Mitochondria

| COX subunit | Accession | Observed tryptic | | | Observed chymotryptic | | | Total |
|---|---|---|---|---|---|---|---|---|
| (Observed MW) | number | peptides | % cov | score | peptides | % cov | score | cov. |
| I (35 kDa) | P00396 | 1 | 2% | 38 | 13 | 17% | 258 | 18% |
| II (25 kDa) | P00404 | 22 | 29% | 287 | 23 | 40% | 449 | 63% |
| III (25 kDa) | P00415 | 2 | 5% | 29 | 3 | 9% | 31 | 13% |
| IV (18 kDa) | P00423 | 27 | 53% | 489 | 12 | 33% | 258 | 62% |
| Va (12 kDa) | P00426 | 20 | 65% | 509 | 6 | 39% | 132 | 65% |
| Vb (13 kDa) | P00428 | 26 | 65% | 414 | 9 | 55% | 109 | 83% |
| VIa-H (12 kDa) | P07471 | 9 | 42% | 104 | 16 | 65% | 204 | 74% |
| VIb (12 kDa) | P00429 | 16 | 76% | 464 | 7 | 61% | 131 | 81% |
| VIc (12 kDa) | P04038 | 10 | 42% | 162 | 4 | 17% | 49 | 48% |
| VIIa H (8 kDa) | P07470 | 5 | 38% | 158 | 9 | 46% | 112 | 50% |
| VIIb (8 kDa) | P13183 | 4^{b} | 27% | na | — | — | — | 27% |
| VIIc (8 kDa) | P00430 | 2 | 28% | 77 | 6 | 49% | 82 | 54% |
| VIIa L^{a} (8 kDa) | P13184 | 3 | 27% | 88 | — | — | — | 27% |
| VIII | P10175 | — | — | — | — | — | — | — |

^{a}Mass spectrometric values listed for VIIa L were measured from a bovine kidney sample.
^{b}Peptides from VIIb were from combined MALDI-TOF MS and LC-MS/MS.

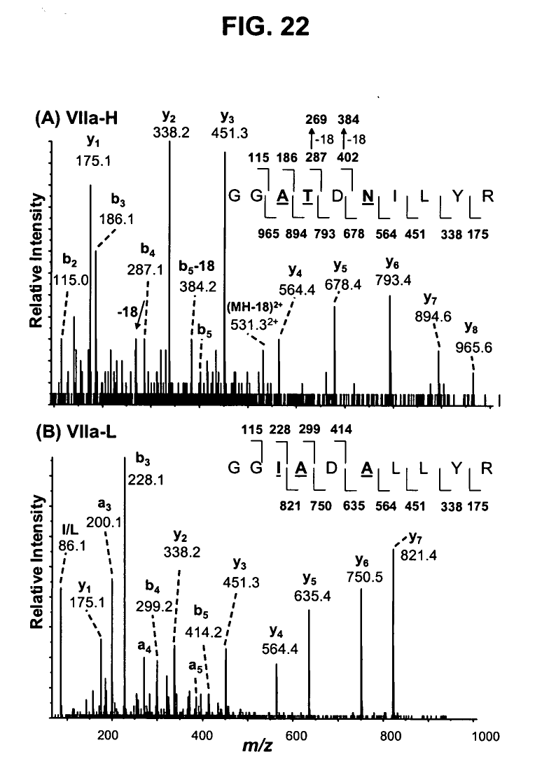

In parallel experiments, Complex IV was immunocaptured from bovine kidney. As expected, well-documented tissue specific variations in Complex IV composition were observed, with subunits VIa, and VIIa migrating slightly differently (Taanman et al., Biochim. Biophys. Acta, 1225: 95-100, 1993; Anthony et al., FEBS Lett., 277: 97-100, 1990). Tissue specific differences could also be identified by LC-MS/MS as exemplified in FIG. 22, where the equivalent peptide from the muscle isoform, VIIa-H, and the non-muscle isoform, VIIa-L, are compared. Four amino acid variations in this peptide alone could be identified by mass spectrometry.

The Complex IV subunits were the major contingent of proteins immunocaptured by this immunocapture mAb and each Coomassie-stained band could easily be assigned to a Complex IV subunit(s). Nevertheless, small amounts of other proteins could be found when the entire length of the lane was cut into 1 mm slices and analyzed by the highly sensitive mass spectrometry methods described. These associated proteins are listed in Table 10 and grouped by enzyme complex when possible. Detailed are the number of proteolytic peptides, their coverage of the protein (%) and the Mascot score for these peptides when identifying proteins. The molecular masses of all proteins found by mass spectrometry were consistent with the gel slice in which they were detected. Interestingly, 9 of 11 proteins of Complex III of the OXPHOS chain were detected. This is expected given the potential for OXPHOS enzymes in general, and Complex III and Complex IV in particular, to associate as a supercomplex (Cruciat et al., J. Biol. Chem., 275: 18093-18098, 2000; Schagger and Pfeiffer, Embo J, 19: 1777-1783, 2000).

TABLE 10
Proteins Identified as Low-abundance Complex IV
Interacting Proteins by LC-MS/MS from SDS-PAGE

| | No. Peptides | Coverage | Score |
|---|---|---|---|
| Complex 1 | | | |
| NDUFV1 | 3 | 6% | 122 |
| NDUFS2 | 1 | 2% | 52 |
| NDUFS3 | 3 | 13% | 121 |
| NDUFA4 | 6 | 35% | 151 |
| Complex III | | | |
| subunit 1 - core1 | 30 | 51% | 900 |
| subunit 2 - core2 | 28 | 44% | 921 |
| subunit 4- cl | 16 | 49% | 316 |
| subunit 5 Rieske | 1 | 3% | 39 |
| subunit 6 | 9 | 51% | 239 |
| subunit 7 | 2 | 19% | 49 |
| subunit 8 | 5 | 66% | 191 |
| subunit 9 | 2 | 50% | 69 |
| subunit 10 | 2 | 24% | 88 |
| Complex V | | | |
| F1 α | 22 | 32% | 964 |
| F1 β | 17 | 34% | 659 |
| F1 γ | 6 | 18% | 273 |
| F1 δ | 1 | 5% | 49 |
| F1F0 f | 2 | 34% | 101 |
| F1F0 F6 | 2 | 25% | 82 |
| Non-OXPHOS Proteins | | | |
| Oxoglutarate dehydrogenase^{H} (Q02218) | 23 | 23% | 841 |
| Actinin alpha 2^{H} (P35609) | 7 | 8% | 303 |
| Band 3 protein (AAD43593) | 16 | 17% | 600 |
| Mitofilin^{H} (Q16891) | 11 | 9% | 364 |
| HSP90 α (BAC82487) | 8 | 10% | 333 |
| Gastrin binding-like protein | 25 | 29% | 843 |
| (CAA10897) | | | |
| v.l.c.acyl-CoA dehydrogenase | 16 | 24% | 740 |
| (AAA74051) | | | |
| AIF^{H} (AAD16436) | 9 | 15% | 349 |
| CAT (NP_659006.1) | 7 | 8% | 259 |
| Fatty acid oxidation β subunit | 13 | 25% | 393 |
| (CAA05840) | | | |
| Desmin (BAA25133) | 3 | 6% | 155 |
| Dihydrolipoamide succ. transf.^{H} | 11 | 16% | 432 |
| (NP_001924) | | | |
| Vimentin (NP_776394) | 4 | 8% | 199 |
| Porin 1 (AAF80101) | 3 | 8% | 144 |
| D-prohibition^{H} (AAF44345) | 9 | 25% | 384 |
| ANT (XWBO) | 12 | 35% | 487 |
| Troponin I (P08057) | 4 | 23% | 149 |

E. Enzymatic Activity Determination of Isolated Complex IV

The possibility that the immunopurified Complex IV was still active after isolation while still bound to the immunocapture support was examined. This analysis used a 96-well, flat-bottom plate containing 250 μL per well of the Complex IV assay mixture and 2 μg or less of isolated Complex IV per assay. Activity was followed spectrophotometrically with a multi-plate reader equipped with the appropriate optic filters. Calculation of the turnover rate of the enzyme when isolated was approximately 35 s⁻¹ which is approximately 8-10 fold lower than the turnover rate in the membrane (250 s⁻¹) or when Complex IV is biochemically purified (350 s⁻¹) (Musatov et al., Biochemistry, 41: 8212-8220, 2002). This was not due to an inhibitory effect of the capture antibody because addition of the free antibody to solubilized mitochondrial membranes in molar ratios as high as 100:1, antibody:Complex IV protein, did not reduce cytochrome c turnover. This reduced turnover rate is probably due to inferior mixing of Complex IV in the assay solution because of its attachment to the bead surface.

F. Analysis of Oxidative Damage to Complex IV

The ability to immunocapture Complex IV from small amounts of mitochondria (or cell extract) greatly facilitated the detection of post-translational modifications and their effects upon activity.

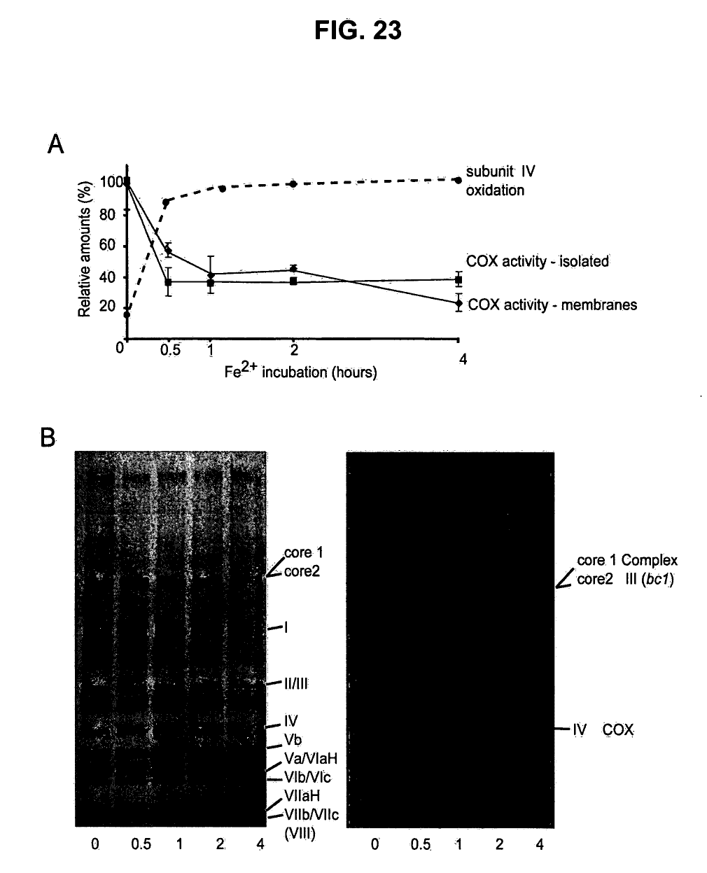

First, mitochondrial membranes were exposed to a hydroxyl radical generating system consisting of ascorbate/Fe²⁺/O₂ for 0, 0.5, 1, 2, or 4 hours using a previously described protocol (Bautista et al., Biochem. Biophys. Res. Commun., 275: 890-894, 2000). The effect of hydroxyl radical exposure on the activity of Complex IV was tested in both mitochondrial membranes (i.e. before isolation) and after immunocapture isolation (see FIG. 23A). There was a significant reduction in specific activity of the enzyme during the first 30 minutes. After these activity measurements the Complex IV was then captured from the same membranes to identify the generation of carbonyl groups. To detect protein carbonyls mitochondria were first derivatized with 2,4-dinitrophenylhydrazine (DNPH), which reacts with carbonyl side chains forming a 2,4-dinitrophenylhydrazone (DNP) moiety. The Complex IV enzyme was isolated by immunocapture and Western blotted with a DNP-specific antibody. Only one carbonyl-containing, DNP-derivatized Complex IV protein was found which was identified as subunit IV by mass spectrometry (see FIG. 23B). In addition Complex III core 1 or core 2 which co purify with Complex IV showed antibody reactivity indicating that they are also oxidatively damaged by the treatment (FIG. 23A).

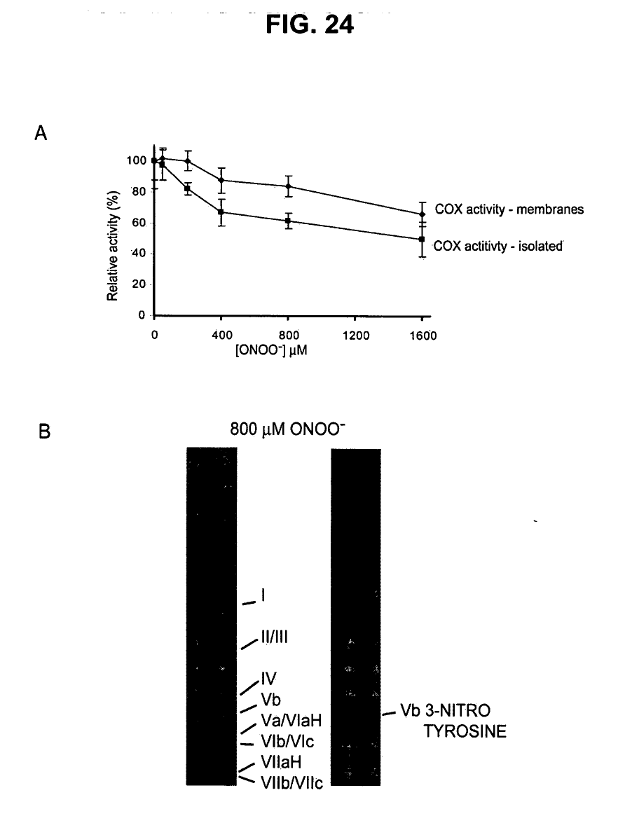

Another potentially important reagent in modifying proteins during oxidative stress is peroxynitrite (ONOO⁻), particularly in heart and brain where substrate NO levels are high. Mitochondrial membranes were exposed to a peroxynitrite at concentrations up to 1600 μM. Following treatment with peroxynitrite, both the mitochondrial membranes and isolated Complex IV from these membranes were analyzed for Complex IV activity (see FIG. 24A). As with hydroxyl radical inhibition, peroxynitrite reaction caused only a partial inhibition of the enzyme (˜50%) even at high concentrations of peroxynitrite. After the enzyme was isolated, a single polypeptide was modified by peroxynitrite that was identified by mass spectrometry as Complex IV subunit Vb (FIG. 24B).

Complex IV deficiencies are a relatively common cause of human diseases with genetic origins, such as Leigh syndrome, MELAS, and MERRF, and are involved in late-onset disorders like Alzheimer's and Huntington's disease (Darin et al., Neuropediatrics, 34: 311-317, 2003; Orth and Schapira, Am. J. Med. Genet., 106: 27-36, 2001). Complex IV defects in these late onset disorders could be attributable to either altered amounts of enzyme through reduced biogenesis, or altered catalytic functioning, for example through the accumulation of post-translational modifications of critical residues induced by oxidative stress.

The ability to screen rapidly for both assembly and oxidative post-translational modifications of proteins greatly facilitates evaluation of oxidative stress during neurodegeneration (e.g., Butterfield, Brain Res., 1000: 1-7, 2004). Attempts to do so by total cell proteomics or even analysis of organelles, such as mitochondria which contains 1000 or more protein components (Taylor et al., Nat. Biotechnol., 21: 281-286, 2003; Da Cruz et al., J. Biol. Chem., 278: 41566-41571, 2003; Sickmann et al., Proc. Natl. Acad. Sci. USA, 100: 13207-13212, 2003), can have only limited success because of the complexity of the systems. Several approaches are available to sub-fractionate mitochondria into more manageable portions and aid in resolving single proteins for peptide analysis, including blue native-PAGE, sucrose gradient technology, and gel filtration (Taylor et al., J. Proteome Res., 1: 451-458, 2002; Buchanan and Walker, Biochem. J., 318(Pt 1): 343-349, 1996; Schagger et al., Electrophoresis, 17: 709-714, 1996). While each of these approaches simplifies the screen for post-translational changes, they require relatively large amounts of material. If the detection of oxidative damage is ever to be used as a diagnostic tool for late-onset diseases, it will require the analysis of minute amounts of protein obtained from blood or small biopsy samples. This example describes a method for isolating Complex IV from very small amounts of mitochondria (250 μg or less) and show that the amount of enzyme obtained is sufficient to do an extensive mass spectrometric analysis.

The mass spectrometric analyses described in this example identified 12 of the 13 Complex IV subunits, including hydrophobic proteins like the mtDNA encoded subunits I, II, and III. Tissue specific differences in the sequence of Complex IV subunit VIIa and VIIa were detected by mass spectrometry (Taanman et al., Biochim. Biophys. Acta, 1225: 95-100, 1993). However, no peptides were generated from the smallest Complex IV subunit, the nuclear encoded subunit VIII. This subunit was present in the immunocaptured complex based on the appearance of a band of appropriate size in highly resolving gel systems and the maintenance of captured Complex IV enzymatic activity. The immunocaptured-immobilized Complex IV had a turnover rate which, while 8-10 fold lower than the activity of reported isolated-free Complex IV (Musatov et al., Biochemistry, 41: 8212-8220, 2002), demonstrated the same characteristics as membrane bound Complex IV when exposed to either of these oxidative stresses or cyanide.

It is particularly advantageous when detecting post-translational modifications of proteins to be able to correlate individual modifications with activity effects. The reaction of peroxynitrite with mitochondria caused a partial loss of Complex IV activity, which was correlated with 3-nitrotyrosine modification of subunit Vb. Subunit Vb is located on the matrix side of the mitochondrial inner membrane adjacent to the core and prosthetic group containing subunits I and II (Tsukihara et al., Science, 272: 1136-1144, 1996). Subunit Vb contains only two tyrosine residues Y³¹ and Y⁸⁹. When analyzing the Fe-catalyzed oxidation of mitochondrial membranes a partial inhibition of Complex IV activity was also observed. As with peroxynitrite modification, carbonyl modification was remarkably specific and limited to subunit IV. The specific residues in subunit IV that have formed carbonyl groups, aldehydes and ketone were not determined. Candidate residues include proline (of which there are 6 in subunit IV), arginine (5), lysine (18), and threonine (5) (Dalle-Donne et al., Trends Mol. Med., 9: 169-176, 2003; Requena et al., Proc. Natl. Acad. Sci. USA, 98: 69-74, 2001). Previous studies have suggested a regulatory role of subunit IV, with evidence that it is phosphorylated by an endogenous mitochondrial kinase in a cAMP-dependent manner. The susceptibility of this subunit to oxidative damage was demonstrated by identification of an endogenous oxidized tryptophan in human and bovine heart samples as shown in a previous study (Taylor et al., J. Biol. Chem., 278: 19587-19590, 2003).

In summary, this example provides a simple immunocapture procedure for isolating a functionally active Complex IV from various mammalian species and from different tissues. The procedure effectively isolates the enzyme complex from mitochondria which had been treated with free radical-generating reagents, and post-translational modification levels can be correlated with the degree of with activity effects. Since Complex IV activity was robust even in the presence of high levels of oxidants it is less sensitive to oxidative damage than other OXPHOS complexes based on activity effects (Murray et al., J. Biol. Chem., 278: 37223-37230, 2003). Isolation of Complex IV from AD brain will identify whether post-translational modifications of these kinds are occurring or if assembly of the enzyme is reduced in conditions of neuronal oxidative stress.

### Example 8

### Quantitation of Complex IV Activity by Immunocapture and Functional Analysis

Complex IV was immunocaptured on sepharose beads as described in Example 7. Activity of the immunocaptured enzyme was then measured by transferring aliquots of the washed beads to individual microwells and the following reaction performed.

The oxidation of ferrocytochrome c (reduced cytochrome c) was in a 96-well plate (Corning) using a Victor2 1460 multi-plate reader (Perkin-Elmer) with a narrow band pass 550 nm filter. Reaction vessels contained 30 μM ferrocytochrome c and assays were carried out at 22° C. in Complex IV assay buffer (25 mM KH₂PO₄, pH 7.2, 2 mM lauryl maltoside). The reaction was initiated by the addition of either immunocaptured cytochrome c oxidase attached to beads. The initial rate of ferrocytochrome c oxidation was followed for 2 minutes. An absorption coefficient of 21.1 mM⁻¹cm⁻¹ for ferrocytochrome c and a light path length of 10.5 cm (Victor 2) were used for calculating the molar amount of ferrocytochrome c oxidized by Complex IV. Ferrocytochrome c was generated by dissolving bovine heart cytochrome c (Sigma) into assay buffer and reducing it with a saturating amount of L-(+)-ascorbic acid (MCB Reagents). Reduced bovine heart cytochrome c was purified over a Sephadex G-15 (Pharmacia) column equilibrated with assay buffer, collected in 1 mL fractions, and the concentration measured at 550 nm in a Beckman DU7.

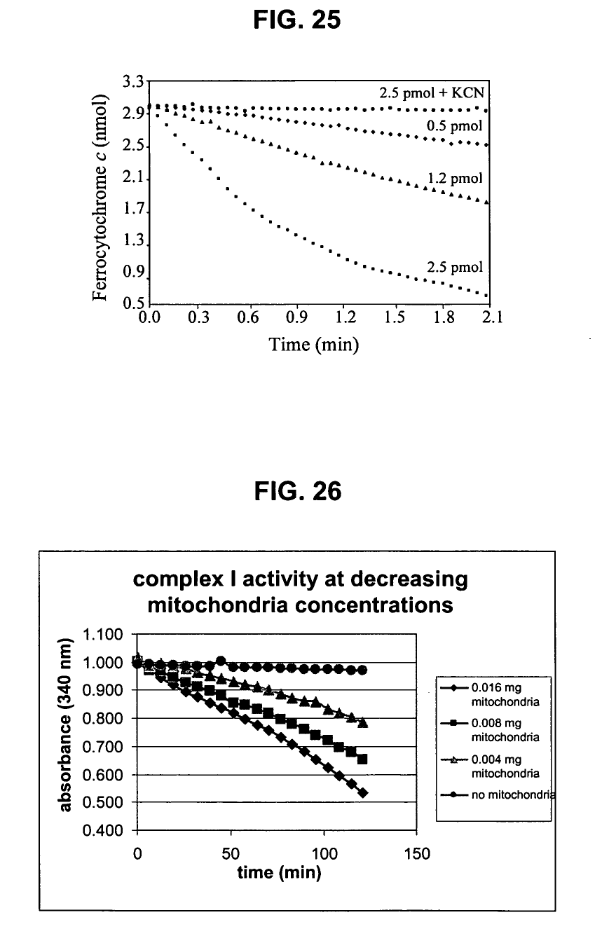

As shown in FIG. 25, the rate of Complex IV enzyme activity per well is dependent on the amount of enzyme captured (pmol). Therefore, this assay can be used to measure the amount of Complex IV in unknown samples. Specificity of immunocapture is confirmed as all immunocaptured oxidase activity is completely inhibited by potassium cyanide, a specific inhibitor of Complex IV.

### Example 9

### Quantitation of Complex I by Microscale Immunocapture and Measurement of Complex I Enzyme Activity

This Example demonstrates Complex I immunocapture and activity measurements in a 96-well plate format.

FIG. 26 shows a dilution series of mitochondria tested in a Complex I microplate activity assay. In brief, 1 μg mAb RAC#24-20D1AB7 was added per well (50 μl of a 20 μg/ml solution in PBS) on a protein G-coated 96-well plate, incubated overnight at 4° C., then washed three times with PBS. Then, heart mitochondria solubilized in lauryl maltoside as previously described and diluted in PBS were added at 16 μg (♦), 8 μg (▪), 4 μg (▴) and 0 μg (●) per well in 50 μl aliquots and incubated at room temperature for 2 hours. The wells then were washed three times with 200 μl/well of 20 mM Tris/HCl, pH 7.5, 50 mM KCl, 0.015% lauryl maltoside. A phospholipid solution (see below) was added at 40 μl/well, incubated for 45 minutes at 4° C. and then 200 μl of assay solution (25 mM KPi pH 7.2, 5 mM MgCl₂, 2 mM KCN, 260 μM NADH, and 200 μM UQ₁) was added directly to the phospholipid solution. The oxidation of NADH was measured by following the decrease of the absorbance at 340 nm. Absorbance changes were measured at 340 nm with a Victor2 from Perkin Elmer for two hours.

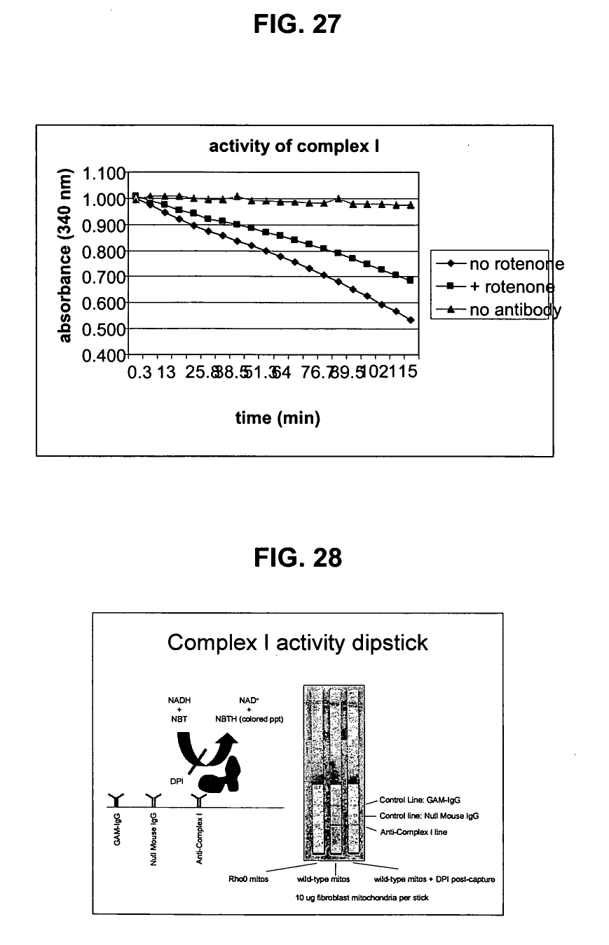

FIG. 27 demonstrates that approximately 30% of immunocaptured NADH:UQ1 oxidoreductase activity is sensitive to rotenone inhibition, which characterizes the antibody-bound mitochondrial protein as Complex I.

The phospholipid stock solution was made by mixing 0.76 ml of 10 mg/ml egg-yolk phosphatidyl choline, 0.38 ml of 5 mg/ml egg-yolk phosphatidylethanolamine, and 0.1 ml of 5 mg/ml cardiolipin in round bottom flask. This mixture was dried under N₂, resuspended in 4.25 ml buffer I (20 mM Tris/HCl pH 7.5, 50 mM KCl), and vortexed. Then, 1.2 ml of 20 mM Tris/HCl, pH 7.5, 50 mM KCl, 10% lauryl maltoside was added gradually (8 aliquots of 0.15 ml each) to clear the solution, followed by the addition of 0.25 ml 20 mM Tris/HCl pH 7.5, 50 mM KCl. This initial phospholipid stock was 1.75 mg/ml in 2.1% lauryl maltoside and was then diluted with 20 mM Tris/HCl pH 7.5, 50 mM KCl to get a working stock solution of 12.5 μg/ml phospholipid in 0.015% lauryl maltoside. This working stock solution was added to the microplate wells as described above.

### Example 10

### Complex I, IV and V Activity Dipsticks

This Example demonstrates dipsticks for the measurement of Complex I, IV and/or V activities.

A. Immunocapture Lateral Flow Devices (e.g., Dipsticks)

Immunocapture lateral flow devices (e.g., dipsticks) specific for Complex I were prepared as follows: First, three parallel narrow zones of three different antibodies were laid down across the long dimension of nitrocellulose sheets, (1.25 in×12 in, Millipore ST, STHF04000, cat # SA3J441H7) wide sheets trips (see FIG. 28 for layout configuration), the antibodies allowed to adsorb to the nitrocellulose and air-dry. The three antibody zones contained: Zone #1, anti-Complex I (mAb RAC#24-20D1AB7); Zone #2, a null mouse IgG (pooled normal mouse IgG, Jackson ImmunoResearch); and Zone #3, Goat-anti-mouse IgG as another null control (Jackson ImmunoResearch). Each antibody was diluted to approximately 2 mg/ml in PBS and applied at approximately 1 μl (2 μg) per linear cm along the long axis of the nitrocellulose sheet. After the antibody solutions had dried, the dipsticks were laminated to an adhesive plastic backing, an absorbent cotton pad affixed to the top of each sheet (the end away from the zones of antibody along the short axis) in direct contact with the nitrocellulose layer. Strips were then cut at 0.5 cm intervals, creating individual dipsticks approximately 0.5×5 cm, with the antibody zones perpendicular to the long axis and a cotton adsorbent pad affixed to one end of the long axis (see FIGS. 28, 29 and 30 for orientation).

Dipsticks specific for Complexes IV and V were each prepared as described above for the Complex I dipstick, except that the anti-Complex I mAb was replaced by anti-Complex IV mAb RAC#11B-7E5BA4, or anti-Complex V mAb MM#1-12F4AD8AF8, respectively.

B. Sample Preparation.

Mitochondria (human heart, human fibroblast or bovine heart) were solubilized at 5 mg/ml mitochondrial protein, 1% wt/vol lauryl maltoside, 100 mM NaCl, 25 mM HEPES pH 7.5, and the protease inhibitors pepstatin (0.5 μg/ml), leupeptin (0.5 μg/ml) and PMSF (1 mM). The samples were incubated at 4° C. for 30 minutes and the insoluble material removed by centrifugation at 16,000×g for 20 minutes. Solubilized mitochondria were diluted to the desired concentration in 150 mM NaCl, 50 mM Tris-HCl, pH 7.5 containing 2.5% bovine serum albumin as a blocking and stabilization agent.

C. Dipstick Operation.

Samples of solubilized mitochondria (50-200 μl) were loaded into microtiter wells and the appropriate dipstick inserted with the stick oriented so the free nitrocellulose-plastic laminate end of the stick was submerged in the sample, the antibody zones were out of the sample and the absorbent-pad end of the stick was farthest out and away from the sample. After the entire sample wicked up into the nitrocellulose (approximately 15 minutes), the stick was transferred to a second well containing 50 μl of wash buffer (150 mM NaCl, 50 mM Tris-HCl, pH 7.5), which was allowed to wick up the stick (approximately 15 minutes). The absorbent pad was then removed and the entire dipstick incubated in a tube containing 3 ml wash buffer for approximately 15 minutes. The washed dipstick (with the immunocaptured OXPHOS complex of interest) was then transferred to the appropriate enzyme reaction buffer to reveal enzyme activity as described below.

D. Enzyme Reaction Buffers and Incubation Conditions:

1. Complex I. The Complex I reaction buffer solution contained 0.1 mg/ml NADH, 2.5 mg/ml NBT in 2 mM Tris-HCl, pH 7.4. Upon incubation with sample containing Complex I (an NAD/NADH diaphorase enzyme) the colorless, soluble NBT was reduced using electrons from NADH to form a highly colored, insoluble formazan deposit at the site of the enzyme. Dipsticks incubated with mitochondrial samples and washed as described above were incubated in reaction buffer (approximately 1 ml) at 37° C. until sufficient color developed (approximately 30 minutes), rinsed with distilled water and air-dried. The reaction could be carried out for more or less time, depending on the concentration of Complex I in the sample. Enzyme specificity was, optionally, verified by including a specific Complex I inhibitor, e.g., the flavin reagent diphenyleneiodonium (DPI) at 32 μM in the reaction buffer. This DPI reagent interacts covalently with the FMN cofactor and blocks electron transport to hydrophilic acceptors such as NBT.

2. Complex IV. The Complex IV reaction buffer solution contained 0.5 mg/ml 3,3′ diaminobenzidine-tetrachloride (DAB) and 0.1 mM reduced cytochrome c in PBS, pH 7.4. Upon incubation with a sample containing Complex IV (cytochrome c oxidase) the reduced cytochrome c oxidized, and the oxidized cytochrome c was then reduced by DAB. The oxidized DAB formed an insoluble, colored precipitate that was localized at the site of enzyme activity. This DAB precipitate absorbs strongly at 450 nm which, optionally, was used to measure progress of the overall reaction. Because Complex IV is the rate-limiting step in the overall reaction, signal generation (precipitated oxidized DAB) was a function of the amount of Complex IV captured. Dipsticks incubated with mitochondrial samples and washed as described above were incubated in reaction buffer (approximately 1 ml) for 30 minutes at 37° C., rinsed with distilled water and air-dried. The reaction could be carried out for more or less time, depending on the concentration of Complex IV in the sample. Enzyme specificity, optionally, was verified by including a specific Complex IV inhibitor, e.g., 1 mM azide, in the reaction buffer solution.

3. Complex V. Dipsticks incubated with mitochondrial samples and washed as described above were incubated in 1 ml ATPase reaction buffer (0.5 mg/ml ATP, 100 mM CaCl₂, 67 mM glycine pH 9,4) for 30 minutes at 37° C., washed well with distilled water, incubated 5 minutes at room temperature in 2% (wt/vol) cobalt chloride in distilled water, washed well in tap water, then washed with three changes of distilled water, incubated for 30 seconds in 2% ammonium sulphide, washed with tap water and air-dried. The initial reaction in ATPase reaction buffer could be carried out for more or less time, depending on the concentration of Complex V in the sample. Enzyme specificity, optionally, was verified by including a specific Complex V inhibitor, e.g., 10 μM oligomycin, in the reaction buffer.

E. Results

FIG. 28 shows that the Complex I activity dipstick can detect an experimental drug-induced Complex I defect in human fibroblasts. Mitochondria were isolated from normal (wild-type) and Rho⁰ human fibroblasts grown in vitro., and 10 μg protein samples were run on Complex I specific dipsticks as described above. Rho⁰ cells lack mtDNA as a result of exposure to 50 ng/ml ethidium bromide for at least 12 cell doublings. Because they have a well-characterized defect, i.e., they lack mtDNA and mtDNA encoded proteins, Rho⁰ cells are a good model system with which to study mitochondrial defects. Because Complexes I, III, IV and V contain essential subunit proteins encoded by mtDNA, these complexes are deficient or present in altered form (as partially assembled subcomplexes) in Rho⁰ cells. As expected, Rho⁰ cells showed no Complex I activity when assessed by the Complex I activity dipsticks (FIG. 28). Moreover, since Complex I is the rate-limiting factor in generation of a signal in this detection system, the amount of signal generated was proportional to the amount of Complex I immunocaptured, which in turn was proportional to the concentration of Complex I in the sample. Specificity of the assay was verified by the reduced signal observed when a partial inhibitor of Complex I activity (DPI) was added during the enzyme reaction of a dipstick used to capture normal fibroblast Complex I (see FIG. 28).

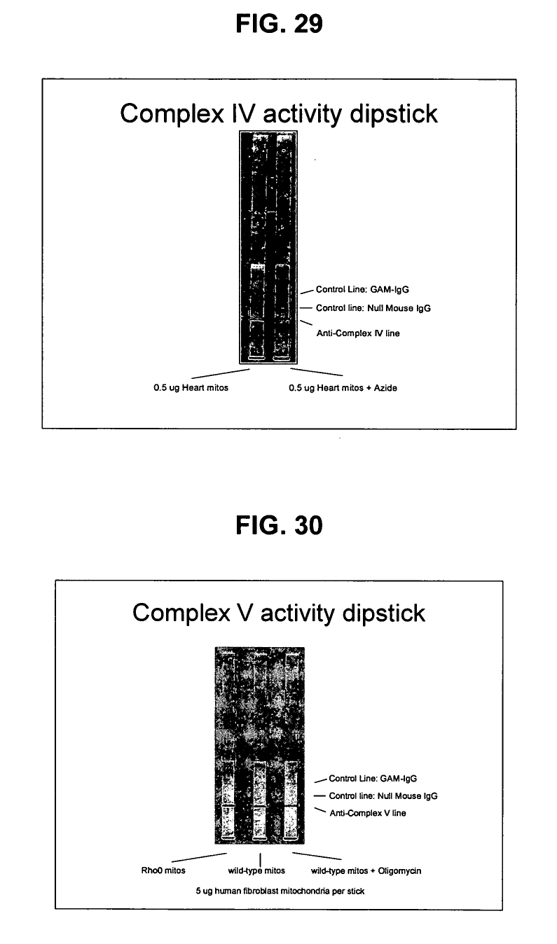

FIG. 29 shows that Complex IV activity was detected by dipstick exposed to samples of heart mitochondria. As demonstrated by FIG. 29, the dipstick measured a signal from a sample containing 0.5 μg of bovine heart mitochondria; thus, dipstick detection of Complex IV is extremely sensitive. Complex IV was detected in samples of human fibroblast mitochondria. Specificity of the Complex IV dipstick assay was verified by the near complete inhibition of oxidase activity in the Complex IV capture zone when a specific inhibitor of Complex IV (3 mM azide) was added to the reaction buffer (see FIG. 29). Moreover, since Complex IV is the rate-limiting factor in generation of a signal in this detection system, the amount of signal generated was proportional to the amount of Complex IV immunocaptured, which was, in turn, proportional to the concentration of Complex IV in the sample.

FIG. 30 shows that Complex V activity dipsticks detect an experimental drug-induced Complex V defect in human fibroblasts. Mitochondria were isolated from normal (wild-type) and Rho⁰ human fibroblasts grown in vitro, and 5 μg protein samples were run on Complex V specific dipsticks as described above. It is known that Complex V is misassembled in Rho⁰ cells and the F1 subcomplex (composed entirely of nuclear DNA-encoded subunits) persists (Garcia et al., Biol. Chem. 275, 11075-11081, 2000). The F1 subcomplex is also known to retain ATPase activity. Indeed, in what may be a compensatory effort, the F1 proteins are up-regulated in Rho⁰ cells (Garcia et al., Biol. Chem. 275, 11075-11081, 2000) and F₁/F₀ ATPase activity increases in these cells. As demonstrated by FIG. 30, an increase in levels of ATPase activity of the F1 subcomplex of Complex V was measure by the Complex V-specific dipsticks. Specificity of the assay was verified by the reduced signal observed when a specific inhibitor of Complex V activity (e.g., oligomycin) was added during the enzyme reaction of a dipstick used to capture normal fibroblast Complex V (see, FIG. 30). Since Complex V is the rate-limiting factor in generation of a signal in this detection system, the amount of signal generated was proportional to the amount of Complex V immunocaptured, which, in turn, was proportional to the concentration of Complex V in the sample.

### Example 11

### Two-Site Quantitative Assay for OXPHOS Protein Quantitation

This Example demonstrates a 2-site immunocapture assay for Complex V.

A. Methods

1. Preparation of the Immunocapture Microwells. Capture mAb #1 (MM#1-12F4AD8AF8, which is a mouse IgG2b antibody specific for Complex V-F1) was immobilized on the surface of high protein binding polystyrene 96-well microwell plates by passive adsorption. The antibody was diluted to 5 μg/ml in PBS and then loaded at 100 μl/well and incubated overnight 4° C. in a humid chamber. Unbound antibody was removed by washing the wells 3 times with an excess of PBS. A blocking agent, 5% (wt/vol) non-fat dry milk in PBS, was then loaded at 400 μl/well and incubated for 2 hours at room temperature to block any open sites on the well surface that might otherwise non-specifically bind Complex V.

2. Immunocapture of Complex V. Human heart mitochondria (obtained from Analytical Biological Services; Wilmington, Del.) were solubilized at 5 mg/ml in 1.0% lauryl maltoside, and the insoluble material removed by centrifugation at 16,000×g for 20 minutes. Solubilized mitochondria were then diluted out to various concentrations ranging from 100 μg/ml to 1600 μg/ml in PBS containing 0.1% lauryl maltoside and loaded into the blocked wells at 100 μl/well. After a 2 hour incubation at room temperature to allow immunocapture of Complex V, the wells were washed three times with an excess 0.015% lauryl maltoside in PBS to remove enraptured Complex V and other mitochondrial proteins.

3. Detection of Immunocaptured Complex V. A second anti-Complex V capture mAb (mAb#2, MM#7-3D5AB1, a mouse IgG1 antibody specific for the beta subunit of Complex V) was diluted to 5 μg/ml in 5% milk in PBS and loaded into the microwells at 100 μl/well. After a 2 hour incubation to allow binding of mAb#2 to any immunocaptured Complex V, the wells were washed 3 times with an excess of PBS to remove unbound mAb #2. Bound mAb#2 was then detected by incubating the wells with 100 μl/well of a 5% milk/PBS solution containing 1 μg/ml of Alexa 488 labeled GAM-IgG1, a secondary antibody that binds specifically to mAb#2 (a mouse IgG1), and not to mAb#1 (a mouse IgG2b). The plates were incubated for 1 hour at room temperature and then washed 3 times with an excess of PBS, leaving behind immunocaptured Complex I, bound mAb#2 and bound Alexa 488 GAM-IgG1.

B. Detection of Microwell Fluorescence.

To improve the fluorescence yield, test wells were treated to release proteins bound to the sides and bottom of the wells. This treatment releases bound Alexa488-GAM-IgG1, making it available for in-solution microplate reading of per-well fluorescence. The release solution was 0.1% SDS in dH₂O loaded at 100 μl/well, and incubated for 20 minutes at room temperature. The amount of fluorescence per well was measured on a Perkin-Elmer Victor 2 using the filter set recommended by the manufacturer.

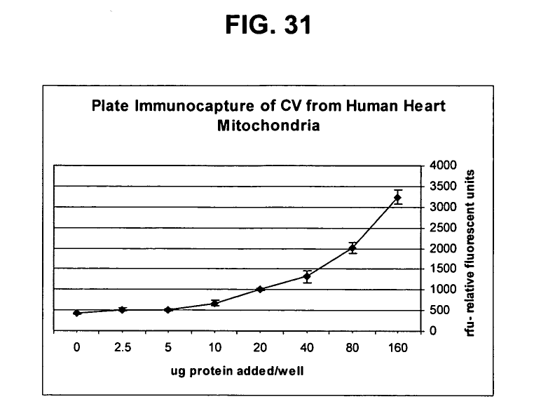

FIG. 31 shows the sensitivity and reproducibility of the 2-site immunocapture assay specific for Complex V. The assay has a dynamic range from 10 μg/well to 160 μg/well and is highly reproducible (error bars show standard deviations from the mean).

While this disclosure has been described with an emphasis upon particular embodiments, it will be obvious to those of ordinary skill in the art that variations of the particular embodiments may be used and it is intended that the disclosure may be practiced otherwise than as specifically described herein. Accordingly, this disclosure includes all modifications encompassed within the spirit and scope of the disclosure as defined by the following claims:

1. (canceled)

2. A monoclonal antibody or antigen-binding fragment selected from the group consisting of:
(a) any one of the following monoclonal antibodies: RAC#24-20D1AB7, RAC#24-18G12BC2AA10, RAC#24-17C8E4E1, RAC#24-17G3D9E12, RAC#29-1D4, RAC#29-4G6BB9, RAC#29-6E1BH7, RAC#24A-20E9DH10C12, RAC#23C-4H12BG12AG2, RAC#23B-1A11BC12AB9, RAC#23C-4H12BC11BC5, RAC#23B-10D2, RAC#23C-11A51H12, RAC#23C-12G8, RAC#23C-17A81A8, RAC#23C-29C2, RAC#11B-7E5BA4, RAC#23C-21H10, RAC#23C-22D5, RAC#23C-22H11G43E1, RAC#23C-28G7, RAC#23C-31E91B82G9, MM#1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9;
(b) a monoclonal antibody that competitively inhibits the specific binding of any one of the monoclonal antibodies if (a); and
(c) an antigen-binding fragments of any one of (a) or (b).

3. A hybridoma expressing an antibody or antigen-binding fragment of claim 2.

4. A method of detecting the presence of all or part of an OXPHOS enzyme complex in a biological sample, comprising:
(a) contacting a monoclonal antibody specific for a native OXPHOS enzyme complex with a biological sample, wherein all or part of an OXPHOS enzyme complex present in the biological sample and the monoclonal antibody form an immunocomplex, comprising immunocaptured OXPHOS enzyme complex;
(b) detecting the formation of the immunocomplex, wherein the formation of the immunocomplex detects the presence of all or part of the OXPHOS enzyme complex in the biological sample.

5. The method of claim 4, further comprising:
(a) quantifying the immunocaptured OXPHOS enzyme complex;
(b) assaying an enzymatic function of the immunocaptured OXPHOS enzyme complex;
(c) detecting a posttranslational modification in the immunocaptured OXPHOS enzyme complex;
(d) separating the immunocomplex from components of the biological sample that are not substantially bound by the antibody; or
(e) two or more of (a), (b), (c), or (d).

6. The method of claim 4, wherein the monoclonal antibody is a monoclonal antibody or antibody fragment of claim 2.

7. (canceled)

8. The method of claim 5(c), wherein the posttranslational modification comprises phosphorylation, oxidative damage, or carbonyl formation.

9. (canceled)

10. The method of claim 5(d), further comprising:
(i) releasing the immunocaptured OXPHOS enzyme complex from the immunocomplex, and separating subunits of the OXPHOS enzyme complex,
(ii) releasing the immunocaptured OXPHOS enzyme complex from the immunocomplex, and isolating the released OXPHOS enzyme complex; or
(iii) both (i) and (ii).

11. (canceled)

12. (canceled)

13. The method of claim 4, wherein the OXPHOS enzyme complex is Complex I, Complex II, Complex, III, Complex IV, Complex V, or a combination of two or more thereof.

14. The method of claim 13, wherein the OXPHOS enzyme complex is Complex I, Complex V or a combination thereof.

15. The method of claim 4, wherein the biological sample:
(a) is a cell lysate;
(b) is a mitochondrial extract;
(c) is a tissue extract;
(d) comprises less than 50 mg total protein;
(e) comprises less than about 1×10⁷ cells;
(f) is from a human; or
(g) is a combination of any two or more of (a) through (f).

16. The method of claim 15, wherein the cell lysate or mitochondrial extract is from a fibroblast, peripheral blood mononuclear cell (PBMC), needle biopsy, or mucosal epithelial cell.

17. (canceled)

18. (canceled)

19. The method of claim 4, wherein detecting the formation of the immunocomplex comprises:
(a) contacting the immunocomplex with a detectable marker that binds specifically to the immunocomplex;
(b) assaying an activity of the immunocaptured OXPHOS enzyme complex;
(c) high-throughput screening; or
(d) a combination of any two or more of (a), (b), or (c).

20. (canceled)

21. The method of claim 4, wherein the antibody is attached to a solid support.

22. The method of claim 21, wherein the solid support is a bead, a microtiter plate, or a dipstick.

23. A method for identifying an agent with potential to cause mitochondrial damage, comprising:
(1) the steps of:
(a) contacting an immunocaptured OXPHOS enzyme complex with a test agent; and
(b) assaying the activity of the immunocaptured OXPHOS enzyme complex in the presence and absence of the test agent, wherein a decrease in the activity of the OXPHOS enzyme complex in the presence of the test agent as compared to in the absence of the test agent indicates that the test agent is an agent with potential to cause mitochondrial damage or
(2) the steps of:
(i) contacting a biological system, comprising at least one OXPHOS enzyme complex, with a test agent;
(ii) immunocapturing at least one OXPHOS enzyme complex from the biological system; and
(iii) determining whether there is a relative change in a level, an activity, the number of subunits, or a posttranslational modification of the OXPHOS enzyme complex as compared to a control biological system that is not contacted with the agent, wherein a relative change in the level, the activity, the number of subunits, or the posttranslational modification of the OXPHOS enzyme complex identifies the test agent as an agent with potential to cause mitochondrial damage,
wherein the immunocaptured OXPHOS enzyme complex is Complex I, Complex II, Complex, III, Complex IV, Complex V, or a combination of any two or more thereof.

24. (canceled)

25. The method of claim 23, wherein the immunocaptured OXPHOS enzyme complex is:
(a) Complex I, Complex IV or a combination thereof;
(b) from a human subject and the method assesses mitochondrial damage in the human subject; or
(c) both (a) and (b).

26. The method of claim 23, wherein the agent is an environmental toxin or a drug.

27. (canceled)

28. The method of claim 26, wherein the drug is used, or is being tested for use, in highly active anti-retroviral therapy.

29. (canceled)

30. The method of claim 25(b), wherein the method is repeated at spaced intervals to assess progressive mitochondrial damage in the human subject.

31. The method of claim 30, wherein assessing progressive mitochondrial damage detects the onset or stage of a mitochondrial disorder.

32. (canceled)

33. The method of claim 23, wherein the biological system comprises a cell.

34. The method of claim 33, wherein the cell is contained within an organism or tissue sample.

35. (canceled)

36. (canceled)

37. The method claim 23, wherein the posttranslational modification comprises phosphorylation, oxidative damage, or carbonyl formation, which is not present in the control biological system.

38-42. (canceled)

43. The method of claim 23, wherein the biological system is a human subject and the method assesses mitochondrial damage in the human subject.

44. The method of claim 43, wherein the method is repeated at spaced intervals to assess progressive mitochondrial damage in the human subject.

45. The method of claim 44, wherein assessing progressive mitochondrial damage detects the onset or stage of a mitochondrial disorder.

46. A method for detecting a deficiency of an OXPHOS enzyme complex in a subject, comprising:
(a) contacting a biological sample from a subject with a plurality of monoclonal antibodies, each of which is specific for a subunit of an OXPHOS enzyme complex, wherein the plurality of monoclonal antibodies form a plurality of immunocomplexes, each immunocomplex comprising a monoclonal antibody and a specifically bound OXPHOS subunit;
(b) detecting the amount of specifically bound OXPHOS subunit for each of the plurality of monoclonal antibodies; and
(c) comparing the amount of each specifically bound OXPHOS subunit with an amount of the same OXPHOS subunit in a corresponding control sample of the OXPHOS enzyme complex, wherein a decrease in the amount of any OXPHOS subunit(s) of the OXPHOS enzyme complex in the subject sample as compared to the control sample indicates the presence of a deficiency of the OXPHOS enzyme complex in the subject.

47. The method of claim 46, wherein the OXPHOS enzyme complex is Complex I, Complex II, Complex, III, Complex IV, or Complex V.

48. (canceled)

49. The method of claim 13, wherein the OXPHOS enzyme complex is Complex I and the antibody is:
(a) a monoclonal antibody that specifically binds to at least one subunit of Complex I;
(b) RAC#24-20D1AB7, RAC#24-18G12BC2AA10, RAC#24-17C8E4E11, RAC#24-17G3D9E12, RAC#29-1D4, RAC#29-4G6BB9, RAC#29-6E1BH7, RAC#24A-20E9DH10C12, or a combination of any two or more thereof; or
(c) both (a) and (b).

50. (canceled)

51. (canceled)

52. The method of claim 47, wherein the OXPHOS enzyme complex is Complex I and the plurality of antibodies:
(a) is a combination of at least two monoclonal antibodies that specifically bind to the 30 kDa, 20 kDa, 15 kDa, or 8 kDa subunits of Complex I; or
(b) comprises RAC#24-20D1AB7, RAC#24-18G12BC2AA10, RAC#24-17C8E4E11, RAC#24-17G3D9E12, RAC#29-1D4, RAC#29-4G6BB9, RAC#29-6E1BH7, RAC#24A-20E9DH10C12 or a combination of any two or more thereof.

53. (canceled)

54. The method of claim 51, further comprises determining a failure of the Complex I subunits to assemble to form a fully assembled Complex I, thereby determining that the deficiency comprises a failure in Complex I assembly.

55. A method for diagnosing late onset mitochondrial disorder in a subject, comprising:
(a) contacting an antibody specific for Complex I with a biological sample, wherein Complex I present in the biological sample and the antibody form an immunocomplex, comprising immunocaptured Complex I;
(b) separating the immunocaptured Complex I from components of the biological sample that are not substantially bound by the antibody; and
(c) detecting the presence of a posttranslational modification in one or more subunits of the immunocaptured Complex I, wherein the presence of a posttranslational modification indicates that the subject has late onset mitochondrial disorder.

56. The method of claim 55, wherein the late onset mitochondrial disorder is late onset diabetes, Huntington's disease, Parkinson's disease, Alzheimer's diseases, amyotrophic lateral sclerosis, or schizophrenia.

57. The method of claim 55, wherein separating the immunocaptured Complex I comprises:
(a) releasing the immunocaptured Complex I from the immunocomplex; and
(b) separating the Complex I subunits from one another by weight difference.

58. The method of claim 57, wherein detecting the presence of a posttranslational modification comprises detecting a difference in an immunocaptured Complex I subunit molecular weight as compared to a control Complex I subunit molecular weight.

59. (canceled)

60. The method of claim 13, wherein the OXPHOS enzyme complex is Complex II and the antibody is:
(a) a monoclonal antibody that specifically binds to at least one subunit of Complex II;
(b) is RAC#23C-4H12BG12AG2; or
(c) both (a) and (b).

61. (canceled)

62. (canceled)

63. The method of claim 13, wherein the OXPHOS enzyme complex is Complex III and the antibody is:
(a) a monoclonal antibody that specifically binds to at least one subunit of Complex III;
(b) RAC#23B-1A11BC12AB9; RAC#23C-4H12BC11BC5; RAC#23B-10D2; RAC#23C-11A51H12; RAC#23C-12G8: RAC#23C-17A81A8; RAC#23C-29C2, or a combination of any two or more thereof; or
(c) both (a) and (b).

64. (canceled)

65. (canceled)

66. The method of claim 46, wherein the OXPHOS enzyme complex is Complex III and the plurality of antibodies comprises RAC#23B-1A11BC12AB9; RAC#23C-4H12BC11BC5; RAC#23B-10D2; RAC#23C-11A51H12; RAC#23C-12G8; RAC#23C-17A81A8; RAC#23C-29C2, or a combination of any two or more thereof.

67. (canceled)

68. The method of claim 13, wherein the OXPHOS enzyme complex is Complex IV and the antibody is:
(a) a monoclonal antibody that specifically binds to at least one subunit of Complex IV;
(b) RAC#11B-7E5BA4; RAC#23C-21H10; RAC#23C-22D5; RAC#23C-22H11G43E1; RAC#23C-28G7; RAC#23C-31E91B82G9, or a combination of any two or more thereof, or
(c) both (a) and (b).

69. (canceled)

70. (canceled)

71. The method of claim 46, wherein the OXPHOS enzyme complex is Complex IV and the plurality of antibodies:
(a) is a combination of at least two monoclonal antibodies that specifically bind to the core 1, core 2, I, II, III, IV, Vb, Va, VIaH, VIb, Vic, VIIaH, VIIb, VIIc or VIII subunit of Complex IV;
(b) comprises RAC#11B-7E5BA4; RAC#23C-21H10; RAC#23C-22D5; RAC#23C-22H11G43E1; RAC#23C-28G7; RAC#23C-31E91B82G9, or a combination of any two or more thereof; or
(c) both (a) and (b).

72. (canceled)

73. (canceled)

74. The method of claim 13, wherein the OXPHOS enzyme complex is Complex V and the antibody:
(a) is a monoclonal antibody that specifically binds to at least one subunit of Complex V;
(b) is MM#1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9, or a combination of any two or more thereof; or
(c) both (a) and (b).

75. (canceled)

76. (canceled)

77. The method of claim 46, wherein the OXPHOS enzyme complex is Complex V and the plurality of antibodies:
(a) is a combination of at least two monoclonal antibodies that specifically bind to the α, β, d, OSCP, or IF₁ subunit of Complex V;
(b) antibodies comprises MM#1-12F4AD8AF8, MM#7-3D5AB1, MM#1-7H10BD4F9, RAC#23C-1G1, RAC#23C-24C9, MM#1-8E12, RAC#25A-5E2D7, RAC#29-2A5, RAC#29-6G5, RAC#29-8C7CC4, RAC#29-9G3, RAC#29-10A3, RAC#29-10C6AC9, or a combination of any two or more thereof; or
(c) both (a) and (b).

78. (canceled)

79. An immunoassay device for determining presence and/or amount of an OXPHOS enzyme complex in a sample, the device comprising:
a sample contact area; and
a respiratory enzyme capture area comprising an immobilized antibody having a binding affinity for an OXPHOS enzyme complex;
wherein a sample applied in the sample contact area flows in a direction of flow from the sample contact area to the respiratory enzyme capture area, and formation of a complex between the immobilized antibody and an OXPHOS enzyme complex is detectable to determine the presence and/or amount of the OXPHOS enzyme complex in the sample,
wherein the OXPHOS enzyme complex is Complex I, Complex II, Complex III, Complex IV, Complex V, or a combination of any two or more thereof.

80. (canceled)

81. The device of claim 79, wherein the immobilized antibody comprises the monoclonal antibody or antigen-binding fragment of claim 2, or any combination of two or more thereof.

82. An immunoassay device comprising a solid support comprising a plurality of discrete capture areas, each discrete capture area containing an immobilized monoclonal antibody specific for an OXPHOS enzyme complex.

83. The device of claim 82, wherein the solid support is a microtitre plate.

84. The device of claim 82, wherein the immobilized monoclonal antibodies are selected from the monoclonal antibodies and antigen-binding fragments of claim 2, or any combination of two or more thereof.

85. A kit comprising the device of claim 79 or the device of claim 82.

86. The kit of claim 85, further comprising a standard curve showing a correlation of the activity of the OXPHOS enzyme complex with expression level of the respiratory enzyme in subjects having normal activity of the OXPHOS enzyme complex.

87. (canceled)
